(19) United States
(12) Patent Application Publication
(10) Pub. No.: US 2022/0378889 A1
(43) Pub. Date: Dec. 1, 2022

(54) NEO-EPITOPE VACCINES AND METHODS OF TREATING CANCER

(71) Applicant: Epivax Therapeutics Inc., Providence, RI (US)

(72) Inventors: William D. Martin, Providence, RI (US); Anne De Groot, Providence, RI (US); Gad Berdugo, New York, NY (US); Guilhem Richard, Providence, RI (US); Dominique Bridon, Providence, RI (US); Leonard Moise, Providence, RI (US); Michael F. Princiotta, Providence, RI (US)

(21) Appl. No.: 17/727,261

(22) Filed: Apr. 22, 2022

Related U.S. Application Data
(63) Continuation-in-part of application No. PCT/US2020/061014, filed on Nov. 18, 2020.
(60) Provisional application No. 62/936,654, filed on Nov. 18, 2019.
(60) Provisional application No. 62/959,439, filed on Jan. 10, 2020.
(60) Provisional application No. 62/982,172, filed on Feb. 27, 2020.

(51) Int. Cl. A61K 39/00; A61P 35/00; A61P 1/00; G01N 33/569

(52) U.S. Cl. CPC A61K 39/001102; A61P 35/00; A61P 1/00; G01N 33/56977

(57) ABSTRACT

The invention relates to improved strategies, compositions, and methods for producing neoplasia vaccines and for their use in methods of treating cancer in a patient. In aspects, a method of treating cancer comprises: (a) administering an effective amount of one or more of the instantly-disclosed peptides or polypeptides comprising one or more identified shared neo-epitopes (including peptides or polypeptide comprising one or more peptides or polypeptides from Table A, B, and/or C and/or fragments and variants thereof); and subsequently (b) administering an effective amount of one or more of the instantly-disclosed subject-specific peptides or polypeptides comprising one or more identified subject-specific neo-epitopes. The peptides or polypeptides administered in step (a) and in step (b) are designed to exclude neo-epitopes that are known or determined (e.g. predicted) to engage regulatory T cells and/or other detrimental T cells (including T cells with potential host cross-reactivity and/or anergic T cells).

### RELATED APPLICATIONS AND INCORPORATION BY REFERENCE

This application is a continuation-in-part of International Application No. PCT/US2020/061014 filed Nov. 18, 2020, and published as International Publication No. WO 2021/101965 on May 27, 2021 and which claims priority to U.S. provisional application Ser. No. 62/936,654, filed Nov. 18, 2019, U.S. provisional application Ser. No. 62/959,439, filed Jan. 10, 2020, and U.S. provisional application Ser. No. 62/982,172, filed Feb. 27, 2020, each incorporated by reference herein in its entirety.

Reference is made to international application PCT/US2020/061009, filed Nov. 18, 2020, U.S. provisional application Ser. No. 62/959,440, filed Jan. 10, 2020, and U.S. provisional application Ser. No. 62/982,173, filed Feb. 27, 2020, each incorporated by reference in its entirety. Reference is made to international application Serial No. PCT/US2020/020089, filed Feb. 27, 2020 and to U.S. provisional application Ser. No. 62/811,207, filed Feb. 27, 2019, each incorporated by reference in its entirety. Reference is made to international application Serial No. PCT/US2020/031357, filed May 5, 2020 and to U.S. provisional application Ser. No. 62/842,800, filed May 3, 2019, U.S. provisional application Ser. No. 62/880,965, filed Jul. 31, 2019, U.S. provisional application Ser. No. 62/933,651, filed Nov. 8, 2019, and U.S. provisional application Ser. No. 62/932,654, filed May 3, 2019, each incorporated by reference in its entirety.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted in ASCII format via EFS-Web and is hereby incorporated by reference in its entirety. Said ASCII copy, created on Nov. 18, 2020, is named Y8652-99007.txt and is 62,319 bytes in size.

### FIELD OF THE INVENTION

Embodiments of the present invention relate to improved strategies, compositions, and methods for producing neoplasia vaccines and for their use in methods of treating cancer in a patient. More particularly, embodiments of the present invention relate to methods for treating cancer in a subject, the method comprising: (a) administering an effective amount of one or more of the instantly-disclosed peptides or polypeptides comprising one or more identified shared neo-epitopes (including peptides or polypeptide comprising, consisting of, or consisting essentially of one or more peptides or polypeptides from Table A, B, and/or C, and/or fragments and variants thereof, as disclosed herein); and subsequently (b) administering an effective amount of one or more of the instantly-disclosed subject-specific peptides or polypeptides comprising one or more identified subject-specific neo-epitopes. Importantly, both the peptides or polypeptides comprising one or more identified shared neo-epitopes administered in step (a) and the subject-specific peptides or polypeptides comprising one or more identified subject-specific neo-epitopes administered in step (b) are designed to exclude neo-epitopes that are known or determined (e.g. predicted) to engage regulatory T cells and/or other detrimental T cells (including T cells with potential host cross-reactivity and/or anergic T cells). The identified and designed shared neo-epitopes can be utilized in “off the shelf” pre-furnished shared neo-epitope warehouses, which can be used to enable the rapid production of cancer neoantigen-based vaccines to a broad population of cancer patients (e.g., but not limited to, bladder cancer patients). The use of the shared neo-epitope warehouse together with the subject-specific peptides or polypeptides comprising one or more identified subject-specific neo-epitopes allows for the optimal utilization of the neoplasia-specific neoantigens in a patient.

### BACKGROUND OF THE INVENTION

There are many existing cancer therapies, including ablation techniques (e.g., surgical procedures, cryogenic/heat treatment, ultrasound, radiofrequency, and radiation) and chemical techniques (e.g., pharmaceutical agents, cytotoxic/chemotherapeutic agents, monoclonal antibodies, and various combinations thereof). However, these therapies are frequently associated with serious risk, toxic side effects, and extremely high costs, as well as uncertain efficacy.

More recently, clinical studies have highlighted the potential of precision cancer immunotherapy to effectively control the cancer of patients by harnessing a patient's own immune system. Such precision cancer immunotherapies include the identification and use of a patient-specific pool of neoplasia-specific neoantigens in a personalized vaccine. However, such vaccines require sequencing of an individual patients' genomes (including both the genome of cancer cells and the genome of non-cancer cells) and the production of personalized compositions that include a combination of neoantigens that have been identified as included in the individual patient. Thus, vaccines containing such patient-specific, neoplasia-specific neoantigens can take a significant amount of time and effort to design and administer.

Further, while several different methodologies for preparing neoplasia vaccines have been employed, recent studies showcase the difficulty of establishing robust CD8+ and CD4+ effector T cell responses to effectively treat the targeted neoplasia. This difficulty may be due to the inadvertent inclusion of suppressive T cell neo-epitopes in neoantigen-based vaccines that may be recognized by, and thus activate, regulatory T cells, which may abrogate effective immune responses against tumor cells. Further, T cells that recognize antigen-derived epitopes sharing TCR contacts with epitopes derived from self may be deleted or rendered anergic during thymic selection before they can be released to the periphery. As such, vaccine components targeting these T cells may be ineffective. On the other hand, vaccine-induced immune response targeting cross-reactive epitopes may induce unwanted autoimmune responses targeting the homologues of the cross-reactive epitopes identified by our homology search. As a result, vaccine safety may be reduced. Thus, the inadvertent inclusion of other detrimental T cell-neo-epitopes in neoantigen-based vaccines that may be recognized by, and thus activate, other detrimental T cells (including T cells with potential host cross-reactivity that may lead to autoimmune responses, as well as anergic T cells) may also lead to ineffective immune responses against tumor cells.

Immune tolerance is regulated by a complex interplay between antigen presenting cells (APC), T cells, B cells, cytokines, chemokines, and surface receptors. Initial self/non-self discrimination occurs in the thymus during neonatal development where medullary epithelial cells express specific self protein epitopes to immature T cells. T cells recognizing self antigens with high affinity are deleted, but autoreactive T cells with moderate affinity sometimes avoid deletion and can be converted to so called ‘natural’ regulatory T cells. These natural regulatory T cells are exported to the periphery and help to control latent autoimmune response.

A second form of tolerance develops in the periphery. In this case activated T cells are converted to an ‘adaptive’ regulatory T cells phenotype through the action of certain immune suppressive cytokines and chemokines such as IL-10, TGF-β and CCL19. The possible roles for these ‘adaptive’ regulatory T cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria.

Naturally occurring regulatory T cells (including both natural regulatory T cells and adaptive regulatory T cells) are a critical component of immune regulation in the periphery. For example, upon activation of natural regulatory T cells through their TCR, natural regulatory T cells express immune modulating cytokines and chemokines. Activated natural regulatory T cells may suppress nearby effector T cells through contact dependent and independent mechanisms. In addition, the cytokines released by these cells including, but not limited to, IL-10 and TGF-β, are capable of inducing antigen-specific adaptive regulatory T cells. However, although regulatory T cells activity is essential for prevention of autoimmunity, excessive regulatory T cells function may abrogate effective immune responses against tumor cells (Nishikawa et al., “Regulatory T Cells in Tumor Immunity,” Int. J. Cancer 127:759-767 (2010)). Indeed, down-regulation of regulatory T cell activity has been used as an effective tool to improve anticancer therapies (Grauer et al., “Elimination of Regulatory T Cells is Essential for an Effective Vaccination with Tumor Lysate-Pulsed Dendritic Cells in a Murine Glioma Model,” Int. J. Cancer 122:1794-1802 (2008); Zhou et al., “Depletion of Endogenous Tumor-Associated Regulatory T Cells Improves the Efficacy of Adoptive Cytotoxic T-Cell Immunotherapy in Murine Acute Myeloid Leukemia,” Blood 114:3793-3802 (2009)). Thus, inadvertent inclusion of suppressive T cell neo-epitopes in neoplasia vaccines, including neoantigen-based vaccines, that may be recognized by, and thus activate, regulatory T cells, must be avoided to prevent the abrogation of an effective immune response against tumor cells.

As such, there is an ongoing need for improved strategies, compositions, and methods for the treatment of cancer and for producing neoantigen-based vaccines. More particularly, there remains an ongoing need for strategies, compositions, and methods for treating cancer in a subject that utilize both a first vaccine that includes shared neo-epitopes and a subsequent second vaccine (e.g., administered about 3 weeks after the first vaccine) that includes subject-specific neo-epitopes, with both the shared neo-epitopes and subject-specific neo-epitopes excluding neo-epitopes that are known or determined (e.g. predicted) to engage regulatory T cells and/or other detrimental T cells (including T cells with potential host cross-reactivity and/or anergic T cells) in the subject. In aspects, the first vaccine will be produce from an “off the shelf” pre-furnished shared neo-epitope warehouse.

### SUMMARY OF THE INVENTION

Accordingly, embodiments of the present invention provide improved strategies, compositions, and methods for producing neoplasia vaccines and for their use in methods of treating a neoplasia (e.g., a cancer) in a subject. More particularly, embodiments of the present invention relate to a methods for treating neoplasia in a subject, the method comprising: (a) administering an effective amount of one or more of the instantly-disclosed peptides or polypeptides comprising one or more identified shared neo-epitopes (including peptides or polypeptide comprising, consisting, or consisting essentially of an amino acid sequence selected from the group consisting of the polypeptides of Table A (SEQ ID NOS: 105-163), Table B (SEQ ID NOS: 164-350), and/or Table C (and/or fragments or variants thereof), and optionally 1 to 12 additional amino acids distributed in any ratio on the N terminus and/or C-terminus of the polypeptide of Table A, Table B, and/or Table C); and subsequently (b) administering an effective amount of one or more of the instantly-disclosed subject-specific peptides or polypeptides comprising one or more identified subject-specific neo-epitopes. In aspects, the peptides or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells). The identified and designed shared neo-epitopes can be utilized in “off the shelf” pre-furnished shared neo-epitope warehouses, which can be used to enable the rapid production of cancer neoantigen-based vaccines to a broad population of cancer patients (e.g., but not limited to, bladder cancer patients). The use of the shared neo-epitope warehouse together with the subject-specific peptides or polypeptides comprising one or more identified subject-specific neo-epitopes allows for the optimal utilization of the neoplasia-specific neoantigens in a patient.

One embodiment is directed to a method of treating a neoplasia in a subject, the method comprising (a) administering an effective amount of one or more peptides or polypeptides comprising one or more identified shared neo-epitopes (including peptides or polypeptide comprising, consisting, or consisting essentially of an amino acid sequence selected from the group consisting of the polypeptides of Table A (SEQ ID NOS: 105-163), Table B (SEQ ID NOS: 164-350), and/or Table C (and/or fragments or variants thereof), and optionally 1 to 12 additional amino acids distributed in any ratio on the N terminus and/or C-terminus of the polypeptide of Table A, Table B, and/or Table C); and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells). One embodiment is directed to a method of inducing an immune response in a subject, the method comprising (a) administering an effective amount of one or more peptides or polypeptides comprising one or more identified shared neo-epitopes (including peptides or polypeptide comprising, consisting, or consisting essentially of an amino acid sequence selected from the group consisting of the polypeptides of Table A (SEQ ID NOS: 105-163), Table B (SEQ ID NOS: 164-350), and/or Table C (and/or fragments or variants thereof); and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells).

In aspects of the above-described methods, the one or more peptides or polypeptides comprising one or more identified shared neo-epitopes comprise, consist of, or consist essentially of one or more peptides or polypeptides from Table A (SEQ ID NOS: 105-163), Table B (SEQ ID NOS: 164-350), and/or Table C (and/or fragments and variants thereof), and optionally 1 to 12 additional amino acids distributed in any ratio on the N terminus and/or C-terminus of the polypeptide of Table A, Table B, and/or Table C. In aspects of the above-described methods, the one or more peptides or polypeptides each have a core amino acid sequence comprising, consisting of, or consisting essentially of one or more peptides or polypeptides from Table A, B, and/or C (and/or fragments and variants thereof), optionally with extensions of 1 to 12 amino acids on the C-terminal and/or the N-terminal of the core sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects of the above-described methods, the one or more peptides or polypeptides each have a core sequence comprising, consisting of, or consisting essentially of one or more peptides or polypeptides from Table A, B, and/or C (and/or fragments and variants thereof), optionally with extensions of 1 to 12 amino acids on the C-terminal and/or the N-terminal, wherein the overall number of these flanking amino acids is 1 to 12, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MEW binding propensity) as said polypeptide core sequence without said flanking amino acids. In aspects, said polypeptide with the flanking amino acids is still able to bind to the same HLA molecule (i.e., retain MEW binding propensity) and retain the same TCR specificity as said polypeptide core sequence without said flanking amino acids. In aspects, the one or more peptides or polypeptides have a core sequence comprising, consisting of, or consisting essentially of one or more peptides or polypeptides from Table A, B, and/or C (and/or fragments and variants thereof), optionally with extensions of 1 to 12 amino acids on the C-terminal and/or the N-terminal, provided: the one or more peptides or polypeptides are encoded by a shared neoplasia-specific mutation that is detected in a neoplasia sample from the subject; the one or more peptides or polypeptides are known or determined (e.g. predicted) to bind to a MHC protein of the subject; and/or the one or more peptides or polypeptides are known or determined (e.g. predicted) to not bind to a MEW protein of the subject that could lead to a detrimental or suppressive immune response. In aspects, said flanking amino acid sequences are those that also flank the peptides or polypeptides included therein in the naturally occurring protein. In aspects, said flanking amino acid sequences as described herein may serve as a MEW stabilizing region. The use of a longer peptide may allow endogenous processing by patient cells and may lead to more effective antigen presentation and induction of T cell responses. In aspects, the peptides or polypeptides can be capped with an N-terminal acetyl and C-terminal amino group. In aspects, the peptides or polypeptides can be either in neutral (uncharged) or salt forms, and may be either free of or include modifications such as glycosylation, side chain oxidation, or phosphorylation.

In aspects of the above-described methods, one or more peptides or polypeptides comprising, consisting of, or consisting essentially of one or more peptides or polypeptides from Table A, B, and/or C (and/or fragments and variants thereof) as described herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus) are not administered to a subject suspected of having or having a neoplasia provided: the one or more peptides or polypeptides are encoded by a shared neoplasia-specific mutation that is not detected in a neoplasia sample from the subject; the one or more peptides or polypeptides are known or determined to not bind to a MHC protein of the subject; and/or the one or more peptides or polypeptides are known or determined to bind to a MEW protein of the subject that could lead to a detrimental or suppressive immune response.

In aspects of the above-described methods, the method further comprises detecting one or more tumor-specific mutations in a neoplasia sample from a subject and/or determining HLA allotypes present in the subject, and administering one or more of peptides or polypeptides comprising, consisting of, or consisting essentially of one or more peptides or polypeptides from Table A, B, and/or C (and/or fragments and variants thereof) as described herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus), provided: the one or more peptides or polypeptides are encoded by a shared neoplasia-specific mutation that is detected in a neoplasia sample from the subject; the one or more peptides or polypeptides are known or determined (e.g. predicted) to bind to a MEW protein of the subject; and/or the one or more peptides or polypeptides are known or determined (e.g. predicted) to not bind to a MEW protein of the subject that could lead to a detrimental or suppressive immune response. In aspects, one or more peptides or polypeptides comprising, consisting of, or consisting essentially of one or more peptides or polypeptides from Table A, B, and/or C (and/or fragments and variants thereof) as described herein are not administered to a subject suspected of having or having a neoplasia provided: the one or more peptides or polypeptides are encoded by a shared neoplasia-specific mutation that is not detected in a neoplasia sample from the subject; the one or more peptides or polypeptides are known or determined to not bind to a MEW protein of the subject; and/or the one or more peptides or polypeptides are known or determined to bind to a MHC protein of the subject that could lead to a detrimental or suppressive immune response.

In aspects of the above-described methods, the one or more peptides or polypeptides comprising one or more identified shared neo-epitopes (including peptides or polypeptide comprising, consisting of, or consisting essentially of one or more peptides or polypeptides from Table A, B, and/or C (and/or fragments and variants thereof) as described herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus) are administered within 1 week of detecting one or more tumor-specific mutations in the neoplasia sample from a subject (e.g., tumor tissue, such as bladder cancer tumor tissue) and/or determining HLA allotypes present in the subject.

In aspects of the above-described methods, the one or more identified shared neo-epitopes are identified by a method comprising: i) assessing identified shared neoplasia-specific mutations to identify known or determined (e.g. predicted) shared neo-epitopes encoded by said shared neoplasia-specific mutations; and ii) assessing the identified shared neo-epitopes encoded by said mutations from step (i) to identify neo-epitopes that are known or determined (e.g. predicted) to engage regulatory T cells and/or other detrimental T cells (including T cells with potential host cross-reactivity and/or anergic T cells), and excluding such identified neo-epitopes that are known or determined (e.g. predicted) to engage regulatory T cells and/or other detrimental T cells (including T cells with potential host cross-reactivity and/or anergic T cells) from the shared neo-epitopes.

In aspects of the above-described methods, the shared neoplasia-specific mutations are neoplasia-specific somatic mutations. In aspects, the neoplasia-specific mutations are single nucleotide variations (SNVs), insertions and deletions (which can generate both in-frame and frameshift mutations), and other large-scale rearrangements such as but not limited to chromosomal inversions, duplications, insertions, deletions, or translocations. In aspects, neoplasia specific mutations, including SNVs, insertions, and deletions, are non-synonymous mutations. In aspects, neoplasia-specific mutations, including SNVs, insertions and deletions (which can be non-synonymous mutations), and other large-scale rearrangements, are mutations of proteins encoded in the neoplasia specimen of the subject diagnosed as having a neoplasia. In aspects, shared neoplasia specific mutations, including SNVs, are non-synonymous mutations. In aspects, shared neoplasia-specific mutations, including SNVs (which can be non-synonymous mutations), indels, and frameshifts, are shared mutations of proteins encoded in the neoplasia specimen of a subject diagnosed as having a neoplasia.

In aspects of the above-described methods, assessing the shared neoplasia-specific mutations in step (i) to identify known or determined (e.g. predicted) shared neo-epitopes encoded by said shared neoplasia-specific mutations includes: a) determining a binding score for a mutated peptide to one or more MHC molecules, wherein said mutated peptide is encoded by at least one of said shared neoplasia-specific mutations; b) determining a binding score for a non-mutated peptide to the one or more MHC molecules, wherein the non-mutated peptide is identical to the mutated peptide except for the encoded at least one of said shared neoplasia-specific mutations; c) determining the percentile rank of the binding scores of both the mutated peptide of step (a) and the non-mutated peptide of step (b) as compared to an expected distribution of binding scores for a sufficiently large enough group randomly generated peptides (e.g., at least 10,000) using naturally observed amino acid frequencies; d) determining the TCR facing amino acid residues of said mutated peptide and said non-mutated peptide; and e) identifying the mutated peptide as a shared neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide and the non-mutated peptide. In further aspects, the one or more MHC molecules are MHC class I molecules and/or MHC class II molecules. In aspects, the mutated peptide and non-mutated peptide are both 9 amino acids in length or the mutated peptide and non-mutated peptide are both 10 amino acids in length. In aspects, the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal. In aspects, the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal. In aspects, the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal. In further aspects, TCR contacts for a 9-mer identified neo-epitope that binds to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) as counted from the amino terminal. In aspects, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal. In further aspects, the TCR contacts for a 9-mer identified neo-epitope that binds to a MHC class I molecule are at any combination of residues at positions 1, 3, 4, 5, 6, 7, and 8 as counted from the amino terminal. In aspects, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal. In further aspects, the TCR contacts for a 10-mer identified neo-epitope that binds to a MHC class I molecule are at any combination of residues at positions 1, 3, 4, 5, 6, 7, 8, and 9 as counted from the amino terminal.

In aspects of the above-described methods, assessing the shared neoplasia-specific mutations in step (i) to identify known or determined (e.g. predicted) shared neo-epitopes encoded by said shared neoplasia-specific mutations comprises in silico testing. In further aspects, said in silico testing to identify known or determined (e.g. predicted) shared neo-epitopes encoded by said mutations in step (i) comprises using the EPIMATRIX® algorithm.

In aspects of the above-described methods, assessing the identified shared neo-epitopes encoded by said shared neoplasia-specific mutations to identify neo-epitopes that are known or determined (e.g. predicted) to engage regulatory T cells and/or other detrimental T cells (including T cells with potential host cross-reactivity and/or anergic T cells) in step (ii) comprises determining whether said identified shared neo-epitopes encoded by said mutations share TCR contacts with proteins derived from either the human proteome or the human microbiome, wherein said identified shared neo-epitopes encoded by said mutations that are determined to share TCR contacts with proteins derived from either the human proteome or the human microbiome are identified as neo-epitopes that are known or determined (e.g. predicted) to engage regulatory T cells and/or other detrimental T cells (including T cells with potential host cross-reactivity and/or anergic T cells). In aspects, TCR contacts for a 9-mer identified shared neo-epitope that bind to a MHC class II molecule are at position 2, 3, 5, 7, and 8 of the identified shared neo-epitope as counted from the amino terminal, wherein the TCR contacts for a 9-mer identified shared neo-epitope that binds to a MHC class I molecule are at position 4, 5, 6, 7, and 8 of the identified shared neo-epitope as counted from the amino terminal, and wherein the TCR contacts for a 10-mer identified shared neo-epitope that bind to a MHC class I molecule are at position 4, 5, 6, 7, 8, and 9 of the identified shared neo-epitope as counted from the amino terminal. In further aspects, TCR contacts for a 9-mer identified neo-epitope that binds to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) as counted from the amino terminal. In aspects, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal. In further aspects, the TCR contacts for a 9-mer identified neo-epitope that binds to a MHC class I molecule are at any combination of residues at positions 1, 3, 4, 5, 6, 7, and 8 as counted from the amino terminal. In aspects, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal. In further aspects, the TCR contacts for a 10-mer identified neo-epitope that binds to a MHC class I molecule are at any combination of residues at positions 1, 3, 4, 5, 6, 7, 8, and 9 as counted from the amino terminal.

In aspects of the above-described methods, assessing the identified shared neo-epitopes encoded by said shared neoplasia-specific mutations to identify neo-epitopes that are known or determined (e.g. predicted) to engage regulatory T cells and/or other detrimental T cells (including T cells with potential host cross-reactivity and/or anergic T cells) in step (iii) comprises in silico testing. In aspects, in silico testing comprises analyzing whether the identified shared neo-epitopes are predicted to engage regulatory T cells and/or other detrimental T cells (including T cells with potential host cross-reactivity and/or anergic T cells) using an algorithm that predicts cross-reactivity with regulatory T cells and other detrimental T cells. In certain aspects, the algorithm is the JANUSMATRIX™ algorithm. In further aspects an identified shared neo-epitope is predicted to engage regulatory T cells and/or other detrimental T cells (including T cells with potential host cross-reactivity and/or anergic T cells) if the cross-reactivity score is greater than a predetermined cutoff, for example that evaluates potential neo-epitopes compared to self antigens and identifies or distinguishes those cross-reactive neo-epitopes that are measured or calculated to bind well to a subject's MHC and/or be frequent in a subject's proteome. In certain aspects, the JANUSMATRIX™ score for the shared neo-epitope is greater than or equal to 2 (and in further aspects, greater than or equal to 3). In aspects, the method further comprises determining whether the identified shared neo-epitopes engage regulatory T cells and/or other detrimental T cells (including T cells with potential host cross-reactivity and/or anergic T cells) in vitro. In aspects, a shared neo-epitope is determined to engage regulatory T cells when said shared neo-epitope results in regulatory T cell activation, proliferation, and/or IL-10 or TGF-β production.

In aspects of the above-described methods, assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) in step (ii) comprises determining whether the identified shared neo-epitopes engage regulatory T cells and/or other detrimental T cells (including T cells with potential host cross-reactivity and/or anergic T cells) cells in vitro. In aspects, a shared neo-epitope is determined to engage regulatory T cells when said shared neo-epitope results in regulatory T cell activation, proliferation, and/or IL-10 or TGF-β production.

In aspects of the above-described methods, the method further includes: iii) designing at least one peptide or polypeptide, said peptide or polypeptide comprising at least one identified shared neo-epitope encoded by said shared neoplasia-specific mutations, provided said shared neo-epitope is not identified in step (ii) as being known or determined to engage regulatory T cells and/or other detrimental T cells (including T cells with potential host cross-reactivity and/or anergic T cells). In aspects, the method further includes iv) providing the at least one peptide or polypeptide designed in step (iii) or a nucleic acid encoding said peptides or polypeptides. In even further aspects, the method further includes v) providing a vaccine comprising the at least one peptide or polypeptide or nucleic acid provided in step (iv).

In aspects of the above-described methods, the one or more peptides or polypeptides comprising one or more identified shared neo-epitopes are administered with a pharmaceutically acceptable adjuvant and/or carrier. In aspects, the adjuvant comprises poly-ICLC.

In aspects of the above-described methods, one or more peptides or polypeptides comprising one or more identified shared neo-epitopes are administered in step (a) provided: the one or more peptides or polypeptides are encoded by a shared neoplasia-specific mutation that is detected in a neoplasia sample from the subject; the one or more peptides or polypeptides are known or determined (e.g. predicted) to bind to a MEW protein of the subject; and/or the one or more peptides or polypeptides are known or determined (e.g. predicted) to not bind to a MEW protein of the subject that could lead to a detrimental or suppressive immune response. In aspects of the above-described methods, one or more peptides or polypeptides comprising one or more identified shared neo-epitopes are not administered in step (a) provided: the one or more peptides or polypeptides are encoded by a shared neoplasia-specific mutation that is not detected in a neoplasia sample from the subject; the one or more peptides or polypeptides are known or determined to not bind to a MHC protein of the subject; and/or the one or more peptides or polypeptides are known or determined to bind to a MEW protein of the subject that could lead to a detrimental or suppressive immune response.

In aspects of the above-described methods, the method further comprises detecting one or more tumor-specific mutations in a neoplasia sample from a subject and/or determining HLA allotypes present in the subject, and administering one or more peptides or polypeptides comprising one or more identified shared neo-epitopes, provided: the one or more peptides or polypeptides are encoded by a shared neoplasia-specific mutation that is detected in a neoplasia sample from the subject; the one or more peptides or polypeptides are known or determined (e.g. predicted) to bind to a MEW protein of the subject; and/or the one or more peptides or polypeptides are known or determined (e.g. predicted) to not bind to a MEW protein of the subject that could lead to a detrimental or suppressive immune response. In aspects, one or more peptides or polypeptides comprising one or more identified shared neo-epitopes are not administered to a subject suspected of having or having a neoplasia provided: the one or more peptides or polypeptides are encoded by a shared neoplasia-specific mutation that is not detected in a neoplasia sample from the subject; the one or more peptides or polypeptides are known or determined to not bind to a MEW protein of the subject; and/or the one or more peptides or polypeptides are known or determined to bind to a MEW protein of the subject that could lead to a detrimental or suppressive immune response.

In aspects of the above-described methods, the one or more peptides or polypeptides comprising one or more identified shared neo-epitopes are administered within 1 week of detecting one or more tumor-specific mutations in the neoplasia sample from a subject and/or determining HLA allotypes present in the subject.

In aspects of the above-described methods, the one or more of subject-specific peptides or polypeptides comprising one or more identified subject-specific neo-epitopes are identified by a method comprising: i) identifying neoplasia-specific mutations in a neoplasia specimen of a subject; ii) assessing the neoplasia-specific mutations identified in step (i) to identify known or determined neo-epitopes encoded by said mutations, wherein said neo-epitopes are known or determined (e.g. predicted) to bind to a MEW protein of the subject; and iii) assessing the identified neo-epitopes encoded by said mutations from step (ii) to identify neo-epitopes that are known or determined to engage regulatory T cells and/or other detrimental T cells (including T cells with potential host cross-reactivity and/or anergic T cells), and excluding such identified neo-epitopes that are known or determined to engage regulatory T cells and/or other detrimental T cells (including T cells with potential host cross-reactivity and/or anergic T cells) from the subject-specific neo-epitopes.

In aspects of the above-described methods, identifying neoplasia-specific mutations in step (i) includes identifying sequence differences between the full or partial genome, exome, and/or transcriptome of a neoplasia specimen from the subject diagnosed as having a neoplasia and a non-neoplasia specimen. In aspects, a non-neoplasia specimen is derived from the subject diagnosed as having a neoplasia. In further aspects, identifying neoplasia-specific mutations or identifying sequence differences comprises Next Generation Sequencing (NGS). In aspects, identifying neoplasia-specific mutations in step (i) comprises selecting from the neoplasia a plurality of nucleic acid sequences, each comprising mutations not present in a non-neoplasia sample. In aspects, identifying neoplasia-specific mutations or identifying sequence differences comprises sequencing genomic DNA and/or RNA of the neoplasia specimen.

In aspects of the above-described methods, the neoplasia-specific mutations are neoplasia-specific somatic mutations. In aspects, the neoplasia-specific mutations are single nucleotide variations (SNVs), insertions and deletions (which can generate both in-frame and frameshift mutations), and other large-scale rearrangements such as but not limited to chromosomal inversions, duplications, insertions, deletions, or translocations. In aspects, neoplasia specific mutations, including SNVs, insertions, and deletions, are non-synonymous mutations. In aspects, neoplasia-specific mutations, including SNVs, insertions and deletions (which can be non-synonymous mutations), and other large-scale rearrangements, are mutations of proteins encoded in the neoplasia specimen of the subject diagnosed as having a neoplasia. In aspects, neoplasia specific mutations, including SNVs, are non-synonymous mutations. In aspects, neoplasia-specific mutations, including SNVs (which can be non-synonymous mutations), indels, and frameshifts, are mutations of proteins encoded in the neoplasia specimen of the subject diagnosed as having a neoplasia

In aspects of the above-described methods, assessing the neoplasia-specific mutations in step (ii) to identify known or determined (e.g. predicted) neo-epitopes encoded by said mutations includes: a) determining a binding score for a mutated peptide to one or more MHC molecules, wherein said mutated peptide is encoded by at least one of said neoplasia-specific mutations; b) determining a binding score for a non-mutated peptide to the one or more MHC molecules, wherein the non-mutated peptide is identical to the mutated peptide except for the encoded at least one of said neoplasia-specific mutations; c) determining the percentile rank of the binding scores of both the mutated peptide of step (a) and the non-mutated peptide of step (b) as compared to an expected distribution of binding scores for a sufficiently large enough group randomly generated peptides (e.g., at least 10,000) using naturally observed amino acid frequencies; d) determining the TCR facing amino acid residues of said mutated peptide and said non-mutated peptide; and e) identifying the mutated peptide as a neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide and the non-mutated peptide. In further aspects, the one or more MHC molecules are MHC class I molecules and/or MHC class II molecules. In aspects, the mutated peptide and non-mutated peptide are both 9 amino acids in length or the mutated peptide and non-mutated peptide are both 10 amino acids in length. In aspects, the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal. In further aspects, TCR contacts for a 9-mer identified neo-epitope that binds to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) as counted from the amino terminal. In aspects, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal. In further aspects, the TCR contacts for a 9-mer identified neo-epitope that binds to a MHC class I molecule are at any combination of residues at positions 1, 3, 4, 5, 6, 7, and 8 as counted from the amino terminal. In aspects, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal. In further aspects, the TCR contacts for a 10-mer identified neo-epitope that binds to a MHC class I molecule are at any combination of residues at positions 1, 3, 4, 5, 6, 7, 8, and 9 as counted from the amino terminal.

In aspects of the above-described methods, assessing the neoplasia-specific mutations in step (ii) to identify known or determined (e.g. predicted) neo-epitopes encoded by said mutations comprises in silico testing. In further aspects, said in silico testing to identify known or determined (e.g. predicted) neo-epitopes encoded by said mutations in step (ii) comprises using the EPIMATRIX® algorithm.

In aspects of the above-described methods, assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) in step (iii) comprises determining whether said identified neo-epitopes encoded by said mutations share TCR contacts with proteins derived from either the human proteome or the human microbiome, wherein said identified neo-epitopes encoded by said mutations that are determined to share TCR contacts with proteins derived from either the human proteome or the human microbiome are identified as neo-epitopes that are known or determined (e.g. predicted) to engage regulatory T cells and/or other detrimental T cells (including T cells with potential host cross-reactivity and/or anergic T cells). In aspects, TCR contacts for a 9-mer identified neo-epitope that bind to a MHC class II molecule are at position 2, 3, 5, 7, and 8 of the identified neo-epitope as counted from the amino terminal, wherein the TCR contacts for a 9-mer identified neo-epitope that binds to a MHC class I molecule are at position 4, 5, 6, 7, and 8 of the identified neo-epitope as counted from the amino terminal, and wherein the TCR contacts for a 10-mer identified neo-epitope that bind to a MHC class I molecule are at position 4, 5, 6, 7, 8, and 9 of the identified neo-epitope as counted from the amino terminal. In further aspects, TCR contacts for a 9-mer identified neo-epitope that binds to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) as counted from the amino terminal. In aspects, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal. In further aspects, the TCR contacts for a 9-mer identified neo-epitope that binds to a MHC class I molecule are at any combination of residues at positions 1, 3, 4, 5, 6, 7, and 8 as counted from the amino terminal. In aspects, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal. In further aspects, the TCR contacts for a 10-mer identified neo-epitope that binds to a MHC class I molecule are at any combination of residues at positions 1, 3, 4, 5, 6, 7, 8, and 9 as counted from the amino terminal.

In aspects of the above-described methods, assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) in step (iii) comprises in silico testing. In aspects, in silico testing comprises analyzing whether the identified neo-epitopes are predicted to engage regulatory T cells and/or other detrimental T cells (including T cells with potential host cross-reactivity and/or anergic T cells) using the JANUSMATRIX™ algorithm. In further aspects an identified neo-epitope is predicted to engage regulatory T cells and/or other detrimental T cells (including T cells with potential host cross-reactivity and/or anergic T cells) if the JANUSMATRIX™ score for the neo-epitope is greater than or equal to 2 (and in further aspects, greater than or equal to 3). In aspects, the method further comprises determining whether the identified neo-epitopes engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production.

In aspects of the above-described methods, assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) in step (iii) comprises determining whether the identified neo-epitopes engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production.

In aspects of the above-described methods, the method further includes: iv) designing at least one subject-specific peptide or polypeptide, said peptide or polypeptide comprising at least one identified neo-epitope encoded by said mutations, provided said neo-epitope is not identified in step (iii) as being known or determined (e.g. predicted) to engage regulatory T cells and/or other detrimental T cells (including T cells with potential host cross-reactivity and/or anergic T cells). In aspects, the method further includes: v) providing the at least one peptide or polypeptide designed in step (iv) or a nucleic acid encoding said peptides or polypeptides. In even further aspects, the method further includes: vi) providing a vaccine comprising the at least one peptide or polypeptide or nucleic acid provided in step (v).

In aspects, the one or more subject-specific peptides or polypeptides comprising one or more identified subject-specific neo-epitopes are administered with a pharmaceutically acceptable adjuvant and/or carrier. In aspects, the adjuvant comprises poly-ICLC.

In aspects of the above-described methods, the one or more subject-specific peptides or polypeptides comprising one or more identified subject-specific neo-epitopes are administered roughly three weeks are administering an effective amount of one or more peptides or polypeptides comprising one or more identified shared neo-epitopes.

In aspects of the above-described methods, the one or more peptides or polypeptides comprising one or more identified shared neo-epitopes comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypetides comprising one or more identified shared neo-epitopes. In aspects, the one or more peptides or polypeptides comprising one or more identified shared neo-epitopes comprises from 3-20 selected peptides or polypeptides comprising one or more identified shared neo-epitopes. In aspects, each peptide or polypeptide comprising one or more identified shared neo-epitopes has a overall length of from 8-100 amino acids. In aspects, each peptide or polypeptide of the plurality of selected peptides or polypeptides comprising one or more identified shared neo-epitopes has an overall length of from 8-40 amino acids, from 8-30 amino acids, from 8-25 amino acids, from 8-23 amino acids, from 8-20 amino acids, or from 8-15 amino acids.

In aspects of the above-described methods, the one or more peptides or polypeptides comprising one or more identified shared neo-epitopes comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides as disclosed herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus). In aspects of the above-described methods, the one or more peptides or polypeptides comprising one or more identified shared neo-epitopes comprise from 3-20 peptides or polypeptides comprising, consisting, or consisting essentially of an amino acid sequence selected from the group consisting of the polypeptides of Table A (SEQ ID NOS: 105-163), Table B (SEQ ID NOS: 164-350), and/or Table C (and/or fragments or variants thereof) as disclosed herein. In aspects of the above-described methods, each peptide or polypeptide comprising, consisting, or consisting essentially of an amino acid sequence selected from the group consisting of the polypeptides of Table A (SEQ ID NOS: 105-163), Table B (SEQ ID NOS: 164-350), and/or Table C (and/or fragments or variants thereof) as disclosed herein has an overall length of from 8-100 amino acids. In aspects, each peptide or polypeptide as disclosed herein has an overall length of from 8-40 amino acids, from 8-30 amino acids, from 8-25 amino acids, from 8-23 amino acids, from 8-20 amino acids, or from 8-15 amino acids.

In aspects of the above-described methods, the the one or more subject-specific peptides or polypeptides comprising one or more identified subject-specific neo-epitopes comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypetides comprising one or more identified subject-specific neo-epitopes as disclosed herein. In aspects, the one or more peptides or polypeptides comprising one or more identified subject-specific neo-epitopes comprises from 3-20 selected peptides or polypeptides comprising one or more identified subject-specific neo-epitopes. In aspects, each peptide or polypeptide comprising one or more identified subject-specific neo-epitopes has an overall length of from 8-100 amino acids. In aspects, each peptide or polypeptide of the plurality of selected peptides or polypeptides comprising one or more identified subject-specific neo-epitopes has an overall length of from 8-40 amino acids, from 8-30 amino acids, from 8-25 amino acids, from 8-23 amino acids, from 8-20 amino acids, or from 8-15 amino acids.

In aspects of the above-described methods, the method further incudes administration of an anti-immunosuppressive agent. In aspects, the anti-immunosuppressive agent comprises a checkpoint blockage modulator, such as a checkpoint blockage inhibitor and immune checkpoint stimulators.

In aspects of the above-described methods, the neoplasia is a solid tumor. In aspects, the neoplasia is bladder cancer, breast cancer, brain cancer, colon cancer, gastric cancer, head and neck cancer, kidney cancer, liver cancer, lung cancer, melanoma, ovarian cancer, pancreatic cancer, prostate cancer, or testicular cancer. In aspects, the neoplasia is bladder cancer.

In embodiments, the invention provides compositions and kits for use in the above-described methods. In embodiments, the invention provides a composition comprising an effective amount of one or more peptides or polypeptides comprising one or more identified shared neo-epitopes, wherein the one or more identified shared neo-epitopes exclude neo-epitopes that are known or determined (e.g. predicted) to engage regulatory T cells and/or other detrimental T cells (including T cells with potential host cross-reactivity and/or anergic T cells), for use in treating a neoplasia in a subject. In embodiments, the invention provides a composition comprising an effective amount of one or more subject-specific peptides or polypeptides comprising one or more identified subject-specific neo-epitopes, wherein the one or more identified subject-specific peptides or polypeptides comprising one or more identified subject-specific neo-epitopes exclude neo-epitopes that are known or determined (e.g. predicted) to engage regulatory T cells and/or other detrimental T cells (including T cells with potential host cross-reactivity and/or anergic T cells), for use in treating a neoplasia in a subject.

In embodiments, the invention provides a composition comprising an effective amount of one or more peptides or polypeptides comprising one or more identified shared neo-epitopes, and a composition comprising an effective amount of one or more subject-specific peptides or polypeptides comprising one or more identified subject-specific neo-epitopes, wherein the peptide or polypeptides comprising one or more identified shared neo-epitopes and the subject-specific peptides or polypeptides comprising one or more identified subject-specific neo-epitopes exclude neo-epitopes that are known or determined (e.g. predicted) to engage regulatory T cells and/or other detrimental T cells (including T cells with potential host cross-reactivity and/or anergic T cells), for use in treating a neoplasia in a subject.

In embodiments, the invention provides kit and compositions that embrace each of the aspects and features set out above.

These and additional embodiments and features of the presently-disclosed subject matter will be clarified by reference to the figures and detailed description set forth herein.

It is understood that both the preceding summary and the following detailed description are exemplary and are intended to provide further explanation of the invention as claimed. Neither the summary nor the description that follows is intended to define or limit the scope of the invention to the particular features mentioned in the summary or description.

### BRIEF DESCRIPTION OF THE DRAWINGS

FIG. 1 is schematic depiction of points assigned to neoantigen candidates in the presently-disclosed ranking systems based on their MEW class I neo-epitope content.

FIG. 2 is schematic depiction of points assigned to neoantigen candidates in the presently-disclosed ranking systems based on the minimal percentile rank of a MHC class I neo-epitope.

FIG. 3 is schematic depiction of points assigned to neoantigen candidates in the presently-disclosed ranking systems based on their MHC class II neo-epitope content.

FIG. 4 is schematic depiction of points assigned to neoantigen candidates in the presently-disclosed ranking systems based on the minimal percentile rank of a MHC class II neo-epitope.

FIG. 5 is schematic depiction of points assigned to neoantigen candidates in the presently-disclosed ranking systems based on their MHC class I Homology Score.

FIG. 6 is a schematic depiction of points assigned to neoantigen candidates in the presently-disclosed ranking systems based on their MHC class II Homology Score.

FIG. 7 is a schematic depiction of points assigned to neoantigen candidates in the presently-disclosed ranking systems based on the expression percentile rank of their originating transcript.

FIG. 8 is a schematic depiction of points assigned to neoantigen candidates in the presently-disclosed ranking systems based on their mutation coverage in the tumor DNA.

FIGS. 9A-9B is a schematic depiction of points assigned to neoantigen candidates in the presently-disclosed ranking systems derived from the mutanome of syngeneic models (FIG. 9A) or patients (FIG. 9B) based on the variant allele frequency (VAF) of the mutation in the tumor DNA.

FIGS. 10A-10B are graphs depicting CT26 tumor growth in PBS control (FIG. 10A) and poly-ICLC (FIG. 10B) groups. Individual mice are shown in lighter shading. Darker shading represents average tumor growth+/−SEM. The average is plotted until half the mice reach endpoint.

FIGS. 11A-11B is a graph depicting CT26 tumor growth in PBS control (FIG. 11A) and ANCER™-peptides (FIG. 11B) groups. Individual mice are shown in lighter shading. Darker shading represents average tumor growth+/−SEM. The average is plotted until half the mice reach endpoint.

FIG. 12 is a graph depicting mean (+/−SEM) CT26 tumor growth in PBS control, anti-poly-ICLC, and ANCER™-peptides groups. Means are plotted until half the mice reach endpoint.

FIGS. 13A-13B are graphs depicting CT26 neoantigen IFNγ response.

FIGS. 14A-14B are graphs depicting CT26 Treg peptides suppress IFNγ responses to CT26 neoantigen peptides.

FIGS. 15A-15B are graphs depicting that T cells display increased polyfunctionality with ANCER™-CT26 vaccine formulation.

FIG. 16 depicts the study design for the large multi-parameters CT26 efficacy experiment (prophetic).

FIG. 17 depicts the expected relative efficacy of each study arm disclosed in FIG. 16.

FIG. 18 depicts the efficacy of an ANCER™ selected peptide vaccine administered with poly-ICLC to control tumor growth in the syngeneic CT26 murine tumor model.

FIG. 19A-19D depicts private vs. shared mutations in bladder cancer. (A) Thirty-nine mutations were found to be present in at least 1% of patients. Neoantigens were designed with ANCER™ around 39 frequently shared mutations. (B) Percentage of TCGA BLCA patients that could receive each ANCER™-designed shared neoantigen based on their mutanome and HLA haplotypes. (C) Frequency of the non-shared mutations encoded by each shared neoantigen among TCGA BLCA patients. (D) Global MEW class I and class II immunogenicity scores (sum of neo-epitope Z-scores) of each ANCER™-designed shared neoantigen. Patient-specific immunogenicity will depend on HLA haplotype.

FIG. 20A-20B depicts neoantigen BLCA patient cumulative coverage by panel of shared antigens. (A) A panel of 10 shared neoantigens, whose sequences were refined by ANCER™, covers ˜25% of the TCGA BLCA population. Expanding this panel to 20 sequences increases the TCGA BLCA coverage up to a third (33%) of patients. (B) Simulating coverage of shared neoantigen panels in small cohorts of patients. In 50 simulated cohorts of 30 patients, a panel of 10 shared neoantigens covered a median of 9 (30%) patients [range=3 to 18]. Expanding this panel to 20 sequences in the simulation increased the coverage to a median of 12 (40%) patients [range=4 to 21].

### DETAILED DESCRIPTION OF THE INVENTION

Particular details of various embodiments of the invention are set forth to illustrate certain aspects and not to limit the scope of the invention. It will be apparent to one of ordinary skill in the art that modifications and variations are possible without departing from the scope of the embodiments defined in the appended claims. More specifically, although some aspects of embodiments of the present invention may be identified herein as preferred or particularly advantageous, it is contemplated that the embodiments of the present invention are not limited to these preferred aspects.

Unless defined otherwise, all technical and scientific terms used herein have the same meaning as commonly understood by one of ordinary skill in the art to which the presently-disclosed subject matter belongs.

Clinical studies have highlighted the potential of personalized cancer immunotherapy to effectively control the cancer of patients across cancer indications. Such precision cancer immunotherapies include the identification and use of a patient-specific pool of neoplasia-specific neoantigens in a personalized vaccine. However, such personalized vaccines require sequencing of an individual patients' genomes (including both the genome of cancer cells and the genome of non-cancer cells) and the production of personalized compositions that comprise a combination of identified neoantigens that are included in the individual patient. Accordingly, such patient-specific, neoplasia-specific neoantigens can take a significant amount of time and effort to design and administer. Further, while several different methodologies for preparing neoplasia vaccines have been employed, recent studies showcase the difficulty of establishing robust CD8+ and CD4+ effector T cell responses to effectively treat the targeted neoplasia. This difficulty may be due to the inadvertent inclusion of suppressive T cell neo-epitopes in neoantigen-based vaccines that may be recognized by, and thus activate, regulatory T cells, which may abrogate effective immune responses against tumor cells, and/or the inadvertent inclusion of other detrimental T cell-neo-epitopes in neoantigen-based vaccines that may be recognized by, and thus activate, other detrimental T cells (including T cells with potential host cross-reactivity that may lead to autoimmune responses, as well as anergic T cells). As such, there remains a need for improved compositions and methods related to neoantigen-based vaccines.

Embodiments of the present invention relate to improved strategies, compositions, and methods for producing shared neoplasia vaccines and overcome these difficulties. The instantly-disclosed strategies, compositions, and methods for treating cancer in a subject utilize both a first vaccine that includes shared neo-epitopes and a subsequent second vaccine (e.g., administered about 3 weeks after the first vaccine) that includes subject-specific neo-epitopes, with both the shared neo-epitopes and subject-specific neo-epitopes excluding neo-epitopes that are known or determined (e.g. predicted) to engage regulatory T cells and/or other detrimental T cells (including T cells with potential host cross-reactivity and/or anergic T cells) in the subject. In aspects, the first vaccine will be produce from an “off the shelf” pre-furnished shared neo-epitope warehouse, which can be used to enable the rapid production of cancer neoantigen-based vaccines to a broad population of cancer patients (e.g., but not limited to, bladder cancer patients). In fact, in aspects, a panel of ten of the instantly-disclosed neo-epitopes covers roughly 25% of the bladder cancer population; a panel of twenty of the instantly-disclosed neo-epitopes covers roughly 33% of the bladder cancer population; and a panel of thirty-nine of the instantly-disclosed neo-epitopes covers roughly 40% of the bladder cancer population. Subsequently, a vaccine comprising subject-specific neo-eptiopes can be designed using the instantly-disclosed strategies and methods and administered to the subject. The combined use of the shared neo-epitope vaccine together with a subsequently administered subject-specific neo-epitope vaccine allows for the optimal utilization of the neoplasia-specific neoantigens in a patient.

Further distinctive features of the instantly-disclosed strategies, compositions, and methods for producing neoplasia vaccines over other vaccine pipelines are the ability to predict both CD4+ and CD8+ T cell shared and subject-specific neo-epitopes and to identify, and subsequently remove, neo-epitopes that may be recognized by and activate regulatory T cells and/or other detrimental T cells (including T cells with potential host cross-reactivity and/or anergic T cells). For example, we used the ANCER™ system to identify, design, and select both shared neo-epitopes (e.g., based on 39 identified “highly frequent” non-synonymous mutations in bladder cancer patients) and subject-specific neo-epitopes based on non-synonymous mutations. The instantly-disclosed identified, designed, and selected shared and subject-specific neo-epitopes are restricted to a wide array of HLA class I and class II alleles. ANCER™, a proprietary platform for the identification, characterization, and triaging of tumor-specific neo-epitopes, leverages EPIMATRIX® (for the identification of determined (e.g. predicted) neo-epitopes encoded by said neoplasia-specific mutations for use in the both shared and subject-specific neoplasia vaccines) and JANUSMATRIX™ (for the identification of neo-epitopes that are determined (e.g. predicted) to engage regulatory T cells and/or other detrimental T cells (including T cells with potential host cross-reactivity and/or anergic T cells), and exclusion of such identified neo-epitopes that are predicted to engage regulatory T cells and/or other detrimental T cells (including T cells with potential host cross-reactivity and/or anergic T cells) from the neo-epitopes for use in the shared and subject-specific neoplasia vaccines), state-of-the-art predictive algorithms that have been extensively validated in prospective vaccine studies for infectious diseases (Moise et al., Hum. Vaccines Immunother 2015, 11(9):2312; Wada et al., Sci. Rep. 2017, 7(1)1283). Distinctive features of ANCER™ over other in silico pipelines are its ability to accurately predict CD4+ T cell epitopes and to identify tolerated or Treg epitopes and/or epitopes that can engage other detrimental T cells including T cells with potential host cross-reactivity and/or anergic T cells). Screening of neoantigen sequences, including shared and subject-specific neoantigen sequences, to identify and remove potential regulatory T cell inducing neo-epitopes and/or potential detrimental T cell inducing neo-epitopes offers the possibility of enriching and designing new vaccines with higher quality candidates while minimizing costs and turnaround times.

Accordingly, the instant invention is directed to improved strategies, compositions, and methods for producing shared and subject-specific neoplasia vaccines. More particularly, embodiments of the present invention relate to improved strategies, compositions, and methods for producing neoplasia vaccines and for their use in methods of treating a neoplasia (e.g., a cancer) in a subject. More particularly, embodiments of the present invention relate to a methods for treating neoplasia in a subject, the method comprising: (a) administering an effective amount of one or more of the instantly-disclosed peptides or polypeptides comprising one or more identified shared neo-epitopes (including, e.g., one or more peptides or polypeptides having a core sequence comprising, consisting of, or consisting essentially of one or more peptides or polypeptides of Table A (SEQ ID NOS: 105-163), Table B (SEQ ID NOS: 164-350) and/or Table C (and/or fragments or variants thereof), and optionally 1 to 12 additional amino acids distributed in any ratio on the N terminus and/or C-terminus of the polypeptide of Table A, Table B, and/or Table C); and subsequently (b) administering an effective amount of one or more of the instantly-disclosed subject-specific peptides or polypeptides comprising one or more identified subject-specific neo-epitopes. In aspects, the peptides or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells), as subsequently discussed in more detail.

### Definitions

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items.

As used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “antigen” refers to any substance that will elicit an immune response. In aspects, an antigen relates to any substance, preferably a peptide or protein, that reacts specifically with antibodies or T-lymphocytes (T cells). According to the present invention, the term “antigen” comprises any molecule which comprises at least one epitope. Preferably, an antigen is a molecule which, optionally after processing, induces an immune reaction, which is preferably specific for the antigen (including cells expressing the antigen). An antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens, e.g., a part of a tumor cell such as a protein or peptide expressed in a tumor cell which may be derived from the cytoplasm, the cell surface or the cell nucleus, in particular those which primarily occur intracellularly or as surface antigens of tumor cells.

As used herein, the term “biological sample” as refers to any sample of tissue, cells, or secretions from an organism.

As used herein, the terms “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, the term “control” is meant a standard or reference condition.

As used herein, the term “disease” is meant any condition or disorder that damages or interferes with the normal function of a cell, tissue, or organ.

As used herein, the term “effective amount” is meant the amount required to ameliorate the symptoms of a disease (e.g., a neoplasia/tumor) relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, “fragment” refers to a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

As used herein, the term “immune response” refers to the concerted action of lymphocytes, antigen presenting cells, phagocytic cells, granulocytes, and/or soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

As used herein, the term “immune synapse” means the protein complex formed by the simultaneous engagement of a given T cell epitope to both a cell surface MEW complex and TCR.

As used herein, the term “isolated” means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the invention.

As used herein, the terms “the major histocompatibility complex (MHC)”, “MHC molecules”, “MEW proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MEW class II. The molecules of the two MHC classes are specialized for different antigen sources. The molecules of MHC class I present endogenously synthesized antigens, for example viral proteins and tumor antigens. The molecules of MEW class II present protein antigens originating from exogenous sources, for example bacterial products. The cellular biology and the expression patterns of the two MEW classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MEW molecules of class I is preferably an HLA-A, HLA-B or HLA-C monomer, and the light chain is β-2-microglobulin. MEW molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC Binding Motif” refers to a pattern of amino acids in a protein sequence that predicts binding to a particular MEW allele.

As used herein, the term “MHC Ligand” means a polypeptide capable of binding to one or more specific WIC alleles. The term “HLA ligand” is interchangeable with the term “MHC Ligand”. Cells expressing MHC/Ligand complexes on their surface are referred to as “Antigen Presenting Cells” (APCs). Similarly, as used herein, the term “MHC binding peptide” relates to a peptide which binds to an MHC class I and/or an MEW class II molecule. In the case of MHC class I/peptide complexes, the binding peptides are typically 8-10 amino acids long although longer or shorter peptides may be effective. In the case of MEW class II/peptide complexes, the binding peptides are typically 10-25 amino acids long and are in particular 13-18 amino acids long, whereas longer and shorter peptides may also be effective.

As used herein, the term “epitope” refers to an antigenic determinant in a molecule such as an antigen, i.e., to a part in or fragment of the molecule that is recognized by the immune system, for example, that is recognized by a T cell, in particular when presented in the context of MEW molecules. An epitope of a protein such as a tumor antigen preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 8, 9, 10, 11, 12, 13, 14, 15, 16, 17, 18, 19, 20, 21, 22, 23, 24, or 25 amino acids in length. It is particularly preferred that the epitope in the context of the present invention is a T cell epitope.

As used herein, the term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide of the present invention, however, can be joined to, linked to, or inserted into another polypeptide (e.g., a heterologous polypeptide) with which it is not normally associated in a cell and still be “isolated” or “purified.” When a polypeptide is recombinantly produced, it can also be substantially free of culture medium, for example, culture medium represents less than about 20%, less than about 10%, or less than about 5% of the volume of the polypeptide preparation.

As used herein the term “neo-epitope” refers to a T cell epitope that is not present in a reference such as a normal non-cancerous or germline cell but is found in cancer cells. This includes, in particular, situations wherein in a normal non-cancerous or germline cell a corresponding epitope is found, however, due to one or more mutations in a cancer cell the sequence of the epitope is changed so as to result in the neo-epitope. This also includes situations wherein in a normal non-cancerous or germline cell no T cell epitope is found, however, due to one or more mutations in a cancer cell the sequence is changed so as to create a new neo-epitope. In aspects, a “neo-epitope” of the present invention may be encoded by a neoplasia-specific mutation that is unique to the neoplasia patient/subject (e.g., epitope that is specific to both the cancer cell and subject from which it is found), and my be referred to herein as a “subject-specific neo-epitope.” In aspects, a “neo-epitope” of the present invention may be encoded by a neoplasia-specific mutation that is present in a neoplasia (e.g., cancer) cell in at least 1%, 2%, 3%, 4%, 5%, or more than 5% of subjects in a population of subjects suffering from the neoplasia (e.g., bladder cancer), and may be referred to herein as a “shared neo-epitope.” In aspects, a “shared neo-epitope” may be present in two or more, three or more, four or more, five or more, etc. subjects in a population of subjects suffering from the neoplasia (e.g., bladder cancer).

As used herein, the terms “neoantigen” or “neo-antigenic” means a class of tumor antigens that arises from a neoplasia-specific mutation(s) which alters the amino acid sequence of genome encoded proteins. “Neoantigens” can include one or more neo-epitopes, including subject-specific or shared neo-epitopes. A “subject-specific neo-epitope” means a neoplasia-specific mutation that is unique to the neoplasia patient/subject (e.g., a mutation that is specific to both the cancer cell and subject from which it is found). A “shared neoplasia-specific mutation” means a neoplasia-specific mutation that is present in a neoplasia (e.g., cancer) cell in at least 1%, 2%, 3%, 4%, 5%, or more than 5% of subjects in a population of subjects suffering from the neoplasia, e.g., the specific type of neoplasia, such as bladder cancer. In aspects, a “shared neoplasia-specific mutation” means a neoplasia-specific mutation that is present in a neoplasia (e.g., cancer) cell in two or more, three or more, four or more, five or more, etc. subjects in a population of subjects suffering from the neoplasia, e.g., the specific type of neoplasia, such as bladder cancer.

As used herein, the term “neoplasia” refers to any disease that is caused by or results in the abnormal proliferation of cells, inappropriately low levels of apoptosis, or both. Neoplasia can be benign, pre-malignant, or malignant. Cancer is an example of a neoplasia. Non-limiting examples of cancer include leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier or diluent” or the like refer to an excipient, carrier or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, the term “regulatory T cell epitope” (“Tregitope”) refers to a “T cell epitope” that causes a tolerogenic response (Weber C A et al., (2009), Adv Drug Deliv, 61(11):965-76) and is capable of binding to MHC molecules and engaging (i.e. interacting with and activating) circulating naturally occurring Tregs (in aspects, including natural Tregs and/or adaptive Tregs). In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, the term “T cell epitope” means an MHC ligand or protein determinant, 7 to 30 amino acids in length, and capable of specific binding to MHC molecules (e.g. human leukocyte antigen (HLA) molecules) and interacting with specific T cell receptors (TCRs). As used herein, in the context of a T cell epitope (e.g., a neo-epitope, Tregitope, etc.) that is known or determined (e.g. predicted) to engage a T cell (e.g., regulatory T cells and/or other detrimental T cells, such as T cells with potential host cross-reactivity and/or anergic T cells), the terms “engage”, “engagement” or the like means that when bound to a MHC molecule (e.g. human leukocyte antigen (HLA) molecules), the T cell epitope is capable of interacting with the TCR of the T cell and activating the T cell (which in the case of an anergic T cell, includes functional inactivation). Generally, T cell epitopes are linear and do not express specific three-dimensional characteristics. T cell epitopes are not affected by the presence of denaturing solvents. The ability to interact with T cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety.

As used herein, the term “T Cell Receptor” or “TCR” refers to a protein complex expressed by T cells that is capable of engaging a specific repertoire of MHC/Ligand complexes as presented on the surface of APCs.

As used herein, the term “vaccine” refers to a pharmaceutical preparation (pharmaceutical composition) or product that upon administration induces an immune response, in particular a cellular immune response, which recognizes and attacks a pathogen or a diseased cell such as a neoplasia (e.g., a cancer cell). A vaccine may be used for the prevention or treatment of a disease. Accordingly, vaccines are medicaments which include antigens and are used in humans or animals for generating specific defense and protective substance by vaccination. The term “personalized neoplasia vaccine” or the like concerns a particular neoplasia patient and means that a neoplasia (e.g. cancer) vaccine is adapted to the needs or special circumstances of an individual neoplasia patient. The term “shared neoplasia vaccine” or the like means that a neoplasia (e.g. cancer) vaccine is adapted to the needs of a population of neoplasia patients.

Methods of Treating a Neoplasia and/or Inducing an Immune Response in a Subject

One embodiment is directed to a method of treating a neoplasia in a subject, the method comprising (a) administering an effective amount of one or more peptides or polypeptides comprising one or more identified shared neo-epitopes; and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells). One embodiment is directed to a method of inducing an immune response in a subject, the method comprising (a) administering an effective amount of one or more peptides or polypeptides comprising one or more identified shared neo-epitopes; and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells). It will be appreciated that the administration to a subject of an effective amount of the presently-disclosed peptides or polypeptides comprising one or more identified shared neo-epitopes or pharmaceutical compositions comprising such in step (a) and an effective amount of the presently-disclosed peptides or polypeptides comprising one or more identified subject-specific neo-epitopes or pharmaceutical compositions comprising such in step (b) can provide therapy for a wide variety of cancers including, but not limited to solid tumors, such as lung, breast, colon, ovarian, brain, liver, pancreas, prostate, malignant melanoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS. In particular aspects, the cancer is bladder cancer

For example, in order to produce an improved neoantigen-based bladder cancer vaccine, we identified private and shared neoantigens from the mutanomes of bladder cancer patients. We determined that 99.02% of non-synonymous mutations (including, e.g., missense, indel, and frameshift mutations) encountered in the mutanomes of studied bladder cancer patients (n=411) are private neoantigens (with a total of 82,572 private neoantigens identified), while 0.98% are shared neoantigens (with a total of 820 shared neoantigens identified). Of the identified shared neoantigens, we determined that thirty-nine non-synonymous mutations (4.7%) are found in at 1% of bladder cancer patient mutanomes. There “highly frequent” mutations serve as potential targets for off-the-shelf vaccines, as long as they encode neo-epitopes.

Subsequently, we used the ANCER™ system to identify, design, and select shared neo-epitopes based on these 39 identified “highly frequent” non-synonymous mutations. These shared neo-epitopes include peptides or polypeptide comprising, consisting of, or consisting essentially of one or more peptides or polypeptides from Table A (SEQ ID NOS: 105-163), Table B (SEQ ID NOS: 164-350), and/or C and/or fragments and variants thereof. These instantly-disclosed identified, designed, and selected shared neo-epitopes are restricted to a wide array of HLA class I and class II alleles. ANCER™, a proprietary platform for the identification, characterization, and triaging of tumor-specific neo-epitopes, leverages EPIMATRIX® (for the identification of determined (e.g. predicted) neo-epitopes encoded by said neoplasia-specific mutations for use in the shared neoplasia vaccine) and JANUSMATRIX™ (for the identification of neo-epitopes that are determined (e.g. predicted) to engage regulatory T cells and/or other detrimental T cells (including T cells with potential host cross-reactivity and/or anergic T cells), and exclusion of such identified neo-epitopes that are predicted to engage regulatory T cells and/or other detrimental T cells (including T cells with potential host cross-reactivity and/or anergic T cells) from the shared neo-epitopes for use in the shared neoplasia vaccine), state-of-the-art predictive algorithms that have been extensively validated in prospective vaccine studies for infectious diseases (Moise et al., Hum. Vaccines Immunother 2015; Wada et al., Sci. Rep. 2017). Distinctive features of ANCER™ over other in silico pipelines are its ability to accurately predict CD4+ T cell epitopes and to identify tolerated or Treg epitopes and/or epitopes that can engage other detrimental T cells including T cells with potential host cross-reactivity and/or anergic T cells). Screening of neoantigen sequences, including shared and subject-specific neoantigen sequences, to identify and remove potential regulatory T cell inducing neo-epitopes and/or potential detrimental T cell inducing neo-epitopes offers the possibility of enriching and designing new vaccines with higher quality candidates while minimizing costs and turnaround times.

As such, in aspects of the above-described methods of treating a neoplasia and/or inducing an iImmune response in a subject, the one or more peptides or polypeptides comprising one or more identified shared neo-epitopes comprise, consist of, or consist essentially of one or more peptides or polypeptides from Table A (SEQ ID NOS: 105-163), Table B (SEQ ID NOS: 164-350), and/or C and/or fragments and variants thereof. In aspects, said at least one peptide or polypetide have a core sequence comprising, consisting of, or consisting essentially of one or more peptides or polypeptides from Table A, B, and/or C (and/or fragments and variants thereof), optionally with extensions of 1 to 12 amino acids on the C-terminal and/or the N-terminal, wherein the overall number of these flanking amino acids is 1 to 12, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, said at least one peptide or polypeptide have a core sequence comprising, consisting of, or consisting essentially of one or more peptides or polypeptides from Table A, B, and/or C (and/or fragments and variants thereof), optionally with extensions of 1 to 12 amino acids on the C-terminal and/or the N-terminal, wherein the overall number of these flanking amino acids is 1 to 12, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the peptide with the flanking amino acids is still able to bind to the same HLA molecule (i.e., retain MHC binding propensity) as said peptide without said flanking amino acids. In aspects, said polypeptide with the flanking amino acids is still able to bind to the same HLA molecule (i.e., retain MHC binding propensity) and retain the same TCR specificity as said polypeptide core sequence without said flanking amino acids. In aspects, said flanking amino acid sequences are those that also flank the peptides or polypeptides included therein in the naturally occurring protein. In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be capped with an N-terminal acetyl and C-terminal amino group. In aspects, the peptides or polypeptides can be either in neutral (uncharged) or salt forms, and may be either free of or include modifications such as glycosylation, side chain oxidation, or phosphorylation

In aspects, said at least one peptide or polypeptide as described herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus, said shared neo-epitopes included in the at least one peptide or polypeptide are encoded by a shared neoplasia-specific mutation detected in a neoplasia sample from the subject and/or wherein said shared neo-epitopes included in the at least one peptide or polypeptide are known or determined (e.g. predicted) to bind to a MHC protein of the subject. In aspects, such at least one designed peptide or polypeptide or a nucleic acid encoding said peptides or polypeptide can be provided in an “off the shelf” pre-furnished shared neo-epitope warehouse. Such designed peptides or polypeptides or nucleic acids encoding said designed peptides or polypeptides can be used to rapidly produce shared neoantigen-based vaccines for application to a broad population of cancer patients, including bladder cancer patients. In aspects of the above-described peptides or polypeptides, the peptides or polypeptides may be isolated, synthetic, or recombinant.

TABLE A
Bladder Cancer Shared Neoepitope Optimal Designed Peptides

| | SEQ ID | | | |
|---|---|---|---|---|
| SEQUENCE | NO. | MUTATION | HLA Restriction | HLA Exclusion |
| LDVLERCPHRPI | 105 | FGFR3_p.S249C | B*0702, B*4402, | |
| | | | B*4403, B*4901, | |
| | | | B*5001, B*5101 | |
| RDPLSEITKQEKDFL | 106 | PIK3CA_p.E545K | A*0101, A*0301, | |
| | | | B*4402, B*4403, | |
| | | | B*4901, B*5101 | |
| RDPLSEITKQEKDFLWS | 107 | PIK3CA_p.E545K | A*0101, A*0301, | A*2402, B*4402, |
| | | | A*3201, B*4403, | B*5701, B*5801 |
| | | | B*4901, B*5101, | |
| | | | DRB1*0301, | |
| | | | DRB1*1301 | |
| AISTRDPLSKITEQEKDF | 108 | PIK3CA_p.E542K | A*0101, A*0301, | |
| | | | A*1101, A*3201, | |
| | | | A*6801, B*0702, | |
| | | | B*5101, B*5701, | |
| | | | DRB1*0301 | |
| YLSTDVGFCTLVCPLH | 109 | ERBB2_p.S310F | A*0101, A*0201, | A*2301 |
| | | | A*2402, B*0702, | |
| | | | B*3501, B*4403, | |
| | | | B*4501, B*5101, | |
| | | | B*5701, B*5801 | |
| NYLSTDVGFCTLVCPLHNQE | 110 | ERBB2_p.S310F | A*0101, A*0201, | A*2301, DRB1*0901 |
| | | | A*2402, A*3201, | |
| | | | B*0702, B*3501, | |
| | | | B*4403, B*4501, | |
| | | | B*5101, B*5701, | |
| | | | B*5801, DRB1*0401, | |
| | | | DRB1*0701, | |
| | | | DRB1*0801, | |
| | | | DRB1*1101 | |
| SCMGGMNQRPILT | 111 | TP53_p.R248Q | A*1101, A*2301, | |
| | | | A*2402, A*3201, | |
| | | | A*6801, B*0702, | |
| | | | B*0801, B*2705 | |
| SCMGGMNQRPILTI | 112 | TP53_p.R248Q | A*1101, A*2301, | B*0801, B*5101 |
| | | | A*2402, A*3201, | |
| | | | A*6801, B*0702, | |
| | | | B*2705, B*4402 | |
| VRVCACPGTDRRTEEENL | 113 | TP53_p.R280T | A*0101, A*0301, | |
| | | | A*1101, A*6801, | |
| | | | B*0702, B*5001 | |
| LRLPALRFIGLKC | 114 | RXRA_p.S427F | A*0201, A*3201, | |
| | | | B*0702, B*0801, | |
| | | | B*2705, B*3501, | |
| | | | B*5101 | |
| LRLPALRFIGLKCLEHLF | 115 | RXRA_p.S427F | A*0201, B*0702, | A*2301, A*2402, |
| | | | B*0801, B*3501, | A*3201, B*1501, |
| | | | B*5101, DRB1*0401, | B*2705, B*5701, |
| | | | DRB1*0701, | DRB1*1101 |
| | | | DRB1*0801, | |
| | | | DRB1*1201, | |
| | | | DRB1*1501 | |
| GRDRRTKEENLR | 116 | TP53_p.E285K | B*0801, B*2705 | |
| SAAALPASERG | 117 | C3orf70_p.S6L | A*6801 | |
| MSAAALPASERG | 118 | C3orf70_p.S6L | A*6801 B*5801 | B*3501, DRB1*1501 |
| FDEAHSIDNVC | 119 | ERCC2_p.N238S | B*4403,B*4901 | |
| KAVVVFDEAHSIDNVC | 120 | ERCC2_p.N238S | A*0201, A*3201, | A*2301 |
| | | | B*4403, B*4901, | |
| | | | B*5701, B*5801, | |
| | | | DRB1*0101, | |
| | | | DRB1*0301, | |
| | | | DRB1*0401 | |
| KNGRPDYIIVTHRP | 121 | AHR_p.Q383H | A*2301, A*2402, | B*2705 |
| | | | B*0702, B*3501, | |
| | | | B*5101 | |
| KNGRPDYIIVTHRPLTDEE | 122 | AHR_p.Q383H | A*2301, A*2402, | B*2705, DRB1*0101, |
| | | | B*0702, B*0801, | DRB1*0701, |
| | | | B*1501, B*3501, | DRB1*0901, |
| | | | B*5101, DRB1*0301, | DRB1*1101, |
| | | | DRB1*0801, | DRB1*1301 |
| | | | DRB1*1501 | |
| ADEAGSVCAGILS | 123 | FGFR3_p.Y373C | B*4402, B*4403, | |
| | | | B*4501, B*5001, | |
| | | | B*5101 | |
| MGHVAAVGCVQYDG | 124 | FBXW7_p.R505G | A*0101, A*0301, | |
| | | | B*1501, B*3501, | |
| | | | B*5001, B*5101, | |
| | | | B*5701, B*5801 | |
| GVREVFKTATRAALQ | 125 | RHOB_p.E172K | A*2301, B*2705, | A*2402, B*0702, |
| | | | B*4402, B*4403, | B*3501, DRB1*0901, |
| | | | B*4501, B*4901, | DRB1*1101 |
| | | | B*5001, DRB1*0101, | |
| | | | DRB1*0301, | |
| | | | DRB1*0701, | |
| | | | DRB1*0801, | |
| | | | DRB1*1001, | |
| | | | DRB1*1201, | |
| | | | DRB1*1301, | |
| | | | DRB1*1501 | |
| LVVVGADGVGKSALT | 126 | KRAS_p.G12D | A*0301, A*1101, | |
| | | | B*4402, DRB1*0301, | |
| | | | DRB1*1001 | |
| YKLVVVGADGVGKSALT | 127 | KRAS_p.G12D | A*0201, A*0301, | DRB1*0701, |
| | | | A*1101, B*4402, | DRB1*0801, |
| | | | DRB1*0301, | DRB1*0901 |
| | | | DRB1*1001 | |
| KQMNDARHGGWTT | 128 | PIK3CA_p.H1047R | A*0101, A*2301, | |
| | | | A*2402, A*3201, | |
| | | | B*4402, B*4403, | |
| | | | B*5701, B*5801 | |
| EYFMKQMNDARHGGWTT | 129 | PIK3CA_p.H1047R | A*0101, A*2301, | B*5001, DRB1*0101, |
| | | | A*2402, A*3201, | DRB1*0401, |
| | | | A*6801, B*1501, | DRB1*1001 |
| | | | B*4402, B*4403, | |
| | | | B*5701, B*5801, | |
| | | | DRB1*0301, | |
| | | | DRB1*0801, | |
| | | | DRB1*1101, | |
| | | | DRB1*1201, | |
| | | | DRB1*1301 | |
| LLGRNSFKVRVCACPGR | 130 | TP53_p.E271K | A*0201, A*0301, | |
| | | | A*1101, B*0801, | |
| | | | B*2705, B*4501, | |
| | | | B*5801 | |
| SGNLLGRNSFKVRVCACPGR | 131 | TP53_p.E271K | A*0201, A*0301, | A*2402, B*0801, |
| | | | A*1101, A*6801, | B*2705 |
| | | | B*4501, B*5801, | |
| | | | DRB1*0101, | |
| | | | DRB1*0701, | |
| | | | DRB1*1001, | |
| | | | DRB1*1301, | |
| | | | DRB1*1501 | |
| DGILYAFQKQTTEDSV | 132 | SF3B1_p.E902K | A*0201, A*6801, | B*5101, DRB1*1501 |
| | | | B*0801, B*3501, | |
| | | | B*5701, B*5801, | |
| | | | DRB1*0101, | |
| | | | DRB1*0301, | |
| | | | DRB1*0401, | |
| | | | DRB1*0701, | |
| | | | DRB1*0801, | |
| | | | DRB1*0901, | |
| | | | DRB1*1001, | |
| | | | DRB1*1101, | |
| | | | DRB1*1301 | |
| DGILYAFQKQTTEDSVML | 133 | SF3B1_p.E902K | A*0201, A*6801, | B*0801, B*3501, |
| | | | B*1501, B*5701, | B*5101, DRB1*15O1 |
| | | | B*5801, DRB1*0101, | |
| | | | DRB1*0301, | |
| | | | DRB1*0401, | |
| | | | DRB1*0701, | |
| | | | DRB1*0801, | |
| | | | DRB1*0901, | |
| | | | DRB1*1001, | |
| | | | DRB1*1101, | |
| | | | DRB1*1301 | |
| MACGFCRAIACQ | 134 | RARS2_p.R6C | B*3501, B*4402, | |
| | | | B*4403, B*5101 | |
| MACGFCRAIACQLSR | 135 | RARS2_p.R6C | B*3501, B*4402, | A*0301, A*1101, |
| | | | B*4403, B*5101, | B*0801, B*2705, |
| | | | DRB1*0101, | B*5801 |
| | | | DRB1*0701, | |
| | | | DRB1*0901, | |
| | | | DRB1*1001, | |
| | | | DRB1*1501 | |
| KTLWKALAKKP | 136 | MROH2B_p.E1109K | A*3201, A*6801 | A*0301, A*1101 |
| LVEADEACSVYAG | 137 | FGFR3_p.G370C | A*0101, B*3501, | |
| | | | B*4402, B*4403, | |
| | | | B*4501, B*4901, | |
| | | | B*5001, B*5801 | |
| LVEADEACSVYAGIL | 138 | FGFR3_p.G370C | A*0101, B*3501, | B*5101 |
| | | | B*4402, B*4403, | |
| | | | B*4501, B*4901, | |
| | | | B*5001, B*5801 | |
| LGRNSFEVCVCACPG | 139 | TP53_p.R273C | A*0101, B*2705, | |
| | | | B*3501, B*4402, | |
| | | | B*4501, B*4901, | |
| | | | B*5001, B*5801 | |
| RVCACPGKDRRTEEENL | 140 | TP53_p.R280K | A*0301, A*1101, | |
| | | | A*6801, B*0702 | |
| NYMCNSFCMGGM | 141 | TP53_p.S241F | A*0101, A*0201, | |
| | | | A*2301, A*2402, | |
| | | | A*6801, B*0801, | |
| | | | B*1501, B*3501, | |
| | | | B*4402, B*4403, | |
| | | | B*5101 | |
| IHYNYMCNSFCMGGM | 142 | TP53_p.S241F | A*0101, A*0201, | A*2301, A*2402, |
| | | | A*3201, A*6801, | B*5101 |
| | | | B*0801, B*1501, | |
| | | | B*3501, B*4402, | |
| | | | B*4403, B*4901, | |
| | | | DRB1*0101, | |
| | | | DRB1*0401, | |
| | | | DRB1*0701, | |
| | | | DRB1*0801, | |
| | | | DRB1*0901, | |
| | | | DRB1*1001, | |
| | | | DRB1*1101, | |
| | | | DRB1*1501 | |
| TGIVMDSGDRVTHTVPI | 143 | ACTB_p.G158R | A*0101, A*0201, | |
| | | | B*0702, B*5801, | |
| | | | DRB1*0101 | |
| RQDIDLGVSGEVFDFS | 144 | NFE2L2_p.R34G | A*0101, A*2402, | B*4402, B*4403, |
| | | | B*5701, DRBP0701, | B*5801 |
| | | | DRB1*1501 | |
| RVVAQDPDAGKAGRLV | 145 | CELSR3_p.E356K | A*0301, A*1101, | DRB1*1001 |
| | | | A*6801, B*0801 | |
| KKKKMPKLCFASRI | 146 | TFPI2_p.R222C | A*0201, A*0301, | |
| | | | A*1101, A*2301, | |
| | | | A*2402, A*6801, | |
| | | | B*0702, B*0801, | |
| | | | B*2705, B*3501, | |
| | | | B*4501, B*5001, | |
| | | | B*5101, DRB1*1101, | |
| | | | DRB1*1201 | |
| RDPLSEITQQEKDFL | 147 | PIK3CA_p.E545Q | A*0101, A*1101, | |
| | | | B*4402, B*4403, | |
| | | | B*4901, B*5101 | |
| GYVLVAINEFS | 148 | ERBB3_p.M91I | A*2301, A*2402, | DRB1*0701, |
| | | | A*3201, B*1501, | DRB1*0901 |
| | | | B*3501, B*4901, | |
| | | | B*5701 | |
| CTYSPALNNMFCQL | 149 | TP53_p.K132N | A*0101, A*0201, | |
| | | | A*1101, A*2301, | |
| | | | A*2402, A*3201, | |
| | | | A*6801, B*0702, | |
| | | | B*0801, B*1501, | |
| | | | B*3501, B*4501, | |
| | | | B*5001, B*5701, | |
| | | | B*5801, DRB1*0301, | |
| | | | DRB1*0401, | |
| | | | DRB1*0701, | |
| | | | DRB1*0801, | |
| | | | DRB1*0901, | |
| | | | DRB1*1001, | |
| | | | DRB1*1101 | |
| HMTEVVRHCPHH | 150 | TP53_p.R175H | A*0101, B*4402, | |
| | | | B*4403, B*4501, | |
| | | | B*5001 | |
| HMTEVVRHCPHHER | 151 | TP53_p.R175H | A*0101, A*1101, | B*2705, DRB1*1301 |
| | | | A*6801, B*4402, | |
| | | | B*4403, B*4501, | |
| | | | B*5001 | |
| SCMGGMNWRPILT | 152 | TP53_p.R248W | A*0101, A*0201, | |
| | | | A*0301, A*2301, | |
| | | | A*2402, A*3201, | |
| | | | A*6801, B*0702, | |
| | | | B*0801 | |
| NSSCMGGMNWRPILTII | 153 | TP53_p.R248W | A*0201, A*0301, | A*0101, B*0702, |
| | | | A*1101, A*2301, | B*0801, B*4901, |
| | | | A*2402, A*3201, | B*5101 |
| | | | A*6801, B*2705, | |
| | | | B*4402, B*5001, | |
| | | | B*5701, B*5801 | |
| HQNSASDNAEASM | 154 | CUL1_p.D483N | A*0101, B*0702, | |
| | | | B*3501, B*5801 | |
| NSFVNDILERI | 155 | HIST2H2BE_p.F71L | A*0201, A*1101, | A*3201, B*4901, |
| | | | A*2301, A*2402, | B*5701, DRB1*0301 |
| | | | A*6801, B*5101 | |
| GPALLGLLSL | 156 | PPCS_p.S113L | B*0702,B*3501, | |
| | | | B*5101 | |
| GPALLGLLSLEAEE | 157 | PPCS_p.S113L | B*0702, B*3501, | A*0201, |
| | | | B*5101, DRB1*0101, | DRB1*0401, |
| | | | DRB1*0701, | DRB1*1501 |
| | | | DRB1*0801, | |
| | | | DRB1*0901, | |
| | | | DRB1*1001, | |
| | | | DRB1*1101, | |
| | | | DRB1*1201, | |
| | | | DRB1*1301 | |
| ITDQSLDKAQAKKHA | 158 | PBX2_p.E70K | A*0101, A*0301, | |
| | | | A*1101, A*6801, | |
| | | | DRB1*0801, | |
| | | | DRB1*0901 | |
| TITDQSLDKAQAKKHAL | 159 | PBX2_p.E70K | A*0101, A*0301, | B*0801 |
| | | | A*1101, A*6801, | |
| | | | DRB1*0801, | |
| | | | DRB1*0901 | |
| VADIKVDGKQVELAL | 160 | RHOA_p.E47K | A*0101, A*0201, | |
| | | | A*3201, B*2705, | |
| | | | B*5701, DRB1*0301 | |
| NYVADIKVDGKQVELAL | 161 | RHOA_p.E47K | A*0101, A*0201, | DRB1*0301 |
| | | | A*0301, A*1101, | |
| | | | A*3201, A*6801, | |
| | | | B*2705, B*5701 | |
| HDCVPVLLEHPLY | 162 | ACSS3_p.S290L | A*0101, A*2301, | |
| | | | B*0702, B*3501, | |
| | | | B*5101 | |
| HDCVPVLLEHPLYILYT | 163 | ACSS3_p.S290L | A*2301,B*0702, | A*0101, A*0201, |
| | | | B*3501, DRB1*0101, | A*0301, A*1101, |
| | | | DRB1*0301, | A*2402, A*3201, |
| | | | DRB1*0401, | B*4402, B*4403, |
| | | | DRB1*0701, | B*4501, B*4901, |
| | | | DRB1*0901, | B*5001, B*5101 |
| | | | DRB1*1001, | |
| | | | DRB1*1301, | |

TABLE B
Bladder Cancer Shared Neoepitope Minimal Epitopes

| | SEQ | | | |
|---|---|---|---|---|
| SEQUENCE | ID NO. | MUTATION | HLA Restriction | HLA Exclusion |
| CVPVLLEHPL | 164 | ACSS3_p.S290L | A*2301 | |
| VPVLLEHPL | 165 | ACSS3_p.S290L | B*0702, B*3501, B*5101 | |
| VPVLLEHPLY | 166 | ACSS3_p.S290L | B*3501, B*5101 | |
| PVLLEHPLY | 167 | ACSS3_p.S290L | A*0101 | |
| IVMDSGDRV | 168 | ACTB_p.G158R | DRB1*0101, A*0101, A*0201, | |
| | | | B*0702 | |
| VMDSGDRVTH | 169 | ACTB_p.G158R | A*0101 | |
| MDSGDRVTHT | 170 | ACTB_p.G158R | B*5801 | |
| SGDRVTHTV | 171 | ACTB_p.G158R | A*0101 | |
| RPDYIIVTH | 172 | AHR_p.Q383H | B*0702 | |
| RPDYIIVTHR | 173 | AHR_p.Q383H | B*0702, B*3501, B*5101 | |
| DYIIVTHRP | 174 | AHR_p.Q383H | A*2301, A*2402 | |
| SAAALPASER | 175 | C3ORF70_p.S6L | A*6801 | |
| AAALPASER | 176 | C3ORF70_p.S6L | A*6801 | |
| VVAQDPDAGK | 177 | CELSR3_p.E356K | A*0301, A*1101, A*6801 | |
| DPDAGKAGRL | 178 | CELSR3_p.E356K | B*0801 | |
| NSASDNAEA | 179 | CUL1_p.D483N | A*0101 | |
| SASDNAEASM | 180 | CUL1_p.D483N | B*0702, B*3501, B*5801 | |
| ASDNAEASM | 181 | CUL1_p.D483N | B*5801 | |
| NYLSTDVGF | 182 | ERBB2_p.S310F | A*2402 | A*2301 |
| NYLSTDVGFC | 183 | ERBB2_p.S310F | A*2301 | |
| YLSTDVGFC | 184 | ERBB2_p.S310F | DRB1*0701, A*0201 | |
| YLSTDVGFCT | 185 | ERBB2_p.S310F | A*0201 | |
| LSTDVGFCT | 186 | ERBB2_p.S310F | A*0101, B*5801 | |
| LSTDVGFCTL | 187 | ERBB2_p.S310F | A*0201, A*2402, B*0702, | |
| | | | B*3501, B*5101, B*5701 | |
| STDVGFCTL | 188 | ERBB2_p.S310F | A*0101, A*0201, A*3201 | |
| STDVGFCTLV | 189 | ERBB2_p.S310F | A*0101, A*0201 | |
| TDVGFCTLV | 190 | ERBB2_p.S310F | B*4403, B*4501 | |
| TDVGFCTLVC | 191 | ERBB2_p.S310F | B*4403 | |
| VGFCTLVCPL | 192 | ERBB2_p.S310F | B*3501, B*5101 | |
| FCTLVCPLH | 193 | ERBB2_p.S310F | DRB1*0401, DRB1*0801, | |
| | | | DRB1*1101 | |
| GYVLVAINEF | 194 | ERBB3_p.M91I | A*2301, A*2402 | |
| YVLVAINEF | 195 | ERBB3_p.M91I | DRB1*1101, A*2301, A*2402, | DRB1*0701, DRB1*0901 |
| | | | A*3201, B*1501, B*3501, | |
| | | | B*4901, B*5701, B*5801 | |
| VVFDEAHSI | 196 | ERCC2_p.N238S | DRB1*0101, DRB1*0301, | A*2301 |
| | | | DRB1*0401, A*0201, A*3201, | |
| | | | B*4901, B*5701, B*5801 | |
| DEAHSIDNV | 197 | ERCC2_p.N238S | B*4403,B*4901 | |
| DEAHSIDNVC | 198 | ERCC2_p.N238S | B*4403 | |
| HVAAVGCVQY | 199 | FBXW7_p.R505G | A*0101, A*0301, B*1501, | |
| | | | B*3501, B*5701 | |
| VAAVGCVQY | 200 | FBXW7_p.R505G | A*0101, B*1501, B*3501, | |
| | | | B*5001, B*5101, B*5701, | |
| | | | B*5801 | |
| VAAVGCVQYD | 201 | FBXW7_p.R505G | B*5801 | |
| VEADEACSV | 202 | FGFR3_p.G370C | B*4402, B*4403, B*4501, | |
| | | | B*4901, B*5001 | |
| VEADEACSVY | 203 | FGFR3_p.G370C | B*4402, B*4403 | |
| EADEACSVY | 204 | FGFR3_p.G370C | A*0101, B*3501 | |
| EADEACSVYA | 205 | FGFR3_p.G370C | B*5801 | |
| ADEACSVYA | 206 | FGFR3_p.G370C | B*4403, B*4501 | |
| ADEACSVYAG | 207 | FGFR3_p.G370C | B*4403 | |
| DEACSVYAG | 208 | FGFR3_p.G370C | B*4402, B*4403, B*5001 | |
| VLERCPHRPI | 209 | FGFR3_p.S249C | B*5101 | |
| LERCPHRPI | 210 | FGFR3_p.S249C | B*0702, B*4402, B*4403, | |
| | | | B*4901, B*5001 | |
| ADEAGSVCA | 211 | FGFR3_p.Y373C | B*4501 | |
| DEAGSVCAG | 212 | FGFR3_p.Y373C | B*4402, B*4403, B*4501, | |
| | | | B*5001 | |
| DEAGSVCAGI | 213 | FGFR3_p.Y373C | B*4402, B*4403 | |
| EAGSVCAGI | 214 | FGFR3_p.Y373C | B*5101 | |
| NSFVNDILE | 215 | HIST2H2BE_p.F71L | A*6801 | |
| NSFVNDILER | 216 | HIST2H2BE_p.F71L | A*1101, A*6801 | |
| SFVNDILER | 217 | HIST2H2BE_p.F71L | A*1101, A*6801 | |
| SFVNDILERI | 218 | HIST2H2BE_p.F71L | A*2301, A*2402 | |
| FVNDILERI | 219 | HIST2H2BE_p.F71L | A*0201, B*5101 | DRB1*0301, A*3201, B*4901, |
| | | | | B*5701 |
| VVVGADGVGK | 220 | KRAS_p.G12D | A*0301, A*1101 | |
| VVGADGVGK | 221 | KRAS_p.G12D | DRB1*1001, A*0301, A*1101 | |
| VGADGVGKS | 222 | KRAS_p.G12D | DRB1*0301 | |
| ADGVGKSAL | 223 | KRAS_p.G12D | B*4402 | |
| KTLWKALAK | 224 | MROH2B_p.E1109K | A*3201, A*6801 | DRB1*0401, A*0301, A*1101 |
| KTLWKALAKK | 225 | MROH2B_p.E1109K | A*0301, A*1101, A*3201, | |
| | | | A*6801 | |
| TLWKALAKK | 226 | MROH2B_p.E1109K | A*0301, A*1101, A*6801 | |
| DIDLGVSGEV | 227 | NFE2L2_p.R34G | A*0101 | |
| IDLGVSGEV | 228 | NFE2L2_p.R34G | DRB1*0701, DRB1*1501 | |
| IDLGVSGEVF | 229 | NFE2L2_p.R34G | B*5701 | |
| LGVSGEVFDF | 230 | NFE2L2_p.R34G | B*5701 | |
| GVSGEVFDF | 231 | NFE2L2_p.R34G | A*3201 | B*5801 |
| GVSGEVFDFS | 232 | NFE2L2_p.R34G | B*5801 | |
| ITDQSLDKA | 233 | PBX2_p.E70K | A*0101 | |
| ITDQSLDKAQ | 234 | PBX2_p.E70K | A*0101 | |
| QSLDKAQAK | 235 | PBX2_p.E70K | A*0301, A*1101 | |
| QSLDKAQAKK | 236 | PBX2_p.E70K | A*0301, A*1101, A*6801 | |
| SLDKAQAKK | 237 | PBX2_p.E70K | A*0301, A*1101 | |
| SLDKAQAKKH | 238 | PBX2_p.E70K | A*0101 | |
| LDKAQAKKH | 239 | PBX2_p.E70K | DRB1*0801, DRB1*0901 | |
| AISTRDPLSK | 240 | PIK3CA_p.E542K | A*0301, A*1101, A*6801 | |
| ISTRDPLSK | 241 | PIK3CA_p.E542K | A*0101, A*0301, A*1101, | |
| | | | A*6801 | |
| ISTRDPLSKI | 242 | PIK3CA_p.E542K | A*3201, B*5101, B*5701 | |
| STRDPLSKI | 243 | PIK3CA_p.E542K | DRB1*0301, A*3201, B*0702, | |
| | | | B*5701 | |
| STRDPLSKIT | 244 | PIK3CA_p.E542K | B*5701 | |
| PLSKITEQEK | 245 | PIK3CA_p.E542K | A*1101 | |
| LSKITEQEK | 246 | PIK3CA_p.E542K | A*6801 | |
| KITEQEKDF | 247 | PIK3CA_p.E542K | A*3201, B*5701 | |
| DPLSEITKQE | 248 | PIK3CA_p.E545K | B*5101 | |
| PLSEITKQEK | 249 | PIK3CA_p.E545K | A*0301 | |
| LSEITKQEK | 250 | PIK3CA_p.E545K | A*0101 | |
| LSEITKQEKD | 251 | PIK3CA_p.E545K | A*0101 | |
| SEITKQEKD | 252 | PIK3CA_p.E545K | B*4402, B*4403, B*4901 | |
| SEITKQEKDF | 253 | PIK3CA_p.E545K | B*4402, B*4403 | |
| ITKQEKDFL | 254 | PIK3CA_p.E545K | B*5701 | DRB1*0301, DRB 1*1301 |
| DPLSEITQQE | 255 | PIK3CA_p.E545Q | B*5101 | |
| PLSEITQQEK | 256 | PIK3CA_p.E545Q | A*1101 | |
| LSEITQQEK | 257 | PIK3CA_p.E545Q | A*0101 | |
| LSEITQQEKD | 258 | PIK3CA_p.E545Q | A*0101 | |
| SEITQQEKD | 259 | PIK3CA_p.E545Q | B*4901 | |
| SEITQQEKDF | 260 | PIK3CA_p.E545Q | B*4402, B*4403 | |
| ITQQEKDFL | 261 | PIK3CA_p.E545Q | B*5701 | DRB1*1301 |
| QMNDARHGGW | 262 | PIK3CA_p.H1047R | A*2301, A*2402, A*3201, | |
| | | | B*4402, B*4403, B*5701 | |
| MNDARHGGW | 263 | PIK3CA_p.H1047R | A*0101, B*5801 | |
| GPALLGLLSL | 264 | PPCS_p.S113L | B*0702, B*3501, B*5101 | |
| MACGFCRAI | 265 | RARS2_p.R6C | B*3501, B*5101 | |
| MACGFCRAIA | 266 | RARS2_p.R6C | B*4402, B*4403 | |
| FCRAIACQL | 267 | RARS2_p.R6C | DRB1*0101, DRB1*0701, | B*0801 |
| | | | DRB1*0901, DRB1*1001, | |
| | | | DRB1*1501 | |
| VADIK VDGKQ | 268 | RHOA_p.E47K | A*0101 | |
| IKVDGKQVE | 269 | RHOA_p.E47K | DRB1*0301 | |
| KVDGKQVEL | 270 | RHOA_p.E47K | A*0201, A*3201, B*2705, | |
| | | | B*5701 | |
| GVREVFKTA | 271 | RHOB_p.E172K | B*0702 | |
| VREVFKTAT | 272 | RHOB_p.E172K | DRB1*0901 | |
| VREVFKTATR | 273 | RHOB_p.E172K | B*2705 | |
| REVFKTATR | 274 | RHOB_p.E172K | B*2705, B*4501, B*4901, | |
| | | | B*5001 | |
| REVFKTATRA | 275 | RHOB_p.E172K | B*4402, B*4403 | |
| VFKTATRAA | 276 | RHOB_p.E172K | DRB1*0901 | |
| VFKTATRAAL | 277 | RHOB_p.E172K | A*2301, B*0702 | A*2402 |
| FKTATRAAL | 278 | RHOB_p.E172K | DRB1*0101, DRB1*0301, | DRB1*0901, DRB1*1101, |
| | | | DRB1*0701, DRB1*0801, | B*0702, B*3501 |
| | | | DRB1*1001, DRB1*1201, | |
| | | | DRB1*1301, DRB1*1501 | |
| LRLPALRFI | 279 | RXRA_p.S427F | B*2705 | DRB1*1201, DRB1*1301 |
| LRLPALRFIG | 280 | RXRA_p.S427F | B*2705 | |
| RLPALRFIGL | 281 | RXRA_p.S427F | A*0201, A*3201 | |
| LPALRFIGL | 282 | RXRA_p.S427F | B*0702, B*0801, B*3501, | |
| | | | B*5101 | |
| DGILYAFQK | 283 | SF3B1_p.E902K | A*6801 | |
| ILYAFQKQT | 284 | SF3B1_p.E902K | DRB1*0701,DRB1*0901, | |
| | | | A*0201 | |
| ILYAFQKQTT | 285 | SF3B1_p.E902K | A*0201 | |
| YAFQKQTTE | 286 | SF3B1_p.E902K | B*0801, B*3501, B*5701, | |
| | | | B*5801 | |
| YAFQKQTTED | 287 | SF3B1_p.E902K | B*3501 | |
| FQKQTTEDS | 288 | SF3B1_p.E902K | DRB1*0101, DRB1*0301, | |
| | | | DRB1*0401, DRB1*0801, | |
| | | | DRB1*1001, DRB1*1101, | |
| | | | DRB1*1301 | |
| KKKKMPKLCF | 289 | TFPI2_p.R222C | A*2402 | |
| KKKMPKLCF | 290 | TFPI2_p.R222C | DRB1*1101, DRB1*1201, | |
| | | | B*0801, B*2705 | |
| KKMPKLCFA | 291 | TFPI2_p.R222C | B*4501, B*5001 | |
| KMPKLCFAS | 292 | TFPI2_p.R222C | A*0201 | |
| KMPKLCFASR | 293 | TFPI2_p.R222C | A*0301, A*1101 | |
| MPKLCFASR | 294 | TFPI2_p.R222C | A*6801, B*0702, B*0801, | |
| | | | B*3501, B*5101 | |
| MPKLCFASRI | 295 | TFPI2_p.R222C | A*2301, A*2402, B*0702, | |
| | | | B*5101 | |
| LLGRNSFKV | 296 | TP53_p.E271K | A*0201 | DRB1*0101, DRB1*0701, |
| | | | | DRB1*1001, DRB1*1301, |
| | | | | DRB1*1501 |
| GRNSFKVRV | 297 | TP53_p.E271K | B*2705 | |
| GRNSFKVRVC | 298 | TP53_p.E271K | B*2705 | |
| RNSFKVRVCA | 299 | TP53_p.E271K | B*5801 | |
| NSFKVRVCA | 300 | TP53_p.E271K | B*4501 | |
| SFKVRVCAC | 301 | TP53_p.E271K | B*0801 | |
| KVRVCACPGR | 302 | TP53_p.E271K | A*0301, A*1101 | |
| GRDRRTKEE | 303 | TP53_p.E285K | B*2705 | |
| GRDRRTKEEN | 304 | TP53_p.E285K | B*2705 | |
| RDRRTKEENL | 305 | TP53_p.E285K | B*0801 | |
| DRRTKEENLR | 306 | TP53_p.E285K | B*2705 | |
| RRTKEENLR | 307 | TP53_p.E285K | B*2705 | |
| CTYSPALNNM | 308 | TP53_p.K132N | A*3201, B*3501, B*5701 | |
| TYSPALNNM | 309 | TP53_p.K132N | A*2301, A*2402 | |
| TYSPALNNMF | 310 | TP53_p.K132N | A*2301, A*2402, B*5701, | |
| | | | B*5801 | |
| YSPALNNMF | 311 | TP53_p.K132N | DRB1*0301, DRB1*0401, | |
| | | | DRB1*0701, DRB1*0801, | |
| | | | DRB1*0901, DRB1*1001, | |
| | | | DRB1*1101, A*0101, A*2301, | |
| | | | A*2402, A*3201, B*1501, | |
| | | | B*5701, B*5801 | |
| YSPALNNMFC | 312 | TP53_p.K132N | A*0101, A*1101, A*6801 | |
| SPALNNMFC | 313 | TP53_p.K132N | B*0702 | |
| PALNNMFCQL | 314 | TP53_p.K132N | A*2301, B*3501 | |
| ALNNMFCQL | 315 | TP53_p.K132N | A*0201, A*1101, A*2301, | |
| | | | A*2402, A*3201, B*0801, | |
| | | | B*1501, B*4402, B*4501, | |
| | | | B*5001 | |
| MTEVVRHCP | 316 | TP53_p.R175H | A*0101 | |
| TEVVRHCPH | 317 | TP53_p.R175H | B*4403, B*4501, B*5001 | |
| TEVVRHCPHH | 318 | TP53_p.R175H | B*4402, B*4403 | |
| SCMGGMNQR | 319 | TP53_p.R248Q | A*1101, A*6801, B*2705 | |
| CMGGMNQRPI | 320 | TP53_p.R248Q | A*2301, A*2402, A*3201 | |
| MGGMNQRPI | 321 | TP53_p.R248Q | B*0801 | |
| MGGMNQRPIL | 322 | TP53_p.R248Q | B*0702, B*0801 | |
| SCMGGMNWR | 323 | TP53_p.R248W | A*6801 | |
| CMGGMNWRP | 324 | TP53_p.R248W | A*0101 | |
| CMGGMNWRPI | 325 | TP53_p.R248W | A*2301, A*2402, A*3201 | |
| MGGMNWRPI | 326 | TP53_p.R248W | B*0702, B*0801 | |
| MGGMNWRPIL | 327 | TP53_p.R248W | B*0702, B*0801 | |
| GMNWRPILT | 328 | TP53_p.R248W | A*0201, A*0301 | |
| GRNSFEVCV | 329 | TP53_p.R273C | B*2705, B*4501 | |
| GRNSFEVCVC | 330 | TP53_p.R273C | B*2705 | |
| RNSFEVCVCA | 331 | TP53_p.R273C | B*5801 | |
| NSFEVCVCA | 332 | TP53_p.R273C | A*0101, B*4501, B*5001 | |
| FEVCVCACP | 333 | TP53_p.R273C | B*4402, B*4901, B*5001 | |
| FEVCVCACPG | 334 | TP53_p.R273C | B*3501 | |
| RVCACPGKDR | 335 | TP53_p.R280K | A*0301, A*1101, A*6801 | |
| CACPGKDRR | 336 | TP53_p.R280K | A*6801 | |
| CPGKDRRTE | 337 | TP53_p.R280K | B*0702 | |
| CPGKDRRTEE | 338 | TP53_p.R280K | B*0702 | |
| VRVCACPGT | 339 | TP53_p.R280T | B*5001 | |
| RVCACPGTDR | 340 | TP53_p.R280T | A*0301, A*1101, A*6801 | |
| CACPGTDRR | 341 | TP53_p.R280T | A*6801 | |
| CPGTDRRTE | 342 | TP53_p.R280T | B*0702 | |
| CPGTDRRTEE | 343 | TP53_p.R280T | B*0702 | |
| GTDRRTEEE | 344 | TP53_p.R280T | A*0101 | |
| GTDRRTEEEN | 345 | TP53_p.R280T | A*0101, B*5801 | |
| NYMCNSFCM | 346 | TP53_p.S241F | A*2301, A*2402, B*0801, | |
| | | | B*3501, B*4402, B*4403, | |
| | | | B*5101 | |
| NYMCNSFCMG | 347 | TP53_p.S241F | A*2301, A*2402, B*3501 | |
| YMCNSFCMG | 348 | TP53_p.S241F | A*0101, A*0201, A*6801, | |
| | | | B*1501 | |
| YMCNSFCMGG | 349 | TP53_p.S241F | A*0201, B*1501 | |
| MCNSFCMGGM | 350 | TP53_p.S241F | B*1501, B*4402, B*4403, | |
| | | | B*5101 | |

TABLE C
Bladder Cancer Shared Neoepitope Preferred Embodiments of Minimal Epitopes

| | SEQ ID | | | |
|---|---|---|---|---|
| SEQUENCE | NO. | MUTATION | HLA restriction | HLA exclusion |
| CVPVLLEHPL | 164 | ACSS3_p.S290L | A*2301 | |
| VPVLLEHPL | 165 | ACSS3_p.S290L | B*0702, B*3501, B*5101 | |
| VPVLLEHPLY | 166 | ACSS3_p.S290L | B*3501, B*5101 | |
| PVLLEHPLY | 167 | ACSS3_p.S290L | A*0101 | |
| IVMDSGDRV | 168 | ACTB_p.G158R | DRB1*0101, A*0101, A*0201, B*0702 | |
| VMDSGDRVTH | 169 | ACTB_p.G158R | A*0101 | |
| MDSGDRVTHT | 170 | ACTB_p.G158R | B*5801 | |
| SGDRVTHTV | 171 | ACTB_p.G158R | A*0101 | |
| RPDYIIVTH | 172 | AHR_p.Q383H | B*0702 | |
| RPDYIIVTHR | 173 | AHR_p.Q383H | B*0702, B*3501, B*5101 | |
| DYIIVTHRP | 174 | AHR_p.Q383H | A*2301, A*2402 | |
| VVAQDPDAGK | 177 | CELSR3_p.E356K | A*0301, A*1101, A*6801 | |
| DPDAGKAGRL | 178 | CELSR3_p.E356K | B*0801 | |
| NSASDNAEA | 179 | CULl_p.D483N | A*0101 | |
| SASDNAEASM | 180 | CULl_p.D483N | B*0702, B*3501, B*5801 | |
| ASDNAEASM | 181 | CULl_p.D483N | B*5801 | |
| NYLSTDVGFC | 183 | ERBB2_p.S310F | A*2301 | |
| TDVGFCTLVC | 191 | ERBB2_p.S310F | B*4403 | |
| DEAHSIDNVC | 198 | ERCC2_p.N238S | B*4403 | |
| VAAVGCVQYD | 201 | FBXW7_p.R505G | B*5801 | |
| VEADEACSV | 202 | FGFR3_p.G370C | B*4402, B*4403, B*4501, B*4901, | |
| | | | B*5001 | |
| VEADEACSVY | 203 | FGFR3_p.G370C | B*4402, B*4403 | |
| EADEACSVY | 204 | FGFR3_p.G370C | A*0101, B*3501 | |
| EADEACSVYA | 205 | FGFR3_p.G370C | B*5801 | |
| ADEACSVYA | 206 | FGFR3_p.G370C | B*4403, B*4501 | |
| ADEACSVYAG | 207 | FGFR3_p.G370C | B*4403 | |
| DEACSVYAG | 208 | FGFR3_p.G370C | B*4402, B*4403, B*5001 | |
| ADEAGSVCA | 211 | FGFR3_p.Y373C | B*4501 | |
| DEAGSVCAG | 212 | FGFR3_p.Y373C | B*4402, B*4403, B*4501, B*5001 | |
| DEAGSVCAGI | 213 | FGFR3_p.Y373C | B*4402, B*4403 | |
| EAGSVCAGI | 214 | FGFR3_p.Y373C | B*5101 | |
| NSFVNDILE | 215 | HIST2H2BE_p.F71L | A*6801 | |
| NSFVNDILER | 216 | HIST2H2BE_p.F71L | A*1101, A*6801 | |
| SFVNDILER | 217 | HIST2H2BE_p.F71L | A*1101, A*6801 | |
| SFVNDILERI | 218 | HIST2H2BE_p.F71L | A*2301, A*2402 | |
| FVNDILERI | 219 | HIST2H2BE_p.F71L | A*0201, B*5101 | DRB1*0301, A*3201, |
| | | | | B*4901, B*5701 |
| DIDLGVSGEV | 227 | NFE2L2_p.R34G | A*0101 | |
| GVSGEVFDFS | 232 | NFE2L2_p.R34G | B*5801 | |
| ITDQSLDKA | 233 | PBX2_p.E70K | A*0101 | |
| ITDQSLDKAQ | 234 | PBX2_p.E70K | A*0101 | |
| SLDKAQAKKH | 238 | PBX2_p.E70K | A*0101 | |
| LDKAQAKKH | 239 | PBX2_p.E70K | DRB1*0801, DRB1*0901 | |
| ISTRDPLSKI | 242 | PIK3CA_p.E542K | A*3201, B*5101, B*5701 | |
| STRDPLSKIT | 244 | PIK3CA_p.E542K | B*5701 | |
| PLSKITEQEK | 245 | PIK3CA_p.E542K | A*1101 | |
| KITEQEKDF | 247 | PIK3CA_p.E542K | A*3201, B*5701 | |
| DPLSEITKQE | 248 | PIK3CA_p.E545K | B*5101 | |
| PLSEITKQEK | 249 | PIK3CA_p.E545K | A*0301 | |
| LSEITKQEKD | 251 | PIK3CA_p.E545K | A*0101 | |
| DPLSEITQQE | 255 | PIK3CA_p.E545Q | B*5101 | |
| PLSEITQQEK | 256 | PIK3CA_p.E545Q | A*1101 | |
| LSEITQQEK | 257 | PIK3CA_p.E545Q | A*0101 | |
| LSEITQQEKD | 258 | PIK3CA_p.E545Q | A*0101 | |
| SEITQQEKD | 259 | PIK3CA_p.E545Q | B*4901 | |
| SEITQQEKDF | 260 | PIK3CA_p.E545Q | B*4402, B*4403 | |
| ITQQEKDFL | 261 | PIK3CA_p.E545Q | B*5701 | DRB1*1301 |
| GPALLGLLSL | 264 | PPCS_p.S113L | B*0702,B*3501, B*5101 | |
| MACGFCRAIA | 266 | RARS2_p.R6C | B*4402, B*4403 | |
| FCRAIACQL | 267 | RARS2_p.R6C | DRB1*0101, DRB1*0701, DRB1*0901, | B*0801 |
| | | | DRB1*1001, DRB1*1501 | |
| VADIKVDGKQ | 268 | RHOA_p.E47K | A*0101 | |
| IKVDGKQVE | 269 | RHOA_p.E47K | DRB1*0301 | |
| KVDGKQVEL | 270 | RHOA_p.E47K | A*0201, A*3201, B*2705, B*5701 | |
| GVREVFKTA | 271 | RHOB_p.E172K | B*0702 | |
| VREVFKTAT | 272 | RHOB_p.E172K | DRB1*0901 | |
| VREVFKTATR | 273 | RHOB_p.E172K | B*2705 | |
| REVFKTATR | 274 | RHOB_p.E172K | B*2705, B*4501, B*4901, B*5001 | |
| REVFKTATRA | 275 | RHOB_p.E172K | B*4402, B*4403 | |
| VFKTATRAA | 276 | RHOB_p.E172K | DRB1*0901 | |
| VFKTATRAAL | 277 | RHOB_p.E172K | A*2301, B*0702 | A*2402 |
| FKTATRAAL | 278 | RHOB_p.E172K | DRB1*0101, DRB1*0301, DRB1*0701, | DRB1*0901, |
| | | | DRB1*0801, DRB1*1001, DRB1*1201, | DRB1*1101, B*0702, |
| | | | DRB1*1301, DRB1*1501 | B*3501 |
| LRLPALRFIG | 280 | RXRA_p.S427F | B*2705 | |
| DGILYAFQK | 283 | SF3Bl_p.E902K | A*6801 | |
| YAFQKQTTE | 286 | SF3Bl_p.E902K | B*0801, B*3501, B*5701, B*5801 | |
| YAFQKQTTED | 287 | SF3Bl_p.E902K | B*3501 | |
| FQKQTTEDS | 288 | SF3Bl_p.E902K | DRB1*0101, DRB1*0301, DRB1*0401, | |
| | | | DRB1*0801, DRB1*1001, DRB1*1101, | |
| | | | DRB1*1301 | |
| KMPKLCFAS | 292 | TFPI2_p.R222C | A*0201 | |
| GRNSFKVRVC | 298 | TP53_p.E271K | B*2705 | |
| RNSFKVRVCA | 299 | TP53_p.E271K | B*5801 | |
| GRDRRTKEE | 303 | TP53_p.E285K | B*2705 | |
| GRDRRTKEEN | 304 | TP53_p.E285K | B*2705 | |
| RDRRTKEENL | 305 | TP53_p.E285K | B*0801 | |
| DRRTKEENLR | 306 | TP53_p.E285K | B*2705 | |
| RRTKEENLR | 307 | TP53_p.E285K | B*2705 | |
| YSPALNNMFC | 312 | TP53_p.K132N | A*0101, A*1101, A*6801 | |
| SPALNNMFC | 313 | TP53_p.K132N | B*0702 | |
| PALNNMFCQL | 314 | TP53_p.K132N | A*2301, B*3501 | |
| TEVVRHCPHH | 318 | TP53_p.R175H | B*4402, B*4403 | |
| MGGMNQRPIL | 322 | TP53_p.R248Q | B*0702, B*0801 | |
| MGGMNWRPIL | 327 | TP53_p.R248W | B*0702, B*0801 | |
| GRNSFEVCVC | 330 | TP53_p.R273C | B*2705 | |
| RNSFEVCVCA | 331 | TP53_p.R273C | B*5801 | |
| RVCACPGKDR | 335 | TP53_p.R280K | A*0301, A*1101, A*6801 | |
| CACPGKDRR | 336 | TP53_p.R280K | A*6801 | |
| CPGKDRRTE | 337 | TP53_p.R280K | B*0702 | |
| CPGKDRRTEE | 338 | TP53_p.R280K | B*0702 | |
| VRVCACPGT | 339 | TP53_p.R280T | B*5001 | |
| RVCACPGTDR | 340 | TP53_p.R280T | A*0301, A*1101, A*6801 | |
| CACPGTDRR | 341 | TP53_p.R280T | A*6801 | |
| CPGTDRRTE | 342 | TP53_p.R280T | B*0702 | |
| CPGTDRRTEE | 343 | TP53_p.R280T | B*0702 | |
| GTDRRTEEE | 344 | TP53_p.R280T | A*0101 | |
| GTDRRTEEEN | 345 | TP53_p.R280T | A*0101, B*5801 | |
| NYMCNSFCMG | 347 | TP53_p.S241F | A*2301, A*2402, B*3501 | |
| YMCNSFCMGG | 349 | TP53_p.S241F | A*0201, B*1501 | |
| MCNSFCMGGM | 350 | TP53_p.S241F | B*1501, B*4402, B*4403, B*5101 | |

In aspects of the above methods of treating a neoplasia and/or inducing an iImmune response in a subject, the shared neo-epitopes are encoded by a shared neoplasia-specific mutation detected in a neoplasia sample from the subject, the shared neo-epitopes are known or determined (e.g. predicted) to bind to a MHC protein of the subject, and/or the shared neo-epitopes are not known or determined (e.g. predicted) to bind to a MHC protein of the subject that could lead to a detrimental or suppressive immune response. For example, in aspects of the above methods, one or more peptides or polypeptides comprising, consisting of, or consisting essentially of one or more peptides or polypeptides from Table A, B, and/or C (and/or fragments and variants thereof) as disclosed herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus), which may be in a pharmaceutical formulation such as a vaccine, are administered to a subject in step (a) provided: the shared neo-epitopes are encoded by a shared neoplasia-specific mutation as disclosed in Table A, B, and/or C (noted by the columns labeled “mutation” in Tables A, B, and C) that is detected/encoded in a neoplasia sample from the subject; the shared neo-epitopes are known or determined (e.g. predicted) to bind to a MHC protein of the subject that could lead to a detrimental or suppressive immune response (noted by the columns labeled “HLA Restriction” in Tables A, B, and C); and/or the shared neo-epitopes are known or determined (e.g. predicted) to not bind to a MHC protein of the subject. In certain aspects, shared neo-epitopes are not administered to a subject in step (a) if the shared neo-epitopes are encoded by a shared neoplasia-specific mutation that is not detected in a neoplasia sample from the subject, the shared neo-epitopes are known or determined (e.g. predicted) to not bind to a MEW protein of the subject, and/or the shared neo-epitopes are known or determined (e.g. predicted) to bind to a MEW protein of the subject that could lead to a detrimental or suppressive immune response (noted by the columns labeled “HLA Restriction” in Tables A, B, and C). For example, in aspects of the above methods, one or more peptides or polypeptides comprising, consisting of, or consisting essentially of one or more peptides or polypeptides from Table A, B, and/or C (and/or fragments and variants thereof) as disclosed herein (e.g., in a pharmaceutical formulation such as a vaccine) are not administered to a subject having or suspected of having bladder cancer in step (a) provided: the shared neo-epitopes are encoded by a shared neoplasia-specific mutation as disclosed in Table A, B, and/or C (noted by the columns labeled “mutation” in Tables A, B, and C) that is not detected and/or encoded in a neoplasia sample from the subject; the shared neo-epitopes are known or determined (e.g. predicted) to not bind to a MEW protein of the subject (with such MHC noted by the columns labeled “HLA restriction” in Tables A, B, and C); and/or the shared neo-epitopes are known or determined (e.g. predicted) to bind to a MHC protein of the subject that could lead to a detrimental or suppressive immune response (with such MHC noted by the columns labeled “HLA Exclusion” in Tables A, B, and C).

As such, in aspects of the above methods of inducing an immune response or method of treating a neoplasia (e.g., bladder cancer) in a subject in need thereof, step (a) of the method further comprises detecting one or more tumor-specific mutations in the neoplasia sample from a subject (e.g., tumor tissue, such as bladder cancer tumor tissue) and/or determining HLA allotypes present in the subject, and administering one or more of the instantly-disclosed peptides or polypeptides comprising one or more identified shared neo-epitopes (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) provided: the shared neo-epitopes are encoded by a shared neoplasia-specific mutation that is detected in the neoplasia sample from the subject, and/or the shared neo-epitopes are known or determined (e.g. predicted) to bind to a MHC protein of the subject. In aspects, the method further optionally comprises administering one or more of the instantly-disclosed peptides or polypeptides comprising one or more identified shared neo-epitopes (including peptides or polypeptide comprising, consisting of, or consisting essentially of one or more peptides or polypeptides from Table A, B, and/or C, and/or fragments and variants thereof, as disclosed herein) provided the shared neo-epitopes are not known or determined (e.g. predicted) to bind to a MEW protein of the subject that could lead to a detrimental or suppressive immune response (with such MHC noted by the columns labeled “HLA Exclusion” in Tables A, B, and C). In aspects of the above methods, the appropriate peptides or polypeptides comprising one or more identified shared neo-epitopes (including peptides or polypeptide comprising, consisting of, or consisting essentially of one or more peptides or polypeptides from Table A, B, and/or C, and/or fragments and variants thereof, as disclosed herein) are administered within 1 week of detecting one or more tumor-specific mutations in the neoplasia sample from a subject (e.g., tumor tissue, such as bladder cancer tumor tissue) and/or determining HLA allotypes present in the subject.

Identifying Shared Neo Epitopes for the Shared-Neoplasia Vaccine of Step (a)

In aspects of the above-described methods of inducing an immune response or method of treating a neoplasia (e.g., bladder cancer) in a subject in need thereof, the one or more identified shared neo-epitopes for administration in step (a) are identified by a method comprising: i) identifying shared neoplasia-specific mutations in a neoplasia specimen of a subject diagnosed as having a neoplasia; ii) assessing the shared neoplasia-specific mutations identified in step (i) to identify known or determined (e.g. predicted) shared neo-epitopes encoded by said mutations for use in the shared neoplasia vaccine; and iii) assessing the identified shared neo-epitopes encoded by said shared neoplasia-specific mutations from step (ii) to identify neo-epitopes that are known or determined (e.g. predicted) to engage regulatory T cells and/or other detrimental T cells (including T cells with potential host cross-reactivity and/or anergic T cells), and excluding such identified neo-epitopes that are known or predicted to engage regulatory T cells and/or other detrimental T cells (including T cells with potential host cross-reactivity and/or anergic T cells) from the shared neo-epitopes for use in the shared neoplasia vaccine. In aspects of the method of identifying shared neo-epitopes for a shared neoplasia vaccine, the method further includes: iv) designing at least one peptide or polypeptide, said peptide or polypeptide comprising at least one identified shared neo-epitope encoded by said shared neoplasia-specific mutations, provided said shared neo-epitope is not identified in step (iii) as being known or determined (e.g. predicted) to engage regulatory T cells and/or other detrimental T cells (including T cells with potential host cross-reactivity and/or anergic T cells). In aspects, the method further includes: v) providing the at least one peptide or polypeptide designed in step (iv) or a nucleic acid encoding said peptides or polypeptides. In aspects, the method further includes: v) providing the at least one peptide or polypeptide designed in step (iv) or a nucleic acid encoding said peptides or polypeptides. In aspects, the method includes: v) providing at least one peptide or polypeptide designed in step (iv) or a nucleic acid encoding said peptides or polypeptides for a subject, wherein said shared neo-epitopes are encoded by a shared neoplasia-specific mutation detected in a neoplasia sample from the subject and/or wherein said shared neo-epitopes are known or determined (e.g. predicted) to bind to a MHC protein of the subject. In aspects, the at least one peptide or polypeptide designed in step (iv) or a nucleic acid encoding said peptides or polypeptides can be provided in an “off the shelf” pre-furnished shared neo-epitope warehouse. In even further aspects, the method further includes vi) providing a vaccine comprising the at least one peptide or polypeptide or nucleic acid provided in step (v). In aspects, the method includes vi) providing a vaccine comprising the at least one peptide or polypeptide or nucleic acid provided in step (v) for a subject, wherein said share neo-epitopes are encoded by a shared neoplasia-specific mutation detected in a neoplasia sample from the subject and/or wherein said shared neo-epitopes are known or determined (e.g. predicted) to bind to a MHC protein of the subject. In aspects, the vaccine can be produced using the “off the shelf” pre-furnished shared neo-epitope warehouse.

In aspects, a method of identifying shared neo-epitopes for a shared neoplasia vaccine for administration in step (a) includes: i) assessing identified shared neoplasia-specific mutations from a neoplasia specimen of a subject (e.g., in aspects said subject being diagnosed as having a neoplasia) to identify known or determined (e.g. predicted) shared neo-epitopes encoded by said shared neoplasia-specific mutations for use in the shared neoplasia vaccine; and ii) assessing the identified shared neo-epitopes encoded by said shared neoplasia-specific mutations from step (i) to identify neo-epitopes that are known or determined (e.g. predicted) to engage regulatory T cells and/or other detrimental T cells (including T cells with potential host cross-reactivity and/or anergic T cells), and excluding such identified neo-epitopes that are known or determined (e.g. predicted) to engage regulatory T cells and/or other detrimental T cells (including T cells with potential host cross-reactivity and/or anergic T cells) from the shared neo-epitopes for use in the shared neoplasia vaccine. In aspects of the method of identifying shared neo-epitopes for a shared neoplasia vaccine, the method further includes: iii) designing at least peptide or polypeptide, said peptide or polypeptide comprising at least one identified shared neo-epitope encoded by said shared neoplasia-specific mutations, provided said shared neo-epitope is not identified in step (ii) as being known or determined (e.g. predicted) to engage regulatory T cells and/or other detrimental T cells (including T cells with potential host cross-reactivity and/or anergic T cells). In aspects, the method further includes: iv) providing the at least one peptide or polypeptide designed in step (iii) or a nucleic acid encoding said peptides or polypeptides. In aspects, the method includes: iv) providing at least one peptide or polypeptide designed in step (iii) or a nucleic acid encoding said peptides or polypeptides for a subject, wherein said share neo-epitopes are encoded by a shared neoplasia-specific mutation detected in a neoplasia sample from the subject and/or wherein said shared neo-epitopes are known or determined (e.g. predicted) to bind to a MHC protein of the subject. In aspects, the at least one peptide or polypeptide designed in step (iv) or a nucleic acid encoding said peptides or polypeptides can be provided in an “off the shelf” pre-furnished shared neo-epitope warehouse. In even further aspects, the method further includes v) providing a vaccine comprising the at least one peptide or polypeptide or nucleic acid provided in step (iv). In aspects, the method includes v) providing a vaccine comprising the at least one peptide or polypeptide or nucleic acid provided in step (iv) for a subject, wherein said share neo-epitopes are encoded by a shared neoplasia-specific mutation detected in a neoplasia sample from the subject and/or wherein said shared neo-epitopes are known or determined (e.g. predicted) to bind to a MHC protein of the subject. In aspects, the vaccine can be produced using the “off the shelf” pre-furnished shared neo-epitope warehouse.

Identifying Shared Neoplasia-Specific Mutations

In aspects, the step of identifying shared neoplasia-specific mutations (e.g., shared cancer-specific mutations) comprises sequencing genomic DNA and/or RNA of a neoplasia specimen (e.g., a neoplasia specimen of the patient or a neoplasia specimen from a subject or each subject within a population of subjects). In aspects, a neoplasia specimen relates to any sample, such as a bodily sample derived from a patient, containing or being expected of containing neoplasia cells (e.g. tumor or cancer cells). In aspects, the bodily sample may be any tissue sample such as blood, a tissue sample obtained from a neoplasia sample (e.g., a primary tumor or from tumor metastases/circulating tumor cells), or any other sample containing neoplasia cells (e.g., tumor or cancer cells). In particular aspects, the neoplasia is bladder cancer.

In aspects, the step of identifying of shared neoplasia-specific mutations comprises comparing the sequence information obtained from the neoplasia specimen (e.g., a neoplasia specimen of the patient or a neoplasia specimen from a subject or each subject within a population of subjects) with a reference sample, such as sequence information obtained from sequencing nucleic acid (e.g., such as DNA or RNA) of normal, non-neoplasia cells (e.g., non-cancerous cells), such as somatic or germline tissue/cells. In aspects, a reference sample may be obtained from the same neoplasia patient as the neoplasia sample is obtained or a different individual. In aspects, a reference sample may be any tissue sample such as blood or a sample from a non-neoplasia tissue. In aspects, normal genomic germline DNA may be obtained from peripheral blood mononuclear cells (PBMCs).

In aspects, shared neoplasia-specific mutations may include all shared neoplasia-specific (e.g. cancer-specific) mutations present in one or more neoplasia cells (e.g., cancer or tumor cells) of a patient, or it may refer to only a portion of the shared neoplasia-specific mutations present in one or more neoplasia cells of a patient. Accordingly, the present invention may involve the identification of all shared neoplasia-specific mutations present in one or more neoplasia cells of a patient, or it may involve the identification of only a portion of the shared neoplasia-specific mutations present in one or more neoplasia cells of a patient. In aspects, the methods of identifying shared neo-epitopes for a shared neoplasia vaccine of the present invention provide for the identification of a number of shared neoplasia-specific mutations which will provide a sufficient number of shared neo-epitopes to be included in the instantly-disclosed strategies, methods, and compositions.

In aspects, the mutations are shared neoplasia-specific mutations (e.g., somatic mutations) in a neoplasia specimen (e.g. a tumor specimen) of a neoplasia patient (e.g. a cancer patient, such as a bladder cancer patient), which may be determined by identifying sequence differences between the genome, exome and/or transcriptome of a neoplasia specimen and the genome, exome and/or transcriptome of a non-neoplasia specimen. In aspects, shared neoplasia-specific mutations, including somatic mutations, are determined in the genome, preferably the entire genome, of a neoplasia specimen. As such, the instant invention may include identifying all or a portion of shared neoplasia-specific mutations of the genome, preferably the entire genome, of one or more neoplasia cells. In aspects, shared neoplasia-specific mutations, including somatic mutations, are determined in the exome, preferably the entire exome, of a neoplasia specimen. As such, the instant invention may include identifying all or a portion of shared neoplasia-specific mutations of the exome, preferably the entire exome of one or more neoplasia cells. In aspects, shared neoplasia-specific mutations, including somatic mutations, are determined in the transcriptome, preferably the entire transcriptome, of a neoplasia specimen. As such, the instant invention may include identifying all or a portion of the shared neoplasia-specific transcriptome, preferably the entire transcriptome, of one or more neoplasia cells.

In aspects, any suitable sequencing method as is known in the art can be used according to the instant invention for determining shared neoplasia-specific mutations is step (i), including but not limited to “conventional” sequencing methodology and Next Generation Sequencing (NGS) technologies. “Next Generation Sequencing” or “NGS” refers to all high throughput sequencing technologies which, in contrast to the “conventional” sequencing methodology known as Sanger chemistry, read nucleic acid templates randomly in parallel along the entire genome by breaking the entire genome into small pieces. As is known in the art, such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are known in the art can be used. Non-limiting examples of such NGS technologies/platforms include, but are not limited to sequencing-by-ligation approaches, ion semiconductor sequencing, pyrosequencing, single-molecule sequencing technologies, nano-technologies for single-molecule sequencing, and electron microscopy based technologies for single-molecule sequencing. Further, in aspects, “Third Generation Sequencing” methods, as are known in the art, could be used for determining neoplasia-specific mutations. In aspects, neoplasia-specific mutations may be determined by direct protein sequencing techniques, as are known in the art. Further, in aspects, neoplasia-specific mutations can be determined by using MEW multimers, as is known in the art.

As such, in aspects of the method of identifying shared neo-epitopes for a shared neoplasia vaccine of step (a), the step of identifying shared neoplasia-specific mutations includes identifying sequence differences between the full or partial genome, exome, and/or transcriptome of a neoplasia specimen from a subject diagnosed as having a neoplasia and a non-neoplasia specimen. In aspects, a non-neoplasia specimen is derived from the subject diagnosed as having a neoplasia. In further aspects, identifying shared neoplasia-specific mutations or identifying sequence differences comprises Next Generation Sequencing (NGS). In aspects, the step of identifying shared neoplasia-specific mutations comprises selecting from the neoplasia a plurality of nucleic acid sequences, each comprising mutations not present in a non-neoplasia sample. In aspects, identifying shared neoplasia-specific mutations comprises sequencing genomic DNA and/or RNA of the neoplasia specimen.

In aspects of the method of identifying shared neo-epitopes for a shared neoplasia vaccine for step (a), the shared neoplasia-specific mutations are neoplasia-specific somatic mutations. In aspects, the neoplasia-specific mutations are single nucleotide variations (SNVs), insertions and deletions (which can generate both in-frame and frameshift mutations), and other large-scale rearrangements such as but not limited to chromosomal inversions, duplications, insertions, deletions, or translocations. In aspects, neoplasia specific mutations, including SNVs, insertions, and deletions, are non-synonymous mutations. In aspects, neoplasia-specific mutations, including SNVs, insertions and deletions (which can be non-synonymous mutations), and other large-scale rearrangements, are mutations of proteins encoded in the neoplasia specimen of the subject diagnosed as having a neoplasia. In aspects, neoplasia specific mutations, including SNVs, are non-synonymous mutations. In aspects, neoplasia-specific mutations, including SNVs (which can be non-synonymous mutations), indels, and frameshifts, are mutations of proteins encoded in the neoplasia specimen of the subject diagnosed as having a neoplasia. In aspects, the subject is diagnosed as having bladder cancer.

Identifying Shared Neo Epitopes

In aspects of the method of identifying shared neo-epitopes for a shared neoplasia vaccine of step (a), the step of assessing the identified shared neoplasia-specific mutations to identify known or determined (e.g. predicted) shared neo-epitopes encoded by said mutations comprises in silico testing. In aspects, in silico testing includes using validated algorithms (e.g., but not limited to, EPIMATRIX®, netMHCpan, NetMHC, netMHCcons, SYFPEITHI, HLA_BIND) to predict which shared neoplasia-specific mutations create shared neo-epitopes, preferably neo-epitopes that can bind to an MHC allotype of a patient. For example, using validated algorithms, bioinformatic analysis of the identified shared neoplasia-specific mutations and their respective cognate native antigens can be performed to predict which identified shared neoplasia-specific mutations create shared neo-epitopes (preferably shared neo-epitopes that can bind to a patient's MHC allotype), and in aspects to predict which identified shared neoplasia-specific mutations create shared neo-epitopes that could bind to a patient's MHC allotype more effectively than the cognate native antigen. Thus, in aspects, assessing identified shared neoplasia-specific mutations from a neoplasia specimen of a subject diagnosed as having a neoplasia to identify known or determined (e.g. predicted) neo-epitopes encoded by said mutations for use in the shared neoplasia vaccine, said neo-epitopes are known or determined (e.g. predicted) to bind to a MHC protein of the subject comprises the use of well-validated algorithms.

In aspects, said in silico testing to identify known or determined (e.g. predicted) shared neo-epitopes encoded by said shared neoplasia-specific mutations comprises using the EPIMATRIX® algorithm. EPIMATRIX® is a proprietary computer algorithm developed by EpiVax, which is used to screen protein sequences for the presence of putative T cell epitopes. The algorithm uses matrices for prediction of 9- and 10-mer peptides binding to MHC molecules. Each matrix is based on position-specific coefficients related to amino acid binding affinities that are elucidated by a method similar to, but not identical to, the pocket profile method (Sturniolo, T. et al., Nat. Biotechnol., 17:555-561, 1999). Input sequences are parsed into overlapping 9-mer or 10-mer frames where each frame overlaps the last by 8 or 9 amino acids, respectively. Thus, in aspects, input sequences of the mutated peptide and the non-mutated peptide are parsed into overlapping 9-mer or 10-mer frames where each frame overlaps the last by 8 or 9 amino acids. Each of the resulting frames form the mutated peptide and the non-mutated peptide are then scored for predicted binding affinity with respect to MHC class I alleles (e.g., but not limited to, HLA-A and HLA-B alleles) and MHC class II alleles (e.g., but not limited to HLA-DRB1 alleles). EPIMATRIX® raw scores are normalized against the scores of a large sample of randomly generated peptides (e.g., but not limited to 10,000 randomly generated peptides). The resulting “Z” scores are normally distributed and directly comparable across alleles. The resulting “Z” score is reported. In aspects, any 9-mer or 10-mer peptide with an allele-specific EPIMATRIX® Z-score that is theoretically the top 5% of any given sample (e.g., having an EPIMATRIX® Z-score above 1.64), is considered a putative T cell epitope. In aspects, EPIMATRIX® identifies the mutated peptide as a neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide. Previous studies have also demonstrated that EPIMATRIX® accurately predicts published MHC ligands and T cell epitopes.

In aspects, assessing the shared neoplasia-specific mutations to identify known or determined (e.g. predicted) shared neo-epitopes encoded by said mutations includes one or more of the following steps:

a) determining a binding score for a mutated peptide to one or more MHC molecules, wherein said mutated peptide is encoded by at least one of said shared neoplasia-specific mutations;

b) determining a binding score for a non-mutated peptide to the one or more MHC molecules, wherein the non-mutated peptide is identical to the mutated peptide except for the encoded at least one of said shared neoplasia-specific mutations;

c) determining the percentile rank of the binding scores of both the mutated peptide of step (a) and the non-mutated peptide of step (b) as compared to an expected distribution of binding scores for sufficiently large enough set (e.g., at least 10,000) randomly generated peptides using naturally observed amino acid frequencies;

d) determining the TCR facing amino acid residues of said mutated peptide and said non-mutated peptide; and

e) identifying the mutated peptide as a shared neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide. In further aspects, the one or more MEW molecules are MHC class I molecules and/or MEW class II molecules.

In aspects, the step of assessing the shared neoplasia-specific mutations to identify known or determined (e.g. predicted) shared neo-epitopes encoded by said mutations comprises in vitro testing. More particularly, determining the binding score of both the mutated peptide of step (a) and the non-mutated peptide of step (b) may comprise in vitro MHC binding assays (as are known in the art) to determine a binding score for the mutated peptide to one or more MEW molecules and to determine a binding score for the non-mutated peptide to the one or more MHC molecules. In aspects, and similar to an in silico analysis, input sequences are parsed into overlapping 9-mer or 10-mer frames where each frame overlaps the last by 8 or 9 amino acids, respectively. Thus, in aspects, input sequences of the mutated peptide from step (a) and the non-mutated peptide from step (b) are parsed into overlapping 9-mer or 10-mer frames where each frame overlaps the last by 8 or 9 amino acids. Each of the resulting frames from the mutated peptide from step (a) and the non-mutated peptide from step (b) are then scored for binding affinity with respect to MEW class I alleles (e.g., but not limited to, HLA-A and HLA-B alleles) in in vitro binding assays, with such binding assays as are known in the art. In the case of testing for epitopes that bind MEW class II alleles (e.g., but not limited to HLA-DRB1 alleles) in in vitro binding assays, input sequences are parsed into overlapping 15-mer or 20-mer frames where each frame overlaps the last by 5 or 10 amino acids, respectively. Thus, in aspects, input sequences of the mutated peptide from step (a) and the non-mutated peptide from step (b) are parsed into overlapping 15-mer or 20-mer frames where each frame overlaps the last by 5 or 10 amino acids. Each of the resulting frames from the mutated peptide from step (a) and the non-mutated peptide from step (b) are then scored for binding affinity with respect to MEW class II alleles (e.g., but not limited to HLA-DRB1 alleles) in in vitro binding assays, with such binding assays as are known in the art.

In aspects, the step of determining the percentile rank of the binding scores of both the mutated peptide of step (a) and the non-mutated peptide of step (b) as compared to an expected distribution of binding scores for a sufficiently large enough set (e.g., at least 10,000) randomly generated peptides using naturally observed amino acid frequencies, the raw binding scores, whether determined by in silico methods or in vitro methods, are adjusted to fit a normal, or Z-distribution. Raw binding scores are normalized based on the average (μ) binding score and standard deviation (σ) of a set of a large number (e.g., 10,000) random 9- or 10-mer amino acid sequences, following the naturally observed amino acid frequencies from UniProtKB/Swiss-Prot, as follows:

$$Normalized binding score = \frac{Raw binding score - μ}{σ} .$$

Normalized binding scores, which may be referred to as binding scores or likelihood of binding, within the top 5% of this normal distribution are defined as “hits”; which are potentially immunogenic and worthy of further consideration. These peptides have a significant chance of binding to MHC molecules with moderate to high affinity and, therefore, have a significant chance of being presented on the surface of both professional antigen presenting cells (APC) such as dendritic cells or macrophages, as well as non-professional APC, where they may be interrogated and potentially bound by passing CD8+ and CD4+ T cells.

In aspects, the mutated peptide and non-mutated peptide are both 9 amino acids in length or the mutated peptide and non-mutated peptide are both 10 amino acids in length. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at position 2, 3, 5, 7, and 8 of the mutated and non-mutated peptide as counted from the amino terminal. In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at position 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at position 4, 5, 6, 7, 8, and 9 of the mutated and non-mutated peptide as counted from the amino terminal. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at any combination of residues at positions 2, 3, 5, 7, and 8 (e.g., but not limited to, positions 3, 5, 7 and 8; positions 2, 5, 7, and 8; positions 2, 3, 5, and 7, etc. of the mutated and non-mutated peptide as counted from the amino terminal. In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at positions 4, 5, 6, 7, and 8; 1, 4, 5, 6, 7 and 8; or 1, 3, 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at any combination of residues at positions 1, 3, 4, 5, 6, 7, 8, and 9 of the mutated and non-mutated peptide as counted from the amino terminal.

In aspects of the method of identifying shared neo-epitopes for a shared neoplasia vaccine, particularly assessing the shared neoplasia-specific mutations to identify known or determined (e.g. predicted) shared neo-epitopes, the identified shared neo-epitopes may be optionally further confirmed by experimental validation for peptide-MHC binding, activation of CD8+ and/or CD4+ T cells, and/or by confirmation of gene expression at the RNA level. Such experimental validation may comprise in vitro and/or in vivo techniques, as are known in the art.

Identification and Removal of Neo-Epitopes from the Shared Neo-Epitopes that are Known or Determined (e.g. Predicted) to Engage Regulatory T cells and/or Other Detrimental T cells

The inadvertent inclusion of regulatory T cell-driving neo-epitopes, as well as neo-epitopes that engage other detrimental T cells (including T cells with potential host cross-reactivity and/or anergic T cells), in vaccine formulations may hinder efforts to induce strong T cell-mediated tumor control. Screening of shared neoantigen sequences to identify and remove potential regulatory T cell inducing neo-epitopes and neo-epitopes that engage other detrimental T cells (including T cells with potential host cross-reactivity and/or anergic T cells) (e.g., using specialized tools, including in silico screening tools), may be critical to designing new shared neoantigen vaccines with higher quality candidates.

As such, in aspects, the step of assessing the identified shared neo-epitopes encoded by said shared neoplasia-specific mutations to identify neo-epitopes that are known or determined (e.g. predicted) to engage (e.g. bind and activate) regulatory T cells and/or other detrimental T cells (including T cells with potential host cross-reactivity and/or anergic T cells) comprises determining whether said identified shared neo-epitopes encoded by said mutations share TCR contacts with proteins derived from either the human proteome or the human microbiome, wherein said identified neo-epitopes encoded by said mutations that are determined to share TCR contacts with proteins derived from either the human proteome or the human microbiome are identified as neo-epitopes that are known or determined (e.g. predicted) to engage regulatory T cells and/or other detrimental T cells (including T cells with potential host cross-reactivity and/or anergic T cells). In aspects, TCR contacts for a 9-mer identified neo-epitope that bind to a MHC class II molecule are at position 2, 3, 5, 7, and 8 of the identified neo-epitope as counted from the amino terminal, wherein the TCR contacts for a 9-mer identified neo-epitope that binds to a MHC class I molecule are at position 4, 5, 6, 7, and 8 of the identified neo-epitope as counted from the amino terminal, and wherein the TCR contacts for a 10-mer identified neo-epitope that bind to a MHC class I molecule are at position 4, 5, 6, 7, 8, and 9 of the identified neo-epitope as counted from the amino terminal. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at any combination of residues at positions 2, 3, 5, 7, and 8 (e.g., but not limited to, positions 3, 5, 7 and 8; positions 2, 5, 7, and 8; positions 2, 3, 5, and 7, etc. of the mutated and non-mutated peptide as counted from the amino terminal. In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at positions 4, 5, 6, 7, and 8; 1, 4, 5, 6, 7 and 8; or 1, 3, 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at any combination of residues at positions 1, 3, 4, 5, 6, 7, 8, and 9 of the mutated and non-mutated peptide as counted from the amino terminal.

In aspects, the step of assessing the identified shared neo-epitopes encoded by said mutations to identify neo-epitopes that are known or determined (e.g. predicted) to engage regulatory T cells comprises conducting a homology screen on each identified shared neo-epitope or epitope sequence presenting a high likelihood of binding to WIC in order to characterize the degree of similarity with self of each of the encoded MHC class I- and WIC class II-restricted identified shared neo-epitopes and their corresponding non-mutated epitopes. MHC class I or WIC class II shared neo-epitopes and MHC class I or MHC class II corresponding non-mutated epitopes with two or more (and in further aspects, three or more) cross-reactive matches in the reference proteome are categorized as exhibiting a high degree of similarity with self and are considered to have a higher likelihood of being tolerated or to engage regulatory T cells and/or other detrimental T cells (including T cells with potential host cross-reactivity and/or anergic T cells).

In aspects, a homology screen is used remove epitopes containing combinations of TCR-facing residues that are commonly found in a reference proteome. In aspects, a homology screen comprises analysis of all the predicted epitopes contained within a given protein sequence and dividing each predicted epitope into its constituent amino acid content of both the MHC-binding agretope and the TCR-binding epitope. In aspects, the TCR-binding epitope (which can be referred to as TCR binding residues, TCR facing epitope, TCR facing residues, or TCR contacts) for a 9-mer identified neo-epitope or epitope that bind to a MHC class II molecule are at position 2, 3, 5, 7, and 8 of the identified neo-epitope, while the MHC-binding agretope (which can be referred to as MHC contacts, WIC facing residues, WIC-binding residues, or MHC-binding face) for a 9-mer identified neo-epitope or epitope that bind to a WIC class II molecule are at position 1, 4, 6, and 9, both as counted from the amino terminal. In aspects, the TCR binding epitope for a 9-mer identified neo-epitope that binds to a MHC class I molecule are at position 4, 5, 6, 7, and 8 of the identified neo-epitope or epitope, while the WIC binding agretope for a 9-mer identified neo-epitope or epitope that bind to a WIC class I molecule are at position 1, 2, 3, and 9, both as counted from the amino terminal. In aspects, the TCR binding epitope for a 10-mer identified neo-epitope that bind to a WIC class I molecule are at position 4, 5, 6, 7, 8, and 9 of the identified neo-epitope, while the MHC binding agretope for a 10-mer identified neo-epitope or epitope that bind to a MHC class I molecule are at position 1, 2, 3, 9, and 10, both as counted from the amino terminal. In aspects, the TCR-binding epitope for a 9-mer identified neo-epitope or epitope that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified neo-epitope or epitope, while the MHC binding agretope for a 9-mer identified neo-epitope or epitope is the complementary face to the TCR facing residues, both as counted from the amino terminal. In aspects, the TCR binding epitope for 9-mer identified neo-epitope or epitope that bind to a MHC class I molecule are at positions 4, 5, 6, 7, and 8; 1, 4, 5, 6, 7 and 8; or 1, 3, 4, 5, 6, 7, and 8 of the identified neo-epitope or epitope, while the MHC binding agretope for a 9-mer identified neo-epitope or epitope is the complementary face to the TCR facing residues, both as counted from the amino terminal. In aspects, the TCR-binding epitope for a 10-mer identified neo-epitope or epitope that bind to a MHC class I molecule are at any combination of residues at positions 1, 3, 4, 5, 6, 7, 8, and 9 of the identified neo-epitope or epitope, while the MHC binding agretope for a 10-mer identified neo-epitope or epitope is the complementary face to the TCR facing residues, both as counted from the amino terminal.

Each sequence is then screened against a database of proteins (e.g., a database of human proteins derived from the UniProt database (UniProt Proteome ID UP000005640, Reviewed/Swiss-Prot set)). Cross-conserved epitopes, or peptides derived from the reference proteome with a compatible MHC binding agretope (i.e. the agretopes of both the input (mutated) peptide and its reference non-mutated counterpart are predicted to bind to the same MHC allele) and exactly the same TCR facing epitope, are returned. The Homology Score of an epitope corresponds to the number of matching cross-conserved MHC binding peptides within the reference proteome. In other words, the Homology Score Hₑ of an epitope e is calculated as follows:

Hₑ=|Xₑ|,

where:

Xₑ corresponds to the set of MHC binding peptides derived from the reference proteome that are restricted to the same MHC class I or MHC class II as epitope e and presenting a TCR facing epitope identical to the epitope e.

By extension, the Homology Score of a given peptide or protein corresponds to the average Homology Score of each individual epitope contained with the peptide or protein. In other words, the Homology Score Hₚ of a peptide p is calculated as follows:

$${H}_{p} = \frac{{Σ}_{e ∈ E} {H}_{e}}{\vert E \vert}$$

where:
E corresponds to the set of MHC class I- or MHC class II-restricted epitopes within peptide p;
   Hₑ corresponds to the Homology Score of epitope e as defined above.

In aspects, an analysis procedure is then run on each mutated sequence to determine if a substring within the amino acid sequence can be found, such that:
at least one MHC class I- or MEW class II-restricted epitope is encoded in the substring, and;
all MEW class I- or MEW class II-restricted neo-epitopes encoded in the substring have no more than two cross-reactive matches in the reference proteome, and;
all MEW class I- or MEW class II-restricted epitopes encoded in the substring have no more than two cross-reactive matches in the reference proteome.

This analysis procedure has the effect of removing amino acid substrings containing putative epitopes that engage regulatory T cells, other detrimental T cells (including T cells with potential host cross-reactivity and/or anergic T cells), and other highly cross-conserved epitopes from the identified shared neo-epitope sequences. The resulting shared neo-epitope sequences will only contain epitopes or neo-epitopes that exhibit low degree of similarity with self-sequences. Shared neo-epitope sequences are discarded from consideration for use in a shared neoplasia-specific vaccine if no substring matching the above criteria can be found. Conversely, the same homology analysis can be performed against a set of known infectious disease-derived epitopes known to be immunogenic, extracted for example from the IEDB database, or against a set of other known immunogenic sequences or common pathogen-derived sequences. This analysis has the purpose of identifying shared neo-epitope candidates that share a high degree of homology with other known or putative effector T cell epitopes. Shared neoantigens containing such shared neo-epitopes can be prioritized in vaccine formulations.

In aspects, the step of assessing the identified shared neo-epitopes encoded by said shared neoplasia-specific mutations to identify neo-epitopes that are known or determined (e.g. predicted) to engage regulatory T cells and/or other detrimental T cells (including T cells with potential host cross-reactivity and/or anergic T cells) comprises in silico testing. In aspects, in silico testing comprises analyzing whether the identified neo-epitopes are predicted to engage regulatory T cells and/or other detrimental T cells (including T cells with potential host cross-reactivity and/or anergic T cells). In aspects, in silico testing comprises using the JANUSMATRIX™ algorithm. JANUSMATRIX™ is a homology analysis tool that compares putative T cell epitopes and their TCR-facing residues across genome sequences rather than linear peptide fragments, and thus considers aspects of antigen recognition that are not captured by raw sequence alignment. In aspects, JANUSMATRIX™ parses the epitopes into 9-mer frames or 10-mer frames and divides each 9-mer or 10-mer into the MHC-binding agretope and the TCR-binding epitope. In aspects, the TCR-binding epitope (which can be referred to as TCR binding residues, TCR facing epitope, TCR facing residues, or TCR contacts) for a 9-mer identified neo-epitope or epitope that bind to a MHC class II molecule are at position 2, 3, 5, 7, and 8 of the identified neo-epitope, while the MHC-binding agretope (which can be referred to as MHC contacts, MHC facing residues, MHC-binding residues, or MHC-binding face) for a 9-mer identified neo-epitope or epitope that bind to a MHC class II molecule are at position 1, 4, 6, and 9, both as counted from the amino terminal. In aspects, the TCR binding epitope for a 9-mer identified neo-epitope that binds to a MHC class I molecule are at position 4, 5, 6, 7, and 8 of the identified neo-epitope or epitope, while the MHC binding agretope for a 9-mer identified neo-epitope or epitope that bind to a MHC class I molecule are at position 1, 2, 3, and 9, both as counted from the amino terminal. In aspects, the TCR binding epitope for a 10-mer identified neo-epitope that bind to a MHC class I molecule are at position 4, 5, 6, 7, 8, and 9 of the identified neo-epitope, while the MHC binding agretope for a 10-mer identified neo-epitope or epitope that bind to a MHC class I molecule are at position 1, 2, 3, 9, and 10, both as counted from the amino terminal. In aspects, the TCR-binding epitope for a 9-mer identified neo-epitope or epitope that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified neo-epitope or epitope, while the MHC binding agretope for a 9-mer identified neo-epitope or epitope is the complementary face to the TCR facing residues, both as counted from the amino terminal. In aspects, the TCR binding epitope for 9-mer identified neo-epitope or epitope that bind to a MHC class I molecule are at positions 4, 5, 6, 7, and 8; 1, 4, 5, 6, 7 and 8; or 1, 3, 4, 5, 6, 7, and 8 of the identified neo-epitope or epitope, while the MHC binding agretope for a 9-mer identified neo-epitope or epitope is the complementary face to the TCR facing residues, both as counted from the amino terminal. In aspects, the TCR-binding epitope for a 10-mer identified neo-epitope or epitope that bind to a MHC class I molecule are at any combination of residues at positions 1, 3, 4, 5, 6, 7, 8, and 9 of the identified neo-epitope or epitope, while the MHC binding agretope for a 10-mer identified neo-epitope or epitope is the complementary face to the TCR facing residues, both as counted from the amino terminal. JANUSMATRIX™ then searches for potentially cross-reactive TCR-facing epitopes across any number of large sequence databases that have been pre-loaded into the tool, including the protein sequences from bacterial and viral organisms that make up the gut microbiome (e.g., the human gut microbiome), autologous proteins from the genome (e.g., the human genome), and viral and bacterial pathogens (e.g., human viral and human bacterial pathogens). JANUSMATRIX™ focuses in 9-mer and/or 10-mer searches because although peptides of different lengths interact with the MHC, most T cell epitopes can be mapped to a minimum of nine or ten amino acids in any given peptide, even if the peptide is longer. In further aspects an identified shared neo-epitope is predicted to engage regulatory T cells and/or other detrimental T cells (including T cells with potential host cross-reactivity and/or anergic T cells) if the output JANUSMATRIX™ score for the neo-epitope is greater than or equal to 2 (and in further aspects, greater than or equal to 3).

In aspects, the method further comprises determining whether the identified neo-epitopes engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production. As described previously, in aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, upon activation, CD8+ Tregs may also exert immune suppressive effects through direct killing of target cells, characterized by the expression upon activation of effector molecules including but not limited to granzyme B and/or perforin.

In aspects of the method of identifying shared neo-epitopes for a shared neoplasia vaccine, the step of assessing the identified shared neo-epitopes encoded by said shared neoplasia-specific mutations to identify neo-epitopes that are known or determined (e.g. predicted) to engage regulatory T cells and/or other detrimental T cells (including T cells with potential host cross-reactivity and/or anergic T cells) comprises determining whether the identified neo-epitopes engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production. As described previously, in aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, upon activation, CD8⁺ Tregs may also exert immune suppressive effects through direct killing of target cells, characterized by the expression upon activation of effector molecules including but not limited to granzyme B and/or perforin. In aspects, cross-reactive or auto-reactive T cell responses will be tested by in vitro priming of T cells using neoepitope peptides containing non-synonymous amino acid substitutions and presented by autologous pAPC. This in vitro immunogenicity protocol may follow the methodology established by Wullner et al. (Wullner D, Zhou L, Bramhall E, Kuck A, Goletz T J, Swanson S, Chirmule N, Jawa V. Considerations for Optimization and Validation of an In vitro PBMC Derived T cell Assay for Immunogenicity Prediction of Biotherapeutics. Clin Immunol 2010 October; 137(1): 5-14, incorporated by reference in its entirety). T cells that expand following in vitro priming to the neoepitope peptides will then be tested for reactivity to the corresponding native or wild type (non-mutated) peptide epitopes. Reactivity to native peptide sequences will be determined by measuring cytokine production including, but not limited to, IFNγ, TNFα, IL-2 and/or markers of T cell effector function including, but not limited to, CD107a and granzyme B.

Designing of Peptides Comprising at Least One Identified Shared Neo-Epitope Encoded by Said Identified Shared Neoplasia-Specific Mutations

In aspects, the method further comprises designing at least one peptide or polypeptide as disclosed herein, said peptide or polypeptide comprising at least one identified shared neo-epitope encoded by said shared neoplasia-specific mutations, provided said shared neo-epitope is not identified in as being known or determined (e.g. predicted) to engage regulatory T cells and/or other detrimental T cells (including T cells with potential host cross-reactivity and/or anergic T cells). In aspects, the at least one designed peptide or polypeptide or a nucleic acid encoding said peptides or polypeptides can be used to produce an “off the shelf” pre-furnished shared neo-epitope warehouse. In aspects of the above-described peptides or polypeptides, the peptides or polypeptides may be isolated, synthetic, or recombinant. In aspects, the peptides or polypeptides can be capped with an N-terminal acetyl and C-terminal amino group. In aspects, the peptides or polypeptides can be either in neutral (uncharged) or salt forms, and may be either free of or include modifications such as glycosylation, side chain oxidation, or phosphorylation.

In even further aspects, the method further includes providing a vaccine comprising the at least designed or provided one peptide or polypeptide or nucleic acid provided. In aspects, the method includes providing a vaccine for a subject comprising the at least one designed or provided peptide or polypeptide or nucleic acid, wherein said share neo-epitopes are encoded by a shared neoplasia-specific mutation detected in a neoplasia sample from the subject and/or wherein said shared neo-epitopes are known or determined (e.g. predicted) to bind to a MHC protein of the subject. In aspects, the vaccine can be produced using the “off the shelf” pre-furnished shared neo-epitope warehouse. In aspects of the above-described peptides or polypeptides, the peptides or polypeptides may be isolated, synthetic, or recombinant.

Identifying Subject Specific Neo-Epitopes for the Subject-Specific Neoplasia Vaccine of Step (b)

In aspects of the above-described methods of inducing an immune response or method of treating a neoplasia (e.g., bladder cancer) in a subject in need thereof, the one or more identified subject-specific neo-epitopes for administration in step (b) are identified by a method comprising: i) identifying neoplasia-specific mutations in a neoplasia specimen of a subject diagnosed as having a neoplasia; ii) assessing the neoplasia-specific mutations identified in step (i) to identify known or determined (e.g. predicted) neo-epitopes encoded by said mutations, wherein said neo-epitopes are known or determined (e.g. predicted) to bind to a WIC protein of the subject; and iii) assessing the identified neo-epitopes encoded by said mutations from step (ii) to identify neo-epitopes that are known or determined (e.g. predicted) to engage regulatory T cells and/or other detrimental T cells (including T cells with potential host cross-reactivity and/or anergic T cells), and excluding such identified neo-epitopes that are known or predicted to engage regulatory T cells and/or other detrimental T cells (including T cells with potential host cross-reactivity and/or anergic T cells) from the subject-specific neo-epitopes for use in the personalized neoplasia vaccine. In aspects of the method of identifying subject-specific neo-epitopes for a personalized neoplasia vaccine, the method further includes: iv) designing at least one subject-specific peptide or polypeptide, said peptide or polypeptide comprising at least one identified neo-epitope encoded by said mutations, provided said neo-epitope is not identified in step (iii) as being known or determined (e.g. predicted) to engage regulatory T cells and/or other detrimental T cells (including T cells with potential host cross-reactivity and/or anergic T cells). In aspects, the method further includes: v) providing the at least one peptide or polypeptide designed in step (iv) or a nucleic acid encoding said peptides or polypeptides. In even further aspects, the method further includes vi) providing a vaccine comprising the at least one peptide or polypeptide or nucleic acid provided in step (v).

In aspects of the above-described methods, the one or more identified subject-specific neo-epitopes for administration in step (b) are identified by a method comprising: i) assessing identified neoplasia-specific mutations from a neoplasia specimen of a subject diagnosed as having a neoplasia identified to identify known or determined (e.g. predicted) neo-epitopes encoded by said mutations for use in the personalized neoplasia vaccine, wherein said neo-epitopes are known or determined (e.g. predicted) to bind to a WIC protein of the subject; and ii) assessing the identified neo-epitopes encoded by said mutations from step (i) to identify neo-epitopes that are known or determined (e.g. predicted) to engage regulatory T cells and/or other detrimental T cells (including T cells with potential host cross-reactivity and/or anergic T cells), and excluding such identified neo-epitopes that are known or determined (e.g. predicted) to engage regulatory T cells and/or other detrimental T cells (including T cells with potential host cross-reactivity and/or anergic T cells) from the subject-specific neo-epitopes for use in the personalized neoplasia vaccine. In aspects of the method of identifying subject-specific neo-epitopes for a personalized neoplasia vaccine, the method further includes: iii) designing at least one subject-specific peptide or polypeptide, said peptide or polypeptide comprising at least one identified neo-epitope encoded by said mutations, provided said neo-epitope is not identified in step (ii) as being known or determined (e.g. predicted) to engage regulatory T cells and/or other detrimental T cells (including T cells with potential host cross-reactivity and/or anergic T cells). In aspects, the method further includes: iv) providing the at least one peptide or polypeptide designed in step (iii) or a nucleic acid encoding said peptides or polypeptides. In even further aspects, the method further includes v) providing a vaccine comprising the at least one peptide or polypeptide or nucleic acid provided in step (iv).

Identifying Subject-Specific Neoplasia-Specific Mutations

In aspects, the step of identifying neoplasia-specific mutations (e.g., cancer-specific mutations) comprises sequencing genomic DNA and/or RNA of a neoplasia specimen (e.g., a neoplasia specimen of the patient). In aspects, a neoplasia specimen relates to any sample, such as a bodily sample derived from a patient, containing or being expected of containing neoplasia cells (e.g. tumor or cancer cells). In aspects, the bodily sample may be any tissue sample such as blood, a tissue sample obtained from a neoplasia sample (e.g., a primary tumor or from tumor metastases/circulating tumor cells), or any other sample containing neoplasia cells (e.g., tumor or cancer cells).

In aspects, the step of identifying of neoplasia-specific mutations comprises comparing the sequence information obtained from the neoplasia specimen with a reference sample, such as sequence information obtained from sequencing nucleic acid (e.g., such as DNA or RNA) of normal, non-neoplasia cells (e.g., non-cancerous cells) cells, such as somatic or germline tissue/cells. In aspects, a reference sample may be obtained from the neoplasia patient or a different individual. In aspects, a reference sample may be any tissue sample such as blood or a sample from a non-neoplasia tissue. In aspects, normal genomic germline DNA may be obtained from peripheral blood mononuclear cells (PBMCs).

In aspects, neoplasia-specific mutations may include all neoplasia-specific (e.g. cancer-specific) mutations present in one or more neoplasia cells (e.g., cancer or tumor cells) of a patient, or it may refer to only a portion of the neoplasia-specific mutations present in one or more neoplasia cells of a patient. Accordingly, the present invention may involve the identification of all neoplasia-specific mutations present in one or more neoplasia cells of a patient, or it may involve the identification of only a portion of the neoplasia-specific mutations present in one or more neoplasia cells of a patient. In aspects, the methods of identifying subject-specific neo-epitopes for a personalized neoplasia vaccine of the present invention provide for the identification of a number of neoplasia-specific mutations which will provide a sufficient number of neo-epitopes to be included in the instantly-disclosed strategies, methods, and compositions.

In aspects, the mutations are neoplasia-specific mutations (e.g., somatic mutations) in a neoplasia specimen (e.g. a tumor specimen) of a neoplasia patient (e.g. a cancer patient), which may be determined by identifying sequence differences between the genome, exome and/or transcriptome of a neoplasia specimen and the genome, exome and/or transcriptome of a non-neoplasia specimen. In aspects, neoplasia-specific mutations, including somatic mutations, are determined in the genome, preferably the entire genome, of a neoplasia specimen. As such, the instant invention may include identifying all or a portion of neoplasia-specific mutations of the genome, preferably the entire genome, of one or more neoplasia cells. In aspects, neoplasia-specific mutations, including somatic mutations, are determined in the exome, preferably the entire exome, of a neoplasia specimen. As such, the instant invention may include identifying all or a portion of neoplasia-specific mutations of the exome, preferably the entire exome of one or more neoplasia cells. In aspects, neoplasia-specific mutations, including somatic mutations, are determined in the transcriptome, preferably the entire transcriptome, of a neoplasia specimen. As such, the instant invention may include identifying all or a portion of the neoplasia-specific transcriptome, preferably the entire transcriptome, of one or more neoplasia cells.

In aspects, any suitable sequencing method as is known in the art can be used according to the instant invention for determining neoplasia-specific mutations is step (i), including but not limited to “conventional” sequencing methodology and Next Generation Sequencing (NGS) technologies. “Next Generation Sequencing” or “NGS” refers to all high throughput sequencing technologies which, in contrast to the “conventional” sequencing methodology known as Sanger chemistry, read nucleic acid templates randomly in parallel along the entire genome by breaking the entire genome into small pieces. As is known in the art, such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are known in the art can be used. Non-limiting examples of such NGS technologies/platforms include, but are not limited to sequencing-by-ligation approaches, ion semiconductor sequencing, pyrosequencing, single-molecule sequencing technologies, nano-technologies for single-molecule sequencing, and electron microscopy based technologies for single-molecule sequencing. Further, in aspects, “Third Generation Sequencing” methods, as are known in the art, could be used for determining neoplasia-specific mutations. In aspects, neoplasia-specific mutations may be determined by direct protein sequencing techniques, as are known in the art. Further, in aspects, neoplasia-specific mutations can be determined by using MEW multimers, as is known in the art.

As such, in aspects of the method of identifying subject-specific neo-epitopes for a personalized neoplasia vaccine, the step of identifying neoplasia-specific mutations includes identifying sequence differences between the full or partial genome, exome, and/or transcriptome of a neoplasia specimen from the subject diagnosed as having a neoplasia and a non-neoplasia specimen. In aspects, a non-neoplasia specimen is derived from the subject diagnosed as having a neoplasia. In further aspects, identifying neoplasia-specific mutations or identifying sequence differences comprises Next Generation Sequencing (NGS). In aspects, the step of identifying neoplasia-specific mutations comprises selecting from the neoplasia a plurality of nucleic acid sequences, each comprising mutations not present in a non-neoplasia sample. In aspects, identifying neoplasia-specific mutations comprises sequencing genomic DNA and/or RNA of the neoplasia specimen.

In aspects of the method of identifying subject-specific neo-epitopes for a personalized neoplasia vaccine, the neoplasia-specific mutations are neoplasia-specific somatic mutations. In aspects, the neoplasia-specific mutations are single nucleotide variations (SNVs), insertions and deletions (which can generate both in-frame and frameshift mutations), and other large-scale rearrangements such as but not limited to chromosomal inversions, duplications, insertions, deletions, or translocations. In aspects, neoplasia specific mutations, including SNVs, insertions, and deletions, are non-synonymous mutations. In aspects, neoplasia-specific mutations, including SNVs, insertions and deletions (which can be non-synonymous mutations), and other large-scale rearrangements, are mutations of proteins encoded in the neoplasia specimen of the subject diagnosed as having a neoplasia. In aspects, neoplasia specific mutations, including SNVs, are non-synonymous mutations. In aspects, neoplasia-specific mutations, including SNVs (which can be non-synonymous mutations), indels, and frameshifts, are mutations of proteins encoded in the neoplasia specimen of the subject diagnosed as having a neoplasia.

Identifying Subject-Specific Neo Epitopes

In aspects of the method of identifying subject-specific neo-epitopes for a personalized neoplasia vaccine, the step of assessing the neoplasia-specific mutations to identify known or determined (e.g. predicted) neo-epitopes encoded by said mutations comprises in silico testing. In aspects, in silico testing includes using validated algorithms (e.g., but not limited to, EPIMATRIX®, netMHCpan, NetMHC, netMHCcons, SYFPEITHI, HLA_BIND) to predict which neoplasia-specific mutations create neo-epitopes, particularly neo-epitopes that can bind to the MHC allotypes of the patient. For example, using validated algorithms, bioinformatic analysis of the identified neoplasia-specific mutations and their respective cognate native antigens can be performed to predict which identified neoplasia-specific mutations create neo-epitopes that can bind to the patient's MHC allotype, and in aspects to predict which identified neoplasia-specific mutations create neo-epitopes that could bind to the patient's MHC allotype more effectively than the cognate native antigen. Thus, in aspects, assessing identified neoplasia-specific mutations from a neoplasia specimen of a subject diagnosed as having a neoplasia identified to identify known or determined (e.g. predicted) neo-epitopes encoded by said mutations for use in the personalized neoplasia vaccine, wherein said neo-epitopes are known or determined (e.g. predicted) to bind to a MEW protein of the subject comprises the use of well-validated algorithms.

In aspects, said in silico testing to identify known or determined (e.g. predicted) neo-epitopes encoded by said mutations comprises using the EPIMATRIX® algorithm. EPIMATRIX® is a proprietary computer algorithm developed by EpiVax, which is used to screen protein sequences for the presence of putative T cell epitopes. The algorithm uses matrices for prediction of 9- and 10-mer peptides binding to MHC molecules. Each matrix is based on position-specific coefficients related to amino acid binding affinities that are elucidated by a method similar to, but not identical to, the pocket profile method (Sturniolo, T. et al., Nat. Biotechnol., 17:555-561, 1999). Input sequences are parsed into overlapping 9-mer or 10-mer frames where each frame overlaps the last by 8 or 9 amino acids, respectively. Thus, in aspects, input sequences of the mutated peptide and the non-mutated peptide are parsed into overlapping 9-mer or 10-mer frames where each frame overlaps the last by 8 or 9 amino acids. Each of the resulting frames form the mutated peptide and the non-mutated peptide are then scored for predicted binding affinity with respect to MHC class I alleles (e.g., but not limited to, HLA-A and HLA-B alleles) and MEW class II alleles (e.g., but not limited to HLA-DRB1 alleles). EPIMATRIX® raw scores are normalized against the scores of a large sample of randomly generated peptides (e.g., but not limited to 10,000 randomly generated peptides). The resulting “Z” scores are normally distributed and directly comparable across alleles. The resulting “Z” score is reported. In aspects, any 9-mer or 10-mer peptide with an allele-specific EPIMATRIX® Z-score that is theoretically the top 5% of any given sample (e.g., having an EPIMATRIX® Z-score above 1.64), is considered a putative T cell epitope. In aspects, EPIMATRIX® identifies the mutated peptide as a neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide. Previous studies have also demonstrated that EPIMATRIX® accurately predicts published MHC ligands and T cell epitopes.

In aspects of the method of identifying subject-specific neo-epitopes for a personalized neoplasia vaccine, assessing the neoplasia-specific mutations to identify known or determined (e.g. predicted) neo-epitopes encoded by said mutations includes one or more of the following steps:

a) determining a binding score for a mutated peptide to one or more MHC molecules, wherein said mutated peptide is encoded by at least one of said neoplasia-specific mutations;

b) determining a binding score for a non-mutated peptide to the one or more MHC molecules, wherein the non-mutated peptide is identical to the mutated peptide except for the encoded at least one of said neoplasia-specific mutations;

c) determining the percentile rank of the binding scores of both the mutated peptide of step (a) and the non-mutated peptide of step (b) as compared to an expected distribution of binding scores for sufficiently large enough set (e.g., at least 10,000) randomly generated peptides using naturally observed amino acid frequencies;

d) determining the TCR facing amino acid residues of said mutated peptide and said non-mutated peptide; and

e) identifying the mutated peptide as a neo-epitope when: 1) the mutated peptide has a determined binding score in the top 5 percentile of the expected distribution and the non-mutated peptide has a determined binding score below the top 10 percentile of the expected distribution; or 2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide. In further aspects, the one or more MHC molecules are MHC class I molecules and/or MHC class II molecules.

In aspects of the method of identifying subject-specific neo-epitopes for a personalized neoplasia vaccine, the step of assessing the neoplasia-specific mutations to identify known or determined (e.g. predicted) neo-epitopes encoded by said mutations comprises in vitro testing. More particularly, determining the binding score of both the mutated peptide of step (a) and the non-mutated peptide of step (b) may comprise in vitro MHC binding assays (as are known in the art) to determine a binding score for the mutated peptide to one or more MHC molecules and to determine a binding score for the non-mutated peptide to the one or more MHC molecules. In aspects, and similar to an in silico analysis, input sequences are parsed into overlapping 9-mer or 10-mer frames where each frame overlaps the last by 8 or 9 amino acids, respectively. Thus, in aspects, input sequences of the mutated peptide from step (a) and the non-mutated peptide from step (b) are parsed into overlapping 9-mer or 10-mer frames where each frame overlaps the last by 8 or 9 amino acids. Each of the resulting frames from the mutated peptide from step (a) and the non-mutated peptide from step (b) are then scored for binding affinity with respect to MEW class I alleles (e.g., but not limited to, HLA-A and HLA-B alleles) in in vitro binding assays, with such binding assays as are known in the art. In the case of testing for epitopes that bind MEW class II alleles (e.g., but not limited to HLA-DRB1 alleles) in in vitro binding assays, input sequences are parsed into overlapping 15-mer or 20-mer frames where each frame overlaps the last by 5 or 10 amino acids, respectively. Thus, in aspects, input sequences of the mutated peptide from step (a) and the non-mutated peptide from step (b) are parsed into overlapping 15-mer or 20-mer frames where each frame overlaps the last by 5 or 10 amino acids. Each of the resulting frames from the mutated peptide from step (a) and the non-mutated peptide from step (b) are then scored for binding affinity with respect to MHC class II alleles (e.g., but not limited to HLA-DRB1 alleles) in in vitro binding assays, with such binding assays as are known in the art.

In aspects of the method of identifying subject-specific neo-epitopes for a personalized neoplasia vaccine, the step of determining the percentile rank of the binding scores of both the mutated peptide of step (a) and the non-mutated peptide of step (b) as compared to an expected distribution of binding scores for a sufficiently large enough set (e.g., at least 10,000) randomly generated peptides using naturally observed amino acid frequencies, the raw binding scores, whether determined by in silico methods or in vitro methods, are adjusted to fit a normal, or Z-distribution. Raw binding scores are normalized based on the average (μ) binding score and standard deviation (σ) of a set of a large number (e.g., 10,000) random 9- or 10-mer amino acid sequences, following the naturally observed amino acid frequencies from UniProtKB/Swiss-Prot, as follows:

“Normalized binding score”=(“Raw binding score”−μ)/σ.

Normalized binding scores, which may be referred to as binding scores or likelihood of binding, within the top 5% of this normal distribution are defined as “hits”; which are potentially immunogenic and worthy of further consideration. These peptides have a significant chance of binding to MHC molecules with moderate to high affinity and, therefore, have a significant chance of being presented on the surface of both professional antigen presenting cells (APC) such as dendritic cells or macrophages, as well as non-professional APC, where they may be interrogated and potentially bound by passing CD8+ and CD4+ T cells.

In aspects, the mutated peptide and non-mutated peptide are both 9 amino acids in length or the mutated peptide and non-mutated peptide are both 10 amino acids in length. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at position 2, 3, 5, 7, and 8 of the mutated and non-mutated peptide as counted from the amino terminal. In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at position 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at position 4, 5, 6, 7, 8, and 9 of the mutated and non-mutated peptide as counted from the amino terminal. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at any combination of residues at positions 2, 3, 5, 7, and 8 (e.g., but not limited to, positions 3, 5, 7 and 8; positions 2, 5, 7, and 8; positions 2, 3, 5, and 7, etc. of the mutated and non-mutated peptide as counted from the amino terminal. In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at positions 4, 5, 6, 7, and 8; 1, 4, 5, 6, 7 and 8; or 1, 3, 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at any combination of residues at positions 1, 3, 4, 5, 6, 7, 8, and 9 of the mutated and non-mutated peptide as counted from the amino terminal.

In aspects of the method of identifying subject-specific neo-epitopes for a personalized neoplasia vaccine, particularly assessing the neoplasia-specific mutations to identify known or determined (e.g. predicted) neo-epitopes, the identified neo-epitopes may be optionally further confirmed by experimental validation for peptide-MHC binding, activation of CD8+ and/or CD4+ T cells, and/or by confirmation of gene expression at the RNA level. Such experimental validation may comprise in vitro and/or in vivo techniques, as are known in the art.

Identification and Removal of Neo-Epitopes that are Known or Determined (e.g. Predicted) to Engage Regulatory T Cells and/or Other Detrimental T Cells

As previously described, the instantly-disclosed data suggest the possibility that tumor-derived neo-epitopes may be recruiting regulatory T cells and/or other detrimental T cells (including T cells with potential host cross-reactivity and/or anergic T cells) to the tumor. As such, the inadvertent inclusion of regulatory T cell-driving neo-epitopes and/or other detrimental T cell-driving neo-epitopes (including T cells with potential host cross-reactivity and/or anergic T cells) in vaccine formulations may hinder efforts to induce strong T cell-mediated tumor control. Screening of neoantigen sequences to identify and remove potential regulatory T cell inducing neo-epitopes and/or other detrimental T cell inducing neo-epitopes (e.g., using specialized tools, including in silico screening tools) may be critical to designing new vaccines with higher quality candidates.

As such, in aspects of the method of identifying subject-specific neo-epitopes for a personalized neoplasia vaccine, the step of assessing the identified neo-epitopes encoded by said mutations to identify neo-epitopes that are known or determined (e.g. predicted) to engage (e.g. bind and activate) regulatory T cells and/or other detrimental T cells (including T cells with potential host cross-reactivity and/or anergic T cells) comprises determining whether said identified neo-epitopes encoded by said mutations share TCR contacts with proteins derived from either the human proteome or the human microbiome, wherein said identified neo-epitopes encoded by said mutations that are determined to share TCR contacts with proteins derived from either the human proteome or the human microbiome are identified as neo-epitopes that are known or determined (e.g. predicted) to engage regulatory T cells and/or other detrimental T cells (including T cells with potential host cross-reactivity and/or anergic T cells). In aspects, TCR contacts for a 9-mer identified neo-epitope that bind to a MHC class II molecule are at position 2, 3, 5, 7, and 8 of the identified neo-epitope as counted from the amino terminal, wherein the TCR contacts for a 9-mer identified neo-epitope that binds to a MHC class I molecule are at position 4, 5, 6, 7, and 8 of the identified neo-epitope as counted from the amino terminal, and wherein the TCR contacts for a 10-mer identified neo-epitope that bind to a MHC class I molecule are at position 4, 5, 6, 7, 8, and 9 of the identified neo-epitope as counted from the amino terminal. In aspects, the step of determining the TCR facing amino acid residues of said mutated peptide and said non-mutated peptide the TCR facing amino acid residues for a 9-mer mutated peptide and a 9-mer non-mutated peptide that bind to a MHC class II molecule comprises identifying the amino acid residues which are at any combination of residues at positions 2, 3, 5, 7, and 8 (e.g., but not limited to, positions 3, 5, 7 and 8; positions 2, 5, 7, and 8; positions 2, 3, 5, and 7, etc. of the mutated and non-mutated peptide as counted from the amino terminal). In aspects, the step of determining the TCR facing amino acid residues of said mutated peptide and said non-mutated peptide for a 9-mer mutated peptide and a 9-mer non-mutated peptide that bind to a MHC class I molecule comprises identifying the amino acid residues which are at positions 4, 5, 6, 7, and 8; 1, 4, 5, 6, 7 and 8; or 1, 3, 4, 5, 6, 7, and 8 of the mutated and non-mutated peptide as counted from the amino terminal. In aspects, the step of determining the TCR facing amino acid residues of said mutated peptide and said non-mutated peptide for a 10-mer mutated peptide and 10-mer non-mutated peptide that bind to a MHC class I molecule comprises identifying the amino acid residues which are at any combination of residues at positions 1, 3, 4, 5, 6, 7, 8, and 9 of the mutated and non-mutated peptide as counted from the amino terminal.

In aspects, the step of assessing the identified neo-epitopes encoded by said mutations to identify neo-epitopes that are known or determined (e.g. predicted) to engage regulatory T cells comprises conducting a homology screen on each identified neo-epitope or epitope sequence presenting a high likelihood of binding to MHC in order to characterize the degree of similarity with self of each of the encoded MHC class I- and MHC class II-restricted identified neo-epitopes and their corresponding non-mutated epitopes. WIC class I or WIC class II neo-epitopes and WIC class I or MHC class II corresponding non-mutated epitopes with two or more (and in further aspects, three or more) cross-reactive matches in the reference proteome are categorized as exhibiting a high degree of similarity with self and are considered to have a higher likelihood of being tolerated or to engage regulatory T cells and/or other detrimental T cells (including T cells with potential host cross-reactivity and/or anergic T cells).

In aspects, a homology screen is used remove epitopes containing combinations of TCR-facing residues that are commonly found in a reference proteome. In aspects, a homology screen comprises analysis of all the predicted epitopes contained within a given protein sequence and dividing each predicted epitope into its constituent amino acid content of both the MHC-binding agretope and the TCR-binding epitope. In aspects, the TCR-binding epitope (which can be referred to as TCR binding residues, TCR facing epitope, TCR facing residues, or TCR contacts) for a 9-mer identified neo-epitope or epitope that bind to a MHC class II molecule are at position 2, 3, 5, 7, and 8 of the identified neo-epitope, while the MHC-binding agretope (which can be referred to as MHC contacts, WIC facing residues, WIC-binding residues, or MHC-binding face) for a 9-mer identified neo-epitope or epitope that bind to a WIC class II molecule are at position 1, 4, 6, and 9, both as counted from the amino terminal. In aspects, the TCR binding epitope for a 9-mer identified neo-epitope that binds to a MHC class I molecule are at position 4, 5, 6, 7, and 8 of the identified neo-epitope or epitope, while the WIC binding agretope for a 9-mer identified neo-epitope or epitope that bind to a WIC class I molecule are at position 1, 2, 3, and 9, both as counted from the amino terminal. In aspects, the TCR binding epitope for a 10-mer identified neo-epitope that bind to a WIC class I molecule are at position 4, 5, 6, 7, 8, and 9 of the identified neo-epitope, while the WIC binding agretope for a 10-mer identified neo-epitope or epitope that bind to a WIC class I molecule are at position 1, 2, 3, 9, and 10, both as counted from the amino terminal. In aspects, the TCR-binding epitope for a 9-mer identified neo-epitope or epitope that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified neo-epitope or epitope, while the WIC binding agretope for a 9-mer identified neo-epitope or epitope is the complementary face to the TCR facing residues, both as counted from the amino terminal. In aspects, the TCR binding epitope for 9-mer identified neo-epitope or epitope that bind to a MEW class I molecule are at positions 4, 5, 6, 7, and 8; 1, 4, 5, 6, 7 and 8; or 1, 3, 4, 5, 6, 7, and 8 of the identified neo-epitope or epitope, while the MEW binding agretope for a 9-mer identified neo-epitope or epitope is the complementary face to the TCR facing residues, both as counted from the amino terminal. In aspects, the TCR-binding epitope for a 10-mer identified neo-epitope or epitope that bind to a MEW class I molecule are at any combination of residues at positions 1, 3, 4, 5, 6, 7, 8, and 9 of the identified neo-epitope or epitope, while the MEW binding agretope for a 10-mer identified neo-epitope or epitope is the complementary face to the TCR facing residues, both as counted from the amino terminal.

Each sequence is then screened against a database of proteins (e.g., a database of human proteins derived from the UniProt database (UniProt Proteome ID UP000005640, Reviewed/Swiss-Prot set)). Cross-conserved epitopes, or peptides derived from the reference proteome with a compatible MEW binding agretope (i.e. the agretopes of both the input (mutated) peptide and its reference non-mutated counterpart are predicted to bind to the same MHC allele) and exactly the same TCR facing epitope, are returned. The Homology Score of an epitope corresponds to the number of matching cross-conserved MEW binding peptides within the reference proteome. In other words, the Homology Score Hₑ of an epitope e is calculated as follows:

Hₑ=|Xₑ|,

where:

Xₑ corresponds to the set of MHC binding peptides derived from the reference proteome that are restricted to the same MHC class I or MHC class II as epitope e and presenting a TCR facing epitope identical to the epitope e.

By extension, the Homology Score of a given peptide or protein corresponds to the average Homology Score of each individual epitope contained with the peptide or protein. In other words, the Homology Score H_p of a peptide p is calculated as follows:

$${H}_{p} = \frac{{Σ}_{e ∈ E} {H}_{e}}{\vert E \vert}$$

where:
E corresponds to the set of MHC class I- or MHC class II-restricted epitopes within peptide p;
Hₑ corresponds to the Homology Score of epitope e as defined above.

In aspects, an analysis procedure is then run on each mutated sequence to determine if a substring within the amino acid sequence can be found, such that
at least one MHC class I- or MHC class II-restricted epitope is encoded in the substring;
all MHC class I- or MHC class II-restricted neo-epitopes encoded in the substring have no more than two cross-reactive matches in the reference proteome, and;
all MHC class I- or MHC class II-restricted epitopes encoded in the substring have no more than two cross-reactive matches in the reference proteome.

This analysis procedure has the effect of removing amino acid substrings containing putative epitopes that engage regulatory T cells, other detrimental T cells (including T cells with potential host cross-reactivity and/or anergic T cells), and other highly cross-conserved epitopes from the identified neo-epitope sequences. The resulting sequences will only contain epitopes or neo-epitopes that exhibit low degree of similarity with self-sequences. Neo-epitope sequences are discarded from consideration for use in a personalized neoplasia-specific vaccine if no substring matching the above criteria can be found. Conversely, the same homology analysis can be performed against a set of known infectious disease-derived epitopes known to be immunogenic, extracted for example from the IEDB database, or against a set of other known immunogenic sequences or common pathogen-derived sequences. This analysis has the purpose of identifying neo-epitope candidates that share a high degree of homology with other known or putative effector T cell epitopes. Neoantigens containing such neo-epitopes can be prioritized in vaccine formulations.

In aspects of the method of identifying subject-specific neo-epitopes for a personalized neoplasia vaccine, the step of assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) comprises in silico testing. In aspects, in silico testing comprises analyzing whether the identified neo-epitopes are predicted to engage regulatory T cells and/or other detrimental T cells (including T cells with potential host cross-reactivity and/or anergic T cells) using the JANUSMATRIX™ algorithm. JANUSMATRIX™ is a homology analysis tool that compares putative T cell epitopes and their TCR-facing residues across genome sequences rather than linear peptide fragments, and thus considers aspects of antigen recognition that are not captured by raw sequence alignment. In aspects, JANUSMATRIX™ parses the epitopes into 9-mer frames or 10-mer frames and divides each 9-mer or 10-mer into the WIC-binding agretope and the TCR-binding epitope. In aspects, the TCR-binding epitope (which can be referred to as TCR binding residues, TCR facing epitope, TCR facing residues, or TCR contacts) for a 9-mer identified neo-epitope or epitope that bind to a WIC class II molecule are at position 2, 3, 5, 7, and 8 of the identified neo-epitope, while the WIC-binding agretope (which can be referred to as WIC contacts, WIC facing residues, WIC-binding residues, or MHC-binding face) for a 9-mer identified neo-epitope or epitope that bind to a MHC class II molecule are at position 1, 4, 6, and 9, both as counted from the amino terminal. In aspects, the TCR binding epitope for a 9-mer identified neo-epitope that binds to a WIC class I molecule are at position 4, 5, 6, 7, and 8 of the identified neo-epitope or epitope, while the MHC binding agretope for a 9-mer identified neo-epitope or epitope that bind to a WIC class I molecule are at position 1, 2, 3, and 9, both as counted from the amino terminal. In aspects, the TCR binding epitope for a 10-mer identified neo-epitope that bind to a MHC class I molecule are at position 4, 5, 6, 7, 8, and 9 of the identified neo-epitope, while the WIC binding agretope for a 10-mer identified neo-epitope or epitope that bind to a MHC class I molecule are at position 1, 2, 3, 9, and 10, both as counted from the amino terminal. In aspects, the TCR-binding epitope for a 9-mer identified neo-epitope or epitope that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified neo-epitope or epitope, while the MHC binding agretope for a 9-mer identified neo-epitope or epitope is the complementary face to the TCR facing residues, both as counted from the amino terminal. In aspects, the TCR binding epitope for 9-mer identified neo-epitope or epitope that bind to a WIC class I molecule are at positions 4, 5, 6, 7, and 8; 1, 4, 5, 6, 7 and 8; or 1, 3, 4, 5, 6, 7, and 8 of the identified neo-epitope or epitope, while the WIC binding agretope for a 9-mer identified neo-epitope or epitope is the complementary face to the TCR facing residues, both as counted from the amino terminal. In aspects, the TCR-binding epitope for a 10-mer identified neo-epitope or epitope that bind to a WIC class I molecule are at any combination of residues at positions 1, 3, 4, 5, 6, 7, 8, and 9 of the identified neo-epitope or epitope, while the MHC binding agretope for a 10-mer identified neo-epitope or epitope is the complementary face to the TCR facing residues, both as counted from the amino terminal. JANUSMATRIX™ then searches for potentially cross-reactive TCR-facing epitopes across any number of large sequence databases that have been pre-loaded into the tool, including the protein sequences from bacterial and viral organisms that make up the gut microbiome (e.g., the human gut microbiome), autologous proteins from the genome (e.g., the human genome), and viral and bacterial pathogens (e.g., human viral and human bacterial pathogens). JANUSMATRIX™ focuses in 9-mer and/or 10-mer searches because although peptides of different lengths interact with the MHC, most T cell epitopes can be mapped to a minimum of nine or ten amino acids in any given peptide, even if the peptide is longer. In further aspects an identified neo-epitope is predicted to engage engage regulatory T cells and/or other detrimental T cells (including T cells with potential host cross-reactivity and/or anergic T cells) if the output JANUSMATRIX™ score for the neo-epitope is greater than or equal to 2 (and in further aspects, greater than or equal to 3).

In aspects, the method further comprises determining whether the identified neo-epitopes engage engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production. As described previously, in aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, upon activation, CD8⁺ Tregs may also exert immune suppressive effects through direct killing of target cells, characterized by the expression upon activation of effector molecules including but not limited to granzyme B and/or perforin.

In aspects of the method of identifying subject-specific neo-epitopes for a personalized neoplasia vaccine, the step of assessing the identified neo-epitopes encoded by said mutations to identify neo-epitopes that are known or determined (e.g. predicted) to engage regulatory T cells and/or other detrimental T cells (including T cells with potential host cross-reactivity and/or anergic T cells) comprises determining whether the identified neo-epitopes engage regulatory T cells and/or other detrimental T cells (including T cells with potential host cross-reactivity and/or anergic T cells) in vitro. In aspects, a neo-epitope is determined to engage regulatory T cells when said neo-epitope results in regulatory T cell activation, proliferation, and/or IL-10 or TGF-β production. As described previously, in aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, upon activation, CD8⁺ Tregs may also exert immune suppressive effects through direct killing of target cells, characterized by the expression upon activation of effector molecules including but not limited to granzyme B and/or perforin. In aspects, cross-reactive or auto-reactive T cell responses can be tested by in vitro priming of T cells using neoepitope peptides containing non-synonymous amino acid substitutions and presented by autologous pAPC. This in vitro immunogenicity protocol may follow the methodology established by Wullner et al. (Wullner D, Zhou L, Bramhall E, Kuck A, Goletz T J, Swanson S, Chirmule N, Jawa V. Considerations for Optimization and Validation of an In vitro PBMC Derived T cell Assay for Immunogenicity Prediction of Biotherapeutics. Clin Immunol 2010 October; 137(1): 5-14, incorporated by reference in its entirety). T cells that expand following in vitro priming to the neoepitope peptides will then be tested for reactivity to the corresponding native or wild type (non-mutated) peptide epitopes. Reactivity to native peptide sequences will be determined by measuring cytokine production including, but not limited to, IFNg, TNFa, IL-2 and/or markers of T cell effector function including, but not limited to, CD107a and granzyme B.

Ranking Polypeptides Comprising Subject-Specific Neo Epitopes Encoded by Said Identified Neoplasia-Specific Mutations

In aspects of the method of identifying subject-specific neo-epitopes for a personalized neoplasia vaccine, the method further comprises ranking peptides or polypeptides comprising said identified subject-specific neo-epitopes, provided said neo-epitope is not identified as being known or determined (e.g. predicted) to engage regulatory T cells and/or other detrimental T cells (including T cells with potential host cross-reactivity and/or anergic T cells), for their usability in an immunogenic composition, such as a personalized neoplasia vaccine. As such, in aspects, the identified subject-specific peptides or polypeptides comprise at least one identified neo-epitope encoded by said identified neoplasia-specific mutations, provided said neo-epitope is not identified as being known or determined (e.g. predicted) to engage regulatory T cells and/or other detrimental T cells (including T cells with potential host cross-reactivity and/or anergic T cells), are ranked for their usability as epitopes in an immunogenic composition, such as a personalized neoplasia vaccine. In aspects, the methods comprise a manual or computer-based analytical process in which peptides or polypeptides comprising the identified subject-specific neo-epitopes are analyzed and selected for their usability in the respective vaccine to be provided. In aspects, said analytical process is a computational algorithm-based process. Preferably, said analytical process comprises determining and/or ranking peptides or polypeptides comprising subject-specific neo-epitopes according to a determination (e.g., prediction) of their capacity of being immunogenic.

In aspects, comprises ranking peptides or polypeptides comprising said identified subject-specific neo-epitopes for their usability as epitopes in an immunogenic composition, such as a personalized neoplasia vaccine comprises determining (e.g., predicting) one or more characteristics associated with the peptides or polypeptides comprising identified subject-specific neo-epitopes, the characteristics including immunogenicity-related features, sequencing-related features, and/or physiochemical-related features.

In aspects, determined immunogenicity-related features of the peptides or polypeptides comprising identified subject-specific neo-epitopes may include one of more of: count of MHC class I neo-epitopes; percentile ranks of the MHC class I neo-epitopes; MHC class I-restricted regulatory T cell and/or other detrimental T cells (including T cells with potential host cross-reactivity and/or anergic T cells) induction potential of the neoantigen (e.g. peptide or polypeptide comprising one or more identified neo-epitopes); Count of MHC class II neo-epitopes; percentile ranks of the MHC class II neo-epitopes; MHC class II-restricted regulatory T cell and/or other detrimental T cells (including T cells with potential host cross-reactivity and/or anergic T cells) induction potential of the neoantigen (e.g. peptide or polypeptide comprising one or more identified neo-epitopes); and/or whether the optimized neoantigen (e.g. peptide or polypeptide comprising one or more identified neo-epitopes)contains both MHC class I and II neo-epitopes.

In aspects, determined sequencing-related features of the peptides or polypeptides comprising identified subject-specific neo-epitopes may include one of more of: expression level of the associated transcript; coverage of the mutation in the tumor DNA, i.e., the number of unique sequencing reads that overlap the genomic position of the mutation; variant allele fraction (VAF) of the mutation in the tumor DNA, i.e., the relative frequency, from 0 to 1, of the observed mutation across sequencing reads; and/or other sequencing metadata, as may be needed.

In aspects, determined physiochemical-related features of the peptides or polypeptides comprising identified subject-specific neo-epitopes may include one of more of: net charge of the optimized neoantigen (e.g. peptide or polypeptide comprising one or more identified neo-epitopes); whether the optimized neoantigen (e.g. peptide or polypeptide comprising one or more identified neo-epitopes) contains at least one charged residue; the count of cysteines (C) within the optimized neoantigen (e.g. peptide or polypeptide comprising one or more identified neo-epitopes); whether the optimized neoantigen (e.g. peptide or polypeptide comprising one or more identified neo-epitopes)contains at least one cysteine (C) and is negatively charged; whether the optimized neoantigen (e.g. peptide or polypeptide comprising one or more identified neo-epitopes) contains a poly-proline motif (‘PP’); whether the optimized neoantigen (e.g. peptide or polypeptide comprising one or more identified neo-epitopes)contains at least one methionine (M); whether the optimized neoantigen (e.g. peptide or polypeptide comprising one or more identified neo-epitopes)contains an N-terminal glutamine (Q); whether the optimized neoantigen (e.g. peptide or polypeptide comprising one or more identified neo-epitopes)contains a glycine (G) and/or proline (P) in the last or second to last positions; whether the optimized neoantigen (e.g. peptide or polypeptide comprising one or more identified neo-epitopes)contains a ‘DG’, DS′, ‘DA’, or ‘DN’ motif; and/or the hydropathy index of the optimized neoantigen (e.g. peptide or polypeptide comprising one or more identified neo-epitopes).

In aspects, the method further comprises ranking, based on the determined characteristics, each of the peptides or polypeptides comprising identified subject-specific neo-epitopes. In aspects, the top 5-30, including every value and range therein, ranked peptides or polypeptides comprising identified subject-specific neo-epitopes are included in the personalized neoplasia vaccine. In aspects, the peptides or polypeptides comprising identified subject-specific neo-epitopes are scored and ranked according to the ranking scheme disclosed in Example 1.

Designing of Subject-Specific Peptides Comprising at Least One Identified Neo Epitope Encoded by Said Identified Neoplasia-Specific Mutations

In aspects of the method of identifying subject-specific neo-epitopes for a personalized neoplasia vaccine, the method further comprises designing at least one subject-specific peptide or polypeptide, said peptide or polypeptide comprising at least one identified neo-epitope encoded by said mutations, provided said neo-epitope is not identified in as being known or determined (e.g. predicted) to engage regulatory T cells and/or other detrimental T cells (including T cells with potential host cross-reactivity and/or anergic T cells).

In aspects of the method of identifying subject-specific neo-epitopes for a personalized neoplasia vaccine, the method further includes: iv) designing at least one subject-specific peptide or polypeptide, said peptide or polypeptide comprising at least one identified neo-epitope encoded by said mutations, provided said neo-epitope is not identified in step (iii) as being known or determined (e.g. predicted) to engage regulatory T cells and/or other detrimental T cells (including T cells with potential host cross-reactivity and/or anergic T cells). In aspects, the method further includes v) providing the at least one peptide or polypeptide designed in step (iv) or a nucleic acid encoding said peptides or polypeptides. In even further aspects, the method further includes vi) providing a vaccine comprising the at least one peptide or polypeptide or nucleic acid provided in step (v).

As used herein, the term “treating” relates to any treatment of a neoplasia (e.g. cancer or a solid tumor, such as bladder cancer), including but not limited to prophylactic treatment and therapeutic treatment. “Treating” includes any effect, e.g., preventing, lessening, reducing, modulating, or eliminating, that results in the improvement of the neoplasia For example, “treating” or “treatment” of a cancer state includes: inhibiting the cancer, i.e., arresting the development of the cancer or its clinical symptoms; or relieving the cancer, i.e., causing temporary or permanent regression of the cancer or its clinical symptoms. “Prevent,” “preventing,” “prevention,” “prophylactic treatment,” and the like, refer to reducing the probability of developing a disease or condition in a subject, who does not have, but is at risk of or susceptible to developing a disease or condition.

A “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In certain aspects of the methods disclosed herein, the subject that is administered an effective amount is a mammal, and more particularly a human.

An “effective amount” is defined herein in relation to the treatment of neoplasia (e.g., a cancer or a solid tumor, such as bladder cancer) is an amount that will decrease, reduce, inhibit, or otherwise abrogate the growth of a neoplasia (e.g. a cancer cell or tumor). The “effective amount” will vary depending the neoplasia and its severity and the age, weight, etc., of the mammal to be treated. The amount, as well as timing and dosing schedule, of a compositions of the present invention administered to the subject will depend on the type and severity of the disease and on the characteristics of the individual, such as general health, age, sex, body weight and tolerance to drugs. It will also depend on the degree, severity and type of neoplasia disease. The skilled artisan will be able to determine appropriate dosages and dosage scheduling depending on these and other factors.

In some aspects of the methods of inducing an immune response or method of treating a neoplasia (e.g., bladder cancer) in a subject in need thereof, the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes, or pharmaceutical compositions can be delivered regionally to a particular affected region or regions of the subject's body. In some embodiments, the presently-disclosed peptides or polypeptides or pharmaceutical compositions can be administered systemically. For example, in some embodiments of a method treating cancer in a subject in need of treatment thereof, the presently-disclosed peptides or polypeptides or pharmaceutical compositions are administered orally. In accordance with the presently disclosed methods, the presently-disclosed peptides or polypeptides or pharmaceutical compositions can be administered orally as a solid or as a liquid. In other embodiments, the presently-disclosed peptides or polypeptides or pharmaceutical compositions are administered intravenously. In accordance with the presently disclosed methods, the presently-disclosed peptides or polypeptides or pharmaceutical compositions can be administered intravenously as a solution, suspension, or emulsion. Alternatively, the presently-disclosed peptides or polypeptides or pharmaceutical compositions also can be administered by inhalation, intravenously, or intramuscularly as a liposomal suspension.

The compositions of the present invention can also be administered in combination with one or more additional therapeutic compounds. Thus, in some aspects the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes or pharmaceutical compositions, are administered with one or more additional therapeutic compounds. It will be appreciated that therapeutic benefits for the treatment of cancer can be realized by combining treatment with the presently-disclosed peptides or polypeptides with one or more additional therapeutic compounds. The term “additional therapeutic compounds” includes other anti-cancer agents or treatments. The choice of such combinations will depend on various factors including, but not limited to, the type of disease, the age and general health of the subject, the aggressiveness of disease progression, and the ability of the subject to tolerate the agents that comprise the combination. For example, the presently-disclosed peptides or polypeptides or pharmaceutical compositions can be combined with other agents and therapeutic regimens that are effective at reducing tumor size (e.g., radiation, surgery, chemotherapy, hormonal treatments, and or gene therapy). Further, in some embodiments, it can be desirable to combine the presently-disclosed peptides or polypeptides or pharmaceutical compositions with one or more agents that treat the side effects of a disease or the side effects of one of the additional therapeutic agents, e.g., providing the subject with an analgesic.

Thus, the term “additional therapeutic compounds” includes a variety of include anti-cancer agents or treatments, such as chemical compounds that are also known as anti-neoplastic agents or chemotherapeutic agents. The agents can be used in combination with the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes or pharmaceutical compositions. Such compounds include, but are not limited to, alkylating agents, DNA intercalators, protein synthesis inhibitors, inhibitors of DNA or RNA synthesis, DNA base analogs, topoisomerase inhibitors, anti-angiogenesis agents, and telomerase inhibitors or telomeric DNA binding compounds. For example, suitable alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride, melphalan, novembichine, phenesterine, prednimustine, trofosfamide, and uracil mustard; nitroso ureas, such as carmustine, chlorozotocin, fotemustine, lomustine, nimustine, and ranimustine.

Chemotherapeutic protein synthesis inhibitors can also be combined with the the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes. Such inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine.

Additionally, protein synthesis inhibitors can also be combined with the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes. Such inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Furthermore, inhibitors of DNA synthesis can be combined with the presently-disclosed peptides or polypeptides. Such inhibitors include alkylating agents such as dimethyl sulfate, mitomycin C, nitrogen and sulfur mustards, intercalating agents, such as acridine dyes, actinomycins, adriamycin, anthracenes, benzopyrene, ethidium bromide, propidium diiodide-intertwining, and agents, such as distamycin and netropsin. Topoisomerase inhibitors, such as coumermycin, nalidixic acid, novobiocin, and oxolinic acid, inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be combined with the presently-disclosed peptides or polypeptides comprising one or more identified shared neo-epitopes or pharmaceutical compositions to provide a suitable cancer treatment.

Thus, current chemotherapeutic agents that can be used in a combination treatment with the presently-disclosed peptides or polypeptides, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes include, but are not limited to, adrimycin, 5-fluorouracil (5FU), etoposide, camptothecin, actinomycin-D, mitomycin, cisplatin, hydrogen peroxide, carboplatin, procarbazine, mechlorethamine, cyclophosphamide, ifosfamide, melphalan, chjlorambucil, bisulfan, nitrosurea, dactinomycin, duanorubicin, doxorubicin, bleomycin, plicomycin, tamoxifen, taxol, transplatimun, vinblastin, and methotrexate, and the like.

The additional therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

Peptides or Polypeptides Comprising One or More Identified Shared Neo Epitopes for Step (a) or One or More Subject-Specific Neo-Epitopes for Step (b)

In aspects, the peptides or polypeptides of the instant invention, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein: e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus and peptides or polypeptides comprising one or more identified subject-specific neo-epitopes, can be of a variety of lengths. For example, peptides or polypeptides that comprise one or more shared neo-epitopes may be designed as described herein. Further, in aspects, the subject-specific peptides or polypeptides comprising the at least one identified neo-epitope comprising, consisting, or consisting essentially of an amino acid sequence of the at least one identified neo-epitope (e.g., a 9-mer identified neo-epitope that bind to a MHC class II molecule and/or a 9-mer or 10-mer identified neo-epitope that bind to a MHC Class I molecule, and/or fragments or variants thereof), and optionally 1 to 12 additional amino acids distributed in any ratio on the N terminus and/or C-terminus of the the at least one identified neo-epitope. In aspects, the instant invention is directed to a peptide or polypeptide have a core amino acid sequence comprising, consisting of, or consisting essentially of the at least one identified neo-epitope, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant invention is directed to a peptide or polypeptide have a core sequence comprising, consisting of, or consisting essentially of the at least one identified neo-epitope (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MHC binding propensity) as said polypeptide core sequence without said flanking amino acids. In aspects, said polypeptide with the flanking amino acids is still able to bind to the same HLA molecule (i.e., retain MHC binding propensity) and retain the same TCR specificity as said polypeptide core sequence without said flanking amino acids. In aspects, said flanking amino acid sequences are those that also flank the the at least one identified neo-epitope in the naturally occurring protein.

In aspects, the peptides or polypeptides as instantly disclosed may comprise additional adjacent amino acids extending in the N- and/or C-terminal directions. In aspects, such additional adjacent sequences may comprise 3 or more, 5 or more, 10 or more 15 or more, 20 or more, and even 50 or more amino acids, including any value or range therebetween, and may flank the peptide or polypeptide sequence N-terminally or C-terminally. In aspects, said peptides or polypeptides can be flanked by amino acid sequences that also flank the shared neo-epitopes (or peptides or polypeptides comprising, consisting of, or consisting essentially of one or more peptides or polypeptides from Table A, B, and/or C, and/or fragments and variants thereof, as disclosed herein) or subject-specific neo-epitopes included therein in the naturally occurring protein. In aspects, the peptides or polypeptides can be capped with an N-terminal acetyl and C-terminal amino group. In aspects, the peptides or polypeptides can be either in neutral (uncharged) or salt forms, and may be either free of or include modifications such as glycosylation, side chain oxidation, or phosphorylation.

In aspects, the peptides or polypeptides of the instant invention, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes, can be “isolated” or “purified”, which means that it is substantially free of cellular material when it is isolated from recombinant and non-recombinant cells, or free of chemical precursors or other chemicals when it is chemically synthesized. A peptide or polypeptide of the present invention, however, can be joined to, linked to, or inserted into another polypeptide (e.g., a heterologous polypeptide) with which it is not normally associated in a cell and still be “isolated” or “purified.”

In aspects, the peptides or polypeptides of the instant invention, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes, may comprise, but are not limited to, about 8 to about 100 amino acid residues, including any value or range therein. In aspects, a peptide or polypeptide may comprise greater than 100 amino acid residues. In aspects, each peptide or polypeptide comprising one or more identified shared neo-epitopes (including peptides or polypeptide comprising, consisting of, or consisting essentially of one or more peptides or polypeptides from Table A, B, and/or C, and/or fragments and variants thereof, as disclosed herein) or each peptide or polypeptide comprising one or more subject-specific neo-epitope has a length of from 8-40 amino acids, from 8-30 amino acids, from 8-25 amino acids, from 8-23 amino acids, from 8-20 amino acids, or from 8-15 amino acids. In aspects, the peptides or polypeptides of the instant invention, including peptides or polypeptides comprising one or more identified shared neo-epitopes (including peptides and polypeptides comprising, consisting of, or consisting essentially of one or more peptides or polypeptides from Table A, B, and/or C and/or fragments and variants thereof) and peptides or polypeptides comprising one or more identified subject-specific neo-epitopes, may comprise at least one identified neo-epitope that is determined (e.g. predicted) to bind to the WIC molecule of the patient being administered the peptide or polypeptide, with each at least one neo-epitope including an extension of amino acids (e.g., of a length of 1-12 amino acids), the extension possibly serving to improve the biochemical properties of the peptides or polypeptides (e.g., but not limited to, solubility or stability) or to improve the likelihood for efficient proteasomal processing of the peptide.

In aspects or the peptides or polypeptides of the instant invention, including peptides or polypeptides comprising one or more identified shared neo-epitopes (including peptides and polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) and peptides or polypeptides comprising one or more identified subject-specific neo-epitopes, each one or more identified neo-epitopes may be spaced by linkers, in particular neutral linkers. The term “linker” refers to a peptide added between two peptide domains such as epitopes or vaccine sequences to connect said peptide domains. In aspects, a linker sequence is used to reduce steric hindrance between each one or more identified neo-epitopes, is well translated, and supports or allows processing of the each one or more identified neo-epitopes. In aspects, the linker should have little or no immunogenic sequence elements. For example, in aspects, the present invention is directed to a concatemeric polypeptide or peptide that comprises one or more of the instantly-disclosed shared and/or subject-specific peptides or polypeptides linked, fused, or joined together (e.g., fused in-frame, chemically-linked, or otherwise bound) to an additional peptide or polypeptide. Such additional peptide or polypeptide may be one or more of the instantly-disclosed shared and/or subject-specific peptides or polypeptides, or may be an additional peptide or polypeptide of interest, such as traditional tumor-associated antigens (TAAs). In aspects a concatemeric peptide is composed of 2 or more, 3 or more, 4 or more, 5 or more 6 or more 7 or more, 8 or more, 9 or more of the instantly-disclosed shared and/or subject-specific peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less subject-specific peptides or polypeptides. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed shared and/or subject-specific peptides or polypeptides linked, fused, or joined together. Each peptide or polypeptide of the concatemeric polypeptide may optionally have one or more linkers, which may optionally be cleavage sensitive sites, adjacent to their N and/or C terminal end. In such a concatemeric peptide, two or more of the peptides (including subject-specific peptides or polypeptides as disclosed herein) may have a cleavage sensitive site between them. Alternatively two or more of the peptides (including subject-specific peptides or polypeptides as disclosed herein) may be connected directly to one another or through a linker that is not a cleavage sensitive site.

As used herein, two peptide or polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (e.g., percent homology equals the number of identical positions/total number of positions×100).

In aspects, the present invention also encompasses peptides or polypeptides comprising at least one identified shared neo-epitope (for administration in step (a)) and peptides or polypeptides comprising at least one identified subject-specific neo-epitope (for administration in step (b)), with the at least one identified shared neo-epitope or subject-specific neo-epitope having a lower degree of identity but having sufficient similarity so as to perform one or more of the same functions. For example, the present invention encompasses peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein having a lower degree of identity but having sufficient similarity so as to perform one or more of the same functions. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie J U et al., (1990), Science, 247(4948):130610, which is herein incorporated by reference in its entirety).

In aspects, a variant of the at the least one identified shared neo-epitope of the peptides or polypeptides for administration in step (a) or a variant of the at least on identified subject-specific neo-epitope of the peptides or polypeptides for administration in step (b) can differ in amino acid sequence by one or more substitutions, deletions, insertions, inversions, fusions, and truncations or a combination of any of these. For example, the peptides or polypeptides comprising, consisting of, or consisting essentially of one or more peptides or polypeptides from Table A (SEQ ID NOS: 105-163), Table B (SEQ ID NOS: 164-350), and/or C, and/or fragments thereof, as disclosed herein can differ in amino acid sequence by one or more substitutions, deletions, insertions, inversions, fusions, and truncations or a combination of any of these. In aspects, a variant of the at least one identified shared neo-epitope of the peptides of polypeptides for administration in step (a) or a variant of the leat one identified subject-specific neo-epitope of the peptides or polypeptides for administration in step (b) can be fully functional (e.g., retain MHC binding propensity and TCR specificity) or can lack function in one or more activities. Fully functional variants typically contain only conservative variation or variation in non-critical residues or in non-critical regions; in this case, typically MHC contact residues provided MHC binding is preserved. In aspects, functional variants can also contain substitution of similar amino acids that result in no change or an insignificant change in function (e.g., retain MHC binding propensity and TCR specificity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues.

In aspects, the present invention also includes fragments of the instantly-disclosed at the least one identified shared neo-epitope of the peptides or polypeptides for administration in step (a), including fragments of one or more peptides or polypeptides from Table A (SEQ ID NOS: 105-163), Table B (SEQ ID NOS: 164-350), and/or C, as disclosed herein, or fragments of the instantly-disclosed at the least one identified subject-specific neo-epitope of the peptides or polypeptides for administration in step (b). In aspects, the present invention also encompasses fragments of the variants of the identified neo-epitopes described herein, including fragments of the variants of one or more peptides or polypeptides from Table A, B, and/or C. In aspects, as used herein, a fragment comprises at least about nine contiguous amino acids. Useful fragments (and fragments of the variants of the identified shared or subject-specific neo-epitopes described herein) include those that retain one or more of the biological activities of the identified neo-epitope, particularly MHC binding propensity and TCR specificity. Biologically active fragments are, for example, about 9, 12, 15, 16, 20 or 30 or more amino acids in length, including any value or range therebetween. In aspects, fragments can be discrete (not fused to other amino acids or polypeptides) or can be within a larger polypeptide. In aspects, several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the at least one identified shared neo-epitope of the peptides or polypeptides (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) for administration in step (a) and the at least one identified subject-specific neo-epitope of the pepides or polypeptides for administration in step (b) can include allelic or sequence variants (“mutants”) or analogs thereof. In aspects, the peptides or polypetides comprising the at least one identified shared neo-epitope or subject-specific neo-epitope can include chemical modifications (e.g., pegylation, glycosylation). In aspects, a mutant retains the same functions performed by a polypeptide encoded by a nucleic acid molecule of the present invention, particularly MHC binding propensity and TCR specificity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

In aspects, a peptide or polypeptide comprising at least one identified shared neo-epitope (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) for administration in step (a) and the the pepides or polypeptides comprising at least one identified shared neo-epitope for administration in step (b) can include a pharmaceutically acceptable salt thereof. A “pharmaceutically acceptable salt” of a peptide or polypeptide means a salt that is pharmaceutically acceptable and that possesses the desired pharmacological activity of the parent peptide or polypetide. As used herein, “pharmaceutically acceptable salt” refers to derivative of the instantly-disclosed peptides or polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

The peptides or polypeptides comprising at least one identified shared neo-epitope (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) for administration in step (a) and the the pepides or polypeptides comprising at least one identified shared neo-epitope for administration in step (b) may be produced by any known methods of producing peptides or polypeptides, including known in vitro and in vivo methods. In vitro production may be done by variety of methods known in the art, which include peptide or polypeptide chemical synthesis techniques, the expression of proteins, polypeptides or peptides through standard molecular biological techniques, the isolation of proteins or peptides from natural sources, in vitro translation, followed by any necessary purification of the expressed peptide/polypeptide. Alternatively, the subject-specific peptides or polypeptides comprising the at least one identified neo-epitope may be produced in vivo by introducing molecules (e.g., DNA, RNA, viral expression systems, and the like) that encode tumor specific neoantigens into a subject, whereupon the encoded tumor specific neoantigens are expressed.

In aspects, the present invention also provides for nucleic acids (e.g., DNA, RNA, vectors, viruses, or hybrids) that encode in whole or in part one or more peptides or polypeptides (including or concatemeric peptides) of the present invention. To be clear, such nucleic acids can be administerd in steps (a) and (b) of the instantly-disclosed methods of treating a neoplasia in a subject or methods of inducing an immune response in a subject, either replacing the use of peptides or polypeptides or used in conjunction therewith. In aspects, a nucleic acid (e.g., a polynucleotide) encoding a peptide or polypeptide comprising the at least one identified shared neo-epitope (including peptides or polypeptide comprising, consisting of, or consisting essentially of one or more peptides or polypeptides from Table A, B, and/or C and/or fragments and variants thereof) or encording a peptide or polypeptide comprising the at least one identified subject-specific neo-epitope may be used to produce the neo-epitopes in vitro or in vivo. The polynucleotide may be, e.g., DNA, cDNA, PNA, CNA, RNA, either single- and/or double-stranded, or native or stabilized forms of polynucleotides as are known in the art. An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

In aspects, the present invention is directed to expression vectors comprising the peptides or polypeptides (including or concatemeric peptides) comprising the at least one identified shared neo-epitope (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein) or the peptides or polypeptides comprising the at least one identified subject-specific neo-epiotpe, as well as host cells containing the expression vectors, are also contemplated. The peptides or polypeptides may be provided in the form of RNA or cDNA molecules encoding the desired neo-epitopes. One or more peptides or polypeptides of the present invention may be encoded by a single expression vector. Such nucleic acid molecules may act as vehicles for delivering neoantigenic peptides/polypeptides to the subject in need thereof, in vivo, in the form of, e.g., DNA/RNA vaccines.

In aspects, the instantly-disclosed peptides or polypeptides can be purified to homogeneity or partially purified. It is understood, however, that preparations in which the peptides or polypeptides comprising at least one identified shared neo-epitope are not purified to homogeneity are useful. The critical feature is that the preparation allows for the desired function of the at least one neo-epitope, even in the presence of considerable amounts of other components. Thus, the present invention encompasses various degrees of purity. In one embodiment, the language “substantially free of cellular material” includes preparations of the subject-specific peptides or polypeptides comprising at least one identified neo-epitope having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therein.

In aspects, when a peptide or polypeptide of the instant invention is recombinantly produced, said peptide or polypeptide can also be substantially free of culture medium, for example, culture medium represents less than about 20%, less than about 10%, or less than about 5% of the volume of the peptide or polypeptide or nucleic acid preparation. The language “substantially free of chemical precursors or other chemicals” includes preparations of the peptide or polypeptide or nucleic acid is separated from chemical precursors or other chemicals that are involved in its synthesis. The language “substantially free of chemical precursors or other chemicals” can include, for example, preparations of the peptide or polypeptide having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

### Pharmaceutical Compositions

In aspects, a peptide or polypeptide (including or concatemeric peptides) comprising one or more identified shared neo-epitopes for administration in step (a) or a peptide or polypeptide comprising one or more identified subject-specific neo-epitopes for administration in step (b) as described herein may be formulated into a pharmaceutical composition, such as a shared neoplasia vaccine for step (a) or a personalized neoplasia vaccine for step (b), and administered to a subject as disclosed herein in order to induce an immune response or to treat the subject's neoplasia.

Thus, a further embodiment is directed to a pharmaceutical composition for use in step (a) of the instantly disclosed methods of teating a neoplasia in a subject or methods of inducing an immune response in a subject, the composition including a plurality of selected peptides or polypeptides comprising one or more identified shared neo-epitopes or one or more nucleic acids encoding said plurality of selected peptides or polypeptides, wherein the one or more identified neo-epitopes induces a neoplasia-specific effector T cell response in a subject. In aspects, the present invention is directed to a pharmaceutical composition for use in step (a), said composition comprising one or more peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, as disclosed herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus), wherein the one or more peptides or polypeptides from Table A, B, and/or C, and/or fragments and variants thereof, induces a bladder cancer-specific effecter T cell response in a subject. In aspects, the plurality of selected peptides or polypeptides comprising the one or more identified neo-epitope or one or more nucleic acids encoding said plurality of selected peptides or polypeptides for use in step (a) are selected and produced by the methods as disclosed herein.

Likewise, a further embodiment is directed to a pharmaceutical composition for use in step (b) of the instantly disclosed methods of teating a neoplasia in a subject or methods of inducing an immune response in a subject, the composition including a plurality of selected peptides or polypeptides comprising one or more identified subject-specific neo-epitopes or one or more nucleic acids encoding said plurality of selected peptides or polypeptides, wherein the one or more identified neo-epitopes induces a neoplasia-specific effector T cell response in a subject. In aspects, the plurality of selected peptides or polypeptides comprising the one or more identified neo-epitope or one or more nucleic acids encoding said plurality of selected peptides or polypeptides for use in step (b) are selected and produced by the methods as disclosed herein.

In aspects, a pharmaceutical composition as described herein may further comprise a pharmaceutically acceptable excipient. A “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Thus, the term “pharmaceutical excipient” is used herein to describe any ingredient other than the compound(s) of the invention. Examples of pharmaceutical excipients include one or more substances which may act as diluents, flavoring agents, solubilisers, lubricants, suspending agents, binders, preservatives, wetting agents, tablet disintegrating agents, or an encapsulating material. The choice of excipient will to a large extent depend on factors such as the particular mode of administration, the effect of the excipient on solubility and stability, and the nature of the dosage form. A “pharmaceutical excipient” includes both one and more than one such excipient.

In aspects, a pharmaceutical composition as described herein may comprise a pharmaceutically acceptable carrier for administration to a human or an animal. As such, the pharmaceutical compositions can be administered orally as a solid or as a liquid, or can be administered intramuscularly or intravenously as a solution, suspension, or emulsion. Alternatively, the pharmaceutical compositions can be administered by inhalation, intravenously, or intramuscularly as a liposomal suspension. In some embodiments, the pharmaceutical composition is formulated for oral administration. In other embodiments, the pharmaceutical composition is formulated for intravenous administration.

In aspects, a pharmaceutical composition as described herein may comprise a pharmaceutically acceptable adjuvant. Such adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In aspects of the pharmaceutical composition, the adjuvant comprises poly-ICLC. The TLR9 agonist CpG and the synthetic double-stranded RNA (dsRNA) TLR3 ligand poly-ICLC are two of the most promising neoplasia vaccine adjuvants currently in clinical development. In preclinical studies, poly-ICLC appears to be the most potent TLR adjuvant when compared to LPS and CpG. This appears due to its induction of pro-inflammatory cytokines and lack of stimulation of IL-10, as well as maintenance of high levels of co-stimulatory molecules in DCs. Poly-ICLC is a synthetically prepared double-stranded RNA consisting of polyI and polyC strands of average length of about 5000 nucleotides, which has been stabilized to thermal denaturation and hydrolysis by serum nucleases by the addition of polyIysine and carboxymethylcellulose. The compound activates TLR3 and the RNA helicase-domain of MDA5, both members of the PAMP family, leading to DC and natural killer (NK) cell activation and mixed production of type I interferons, cytokines, and chemokines.

In aspects of the pharmaceutical composition as described herein (for use in either step (a) or step (b) of the instantly-disclosed methods of treating a neoplasia or methods of inducing an immune response), the plurality of selected peptides or polypeptides comprising one or more identified shared neo-epitopes (for step (a)) or subject-specific neo-epitopes (for step (b)) comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypetides, each comprising one or more identified shared or subject-specific neo-epitopes, respectively. For example, in aspects, a pharmaceutical composition for admistration in step (a) can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides (including up to 40 peptides or polypetides), including any value or range therebetween, comprising, consisting of, or consisting essentially of one or more peptides or polypeptides from Table A (SEQ ID NOS: 105-163), Table B (SEQ ID NOS: 164-350), and/or C and/or fragments and variants thereof, as described herein (e.g., one or more peptides or polypeptides having a core sequence comprising, consisting of, or consisting essentially of one or more peptides or polypeptides from Table A (SEQ ID NOS: 105-163), Table B (SEQ ID NOS: 164-350), and/or Table C (and/or fragments or variants thereof), optionally with extensions of 1 to 12 amino acids on the C-terminal and/or the N-terminal, wherein the flanking amino acids can be distributed in any ratio to the C-terminus and the N-terminus). In aspects, such polypetide may be spaced by linkers, in particular neutral linkers, as previously described. In aspects, the plurality of selected peptides or polypeptides comprising one or more identified neo-epitopes comprises from 3-20 selected peptides or polypeptides as disclosed herein, each comprising one or more identified shared neo-epitopes.

In aspects of the pharmaceutical composition, the one or more nucleic acids encoding said plurality of selected peptides or polypeptides are DNA, RNA, or mRNA.

In aspects of the pharmaceutical composition, the pharmaceutical composition further comprises an anti-immunosuppressive agent. In aspects, the anti-immunosuppressive agent comprises a checkpoint blockage inhibitor or other additional therapeutic adjuvants as described below.

### EXAMPLES

The following examples are intended to illustrate specific features and aspects of the instant invention and should not be construed as limiting the scope thereof.

### Example 1

### Tumor Growth Inhibition Post Vaccination with a Tregitope-Depleted Personalized Cancer Vaccine

Clinical studies have highlighted the potential of precision cancer immunotherapy to effectively control the tumor of patients across cancer indications. However, recent studies showcase the difficulty of establishing robust CD8+ and CD4+ T cell responses. We hypothesize that poor cancer vaccine performance may be due in part to the inadvertent inclusion of suppressive T cell neo-epitopes in neoantigen vaccines that may be recognized by regulatory T cells (Tregs).

To test this hypothesis, we used the ANCER™ system to identify and select neo-epitopes from the CT26 syngeneic colon cancer mouse model. ANCER™, a proprietary platform for the identification, characterization, and triaging of tumor-specific neo-epitopes, leverages EPIMATRIX® (for the identification of determined (e.g. predicted) neo-epitopes encoded by said neoplasia-specific mutations for use in the personalized neoplasia vaccine) and JANUSMATRIX™ (for the identification of neo-epitopes that are determined (e.g. predicted) to engage regulatory T cells, and exclusion of such identified neo-epitopes that are predicted to engage regulatory T cells from the subject-specific neo-epitopes for use in the personalized neoplasia vaccine), state-of-the-art predictive algorithms that have been extensively validated in prospective vaccine studies for infectious diseases (Moise et al., Hum. Vaccines Immunother 2015; Wada et al., Sci. Rep. 2017). Distinctive features of ANCER™ over other in silico pipelines are its ability to accurately predict CD4+ T cell epitopes and to identify tolerated or Treg epitopes.

1.1 Bioinformatics Design of a Tregitope-Depleted CT26 Vaccine

CT26 mutanomes and transcriptomes were retrieved from private and public sources (Castle et al. BMC Genomics 2014). The 3,267 and 3,023 variants from the private and public mutanomes, respectively, were screened to extract 1,787 SNVs shared in both datasets. Of these, 1,002 mutations were contained in genes showing evidence of expression based on transcriptomic data. In aspects, this step removing variants not detected in the transcriptomic data may be omitted in other analyses, such as cases where this information is not available. To further narrow the list of potential candidates, our analysis focused on 378 variants with at least 30× coverage in the tumor DNA.

Pairs of mutated and wild-type, or normal, 23-mer amino acid sequences were extracted for each of the 378 variants under study. Mutated sequences were designed with the mutation in the center surrounded by 11-mer flanks. This length allows for the characterization of every 9- and 10-mer frames overlapping with the mutation while adding flanking residues for peptide design.

Each pair of mutated and normal peptides were uploaded to the ANCER™ platform for neo-epitope identification and characterization. Each peptide was first parsed into overlapping 9- and 10-mer frames. We then evaluated each frame for its likelihood to bind to Balb/c MHC class I (H2-Dd and H2-Kd) and MHC class II (I-Ad, I-Ed) alleles. For human analyses, each frame would be evaluated for its likelihood to bind to the patient's MHC class I (HLA-A, HLA-B) and MHC class II (HLA-DRB1) alleles. Our process, to this date, supports 1,284 HLA-A, 933 HLA-B, and 612 HLA-DRB1 alleles. In the event that a patient expresses an MHC allele for which predictions are not readily available, a homology analysis can be performed between the patient allele and our reference alleles. For this analysis, we extract the amino acid sequences from the MHC binding pockets of the patient allele and compare them to the binding pockets from alleles we reliably model. Any non-supported patient allele whose MHC binding pockets are at least 90% homologous with the MHC binding pockets of a reference allele can be included in our analysis, where the model for the homologous reference allele is used to assess a frame's likelihood of binding to the patient allele. Performing these homology analyses allows us to extend our support to an additional 1,412 HLA-A, 1,872 HLA-B, and 949 HLA-DRB1 alleles.

Each frame-by-allele “assessment” is a statement about (i.e., determination of) predicted MEW binding affinity. Raw binding scores are adjusted to fit a normal, or Z-distribution. Raw binding scores are normalized based on the average (μ) binding score and standard deviation (σ) of a set of 10,000 random 9- or 10-mer amino acid sequences, following the naturally observed amino acid frequencies from UniProtKB/Swiss-Prot (web.expasy.org/docs/relnotes/relstat.html), as follows:

$$Normalized binding score = \frac{Raw binding score - μ}{σ} .$$

Normalized binding scores, herein referred to as binding scores or likelihood of binding, within the top 5% of this normal distribution are defined as “hits”; that is to say, potentially immunogenic and worthy of further consideration. These peptides have a significant chance of binding to MHC molecules with moderate to high affinity and, therefore, have a significant chance of being presented on the surface of both professional antigen presenting cells (APC) such as dendritic cells or macrophages, as well as non-professional APC, where they may be interrogated by passing T cells.

T cell epitopes predicted in mutated sequences are compared to normal matched sequences in order to identify neo-epitopes. T cell epitopes from mutated sequences are labeled as neo-epitopes if:
Their likelihood of binding to MHC falls within the top 5 percentile of our expected distribution and the likelihood of binding to MEW of the normal matched sequence falls below the top 10 percentile of the expected distribution, or;
Their likelihood of binding to MHC falls within the top 5 percentile of our expected distribution and the likelihood of binding to MEW of the normal matched sequence falls within the top 10 percentile of the expected distribution, and there is a least one mismatched TCR-facing amino acid between the mutated and non-mutated peptides.

TCR facing amino acid residues for a 9-mer mutated peptide and a 9-mer non-mutated peptide that bind to a MEW class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal.

Additionally, sequences presenting a high likelihood of binding to MEW are screened using a customized homology search to remove epitopes containing combinations of TCR-facing residues that are commonly found in a reference proteome. This homology screen first considers all the predicted epitopes contained within a given protein sequence and divides each predicted epitope into its constituent agretope and epitope. Each sequence is then screened against a database of murine proteins derived from the UniProt database (UniProt Proteome ID UP000000589, Reviewed/Swiss-Prot set). For human analyses, each sequence would be then screened against a database of human proteins derived from the UniProt database (UniProt Proteome ID UP000005640, Reviewed/Swiss-Prot set).

Cross-conserved epitopes, or peptides derived from the reference proteome with a compatible MHC binding agretope (i.e. the agretopes of both the input (mutated) peptide and its reference non-mutated counterpart are predicted to bind to the same MHC allele) and exactly the same TCR facing epitope, are returned. The Homology Score of an epitope corresponds to the number of matching cross-conserved MEW binding peptides within the reference proteome. In other words, the Homology Score Hₑ of an epitope e is calculated as follows:

Hₑ=|Xₑ|,

where:

Xₑ corresponds to the set of MHC binding peptides derived from the reference proteome that are restricted to the same MEW class I or MHC class II as epitope e and presenting a TCR facing epitope identical to the epitope e.

By extension, the Homology Score of a given peptide or protein corresponds to the average Homology Score of each individual epitope contained with the peptide or protein. In other words, the Homology Score Hₚ of a peptide p is calculated as follows:

$${H}_{p} = \frac{{Σ}_{e ∈ E} {H}_{e}}{\vert E \vert}$$

where:
E corresponds to the set of MHC class I- or MHC class II-restricted epitopes within peptide p;
   Hₑ corresponds to the Homology Score of epitope e as defined above.

T cells that recognize antigen-derived epitopes sharing TCR contacts with epitopes derived from self may be deleted or rendered anergic during thymic selection before they can be released to the periphery. As such, vaccine components targeting these T cells may be ineffective. On the other hand, vaccine-induced immune response targeting cross-reactive epitopes may induce unwanted autoimmune responses targeting the homologues of the cross-reactive epitopes identified by our homology search. As a result, vaccine safety may be reduced. A review of MHC class II-restricted T cell epitopes contained in the IEDB database (iedb.org) indicates that there is a statistically significant relationship between high Homology Scores and observed production of IL-10 and a statistically significant inverse relationship between high Homology Scores and observed production of IL-4 (see, e.g., Moise et al. iVax: An integrated toolkit for the selection and optimization of antigens and the design of epitope-driven vaccines. Human Vaccin Immunother. 2015; 11(9):2312-21, herein incorporated by reference in its entirety). Conversely, the same homology analysis can be performed against a set of known infectious disease-derived epitopes known to be immunogenic, extracted for example from the IEDB database, or against a set of other known immunogenic sequences or common pathogen-derived sequences. This analysis has the purpose of identifying neo-epitope candidates that share a high degree of homology with other known or putative effector T cell epitopes. Peptides or polypeptides containing such neo-epitopes (or nucleic acid encoding said peptides or polypeptides) can be prioritized in vaccine formulations.

Each mutated sequence undergoes the homology screen described above in order to characterize the degree of similarity with self of each of the encoded MHC class I- and MHC class II-restricted epitopes and neo-epitopes. MHC class I or MHC class II epitopes and MHC class I or MHC class II neo-epitopes with two or more cross-reactive matches in the reference proteome are categorized as exhibiting a high degree of similarity with self and are considered to have a higher likelihood of being tolerated or to engage regulatory T cells and/or other detrimental T cells (including T cells with potential host cross-reactivity and/or anergic T cells). An optimization procedure is then run on each mutated sequence to determine if a substring within the amino acid sequence can be found, such that:
At least one MHC class I- or MHC class II-restricted epitope is encoded in the substring, and;
All MHC class I- or MHC class II-restricted neo-epitopes encoded in the substring have no more than two cross-reactive matches in the reference proteome, and;
All MHC class I- or MHC class II-restricted epitopes encoded in the substring have no more than two cross-reactive matches in the reference proteome.

This procedure has the effect of removing amino acid substrings containing putative Tregitopes and/or other putative detrimental T cell epitopes (including epitopes that engage T cells with potential host cross-reactivity and/or anergic T cells) and other highly cross-conserved epitopes from mutated sequences. The resulting optimized sequences will only contain epitopes or neo-epitopes that exhibit low degree of similarity with self-sequences. Mutated sequences are discarded from consideration if no substring matching the above criteria can be found.

One hundred thirty-five of the 378 analyzed mutated sequences could be optimized to yield amino acid sequences that contained MHC class I and/or MHC class II restricted neo-epitopes displaying a low degree of self-similarity. These 135 sequences (peptides or polypeptides comprising one or more identified neo-epitopes) were then ranked according to one or more of the following features:

Immunogenicity-related features:
Count of MHC class I neo-epitopes;
Minimal percentile rank of a MHC class I neo-epitope;
MHC class I-restricted Treg induction potential of the neoantigen (e.g. peptide or polypeptide comprising one or more identified neo-epitopes);
Count of MHC class II neo-epitopes (in aspects, which may include one or more of);
Minimal percentile rank of a MHC class II neo-epitope;
MHC class II-restricted Treg induction potential of the neoantigen (e.g. peptide or polypeptide comprising one or more identified neo-epitopes);
Whether the optimized neoantigen (e.g. peptide or polypeptide comprising one or more identified neo-epitopes) contains both MEW class I and II neo-epitopes.

Sequencing-related features (in aspects, which may include one or more of):
Expression level of the associated transcript;
Coverage of the mutation in the tumor DNA, i.e. the number of unique sequencing reads that overlap the genomic position of the mutation;
Variant allele fraction (VAF) of the mutation in the tumor DNA, i.e. the relative frequency, from 0 to 1, of the observed mutation across sequencing reads;
Other sequencing metadata, as needed.

Physicochemical-related features (in aspects, which may include one or more of):
Net charge of the optimized neoantigen (e.g. peptide or polypeptide comprising one or more identified neo-epitopes);
Whether the optimized neoantigen (e.g. peptide or polypeptide comprising one or more identified neo-epitopes) contains at least one charged residue;
The count of cysteines (C) within the optimized neoantigen (e.g. peptide or polypeptide comprising one or more identified neo-epitopes);
Whether the optimized neoantigen (e.g. peptide or polypeptide comprising one or more identified neo-epitopes) contains at least one cysteine (C) and is negatively charged;
Whether the optimized neoantigen (e.g. peptide or polypeptide comprising one or more identified neo-epitopes) contains a poly-proline motif (‘PP’);
Whether the optimized neoantigen (e.g. peptide or polypeptide comprising one or more identified neo-epitopes) contains at least one methionine (M);
Whether the optimized neoantigen (e.g. peptide or polypeptide comprising one or more identified neo-epitopes) contains an N-terminal glutamine (Q);
Whether the optimized neoantigen (e.g. peptide or polypeptide comprising one or more identified neo-epitopes) contains a glycine (G) and/or proline (P) in the last or second to last positions;
Whether the optimized neoantigen (e.g. peptide or polypeptide comprising one or more identified neo-epitopes) contains a ‘DG’, DS′, ‘DA’, or ‘DN’ motif;
The hydropathy index of the optimized neoantigen (e.g. peptide or polypeptide comprising one or more identified neo-epitopes).

Scores can be assigned to neoantigens (e.g. peptide or polypeptide comprising one or more identified neo-epitopes) according to the following scoring scheme (in aspects, scores (e.g., points and/or percentages) that are italicized and bolded may be subject to adjustment; in aspects, the scoring scheme may include one of more of the following scoring steps/penalizing steps:).

Count of MHC class I neo-epitopes (maximum of 20 points):
Neoantigens (e.g. peptide or polypeptide comprising one or more identified neo-epitopes) containing one or less MHC class I neo-epitopes are assigned 0% of the points (i.e. 0 point)
Neoantigens (e.g. peptide or polypeptide comprising one or more identified neo-epitopes) containing two MHC class I neo-epitopes are assigned 80% of the points (i.e. 16 points)
Neoantigens (e.g. peptide or polypeptide comprising one or more identified neo-epitopes) containing three or more MHC class I neo-epitopes are assigned 100% of the points (i.e. 20 points)

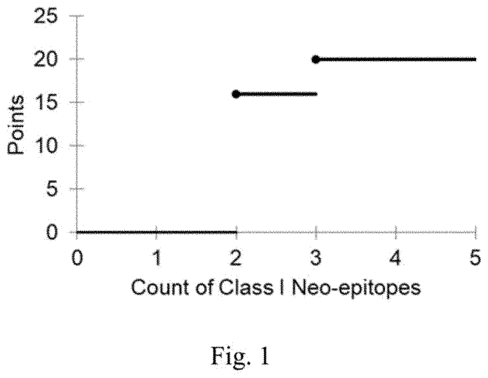

FIG. 1 is an exemplary graph depicting points assigned to neoantigen (e.g. peptide or polypeptide comprising one or more identified neo-epitopes) candidates based on their MHC class I neo-epitope content.

Minimal percentile rank of a MHC class I neo-epitope (maximum of 20 points):
Neoantigens (e.g. peptide or polypeptide comprising one or more identified neo-epitopes) where the minimal percentile rank of a MHC class I neo-epitope falls between 5% (inclusive) and 2.5% (exclusive) are assigned 0% of the points (i.e. 0 point)
Neoantigens (e.g. peptide or polypeptide comprising one or more identified neo-epitopes) where the minimal percentile rank of a MHC class I neo-epitope falls between 1% (exclusive) and 2.5% (inclusive) are assigned 50% of the points (i.e. 10 points)
Neoantigens (e.g. peptide or polypeptide comprising one or more identified neo-epitopes) where the minimal percentile rank of a MHC class I neo-epitope falls within 1% (inclusive) are assigned 100% of the points (i.e. 20 points)

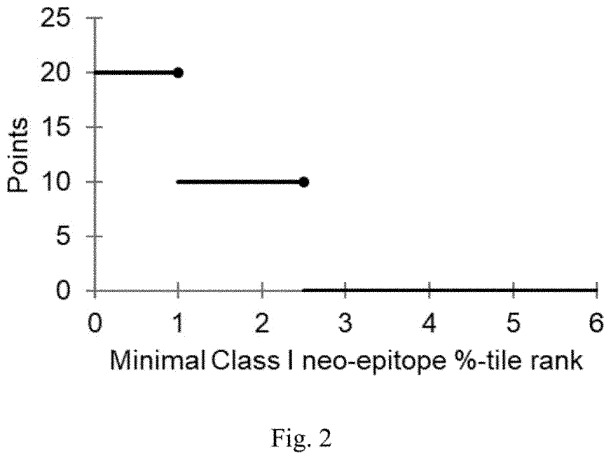

FIG. 2 is an exemplary graph depicting points assigned to neoantigen (e.g. peptide or polypeptide comprising one or more identified neo-epitopes) candidates based on the minimal percentile rank of a MHC class I neo-epitope.

Count of MHC class II neo-epitopes (maximum of 10 points):
Neoantigens (e.g. peptide or polypeptide comprising one or more identified neo-epitopes) containing one or less MHC class II neo-epitopes are assigned 0% of the points (i.e. 0 point)
Neoantigens (e.g. peptide or polypeptide comprising one or more identified neo-epitopes) containing 2 MHC class II neo-epitopes are assigned 80% of the points (i.e. 8 points)
Neoantigens (e.g. peptide or polypeptide comprising one or more identified neo-epitopes) containing three or more MHC class II neo-epitopes are assigned 100% of the points (i.e. 10 points)

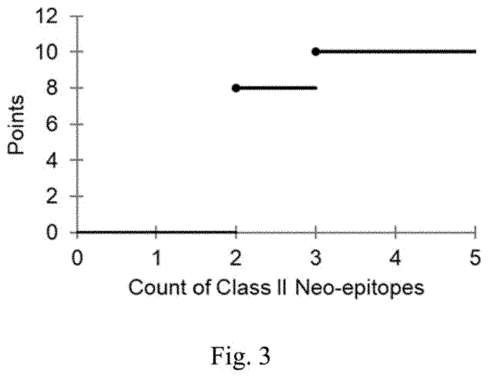

FIG. 3 is an exemplary graph depicting points assigned to neoantigen (e.g. peptide or polypeptide comprising one or more identified neo-epitopes) candidates based on their MHC class II neo-epitope content.

Minimal percentile rank of a MHC class II neo-epitope (maximum of 5 points):
Neoantigens (e.g. peptide or polypeptide comprising one or more identified neo-epitopes) where the minimal percentile rank of a MHC class II neo-epitope falls between 5% (inclusive) and 2.5 (exclusive) are assigned 0% of the points (i.e. 0 point)
Neoantigens (e.g. peptide or polypeptide comprising one or more identified neo-epitopes) where the minimal percentile rank of a MHC class II neo-epitope falls between 1% (exclusive) and 2.5% (inclusive) are assigned 50% of the points (i.e. 2.5 points)
Neoantigens (e.g. peptide or polypeptide comprising one or more identified neo-epitopes) where the minimal percentile rank of a MHC class II neo-epitope falls within 1% (inclusive) are assigned 100% of the points (i.e. 5 points)

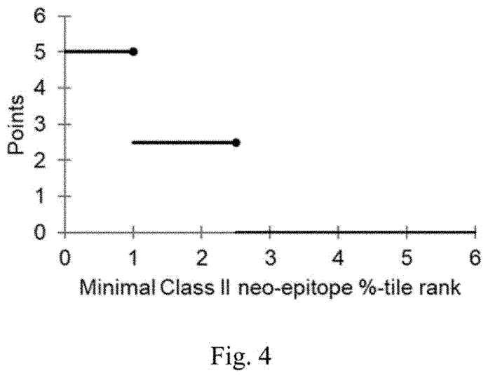

FIG. 4 is an exemplary graph depicting points assigned to neoantigen (e.g. peptide or polypeptide comprising one or more identified neo-epitopes) candidates based on the minimal percentile rank of a MHC class II neo-epitope.

Presence of both MHC class I and II neo-epitopes (maximum of 20 points):
Neoantigens (e.g. peptide or polypeptide comprising one or more identified neo-epitopes) containing MHC class I neo-epitopes only or MHC class II neo-epitopes only are assigned 0% of the points (i.e. 0 point)
Neoantigens (e.g. peptide or polypeptide comprising one or more identified neo-epitopes) containing at least one MHC class I neo-epitope and at least one MHC class II neo-epitope are assigned 100% of the points (i.e. 20 points)

MHC class I-restricted Treg induction potential (maximum of 5 points):
Neoantigens (e.g. peptide or polypeptide comprising one or more identified neo-epitopes) with a MHC class I-restricted average depth of coverage within the reference proteome, or MHC class I Homology Score (as calculated above), between 0 (inclusive) and 0.25 (exclusive) are assigned 100% of the points (i.e. 5 points)
Neoantigens (e.g. peptide or polypeptide comprising one or more identified neo-epitopes) with a MHC class I-restricted average depth of coverage within the reference proteome, or MHC class I Homology Score, between 0.25 (inclusive) and 0.5 (exclusive) are assigned 50% of the points (i.e. 2.5 points)
Neoantigens (e.g. peptide or polypeptide comprising one or more identified neo-epitopes) with a MHC class I-restricted average depth of coverage within the reference proteome, or MHC class I Homology Score, between 0.5 (inclusive) and 1 (exclusive) are assigned 10% of the points (i.e. 0.5 points)
Neoantigens (e.g. peptide or polypeptide comprising one or more identified neo-epitopes) with a MHC class I-restricted average depth of coverage within the reference proteome, or MHC class I Homology Score, above 1 (inclusive) are assigned 0% of the points (i.e. 0 point)

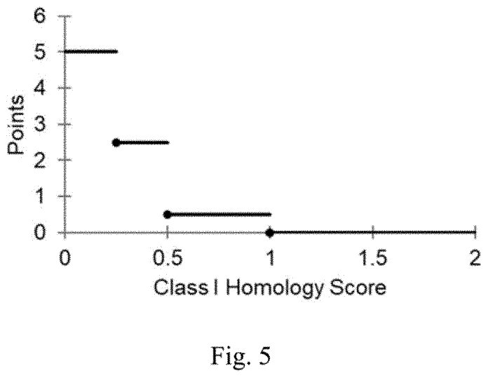

FIG. 5 is an exemplary graph depicting points assigned to neoantigen (e.g. peptide or polypeptide comprising one or more identified neo-epitopes) candidates based on their WIC class I Homology Score.

MHC class II-restricted Treg induction potential (maximum of 20 points):
Neoantigens (e.g. peptide or polypeptide comprising one or more identified neo-epitopes) with a WIC class II-restricted average depth of coverage within the reference proteome, or MHC class II Homology Score, between 0 (inclusive) and 0.25 (exclusive) are assigned 100% of the points (i.e. 20 points)
Neoantigens (e.g. peptide or polypeptide comprising one or more identified neo-epitopes) with a WIC class II-restricted average depth of coverage within the reference proteome, or MHC class II Homology Score, between 0.25 (inclusive) and 0.5 (exclusive) are assigned 50% of the points (i.e. 10 points)
Neoantigens (e.g. peptide or polypeptide comprising one or more identified neo-epitopes) with a WIC class II-restricted average depth of coverage within the reference proteome, or MHC class II Homology Score, between 0.5 (inclusive) and 1 (exclusive) are assigned 10% of the points (i.e. 2 points)
Neoantigens (e.g. peptide or polypeptide comprising one or more identified neo-epitopes) with a WIC class II-restricted average depth of coverage within the reference proteome, or MHC class II Homology Score, above 1 (inclusive) are assigned 0% of the points (i.e. 0 point)

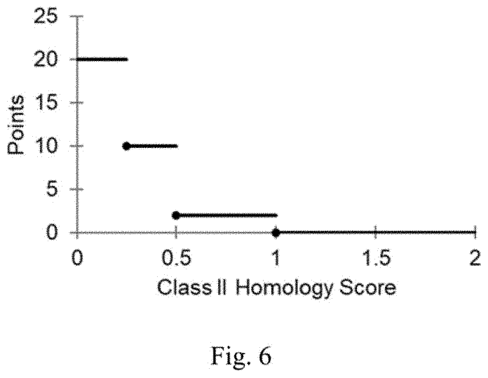

FIG. 6 is an exemplary graph depicting points assigned to neoantigen (e.g. peptide or polypeptide comprising one or more identified neo-epitopes) candidates based on their WIC class II Homology Score.
Transcript expression (in e.g. Transcript Per Million, TPM, which is calculated as is known in the art) (maximum of 30 points):
   Neoantigens (e.g. peptide or polypeptide comprising one or more identified neo-epitopes) derived from a transcript whose expression lies in the top 10% of the TPMs are assigned 100% of the points (i.e. 30 points)
   Neoantigens (e.g. peptide or polypeptide comprising one or more identified neo-epitopes) derived from a transcript whose expression lies below the top 25% of the TPMs are assigned 0% of the points (i.e. 0 points)
   Neoantigens (e.g. peptide or polypeptide comprising one or more identified neo-epitopes) derived from a transcript whose expression lies between the top 25% and 10% of the TPMs are assigned a linearly distributed percent of points

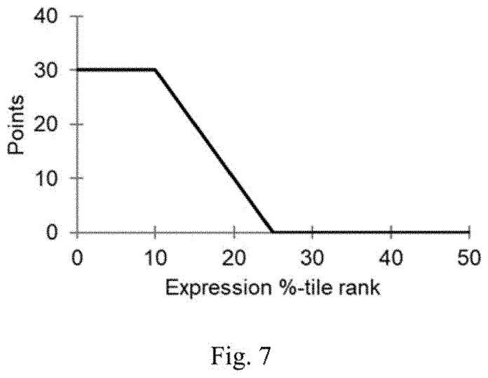

FIG. 7 is an exemplary graph depicting points assigned to neoantigen (e.g. peptide or polypeptide comprising one or more identified neo-epitopes) candidates based on the expression percentile rank of their originating transcript.

Coverage, calculated as is known in the art (maximum of 1 point):
Neoantigens (e.g. peptide or polypeptide comprising one or more identified neo-epitopes) containing a mutation with a depth of coverage in the tumor DNA of less than 20 are assigned 0% of the points (i.e. 0 point)
Neoantigens (e.g. peptide or polypeptide comprising one or more identified neo-epitopes) containing a mutation with a coverage in the tumor DNA of between 20 and 50 (strictly below) are assigned 50% of the points (i.e. 0.5 point)
Neoantigens (e.g. peptide or polypeptide comprising one or more identified neo-epitopes) containing a mutation with a coverage in the tumor DNA of 50 or more are assigned 100% of the points (i.e. 1 point)

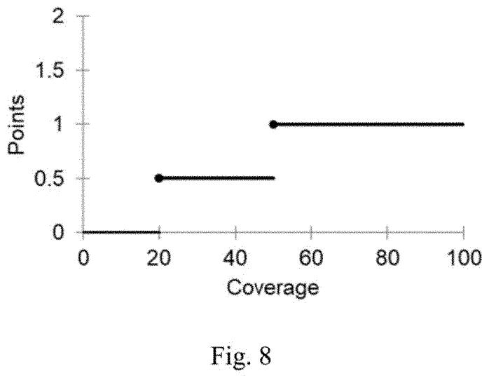

FIG. 8 is an exemplary graph depicting points assigned to neoantigen (e.g. peptide or polypeptide comprising one or more identified neo-epitopes) candidates based on their mutation coverage in the tumor DNA.

Variant allele fraction (VAF), calculated as is known in the art (maximum of 20 points):
For neoantigens (e.g. peptide or polypeptide comprising one or more identified neo-epitopes) derived from the mutanome of syngeneic models:
   Neoantigens (e.g. peptide or polypeptide comprising one or more identified neo-epitopes) containing a mutation with a VAF below 0.5 are assigned 0% of the points (i.e. 0 point)
   Neoantigens (e.g. peptide or polypeptide comprising one or more identified neo-epitopes) containing a mutation with a VAF between 0.5 and 0.75 (strictly below) are assigned 50% of the points (i.e. 10 points)
   Neoantigens (e.g. peptide or polypeptide comprising one or more identified neo-epitopes) containing a mutation with a VAF equal to or more than 0.75 are assigned 100% of the points (i.e. 20 points)
For neoantigens (e.g. peptide or polypeptide comprising one or more identified neo-epitopes) derived from the mutanome of patients:
   Neoantigens (e.g. peptide or polypeptide comprising one or more identified neo-epitopes) containing a mutation with a VAF below 0.1 are assigned 0% of the points (i.e. 0 point)
   Neoantigens (e.g. peptide or polypeptide comprising one or more identified neo-epitopes) containing a mutation with a VAF between 0.1 and 0.25 (strictly below) are assigned 50% of the points (i.e. 10 points)
   Neoantigens (e.g. peptide or polypeptide comprising one or more identified neo-epitopes) containing a mutation with a VAF equal to or more than 0.25 are assigned 100% of the points (i.e. 20 points)

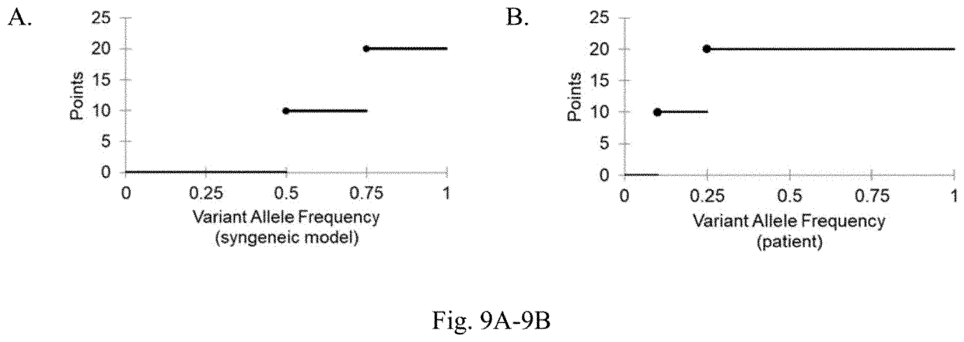

FIGS. 9A-B are exemplary graphs depicting points assigned to neoantigen (e.g. peptide or polypeptide comprising one or more identified neo-epitopes) candidates derived from the mutanome of syngeneic models (FIG. 9A) or patients (FIG. 9B) based on the variant allele frequency (VAF) of the mutation in the tumor DNA.

Points are then summed and normalized to a 100-point scale, where a perfect neoantigen (e.g. peptide or polypeptide comprising one or more identified neo-epitopes), in other words, a neoantigen that is assigned the maximum number of points, would score 100.

Severe penalties (currently set to a deduction of 100 points, which may be assigned before or after the 100-point normalization) can be assigned to a candidate neoantigen (e.g. peptide or polypeptide comprising one or more identified neo-epitopes) if:
The neoantigen (e.g. peptide or polypeptide comprising one or more identified neo-epitopes) has no charged residues, or
The neoantigen (e.g. peptide or polypeptide comprising one or more identified neo-epitopes) has a null net charge, or
The neoantigen (e.g. peptide or polypeptide comprising one or more identified neo-epitopes) contains at least two cysteines, or
The neoantigen (e.g. peptide or polypeptide comprising one or more identified neo-epitopes) contains at least one cysteine and is negatively charged, or
The neoantigen (e.g. peptide or polypeptide comprising one or more identified neo-epitopes) contains an N-terminal glutamine, or
The neoantigen (e.g. peptide or polypeptide comprising one or more identified neo-epitopes) contains a poly-proline motif, or
The neoantigen (e.g. peptide or polypeptide comprising one or more identified neo-epitopes) has an hydropathy index greater or equal to 2.

Moderate penalties (currently set to a deduction of 10 points) can be assigned to a candidate neoantigen (e.g. peptide or polypeptide comprising one or more identified neo-epitopes) if:
The neoantigen (e.g. peptide or polypeptide comprising one or more identified neo-epitopes) contains one cysteine

Minor penalties (currently set to a deduction of 1 point) can be assigned to a candidate neoantigen (e.g. peptide or polypeptide comprising one or more identified neo-epitopes) if:
The neoantigen (e.g. peptide or polypeptide comprising one or more identified neo-epitopes) is negatively charged, or
The neoantigen (e.g. peptide or polypeptide comprising one or more identified neo-epitopes) contains at least one methionine, or
The neoantigen (e.g. peptide or polypeptide comprising one or more identified neo-epitopes) contains a glycine and/or proline in the last or second to last positions, or
The neoantigen (e.g. peptide or polypeptide comprising one or more identified neo-epitopes) contains a ‘DG’, DS′, ‘DA’, or ‘DN’ motif

Alternatively, scores can be assigned according to following scoring scheme (to be clear, this process was not used to rank the CT26 peptides):

Sₚ=(C1ₚ+C2ₚ)×Fₚ×Eₚ or,

Sₚ=(C1ₚ+C2ₚ)×Fₚ,

where:
Sₚ corresponds to the score of peptide p;
C1ₚ corresponds to the MHC class I-restricted immunogenic potential of peptide p;
C2ₚ corresponds to the MHC class II-restricted immunogenic potential of peptide p;
Fₚ corresponds to the observed frequency of the mutation encoded by peptide p in the tumor biopsy;
Eₚ corresponds to expression of the mutation encoded by peptide p in the tumor biopsy (which in aspects is expression of the gene containing the mutation), expressed as a percentile rank of the observed expression distribution.

The MHC class I-restricted immunogenic potential C1ₚ of peptide p is calculated as follows:

$$C {1}_{p} = {α}_{1} ( Z {1}_{p} - ( Z {1}_{p} \times \frac{H {1}_{p}}{{β}_{1}} ) ) , o r as$$

$$C {1}_{p} = {α}_{1} {\sum }_{e ∈ E {1}_{p}} ( Z {1}_{e} - ( Z {1}_{e} \times \frac{H {1}_{e}}{{β}_{1}} ) ) ,$$

where:
α₁ (e.g., usually set to 1) and β₁ (e.g., usually set to 2, which corresponds to situations in the above-defined methods wherein “epitopes with two or more cross-reactive matches in the reference proteome are categorized as exhibiting a high degree of similarity with self”) are predefined constants;
E1ₚ corresponds to the set of MHC class I-restricted neo-epitopes within peptide p;
Z1ₚ corresponds to the sum of the percentile ranks of each MHC class I-restricted neo-epitope within peptide p, expressed using standard Z-Scores;
Z1ₑ corresponds to the percentile rank of the MHC class I-restricted neo-epitope e, expressed using standard Z-Scores;
H1ₚ is the MHC class I-restricted Homology Score of peptide p, as defined above;
H1ₑ is the MHC class I-restricted Homology Score of neo-epitope e, as defined above.

The MHC class II-restricted immunogenic potential C2ₚ of peptide p is calculated as follows:

$$C {2}_{p} = {α}_{2} ( Z {2}_{p} - ( Z {2}_{p} \times \frac{H {2}_{p}}{{β}_{2}} ) ) , or as$$

$$C {2}_{p} = {α}_{2} {\sum }_{e ∈ E {2}_{p}} ( Z {2}_{e} - ( Z {2}_{e} \times \frac{H {2}_{e}}{{β}_{2}} ) ) ,$$

where:
α₂ (e.g., usually set to 1) and β₂ (e.g., usually set to 2, which corresponds to situations in the above-defined methods wherein “epitopes with two or more cross-reactive matches in the reference proteome are categorized as exhibiting a high degree of similarity with self”) are predefined constants;
E2ₚ corresponds to the set of MHC class II-restricted neo-epitopes within peptide p;
Z2ₚ corresponds to the sum of the percentile ranks of each MHC class II-restricted neo-epitope within peptide p, expressed using standard Z-Scores;
Z2ₑ corresponds to the percentile rank of the MEW class II-restricted neo-epitope e, expressed using standard Z-Scores;
H2ₚ is the MEW class II-restricted Homology Score of peptide p, as defined above;
H2ₑ is the MHC class II-restricted Homology Score of neo-epitope e, as defined above.

Candidate neoantigens (e.g. peptide or polypeptide comprising one or more identified neo-epitopes) are then ranked according to their score, from high to low. The 20 highest ranking neoantigens (e.g. peptide or polypeptide comprising one or more identified neo-epitopes) were selected for our CT26 ANCER™ vaccine and are shown in Table 1.

TABLE 1
Candidate neoantigens (e.g. peptide or polypeptide comprising one or
more identified neo-epitopes)

| | | Sequence | | | | | |
|---|---|---|---|---|---|---|---|
| | | MHC class 1 neo-epitopes | | | | | |
| | | (restriction) | | | | | SEQ |
| | | MHC class 11 neo-epitopes | | Ref | AA | Alt | ID |
| Pep ID | Locus | (restriction) | Gene | AA | Pos | AA | NO: |
| EO_CT26_01 | CHR4: 86583172 | Ac-LQARLTSYE [symbol] LK-NH2 | Haus6 | Ala | 821 | Thr | 1 |
| | | ARLTSYE [symbol] L (Dd) | | | | | 2 |
| | | ARLTSYE [symbol] L (Kd) | | | | | 3 |
| | | ARLTSYE [symbol] L (Ad) | | | | | 4 |
| EO_CT26_02 | CHR11: 69649178 | Ac-ETPEACRQAR [symbol] YLEFSE-NH2 | Fxr2 | Ser | 287 | Asn | 5 |
| | | EACRQAR [symbol] Y (Ad) | | | | | 6 |
| | | ACRQAR [symbol] YL (Kd) | | | | | 7 |
| | | RQAR [symbol] YLEF (Ad) | | | | | 8 |
| EO_CT26_03 | CHR2: 128676212 | AC-SSRVQYVV [symbol] PAVKIVF-NH2 | Anapcl | Asp | 241 | Asn | 9 |
| | | RVQYVV [symbol] PA (Ad) | | | | | 10 |
| | | QYVV [symbol] PAVKI (Kd) | | | | | 11 |
| EO_CT26_04 | CHR2: 158851764 | Ac-TSKYYMRDV [symbol] AIESA-NH2 | Dhx35 | Thr | 646 | Ile | 12 |
| | | SKYYMRDV [symbol] (Kd) | | | | | 13 |
| | | KYYMRDV [symbol] AI (Kd) | | | | | 14 |
| | | YYMRDV [symbol] AI (Dd) | | | | | 15 |
| | | YYMRDV [symbol] AI (Kd) | | | | | 16 |
| | | YYMRDV [symbol] AI (Ad) | | | | | 17 |
| EO_CT26_05 | CHR6: 3377051 | Ac-PALLIK [symbol] MYNKLIS-NH2 | Samd91 | Arg | 70 | His | 18 |
| | | PALLIK [symbol] MY (Ad) | | | | | 19 |
| | | LLIK [symbol] MYNK (Ed) | | | | | 20 |
| | | LIK [symbol] MYNKL (Kd) | | | | | 21 |
| | | K [symbol] MYNKLIS (Ad) | | | | | 22 |
| EO_CT26_06 | CHR5: 100037938 | Ac-LSWDTSKK [symbol] LTEYLSRF-NH2 | Hnrnpdl | Asp | 163 | Asn | 23 |
| | | SWDTSKK [symbol] L (Kd) | | | | | 24 |
| | | TSKK [symbol] LTEYL (Kd) | | | | | 25 |
| | | SKK [symbol] LTEYL (Kd) | | | | | 26 |
| EO_CT26_07 | CHR12: 98815985 | NNVHYLNDGD [symbol] IIYHTAS-NH2 | Eml5 | Asp | 1396 | Ala | 27 |
| | | HYLNDGD [symbol] I (Kd) | | | | | 28 |
| | | HYLNDGD [symbol] II (Kd) | | | | | 29 |
| | | GD [symbol] IIYHTA (Ad) | | | | | 30 |
| EO_CT26_08 | CHRX: 60293650 | Ac-PQPDLYRFV [symbol] RISI-NH2 | Atpllc | Gly | 223 | Arg | 31 |
| | | DLYRFV [symbol] RI (Ed) | | | | | 32 |
| | | LYRFV [symbol] RISI (Kd) | | | | | 33 |
| EO_CT26_09 | CHR12: 98785005 | Ac-DTKCTKADC [symbol] FTHMSR-NH2 | Zc3hl4 | Pro | 653 | Leu | 34 |
| | | TKCTKADC [symbol] (Ad) | | | | | 35 |
| | | KADC [symbol] FTHM (Kd) | | | | | 36 |
| EO_CT26_10 | CHR15: 3275728 | EEDGI [symbol] VWTLLNGN-NH2 | Seppl | Asp | 122 | Ala | 37 |
| | | DGI [symbol] VWTLL (Dd) | | | | | 38 |
| | | DGI [symbol] VWTLL (Kd) | | | | | 39 |
| EO_CT26_11 | CHR7: 55873449 | Ac-ATVHSSMN [symbol] MLEE-NH2 | Cyfipl | Glu | 71 | Lys | 40 |
| | | TVHSSMN [symbol] M (Kd) | | | | | 41 |
| | | VHSSMN [symbol] ML (Ed) | | | | | 42 |
| EO_CT26_12 | CHR12: 91825363 | ILGYRYW [symbol] GIGVLQSC-NH2 | Sel1l | Ala | 299 | Thr | 43 |
| | | GYRYW [symbol] GIGV (Kd) | | | | | 44 |
| | | RYW [symbol] GIGVL (Dd) | | | | | 45 |
| | | RYW [symbol] GIGVL (Kd) | | | | | 46 |
| | | RYW [symbol] GIGVLQ (Kd) | | | | | 47 |
| EO_CT26_13 | CHR6: 52729334 | Ac-FCYVT [symbol] KGEIRGAS-NH2 | Tax1bp1 | His | 107 | Tyr | 48 |
| | | CYVT [symbol] KGEI (Kd) | | | | | 49 |
| EO_CT26_14 | CHR2: 109298148 | Ac-VKICNMQKA [symbol] IL-NH2 | Kif18a | Glu | 383 | Ala | 50 |
| | | KICNMQKA [symbol] (Ad) | | | | | 51 |
| | | KICNMQKA [symbol] I (Kd) | | | | | 52 |
| EO_CT26_15 | CHR10: 122089020 | Ac-RQFPVVEA [symbol] WTMLHDE-NH2 | Tmem5 | Ser | 259 | Asn | 53 |
| | | VVEA [symbol] WTML (Dd) | | | | | 54 |
| EO_CT26_16 | CHR2: 180713221 | Ac-MSYAEK [symbol] DEITKD-NH2 | Gid8 | Pro | 7 | Ser | 55 |
| | | SYAEK [symbol] DEI (Kd) | | | | | 56 |
| EO_CT26_17 | CHR7: 45442527 | Ac-RIQEFVR [symbol] HFY-NH2 | Gys1 | Gly | 310 | Ser | 57 |
| | | RIQEFVR [symbol] H (Kd) | | | | | 58 |
| EO_CT26_18 | CHR5: 129697821 | Ac-KVGLTVKTYE [symbol] LERNIP-NH2 | Sept14 | Leu | 97 | Phe | 59 |
| | | LTVKTYE [symbol] L (Kd) | | | | | 60 |
| | | KTYE [symbol] LERN (Ed) | | | | | 61 |
| | | KTYE [symbol] LERNI (Kd) | | | | | 62 |
| | | TYE [symbol] LERNI (Dd) | | | | | 63 |
| | | TYE [symbol] LERNI (Kd) | | | | | 64 |
| EO_CT26_19 | CHR7: 65663891 | Ac-NSSTYW [symbol] GNPEMETLQ-NH2 | Tarsl2 | Glu | 353 | Lys | 65 |
| | | TYW [symbol] GNPEM (Kd) | | | | | 66 |
| | | TYW [symbol] GNPEME (Kd) | | | | | 67 |
| | | [symbol] GNPEMETL (Kd) | | | | | 68 |
| EO_CT26_20 | CHR11: 58188928 | Ac-RKSYYMQ [symbol] YFLDTV | Gm12250 | Asn | 390 | Lys | 69 |
| | | KSYYMQ [symbol] YFL (Kd) | | | | | 70 |
| | | SYYMQ [symbol] YFL (Kd) | | | | | 71 |
| | | SYYMQ [symbol] YFLD (Kd) | | | | | 72 |
| | | YYMQ [symbol] YFLDT (Kd) | | | | | 73 |

[symbol]

indicates data missing or illegible when filed

1.2 In Vivo Study Design

The effectiveness of the ANCER™-designed CT26 vaccine was tested in Balb/c mice. Mice were separated into three groups: 1) PBS control; 2) poly-ICLC (vehicle); 3) ANCER™-selected CT26 Neoantigen Peptides+poly-ICLC.
Group 1: PBS, sc injections at days 5, 8, 12, 15, 19, 22, and 26.
Group 2: poly-ICLC, 50 μg, sc injections at days 5, 8, 12, 15, 19, 22, and 26.
Group 3: 5 μg/ANCER™-selected CT26 Neoantigen Peptide (100 μg total ANCER™-selected CT26 Neoantigen Peptides)+50 μg poly-ICLC, sc injections at days 5, 8, 12, 15, 19, 22, and 26.

All mice (N=10 mice per group) were injected 3*10⁵ CT26 tumor cells in 0% Matrigel at day 0. Mice were sacrificed once the tumor volume reached 2,000 mm³ or at day 45, whichever came first.

1.3 Poly-ICLC Fails to Control Tumor Burden in the CT26 Model

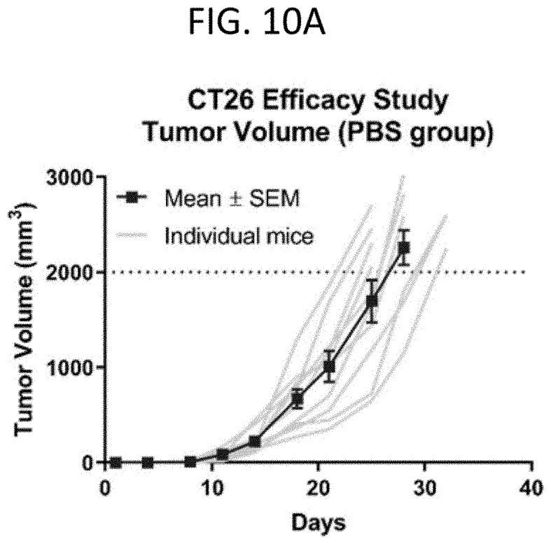

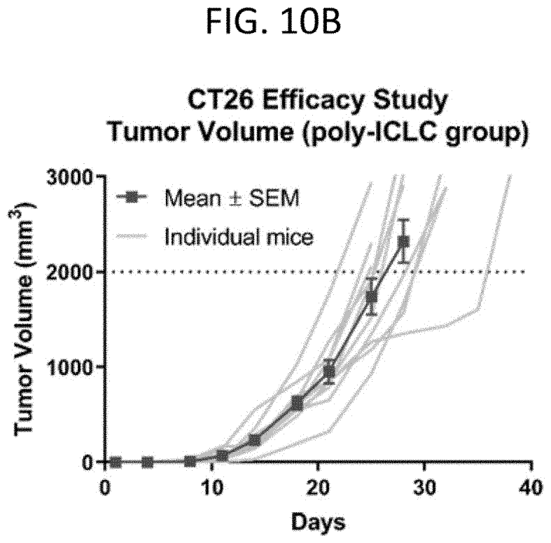

FIGS. 10A-B depict. CT26 tumor growth in PBS control (FIG. 10A) and poly-ICLC (FIG. 10B) groups. Individual mice are shown in lighter shading. Darker shading represents average tumor growth+/−SEM. The average is plotted until half the mice reach endpoint. Group 2 (mice immunized with poly-ICLC) showed no reduction in tumor burden compared to Group 1 (mice immunized with PBS). These results are consistent with previously reported experiments by Charles River. Less than 50% of mice from Group 1 survived past day 28, with seven out of ten mice (70%) reaching a tumor volume of at least 2,000 mm³ by that day. None of the mice from Group 1 survived the 45 days of the experiment. Similarly, less than 50% of mice from Group 2 survived past day 28, with six out of ten mice (60%) reaching a tumor volume of at least 2,000 mm³ by that day. None of the mice from Group 2 survived the 45 days of the experiment.

1.4 ANCER™-Selected CT26 Neoantigen Peptide Vaccine Reduces Tumor Burden by up to 45%

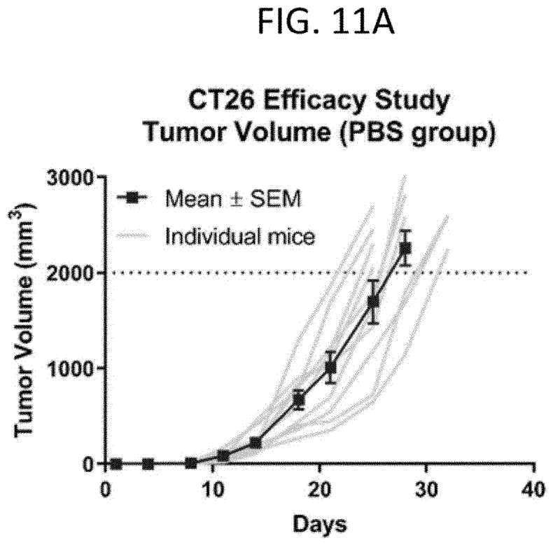

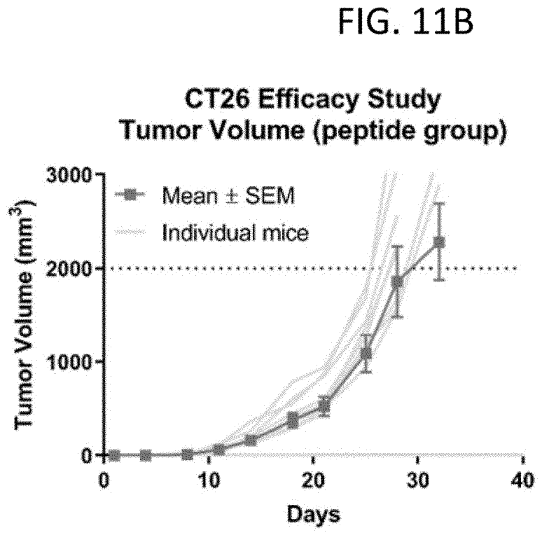

FIGS. 11A-B depict CT26 tumor growth in PBS control (FIG. 11A) and ANCER™-selected CT26 Neoantigen Peptides (FIG. 11B) groups. Individual mice are shown in lighter shading. Darker shading represents average tumor growth+/−SEM. The average is plotted until half the mice reach endpoint. Group 3 (mice immunized with the ANCER™-selected CT26 Neoantigen Peptide vaccine+poly-ICLC) showed a prolonged survival compared to Group 1. In contrast with Group 1 where 70% of mice reached endpoint by day 28, only 50% of mice from Group 3 reached endpoint by that day. Less than 50% of mice from Group 3 survived past day 32, with 8 out of ten mice (80%) reaching a tumor volume of at least 2,000 mm³ by that day. Two of the mice (20%) from Group 3 survived the 45 days of the experiment.

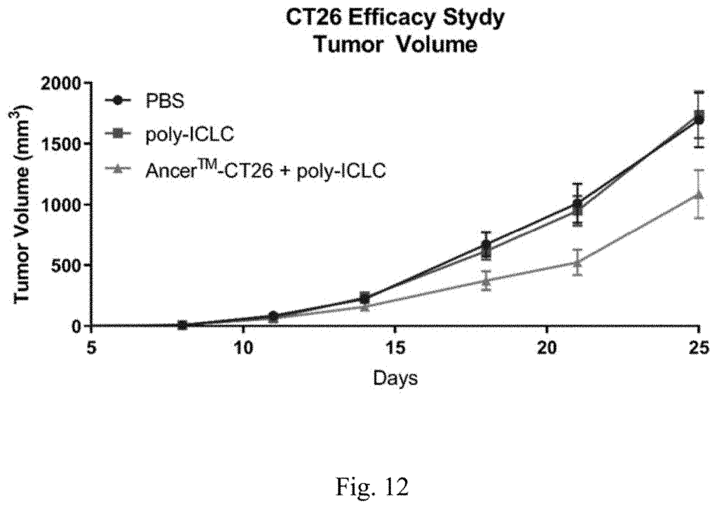

FIG. 12 depicts the mean (+/−SEM) CT26 tumor growth in PBS control, poly-ICLC, and ANCER™-selected CT26 Neoantigen Peptides groups. Means are plotted until half the mice reach endpoint. In addition, tumor growths were reduced in Group 3 compared to Groups 1 and 2. The highest tumor growth inhibitions were observed at days 21 and 25, where tumor burden was reduced by 45% and 38%, respectively, in Group 3 compared to Group 2.

### Example 2

### Suppression of IFN-γ Responses When Co-Administrating Self-Like Neo-Epitopes Along With a Peptide Vaccine

Using the ANCER™ platform, CT26 variants were identified and ranked as potential vaccine candidate peptides. From this this list, 20 neoantigens were selected to be utilized in the development of a peptide-based vaccine developed for the CT26 colorectal cancer syngeneic mouse model (see above).

To demonstrate the importance to identify and eliminate putative “self-like” neo-epitopes due to their potential to dampen immunogenicity responses to vaccine candidates, we have identified CT26 neo-epitopes exhibiting high degree of self-similarity based on JANUSMATRIX™ and tested how their inclusion in vaccine formulations alter their immunogenicity.

2.1 Selection of CT26 Self-like Sequences

The 378 variants extracted from the private and public CT26 mutanomes were screened with the JANUSMATRIX™ algorithm to identify neoantigen sequences that displayed a high degree of similarity with murine sequences. Thirty-five and 24 out of these 378 SNVs generated putative “self-like” regulatory T cell neo-epitopes restricted to MHC class II and MHC class I, respectively. Ten of these MHC class-II-restricted sequences were filtered out due to potential manufacturability issues or due to the presence of putative Treg neo-epitopes with limited potential of binding to MHC.

The remaining 25 MHC class II-restricted sequences were manually reviewed to prioritize neoantigens containing the most highly cross-conserved neo-epitopes. In other words, neoantigens encoding MHC class II-restricted neo-epitopes with the highest number of homologous matches with compatible TCR faces within the reference murine proteome were prioritized over the remaining neoantigens. Ten MHC class II “self-like” neoantigens were selected from this list (as shown in Table 2) to be used in in vivo immunogenicity studies. Sequences in Table 2 are shown with optional N and C-terminal caps (Ac and NH₂, respectively).

TABLE 2
Highly cross-conserved MHC class II-restricted neo-epitopes

| | | Sequence | | | | | |
|---|---|---|---|---|---|---|---|
| | | MHC class II | | | | | SEQ |
| | | tolerated/tolerogenic | | Ref | AA | Alt | ID |
| Pep ID | Locus | epitopes (restriction) | Gene | AA | Pos | AA | NO: |
| EO_CT26_Treg_01 | CHR2: 109894549 | Ac-PQKLQALQR [symbol] LQSE-NH2 | Lin7c | Val | 41 | Ala | 74 |
| | | KLQALQR [symbol] L (Ad) | | | | | 75 |
| EO_CT26_Treg_02 | CHR2: 156167568 | Ac-PHSIKLSRRRSRSK [symbol] PFRKDKSPVR- | Rbm39 | Ser | 129 | Asn | 76 |
| | | NH2 | | | | | |
| | | IKLSRRRSR (Ed) | | | | | 77 |
| | | KLSRRRSRS (Ed) | | | | | 78 |
| | | LSRRRSRSK (Ed) | | | | | 79 |
| | | SRRRSRSK [symbol] (Ed) | | | | | 80 |
| | | SK [symbol] PFRKDK (Ed) | | | | | 81 |
| EO_CT26_Treg_03 | CHR2: 76712742 | Ac-HPWLKQRID [symbol] VSTK-NH2 | Ttn | Arg | 31554 | Lys | 82 |
| | | WLKQRIDK [symbol] V (Ed) | | | | | 83 |
| EO_CT26_Treg_04 | CHR3: 40805884 | Ac-HSVEML [symbol] KPRRSLDEN-NH2 | Plk4 | Ser | 405 | Leu | 84 |
| | | ML [symbol] KPRRSL (Ad, Ed) | | | | | 85 |
| EO_CT26_Treg_05 | CHR3: 90025194 | Ac-KTPSS [symbol] ENPSSNLD-NH2 | Ubap21 | Met | 295 | Leu | 86 |
| | | SS [symbol] ENDSSN (Ed) | | | | | 87 |
| EO_CT26_Treg_06 | CHR5: 106983158 | Ac-SRVQAAQAQHS [symbol] DSLYKRDND-NH2 | Cdc7 | Glu | 500 | Lys | 88 |
| | | QAAQAQHS [symbol] (Ad) | | | | | 89 |
| | | HS [symbol] DSLYKR (Ed) | | | | | 90 |
| | | Ac-LEGVRLENEKS [symbol] NVIAKKTGNK-NH2 | Ibtk | Ile | 747 | Ser | 91 |
| EO_CT26_Treg_07 | CHR9: 85719917 | VRLENEK [symbol] N (Ed) | | | | | 92 |
| | | EK [symbol] NVIAKK (Ed) | | | | | 93 |
| | | K [symbol] NVIAKKT (Ed) | | | | | 94 |
| EO_CT26_Treg_08 | CHR11: 40728456 | Ac-DTTLLAS [symbol] KKAKKSVSKK-NH2 | Hmmr | Ala | 66 | Thr | 95 |
| | | LLAS [symbol] KKAK (Ed) | | | | | 96 |
| | | S [symbol] KKAKKSV (Ed) | | | | | 97 |
| EO_CT26_Treg_09 | CHR12: 98794154 | Ac-LLVSSLKIW [symbol] KKRDRRCAIH-NH2 | Eml5 | Gly | 44 | Arg | 98 |
| | | SSLKIW [symbol] KK (Ed) | | | | | 99 |
| | | SLKIW [symbol] KKR (Ed) | | | | | 100 |
| | | LKIW [symbol] KKRD (Ed) | | | | | 101 |
| | | W [symbol] KKRDRRC (Ed) | | | | | 102 |
| EO_CT26_Treg_10 | CHR16: 57513281 | Ac-PMNELDKVV [symbol] KHKES-NH2 | Falip1l | Glu | 146 | Lys | 103 |
| | | ELDKVV [symbol] KH | | | | | 104 |

[symbol]

indicates data missing or illegible when filed

In addition, ten MHC class I “self-like” neoantigens were selected (as shown in Table 3) to be used in follow-up in vivo studies. Sequences in Table 3 are shown with optional N and C-terminal caps (Ac and NH₂, respectively).

TABLE 3
Highly cross-conserved MHC class I-restricted neo-epitopes

| | | Sequence | | | | | |
|---|---|---|---|---|---|---|---|
| | | MHC class 1 | | | | | |
| | | tolerated/tolerogenic | | Ref | AA | Alt | SEQ ID |
| Pep ID | Locus | epitopes (restriction) | Gene | AA | Pos | AA | NO: |
| EO_CT26_Treg_MH | CHR1: 163996661 | Ac-VLD [symbol] GCGSGLLGITA-NH2 | Mettll8 | Leu | 184 | Phe | 351 |
| CI_01 | | [symbol] GCGSGLLG (Dd) | | | | | 352 |
| | | CGSGLLGIT (Dd) | | | | | 353 |
| | | GSGLLGITA (Dd) | | | | | 354 |
| EO_CT26_Treg_MH | CHR2: 109894549 | Ac-PQKLQALQR [symbol] LQSE-NH2 | Lin7C | Val | 41 | Ala | 74 |
| CI_02 | | KLQALQR [symbol] L (Dd) | | | | | 75 |
| EO_CT26_Treg_MH | CHR3: 29979653 | Ac-NMDTRPSSD [symbol] SLQHA-NH2 | Mecom | Gly | 301 | Ser | 355 |
| CI_03 | | TRPSSD [symbol] SL (Dd) | | | | | 356 |
| EO_CT26_Treg_MH | CHR3: 138148475 | Ac-RRDVARSSLRL [symbol] IDC-NH2 | Trmt10a | Val | 105 | Ile | 357 |
| CI_04 | | VARSSLRL [symbol] (Dd) | | | | | 358 |
| EO_CT26_Treg_MH | CHR5: 44192134 | Ac-MSNNFVEIK [symbol] SVFKK-NH2 | Tapt1 | Gly | 285 | Glu | 359 |
| CI_05 | | NFVEIK [symbol] SV (Kd) | | | | | 360 |
| EO_CT26_Treg_MH | CHR5: 137327443 | Ac-PSRAIPLG [symbol] IIAVAY-NH2 | S1c12a9 | Thr | 302 | Ile | 361 |
| CI_06 | | RAIPLG [symbol] II (Dd) | | | | | 362 |
| | | AIPLG [symbol] IIA (Dd) | | | | | 363 |
| EO_CT26_Treg_MH | CHR7_55873449 | Ac-VHSSMN [symbol] MLEEGQEY-NH2 | Cyfip1 | Glu | 71 | Lys | 364 |
| CI_07 | | SMN [symbol] MLEEG (Kd) | | | | | 365 |
| EO_CT26_Treg_MH | CHR13_12409236 | Ac-QHFGVE [symbol] TVSSLLN-NH2 | Heatr1 | Glu | 552 | Lys | 366 |
| CI_08 | | GVE [symbol] TVSSL (Kd) | | | | | 367 |
| EO_CT26_Treg_MH | CHR17_84429178 | Ac-IKCLMENL [symbol] DEISQA-NH2 | Thada | Glu | 892 | Lys | 368 |
| CI_09 | | LMENL [symbol] DEI (Kd) | | | | | 369 |
| EO_CT26_Treg_MH | CHR19_53635752 | AC-QQRKFKASR [symbol] SILSEM-NH2 | Smc3 | Asp | 733 | Ala | 370 |
| CI_10 | | KFKASR [symbol] SI (Kd) | | | | | 371 |
| | | FKASR [symbol] SIL (Dd) | | | | | 372 |

[symbol]

indicates data missing or illegible when filed

2.2 Study Design

Balb/c mice were separated into three groups: A) Vehicle control; B) ANCER™-selected CT26 Neoantigen Peptides+adjuvant; C) ANCER™-selected CT26 Neoantigen Peptides+JANUSMATRIX™ MHC class II selected peptides+adjuvant. All vaccines were formulated with the addition of the adjuvant Poly-ICLC. Poly-ICLC, also known as Hiltonol, is a synthetic double-stranded RNA (dsRNA) agonist for pattern recognition receptors (PRRs), and TLR3 agonist. Groups A, B, and C received an initial vaccination with subsequent boosts of vaccine at 2- and 4-weeks post initial vaccination. All mice are sacrificed at 7-10 days after the final boost and spleens were harvested for splenocyte isolation and IFNγ ELISpot assay.

### Group A: 50 ug Poly-ICLC in 200 uL

Group B: 20 ANCER™-selected CT26 Neoantigen Peptides at 5 ug/peptide, 100 ug total peptide, 50 ug Poly-ICLC, 200 uL
Group C: 20 ANCER™-selected CT26 Neoantigen Peptides+10 JANUSMATRIX™ MHC class II self-like peptides at 5 ug/peptide, 150 ug total peptide, 50 ug Poly-ICLC, 200 uL

Isolated splenocytes were plated and stimulated with ANCER™-selected CT26 Neoantigen Peptides (CT26_pool), JANUSMATRIX™ selected peptides (CT26-Treg_pool), class I peptide pool, as well as individual ANCER™-selected CT26 Neoantigen peptide CT26-1 (CT26_peptide 1) and CT26-20 (CT26_peptide 1). Plates were incubated overnight and then read. A positive result was defined as spot forming cells >50 SFC/million splenocytes over background, and a Stimulation index>2-fold over background. Statistical significance was determined by Student's T-test; per mouse—antigen vs. no antigen stimulus, as well as group comparisons (p<0.05).

2.3 ANCER™-Selected CT26 Neoantigen Peptides are Immunogenic

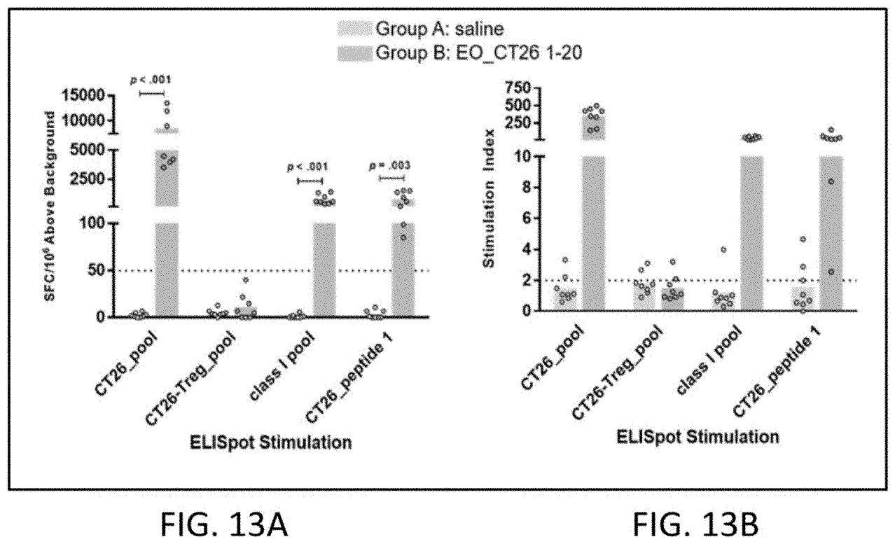

FIGS. 13A-B depict ANCER™-selected CT26 Neoantigen Peptide IFNγ response. CT26 neoantigen peptide pool, Class I only pool, and individual peptide CT26-1 elicited a significant epitope-specific IFNγ response in mice who were vaccinated with CT26 peptides. JanuxMatrix™ selected peptides were not recognized and no positive results were measured in this stimulation condition. ANCER™-selected CT26 Neoantigen Peptides were determined to be immunogenic in mice that were vaccinated with ANCER™-selected CT26 Neoantigen Peptides+Poly-ICLC. Recall responses to the CT26 pool stimulated a significantly increased epitope-specific IFNγ response compared to cells stimulated with media only (FIG. 13). No IFNγ response was measured in the cells stimulated with the CT26-Treg peptide pool demonstrating that irrelevant peptides are not recognized in mice vaccinated with only CT26 peptides. In addition, the CT26 neoantigens were able to stimulate a CD8+ specific T cell response as demonstrated by the positive response to the class I pool. Responses to individual epitopes varied. While CT26-1 (class I and II) epitope appears immunogenic, CT26-20 (Class I) was not (data not shown).

2.4 ANCER™-Selected CT26 MHC Class II “Self-Like” Treg Neoantigen Peptides Suppress Neoantigen Immune Responses

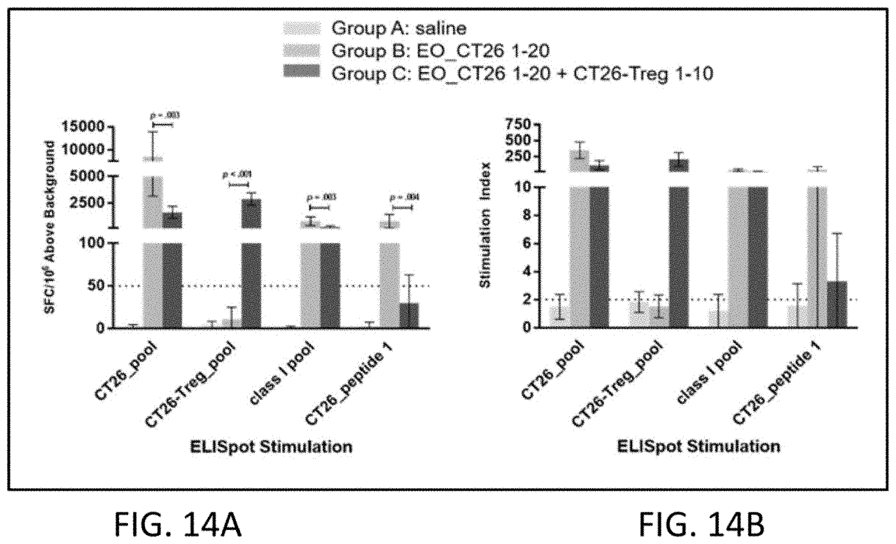

FIGS. 14A-B depict CT26 MHC class II “self-like” peptides suppress IFNγ Responses to ANCER™-selected CT26 Neoantigen Peptides. The ANCER™-selected CT26 Neoantigen Peptide pool elicited a strong IFNγ response in group B vaccinated mice that was not seen in group A demonstrating epitope specific responses (FIGS. 14A-B). The CT26 peptide pool and Class I pool were also able to stimulate strong epitope specific IFNγ responses in group B mice when compared to group A, but the addition of JANUSMATRIX™ selected WIC class II epitopes in the vaccines administered to group C significantly reduces IFNγ responses.

Ancer™-selected CT26 Neoantigen Peptide responses are suppressed in group C mice who were immunized with both ANCER™-selected CT26 Neoantigen Peptides and JANUSMATRIX™ selected neoantigens compared to group B who only received ANCER™-selected CT26 Neoantigen Peptides.

### Conclusion for Examples 1 and 2

The aim of this study was to evaluate the immunogenicity of selected neoepitopes from CT26, a colorectal cancer cell model, as preclinical proof on concept study that the ANCER™ platform can successfully predict peptides from the mutanome that can be used in a neo-epitope cancer vaccine. This data demonstrates that vaccination with ANCER™-selected CT26 neoantigen peptides stimulates a strong, de novo epitope-specific IFNγ response in naïve Balb/C mice. This includes Class I and II peptide pools as well as Class I only peptide pools, suggesting that ANCER™ successfully identified immunogenic CD8+ T cell epitopes. Furthermore, identified CT26 neoantigen peptides that contained putative “self-like” regulatory T cell neo-epitopes demonstrated immunosuppressive capabilities by dampening the IFNγ response seen in response to stimulation with ANCER™-selected CT26 neoantigen peptide pools. Thus, it is valuable to consider any neo-epitopes that may have the ability to induce tolerance as opposed to immunogenicity. Analysis of the WIC- and TCR-facing residues of T cell epitopes by ANCER™ enables prediction of epitope phenotype and can help eliminate any potential immune tolerance within the vaccine.

### Example 3

### T Cells Display Increased Polyfunctionality with ANCER™-CT26 Vaccine Formulation

3.1 Methods

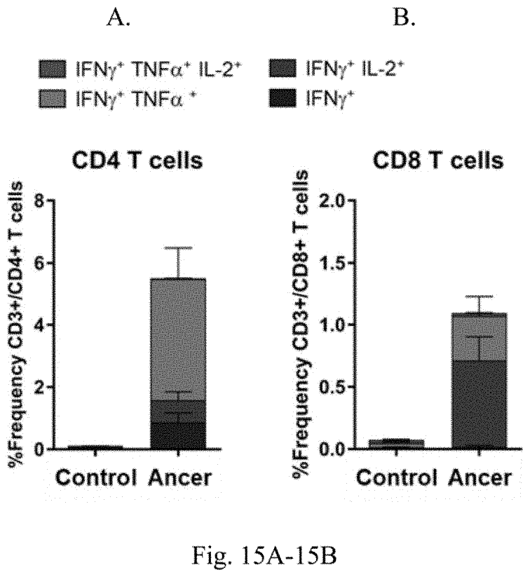

To assess the polyfunctionality of CD4+ and CD8+ T cells, cytokine production after ANCER™-selected CT26 neoantigen peptide (“Ancer™-CT26”) stimulation was assessed in each cell type via flow cytometry. After stimulation with ANCER™-CT26, mice vaccinated with ANCER™-CT26 displayed an increase in the frequency of cytokine producing CD4+ and CD8+ T cells, with the IFNγ+ TNFα+ producing CD4+ population showing the greatest increase (FIGS. 15A-B). FIGS. 15A-B shows that ANCER™-CT26 immunization stimulates multi-functional CD4 and CD8 T cells. Flow cytometry evaluation of splenocytes collected from naïve mice immunized with poly-ICLC or ANCER™-CT26/poly-ICLC. Average+SEM. Other populations secreting IFNγ are also increased, although not as drastically. CD8+ T cells displayed a similar pattern of cytokine expression, with the highest frequency of cytokine secreting cells being IFN+IL-2+ and lesser increases in other IFNγ+ secreting populations.

3.2 Results.

The aim of this study was to evaluate the immunogenicity of selected neo-epitopes from CT26, a mouse colorectal carcinoma model, to demonstrate that our ANCER™ platform can successfully predict peptides from a mutanome for use in a neo-epitope vaccine (e.g. a shared or personalized neo-epitope vaccine). The data demonstrate that vaccination with ANCER™-CT26 stimulates a strong, de novo epitope-specific IFNγ response in naïve female BALB/c mice. Furthermore, identified CT26-Treg-neoantigen peptides demonstrated immunosuppressive activity, dampening the IFNγ response seen in response to stimulation with ANCER™-CT26. This demonstrates the value of identifying neo-epitopes that may have the ability to induce tolerance as opposed to immunogenicity. Analysis of the MHC- and TCR-facing residues of T cell epitopes by ANCER™ enables prediction of the phenotype of the T cell response and can help eliminate any potential immune tolerance within the vaccine.

### Example 4

### (Prophetic) Identification of Treg Inducing Neo-Epitopes and Characterization of their Effect on the T Cell Compartment

4.1 Methods

In this experiment, we are demonstrating the identification of Tregitope sequences, inducing regulatory T cells, from a pool of computationally derived putative self-like neo-epitopes. Naïve Balb/c animals will be immunized with a mixture of 20 neoantigen peptides that are void of Tregitopes and 10 neoantigen peptides predicted to induce Treg responses. Spleens will be collected at day 35. Splenocytes will be cultured in the presence of:
The 20 (Teffector “Teff”) neoantigen peptides
The 20 (Teff) neoantigen peptides+the 10 (Treg) self-like neoantigen peptides
The 20 (Teff) neoantigen peptides+1 (Treg) self-like neoantigen peptide (one culture for each of the 10 self-like sequences)

Readout: flow cytometry.

4.2 Expected Results
Culture with the 20 Teff neoantigens will induce the proliferation of CD3+CD25^{int} FoxP3^{low} CD8+ T cells (activated CD8+ T cells) and CD3+CD25^{int} Foxp3^{low} CD4+ T cells (activated CD4+ T cells).
Culture with the 20 Teff and 10 Treg neoantigens will reduce the proliferation of CD3+CD25^{int} FoxP3^{low} CD8+ T cells (activated CD8+ T cells) and CD3+CD25int Foxp3low CD4+ T cells (activated CD4+ T cells) compared to control (20 Teff neoantigens only). CD3+CD25^{high} Foxp3^{high} CD4+ T cells (Tregs) may be expanded.
Some cultures with the 20 Teff and 10 Treg neoantigens will reduce the proliferation of CD3+CD25^{int}FoxP3^{low} CD8+ T cells (activated CD8+ T cells) and CD3+CD25^{int} Foxp3^{low} CD4+ T cells (activated CD4+ T cells) compared to control (20 Teff neoantigens only). CD3+CD25^{high} Foxp3^{high} CD4+ T cells (Tregs) may be expanded. Self-like neoantigens that can reduce the proliferation of activated CD8+ and CD4+ T cells will be categorized as Tregitopes.

### Example 5

### (Prophetic) Tumor Growth Inhibition Post Vaccination when Co-Administrating Self-Like Neo-Epitopes Along with a Peptide Vaccine

5.1 Methods

In this experiment, we are demonstrating the lowering of tumor growth inhibition of a mice CT26 neoepitope vaccine by adding identified Tregitopes to a mixture of 20 neoantigens peptides that are void of tregitopes (at least, they have low potential for inducing Tregs). The experiment is similar to Example 4, but tumor growth inhibition is measured. A neo-epitope-based vaccine is engineered for the CT26 tumor line. Twenty neoantigens, with low potential for inducing Tregs, are selected for the vaccine. CT26-bearing mice are immunized with saline, a neo-epitope-based vaccine encoding 20 neoantigens with polyICLC and with or without 10 self-like neo-epitopes (N=12 mice per group). Tumor volumes are monitored for up to 60 days.

5.2 Expected Results
Median tumor growth should be reduced by more than 50% in mice immunized with the vaccine compared to mice receiving saline.
Median tumor growth should be reduced by less than 50% in mice immunized with the vaccine in presence of the mixture of 10 self-like neo-epitopes.
Ideally median tumor growth should be reduced by less than 30% in mice immunized with the vaccine in presence of the mixture of 10 self-like neo-epitopes (therefore, presence of self-like neo-epitopes in that model should decrease median tumor growth by at least 20%, preferably more).

### Example 6

### (Prophetic) Tumor Growth Inhibition Post Vaccination when Co-Administrating Self-Like Neo-Epitopes Along with a Peptide Vaccine

6.1 Methods

In this experiment, we are demonstrating the lowering of tumor growth inhibition of a mice CT26 neoepitope vaccine by adding a specific Tregitope identified in a previous study to a mixture of 20 neoantigens peptides that are void of Tregitopes (at least, they have low potential for inducing Tregs). The experiment is similar to Example 2, but tumor growth inhibition is measured instead of immunogenicity. A neo-epitope-based vaccine is engineered for the CT26 tumor line. Twenty neoantigens, with low potential for inducing Tregs, are selected for the vaccine. CT26-bearing mice are immunized with saline, a neo-epitope-based vaccine encoding 20 neoantigens with polyICLC and with or without a specific well characterized Tregitope (N=12 mice per group). Tumor volumes are monitored for up to 60 days.

6.2 Expected Results:
Median tumor growth should be reduced by more than 50% in mice immunized with the vaccine compared to mice receiving saline.
Median tumor growth should be reduced by less than 50% in mice immunized with the vaccine in presence of the specific well characterized Tregitope.
Ideally median tumor growth should be reduced by less than 30% in mice immunized with the vaccine in presence of specific well characterized Tregitope (therefore, presence of tregitopes in that model should decrease median tumor growth by at least 20%, preferably more).

### Example 7

### (Prophetic) Tumor Growth Inhibition Post Vaccination when Comparing a Peptide Vaccine Designed to have Low Potential for Inducing Tregs and a Peptide Vaccine where Potential for Inducing Tregs

7.1 Methods

In this experiment, we are demonstrating the lowering of tumor growth inhibition of a mice CT26 neoepitope vaccine by comparing a mixture of 20 neoantigens peptides that are void of Tregitopes (at least, they have low potential for inducing Tregs) and a mixture of 20 neoantigens corresponding to the same tumor line whereby self-like neo-epitopes (including Tregitopes) are not removed. A neo-epitope-based vaccine is engineered for the CT26 tumor line. Twenty neoantigens, with low potential for inducing Tregs, are selected for the vaccine. Twenty other neoantigens are also selected according to method known to the art. It is expected that the 20 neoantigen designed using methods known to the art will include a certain number (at least one) of self like peptide and/or Tregitope known to reduce their immunogenicity and therefore the strength of their anti-tumor effect. CT26-bearing mice are immunized with saline, a neo-epitope-based vaccine encoding 20 neoantigens with polyICLC and a neo-epitope-based vaccine encoding 20 neoantigens with self and Tregitope present (N=12 mice per group). Tumor volumes are monitored for up to 60 days.

7.2 Expected Results
Median tumor growth should be reduced by more than 50% in mice immunized with the vaccine compared to mice receiving saline.
Median tumor growth should be reduced from the vaccine corresponding to the neoantigen comprising self-peptides and Tregitope compared to the vaccine that are void of Tregitopes (ideally, we will observe a 10& reduction, preferably 20%, more preferably, 30%, etc., between the groups).

### Example 8

### (Prophetic) Determine Tolerance-Inducing Nature of ANCER™ Selected Self-Like Epitopes in CT26 Mice

8.1 Methods

The ANCER™ pipeline uses the JANUSMATRIX™ algorithm to filter out Treg-neoAg in order to improve immune responses to cancer vaccines. JANUSMATRIX™ has been validated in prospective vaccine studies for infectious diseases. The goal of the studies proposed in this aim is to demonstrate that Treg-neoAg are capable of inducing Tregs. In preliminary studies using the CT26 model, we demonstrated that a pool of Treg-neoAg co-administered with an optimally designed neo-epitope vaccine diminished IFNγ ELISpot responses by 5-fold compared to vaccination without the Treg-neoAg in naïve BALB/c mice (p=0.003). Building on this finding, we will evaluate the immunosuppressive capacity of each of the Treg-neoAg by evaluating the induction of Treg responses in naïve mice peptide-immunized with individual Treg-neoAg, co-administered with ANCER™-selected CT26 neoantigen peptides (“Ancer™-CT26”). Treg-neoAg demonstrated to suppress IFNγ responses to ANCER™-CT26 and that are recognized by T cells with a Treg phenotype will be selected for further studies to evaluate their impact on vaccine efficacy in the CT26 model.

8.2 Immunizations

Groups of BALB/c mice (N=6) will be primed by subcutaneous (s.c.) injection of each of the 10 CT26-Treg-neoAg individually, together with ANCER™-CT26 (5 μg/peptide) formulated with the poly-ICLC adjuvant (50 μg) and subsequently boosted twice at two-week intervals. A comparator group will receive a similarly formulated vaccine containing ANCER™-CT26 only following the same immunization schedule. Controls will include matched groups that receive saline and poly-ICLC alone.

8.3 Expected Results

Evaluation of immunosuppressive function of self-like epitopes. One week following the final immunizations, the suppressive effect of individual Treg-neoAg will be evaluated by IFNγ ELISpot assay. Splenocytes will be stimulated with ANCER™-CT26. Reduction of IFNγ-secreting ANCER™-CT26-specific cell numbers in mice immunized with a Treg-neoAg in comparison with mice that received ANCER™-CT26 alone will identify the Treg-neoAg as a potential Treg-inducing epitope, pending confirmation of the T cell phenotype by flow cytometry (see below).

The frequency of epitope-specific splenocytes will be determined using the colorimetric Mabtech IFNγ ELISpot Kit with pre-coated plates according to the manufacturer's protocol. Washed splenocytes in RPMI 1640 (Gibco) supplemented with 10% fetal calf serum will be added at 2.5×10⁵ cells per well. Antigen stimulations will be done in triplicate and include (i) the 20 ANCER™-CT26 peptides added at 10 μg/ml, equivalent to 0.5 μg/ml per peptide, and (ii) individual Treg-neoAg at 0.5 μg/ml. Triplicate wells will be stimulated with 2 μg/ml Concanavalin A as a positive control, and six replicate wells with medium containing 0.02% DMSO will be used for background determination. Raw spot counts will be recorded by ZellNet Consulting, Inc. using a Zeiss high-resolution automated ELISpot reader system and companion KS ELISpot software. Results will be calculated as the average number of SFC in the peptide wells, adjusted to one million cells. A response in immunized mice will be considered positive if the number of average spots is (i) at least twice as high as background (stimulation index>2), (ii) greater than 50 SFC above background per million splenocytes (1 response per 20,000 cells), and (iii) statistically different (p<0.05) from that of mock immunized mice by the Mann-Whitney U test. Differences in SFC numbers between immunization groups (Ancer™-CT26 with and without Treg-neoAg) will be evaluated for statistical significance (p<0.05) by the Mann-Whitney U test.

### Example 9

### Epitope-Specific T Cell Phenotyping

9.1 Methods

The phenotype of T cells recognizing Treg-neoAg will be determined using tetramer technology and flow cytometry. PE or APC-labeled MEW II tetramers (I-A^{d}/I-E^{d}) containing the Treg-neoAg will be generated by the NIH Tetramer Core Facility. Splenocytes will be incubated with fixable viability stain 450 to discriminate dead from live cells, stained with tetramers corresponding to the Treg-neoAg used in mouse immunizations, and then stained with a defined panel of antibodies against cell surface markers for simultaneous detection and discrimination of Tregs from other cell types (CD45, CD3, CD4, CD8, CD14, CD19, CD25, CTLA-4, GITR, CD103, ICOS). Tregs will be further functionally discriminated from Teff/Th1 cells by intracellular staining for FoxP3, T-bet, IL-10 and TGF-β1 following stimulation with PMA-ionomycin, blocking of secretion with brefeldin A, fixation and permeabilization after surface staining. Flow cytometry measurements will be made on a BD Fortessa cytometer. Between 500,000 and 1,000,000 events will be collected per sample. Data analysis: Collected data will be analyzed using FlowJo software. Cells will be gated on lymphocyte/singlet/live/CD45⁺CD3⁺CD4⁺CD8⁻CD14⁻CD19⁻ events. Epitope-specific Treg cells will be defined as tetramer-binding CD4⁺CD25⁺FoxP3⁺ T-bet⁻ cells with elevated CTLA-4, GITR, CD103 and/or ICOS frequency and/or MFI in mice immunized with a Treg-neoAg over mice that do not receive the Treg-neoAg. A lower threshold corresponding to 2 standard deviations above background will be built for each population pattern based on a Poisson model. Values below this threshold will be set to 0. For comparisons of response patterns and not magnitude, the proportion of each individual response pattern within the total response will be calculated. Comparisons between groups will be based on a Wilcoxon rank sum test. Statistical significance will be defined as p<0.05 for pairwise comparisons and p<0.01 for multiple comparisons.

9.2 Expected Results

It is unlikely that all 10 Treg-neoAg contribute equally to the 5-fold reduced IFNγ response shown in FIG. 14. Hence, we expect to identify an estimated 3 to 5 individual Treg-neoAg that significantly reduce IFNγ responses to ANCER™-CT26 and generate a Treg response. Treg-neoAg that do not elicit a reduced response with statistical significance may contribute minimally to the 5-fold reduction observed when all 10 Treg-neoAg are delivered together; alternatively, they may be tolerated as a result of clonal deletion or anergy. It is unlikely, but possible, that none of the Treg-neoAg individually reduce IFNγ responses. Even if this were the case, we will still have learned that “dead end” tolerated epitopes need to be avoided in neo-epitope vaccines.

### Example 10

### (Prophetic) Comparing Capped Against Uncapped Peptides

It is well known that modification to the N or C terminus of an immunogenic peptides can induce properties different form the parent molecules. The parameters that could be modified (but not limited to) are:
Half-lives of the peptides
Cellular targeting
Cellular distribution
Endosomal escape and mechanism of translocation
Kinetic and yield of release into the cell
Presentation to the HLA

It is also well known to the art that complex capping can dramatically change the overlap properties of the immune peptides. Members of the complex capping family include lipopeptides and analogs. In the current example, we intend to compare the effect of modifying N-terminus with acetylation and C term with amidation and compare their in vivo induction of TCell responses and their anti-tumor growth effect in a CT26 mice mode.

### Example 11

### (Prophetic) Confirm Decreased Tumor Burden after Vaccination of CT26 Mice without Putative Treg Epitopes

11.1 Methods

The goal of these studies is to demonstrate the harmful impact Treg-neoAg have on neo-epitope vaccine efficacy using the CT26 model. Two approaches to vaccination with Treg-neoAg will be assessed. One will use Treg-inducing Treg-neoAg. The second will use neo-epitopes discovered using ANCER™ without the JANUSMATRIX™ algorithm. Assessment of a vaccine designed without JANUSMATRIX™, a unique feature of ANCER™ over other pipelines, will establish its importance to the neo-epitope selection process. Additionally, immunogenicity of these vaccines will be assessed and compared with the optimally designed ANCER™ CT26 vaccine.

11.2 Evaluate Therapeutic Efficacy of CT26 Neo-Epitope Vaccine in Tumor-Bearing Mice

We will first optimize dosing schedule for peptide-adjuvant vaccinations by immunizing tumor-bearing animals at two different times post-tumor implantation and with three different boosting schedules. BALB/c mice will be injected s.c. with 3×10⁵ CT26 tumor cells. We will vaccinate three groups of tumor-bearing mice (10 mice/group) s.c. with ANCER™-CT26 formulated with poly-ICLC 4 days following tumor implantation with the following schedule: Group 1 on days 4, 12, 20; Group 2 on days 4, 18; Group 3 on days 4, 7, 10, 13, 16, 19, 22. Two additional groups will be vaccinated 10 days following tumor implantation with the following schedule: Group 4 on days 10, 18, 26; Group 5 on days 10, 13, 16, 19, 22. We will use the optimal dosing schedule determined in the first part of this Aim to immunize CT26 tumor-bearing mice with ANCER™-CT26/poly-ICLC. Comparator groups of tumor implanted mice will receive ANCER™-CT26/poly-ICLC with the validated Treg-neoAg from Aim 1, or with a similarly formulated vaccine containing CT26 neo-epitope peptides discovered without JANUSMATRIX™ using the same immunization schedule. Controls will include matched groups of tumor implanted mice that receive saline or poly-ICLC alone. A corresponding set of mice will receive combined vaccine and CPI therapy using anti-PD-1 antibody (RMP1-14) biweekly over two weeks at a 5 mg/kg dose and will be compared to anti-PD-1 monotherapy.

11.3 Tumor Progression

Tumor progression will be followed biweekly by caliper measurement to the study endpoint, either a tumor volume of 2,000 mm³ or 45 days, whichever comes first. Survival curves will be generated from the outcome of the experiment. Efficacy of each vaccine candidate will be defined as increased survival over untreated animals. Survival function will be compared using a log rank test. Two-way ANOVA will be used to compare the different vaccine treated groups for their therapeutic effect. Tukey's post-hoc analysis will be used to examine whether each vaccine has a differentiated therapeutic effect in comparison with the other groups. A sample size of 10 mice per group (n=10) will have a power of 0.80 to detect a 5.97-day difference in the median time to endpoint (TTE) between the vaccine groups with a standard deviation of 28.8% of the mean, given a type I error of 0.05.

11.4 Immune Monitoring Studies

TILs and splenocytes from an additional six mice per vaccine and control group will be analyzed for responses to neoantigens at day 21 post-implantation by ex vivo ELISpot assay for IFNγ, IL-2 and TNFβ. Flow cytometric analysis will be used to characterize CD4⁺ and CD8⁺ T cell splenic and TIL populations (CD45/CD3/CD4/CD8 expression), Treg numbers and frequencies (CD25/FoxP3 expression), Treg function (CD25 MFI), CD8⁺ T cell effector and exhaustion states (GzmA/LAG-3 expression and PD-1 MFI). Because tumor associated Tregs are implicated in controlling suppressor macrophage populations, we will perform IHC of tumors for changes in M1 and M2 frequency (F4/80/iNOS/Arg-1).

11.5 Expected Results

We anticipate that ANCER™-CT26 will significantly reduce tumor growth in comparison with untreated mice and that anti-PD-1 combination therapy will further enhance tumor control, potentially exerting full control. Tumor growth in mice that receive the vaccine containing Treg-inducing epitopes is expected to be significantly greater than in mice vaccinated with no Treg-inducing epitopes; because Treg induction promotes tumor growth, tumor control in these mice may resemble or possibly be worse than in untreated mice. CPI therapy may improve tumor control in these mice but will not approximate the combination effect seen in mice that receive ANCER™-CT26. Mice treated with vaccine designed without JANUSMATRIX™ are expected to respond similarly without or with CPI. We expect immune responses in the tumor and systemically to correlate with these tumor growth expectations, i.e. elevated multi-cytokine producing CD4 and CD8 T cells in the tumor in mice that receive ANCER™-CT26 and lower Treg frequencies; elevated IFNγ responses to ANCER™-CT26 peptides in the spleen. A potential challenge is tumor control in mice treated with ANCER™-CT26 that does not allow for discrimination with the “sub-optimal” vaccines. This may be a challenge even though we have preliminary data in the CT26 model (data not shown) that shows the optimal vaccine dosing regimen significantly slows tumor growth in comparison with untreated mice. We can increase the vaccine dose or the number of doses to address this. Additionally, if tumor growth is too fast to observe differences between the vaccines, we can titrate down the number of CT26 cells implanted.

### Example 12

### (Prophetic) Large Multi-Parameters Mice CT26 Study Comprising 7 Independent Studies

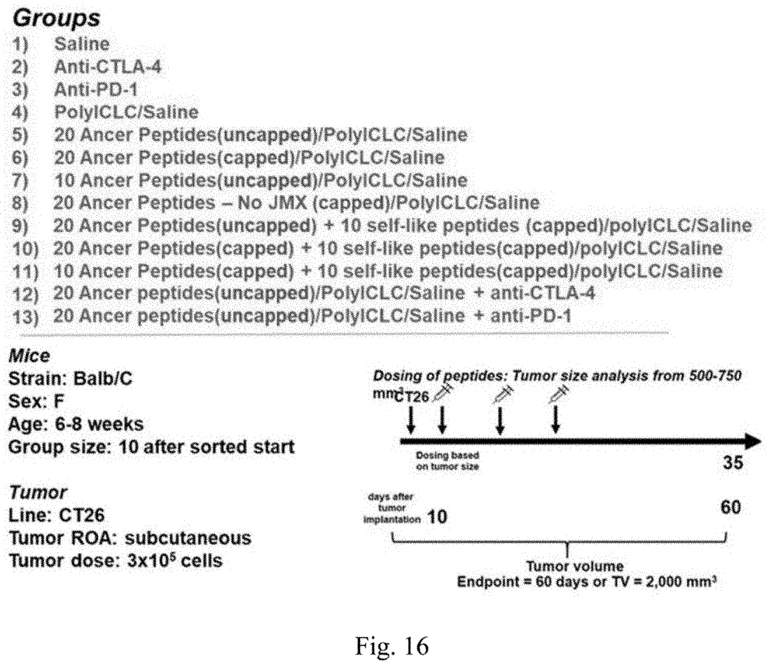

It is our intent to run a large mice CT26 tumor study with adequate controls. The study should be giving us insights on 8 independent study groups and therefore produce 8 adequately controlled studies. Design of the overall study with controls as disclosed in FIG. 16.

12.1 Study #1: Efficacy of Our Vaccine in Tumor Growth Inhibition Models (Comprising Groups 1, 4 and 5)

The goal is to demonstrate proof-of-concept that ANCER™ selects neoantigens that control tumor growth.

Mouse groups that will demonstrate this include:
Saline
PolyICLC/Saline
20 ANCER™ Peptides(uncapped)/PolyICLC/Saline

12.2 Study #2: Synergy with Anti-PD1 (Comprising Groups 1, 3, 4, 5 and 13)

The goal is to determine the contributions of vaccination and check-point inhibitor therapy to tumor control.
Mouse groups that will demonstrate this include:
   Saline
   Anti-PD-1
   PolyICLC/Saline
   20 ANCER™ Peptides(uncapped)/PolyICLC/Saline
   20 ANCER™ peptides(uncapped)/PolyICLC/Saline+anti-PD-1

12.3 Study #3: Synergy with Anti-CTLA4 (Comprising Groups 1, 2, 4, 5 and 12)

The goal is to determine the contributions of vaccination and check-point inhibitor therapy to tumor control.
Mouse groups that will demonstrate this include:
   Saline
   Anti-CTLA-4
   PolyICLC/Saline
   20 ANCER™ Peptides(uncapped)/PolyICLC/Saline
   20 ANCER™ peptides(uncapped)/PolyICLC/Saline+anti-CTLA4

12.4 Study #4: Effect of Self or/and Tregitopes (Comprising Groups 1, 4, 5, 6, 9 and 10, 11)

The goal is to show the effect Treg inducing epitopes have on neoantigen vaccine efficacy.
Mouse groups that will demonstrate this include:
   Saline
   PolyICLC/Saline
   20 ANCER™ Peptides(uncapped)/PolyICLC/Saline
   20 ANCER™ Peptides(capped)/PolyICLC/Saline
   20 ANCER™ Peptides(uncapped)+10 self-like peptides (capped)/polyICLC/Saline
   20 ANCER™ Peptides(capped)+10 self-like peptides(capped)/polyICLC/Saline
   10 ANCER™ Peptides(capped)+10 self-like peptides(capped)/polyICLC/Saline

12.5 Study #5: Effect of Filtering Out Self or/and Tregitopes (Comprising 1, 4, 6 and 8)

The goal is to show the effect of JANUSMATRIX™ on neoantigen selection and vaccine efficacy. JANUSMATRIX™ is a key tool that differentiates the instantly-disclosed strategies, methods, and compositions from the competition. Twenty sequences will be selected with ANCER™, but without taking JANUSMATRIX™ results into consideration.
Mouse groups that will demonstrate this include:
   Saline
   PolyICLC/Saline
   20 ANCER™ Peptides(capped)/PolyICLC/Saline
   20 ANCER™ Peptides—no JMX(capped)/PolyICLC/Saline

12.6. Study #6: Effect of Capping Peptides (Comprising 1, 4, 5 and 6)

The goal is to compare efficacy of vaccines containing capped or uncapped peptides. The default is uncapped. Capping may alter peptide properties, such as half-life or antigen processing, that enhance vaccine efficacy.
Mouse groups that will demonstrate this include:
   Saline
   PolyICLC/Saline
   20 ANCER™ Peptides(uncapped)/PolyICLC/Saline
   20 ANCER™ Peptides(capped)/PolyICLC/Saline

12.6 Expected Results

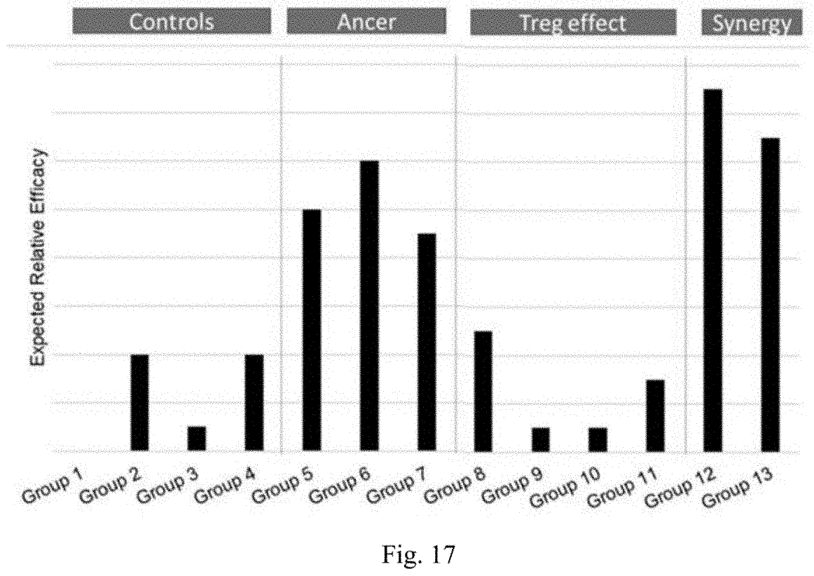

The efficacies observed for each arm included in the study is expected to follow the ranges described in FIG. 17.

FIG. 17. Expected relative efficacy of each study arm. Relative efficacy is shown for the control arms (Groups 1, 2, 3, 4), arms testing ANCER-derived vaccines (Groups 5, 6, 7), arms testing the effect of Treg epitopes in vaccines (Groups 8, 9, 10, 11), and arms testing synergy between an ANCER-derived vaccines and checkpoint inhibitors (Groups 12, 13).

### Example 13

### Anti-Tumor Activity with or without Poly-ICLC Using the CT26 Murine Tumor Model

13.1 Methods

The goal of this experiment was to determine the requirement for the inclusion of the adjuvant poly-ICLC in a peptide vaccine containing ANCER™ selected neoantigens. For this experiment, female BALB/c mice were implanted subcutaneously with 3×10⁵ CT26 murine colon tumor cells. Tumor growth was monitored by caliper measurement and mice were sacrificed 45 days after tumor implantation or when tumor volumes reached 2000 mm³, whichever came first.

Four vaccine groups were tested (n=10 mice/group):

Group 1=PBS only

Group 2=PBS with 50 μg poly-ICLC

Group 3=20 ANCER™ selected (uncapped) peptides with 50 μg poly-ICLC

Group 4=20 ANCER™ selected (uncapped) peptides only

Peptide vaccines were comprised of 20 different ANCER™ selected peptides at a concentration of 5 μg/peptide for a total dose of 100 μg peptide per immunization. Mice were immunized subcutaneously in the opposite flank from tumor implantation twice per week for three weeks, starting at day 4 post-tumor implantation.

13.2 Results

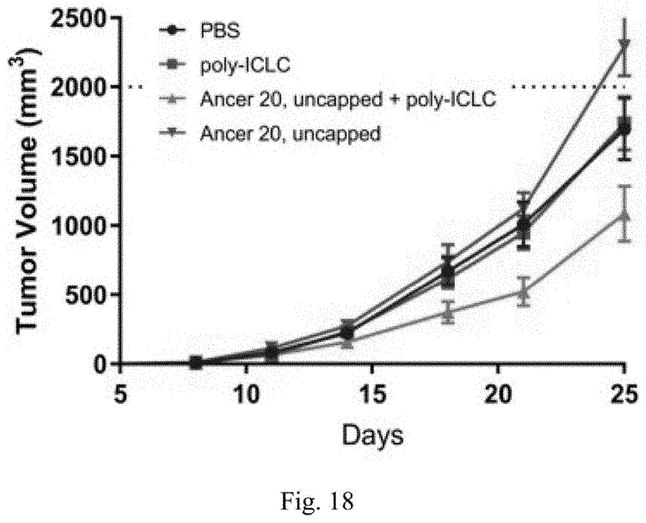

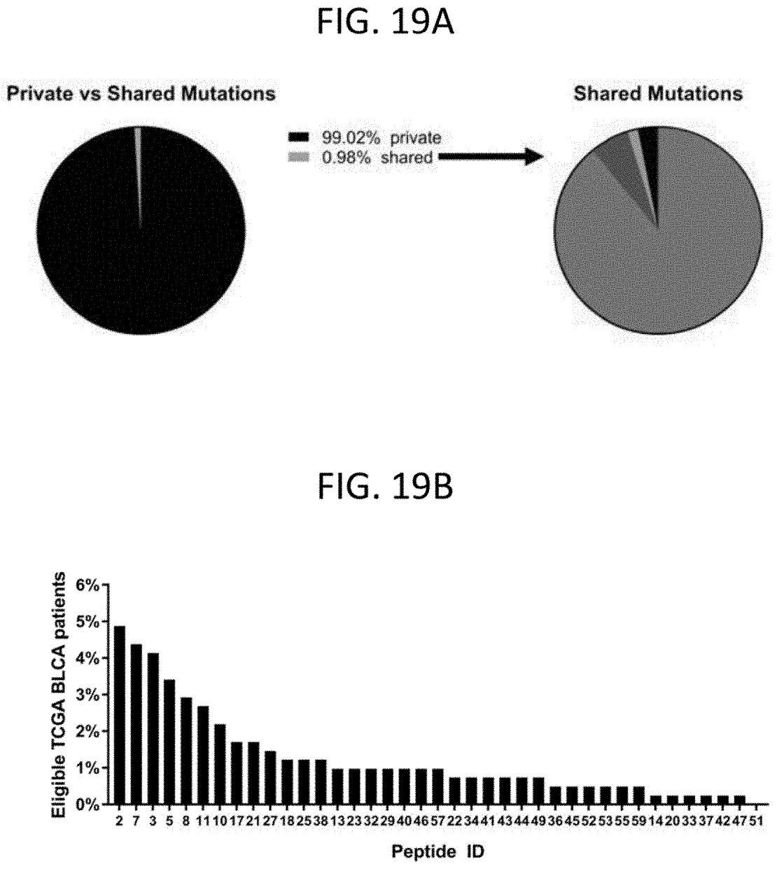

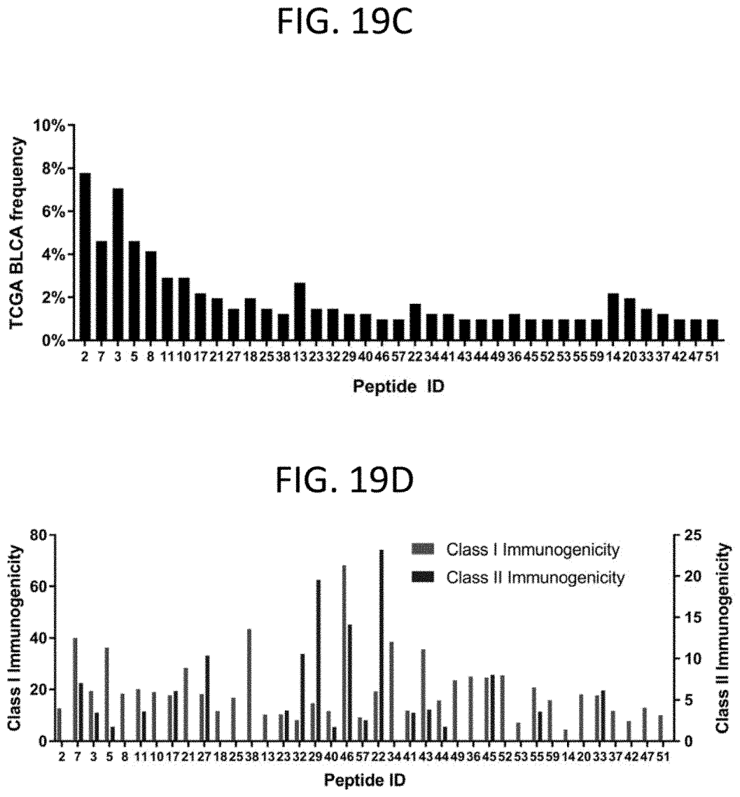

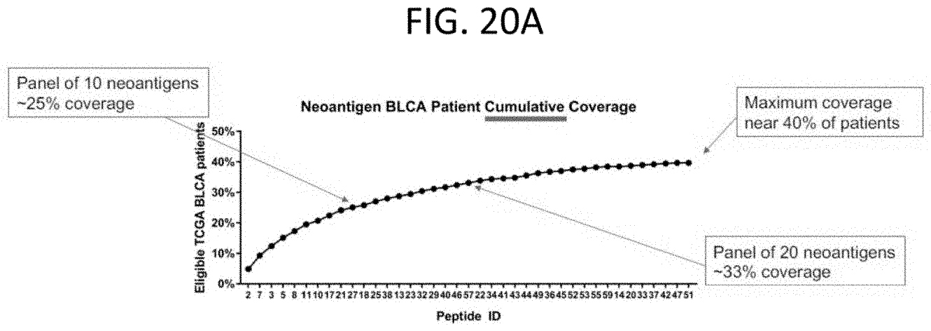

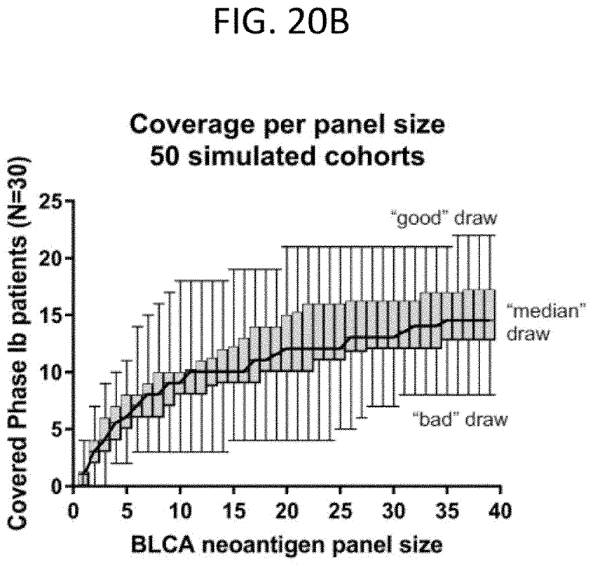

FIG. 18 is a graph depicting efficacy of an ANCER™ selected peptide vaccine administered with poly-ICLC to control tumor growth in the syngeneic CT26 murine tumor model. Peptide vaccine administered in the absence of poly-ICLC failed to control tumor growth relative to animals receiving vehicle (PBS or poly-ICLC alone. The data demonstrate that at day 25 post-tumor implantation, the only group demonstrating statistically significant control of tumor growth was that receiving both ANCER™ selected peptides and poly-ICLC. Poly-ICLC alone showed no difference in tumor growth kinetics relative to mice receiving PBS alone. Mice receiving the ANCER™ selected peptides in the absence of poly-ICLC also failed to control tumor growth relative to non-peptide controls or poly-ICLC alone. These results demonstrate the need for the inclusion of an adjuvant, such as poly-ICLC, with peptide vaccination in order to control tumor growth.

### Example 14 (Prophetic)

### Produce a Neo-Epitope Vaccine with Optimal Immunogenicity in the MB49 Mouse Bladder Model

14.1 Methods

In neo-epitope vaccine production, the process is the product. No two neo-epitope vaccine are the same because mutanomes and HLA types differ from person to person. Rather, the process that produces an efficacious vaccine does not vary. Therefore, we propose to demonstrate the instantly-disclosed neo-epitope vaccine production process succeeds end-to-end in more than one cancer model. We selected the murine transitional MB49 tumor model because it is quite similar to human bladder cancer which makes it an interesting model to study novel immunotherapies. The MB49 line is derived from C57BL/6 mouse bladder epithelial cells that were transformed by a single 24-hour treatment with the chemical carcinogen 7, 12-dimethylbenz[a]anthracene (DMBA) on the second day of a long-term primary culture. After injection in the bladder, the mouse forms urothelial carcinomas within 3-7 days with infiltrating lymphocytes detectable by flow cytometry. These tumors express PD-L1, which is upregulated by IFNγ. In this aim, we propose to sequence the MB49 tumor model, identify mutations generating Teff and Treg neo-epitopes, design ANCER™-selected MB49 neo-epitope vaccines (“Ancer™-MB49”) with and without JANUSMATRIX™, and verify the anti-tumor effect of the ANCER™-MB49 vaccines in tumor-bearing animals.

14.2 Define the MB49 Expressed Mutanome.

We will sequence the MB49 exome and transcriptome using standard next generation sequencing technology and use well-established bioinformatic methods to identify tumor-specific mutations. Single nucleotide variants, in-frame insertions and deletions (e.g. indels), and out-of-frame insertions and deletions (e.g. frameshifts) resulting in non-synonymous mutations will advance to neo-epitope prediction.

14.3 Predict MB49 Neo-Epitopes.

Using the ANCER™ platform, we will identify tumor-specific CD8⁺ and CD4⁺ T cell neo-epitopes with potential to induce protective Teffs and inhibitory Tregs. NeoAg sequences will be designed around each mutation, if possible, in order to include these high-quality MEW class I and II neo-epitopes. After ranking the neoAg based on defined criteria such as (i) MHC class I and II binding potential and TCR-face homology, (ii) transcript abundance and clonality and (iii) hydrophobicity (e.g., as disclosed herein), the top 20 Teff neoAg will be selected for vaccine design.

14.4 Vaccine Production.

Similar to the work realized with the CT26 model, we will synthesize the top 20 ANCER™-selected MB49 neo-epitopes (Ancer™-MB49) corresponding to the top 20 neoAg identified by our bioinformatics and formulate them with poly-ICLC as an exemplary adjuvant. More specifically, using a multiple peptide synthesizer we will synthesize the peptides (15 to 25 amino acid residues on the average) as trifluoroacetate salts using a solid-phase peptide synthesis (SPPS) process. While the peptides included in the vaccine are fully synthetic, they are however entirely composed of natural, unmodified amino acids. All peptides will be manufactured to non-GMP quality standard with certificates of analysis. The 20 peptides will be dissolved in water or isotonic dextrose containing up to 4% dimethyl sulfoxide depending on the solubility of individual peptides then admixed with poly-ICLC (0.5 mg).

14.5 Evaluation of Therapeutic Efficacy of ANCER™-MB49 in Tumor-Bearing Mice.

C57BL/6 mice will be instilled with 1×10⁵ tumor cells into the bladder under anesthesia via a catheter. Tumors in the natural environment of the bladder thrive better than in s.c. models, growing within 3 days. Survival in this model is up to 3 weeks. A group of 10 mice will be primed by s.c. injection of ANCER™-MB49 formulated with poly-ICLC. A comparator group of tumor instilled mice will receive a similarly formulated vaccine containing MB49 neo-epitope peptides discovered without JANUSMATRIX™. The immunization schedule will be as determined in the dosing schedule studies performed in Aim 2. Controls will include matched groups of tumor implanted mice that receive saline or poly-ICLC alone. Corresponding groups of mice will receive vaccines in combination with anti-PD-1 (RMP1-14) administered biweekly over two weeks at a 5 mg/kg dose and will be compared to anti-PD-1 monotherapy.

With luciferase transduction, tumor progression will be followed biweekly by intravital imaging (Xenogen IVIS 200 optical imaging system) to the study endpoint, either a tumor volume of 2,000 mm³ or 45 days, whichever comes first. Survival curves will be generated from the outcome of the experiment. Efficacy of each vaccine candidate will be determined at a later time.

14.6 Immune Monitoring Studies.

TILs and splenocytes from an additional six mice per vaccine and control group will be analyzed for responses to neoantigens at day 21 post-tumor implantation following the same protocol described for the experiments described previously.

14.7 Expected Results.

We anticipate that ANCER™-MB49 will significantly reduce tumor growth in comparison with untreated mice or mice treated with the vaccine designed without JANUSMATRIX™ and that anti-PD-1 combination therapy will further enhance tumor control, potentially exerting full control.

### Example 15 (Prophetic)

### In Vitro HLA Peptide Binding Assay

HLA binding of individual tumor-specific mutated peptides (e.g., a peptide or polypeptide comprising one or more identified neo-epitopes, as disclosed herein) identified from patient whole exome sequencing data will be validated by testing in an in vitro competitive binding assay. In this assay, binding affinity of the test peptide is established by measuring inhibition of HLA binding by a control peptide of know binding affinity. Test peptides are incubated at several concentrations with control peptide at a set concentration along with the corresponding HLA molecule. The level of inhibition of control peptide binding to the HLA molecule is measured at each test peptide concentration and these data are used to establish the binding affinity of the test peptide for the specific HLA molecule evaluated in the assay.

### Example 16 (Prophetic)

### In Vitro Immunogenicity Protocol

Upon verification of peptide binding to patient-specific HLA molecules, we will determine the ability of patient's T cells to recognize and respond to tumor-specific mutated peptides (e.g., the subject-specific peptides or polypeptides comprising the at least one identified neo-epitope comprising, consisting, or consisting essentially of an amino acid sequence of the at least one identified neo-epitope (and/or fragments or variants thereof), and optionally 1 to 12 additional amino acids distributed in any ratio on the N terminus and/or C-terminus of the the at least one identified neo-epitope). Mutated peptides that have been confirmed to bind patient HLA molecules will be synthesized and used to pulse patient-derived professional antigen presenting cells (pAPC), such as autologous dendritic cells or CD40L-expanded autologous B cells. Weekly in vitro re-stimulations with peptide-pulsed autologous pAPC in the presence of IL-2 and IL-7 will be used to expand patient-derived T cells. After several weeks of culture, expanded T cells will be tested for peptide-HLA specific reactivity by ELISpot assay to measure IFN-γ release. Further characterization of peptide-specific T cell responses may be performed using in vitro killing assays such as chromium release assays or comparable methods using patient T cell clones. T cell clones will be generated by in vitro stimulation using peptide-pulsed autologous pAPC and including the additional step of cloning by limiting dilution following standard protocols.

### Example 17 (Prophetic)

### Evaluation of Inhibitory Treg Peptide Sequences

The ability of peptide sequences to activate inhibitory regulatory T cell (Treg) responses capable of suppressing effector T cell function will be evaluated using an in vitro assay referred to as the tetanus toxoid bystander suppression assay (TTBSA). This assay is based on the ability of Tregs to suppress the function of memory T cells specific to tetanus toxoid. Incubation of peripheral blood mononuclear cells (PBMCs) from patients with a history of immunization with tetanus toxoid results in expansion of tetanus toxoid specific CD4⁺ effector T cells. When peptides recognized by Tregs are added in vitro along with tetanus toxoid, activation and proliferation of the tetanus toxoid specific CD4⁺ effector T cells is inhibited by the Tregs in a dose dependent manner. This inhibition of effector T cell activation and proliferation is used as a measure of peptide-specific Treg activity.

### Example 18 (Prophetic)

### Evaluation of Inhibitory Treg Peptide Sequences

Cross-reactive or auto-reactive T cell responses will be tested by in vitro priming of T cells using neoepitope peptides containing non-synonymous amino acid substitutions and presented by autologous pAPC. This in vitro immunogenicity protocol may follow the methodology established by Wullner et al. (Wullner D, Zhou L, Bramhall E, Kuck A, Goletz T J, Swanson S, Chirmule N, Jawa V. Considerations for Optimization and Validation of an In vitro PBMC Derived T cell Assay for Immunogenicity Prediction of Biotherapeutics. Clin Immunol 2010 October; 137(1): 5-14, incorporated by reference in its entirety). T cells that expand following in vitro priming to the neoepitope peptides will then be tested for reactivity to the corresponding native or wild type (non-mutated) peptide epitopes. Reactivity to native peptide sequences will be determined by measuring cytokine production including, but not limited to, IFNγ, TNFα, IL-2 and/or markers of T cell effector function including, but not limited to, CD107a and granzyme B

Although embodiments of the invention have been exemplified and described with specificity, a person of ordinary skill in the art will understand that additional aspects and embodiments are within the scope of the claims as defined by the appended claims.

The invention is further described by the following numbered paragraphs:

1. A method of treating a neoplasia in a subject, the method comprising (a) administering an effective amount of one or more peptides or polypeptides comprising one or more identified shared neo-epitopes; and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells).

2. The method of numbered paragraph 1, wherein the one or more peptides or polypeptides comprising one or more identified shared neo-epitopes comprise, consist of, or consist essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, provided:

the one or more peptides or polypeptides are encoded by a shared neoplasia-specific mutation that is detected in a neoplasia sample from the subject;

the one or more peptides or polypeptides are known or determined (e.g. predicted) to bind to a MHC protein of the subject; and/or

the one or more peptides or polypeptides are known or determined (e.g. predicted) to not bind to a MHC protein of the subject that could lead to a detrimental or suppressive immune response.

3. The method of a numbered paragraph 2, wherein one or more peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, are not administered to a subject suspected of having or having a neoplasia provided:

the one or more peptides or polypeptides are encoded by a shared neoplasia-specific mutation that is not detected in a neoplasia sample from the subject;

the one or more peptides or polypeptides are known or determined to not bind to a MEW protein of the subject; and/or

the one or more peptides or polypeptides are known or determined to bind to a MHC protein of the subject that could lead to a detrimental or suppressive immune response.

4. The method according to any one of numbered paragraphs 1-3, the method further comprising

detecting one or more tumor-specific mutations in a neoplasia sample from a subject and/or determining HLA allotypes present in the subject, and administering one or more of peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, provided:

the one or more peptides or polypeptides are encoded by a shared neoplasia-specific mutation that is detected in a neoplasia sample from the subject;

the one or more peptides or polypeptides are known or determined (e.g. predicted) to bind to a MHC protein of the subject; and/or

the one or more peptides or polypeptides are known or determined (e.g. predicted) to not bind to a MEW protein of the subject that could lead to a detrimental or suppressive immune response.

5. The method of numbered paragraph 4, wherein one or more peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, are not administered to a subject suspected of having or having a neoplasia provided:

the one or more peptides or polypeptides are encoded by a shared neoplasia-specific mutation that is not detected in a neoplasia sample from the subject;

the one or more peptides or polypeptides are known or determined to not bind to a MEW protein of the subject; and/or

the one or more peptides or polypeptides are known or determined to bind to a MHC protein of the subject that could lead to a detrimental or suppressive immune response.

6. The method of any one of numbered paragraphs 1-5, wherein the one or more peptides or polypeptides comprising one or more identified shared neo-epitopes (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C,) are administered within 1 week of detecting one or more tumor-specific mutations in the neoplasia sample from a subject (e.g., tumor tissue, such as bladder cancer tumor tissue) and/or determining HLA allotypes present in the subject.

7. The method of numbered paragraph 1, wherein the one or more identified shared neo-epitopes are identified by a method comprising:

i) assessing identified shared neoplasia-specific mutations from a neoplasia specimen of a subject to identify known or determined shared neo-epitopes encoded by said shared neoplasia specific mutations; and

ii) assessing the identified shared neo-epitopes encoded by said mutations from step (i) to identify neo-epitopes that are known or determined to engage regulatory T cells, and excluding such identified neo-epitopes that are known or determined to engage regulatory T cells from the shared neo-epitopes.

8. The method of numbered paragraph 7, wherein said shared neoplasia-specific mutations are shared neoplasia-specific somatic mutations.

9. The method of any one of numbered paragraphs 7 or 8, wherein said shared neoplasia-specific somatic mutations are single nucleotide variations (SNVs), in-frame insertions, in-frame deletions, out-of-frame insertions, and out-of-frame deletions.

10. The method of any one of numbered paragraphs 7 or 8, wherein said shared neoplasia-specific somatic mutations are mutations of proteins encoded in the neoplasia specimen of the subject diagnosed as having a neoplasia.

11. The method of any one of numbered paragraphs 7-10, wherein assessing the shared neoplasia-specific mutations in step (i) to identify known or determined shared neo-epitopes encoded by said shared neoplasia-specific mutations comprises:

a) determining a binding score for a mutated peptide to one or more WIC molecules, wherein said mutated peptide is encoded by at least one of said shared neoplasia-specific mutations;

b) determining a binding score for a non-mutated peptide to the one or more WIC molecules, wherein the non-mutated peptide is identical to the mutated peptide except for the encoded at least one of said shared neoplasia-specific mutations;

c) determining the percentile rank of the binding scores of both the mutated peptide of step (a) and the non-mutated peptide of step (b) as compared to an expected distribution of binding scores for at least 10,000 randomly generated peptides using naturally observed amino acid frequencies;

d) determining the TCR facing amino acid residues of said mutated peptide and said non-mutated peptide; and

e) identifying the mutated peptide as a shared neo-epitope when:
1) the mutated peptide has a determined binding score in the top 5 percentile of the expected distribution and the non-mutated peptide has a determined binding score below the top 10 percentile of the expected distribution; or
2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide.

12. The method of numbered paragraph 11, wherein the mutated peptide and non-mutated peptide are both 9 amino acids in length or the mutated peptide and non-mutated peptide are both 10 amino acids in length.

13. The method of numbered paragraph 11, wherein the one or more MHC molecules are MHC class I molecules and/or MHC class II molecules.

14. The method of numbered paragraph 12, wherein the one or more MHC molecules are MHC class I molecules and/or MHC class II molecules.

15. The method of numbered paragraph 14, wherein the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal.

16. The method of any one of numbered paragraphs 7-15, wherein assessing the shared neoplasia-specific mutations in step (i) to identify known or determined shared neo-epitopes encoded by said mutations comprises in silico testing.

17. The method of numbered paragraph 16, wherein said in silico testing to identify known or determined shared neo-epitopes encoded by said shared neoplasia-specific mutations in step (ii) comprises using an algorithm to screen protein sequences for putative T cell epitopes.

18. The method of any one of numbered paragraphs 7-17, wherein assessing the identified shared neo-epitopes encoded by said shared neoplasia-specific mutations to identify neo-epitopes that are known or determined to engage regulatory T cells and/or other detrimental T cells (including T cells with potential host cross-reactivity and/or anergic T cells) in step (ii) comprises determining whether said identified shared neo-epitopes encoded by said mutations share TCR contacts with proteins derived from either the human proteome or the human microbiome, wherein said identified shared neo-epitopes encoded by said mutations that are determined to share TCR contacts with proteins derived from either the human proteome or the human microbiome are identified as neo-epitopes that are known or determined to engage regulatory T cells and/or other detrimental T cells (including T cells with potential host cross-reactivity and/or anergic T cells).

19. The method of numbered paragraph 18, wherein TCR contacts for a 9-mer identified shared neo-epitope that bind to a WIC class II molecule are at position 2, 3, 5, 7, and 8 of the identified shared neo-epitope as counted from the amino terminal, wherein the TCR contacts for a 9-mer identified shared neo-epitope that binds to a WIC class I molecule are at position 4, 5, 6, 7, and 8 of the identified shared neo-epitope as counted from the amino terminal, and wherein the TCR contacts for a 10-mer identified shared neo-epitope that bind to a WIC class I molecule are at position 4, 5, 6, 7, 8, and 9 of the identified shared neo-epitope as counted from the amino terminal.

20. The method of any one of numbered paragraphs 7-19, wherein assessing the identified shared neo-epitopes encoded by said mutations to identify neo-epitopes that are known or determined to engage regulatory T cells and/or other detrimental T cells (including T cells with potential host cross-reactivity and/or anergic T cells) in step (ii) comprises in silico testing.

21. The method of numbered paragraph 20, wherein said in silico testing comprises analyzing whether the identified shared neo-epitopes are predicted to engage regulatory T cells and/or other detrimental T cells (including T cells with potential host cross-reactivity and/or anergic T cells) using an algorithm that predicts cross-reactivity with regulatory T cells.

22. The method of numbered paragraph 21, wherein an identified shared neo-epitope is predicted to engage regulatory T cells and/or other detrimental T cells (including T cells with potential host cross-reactivity and/or anergic T cells) if the score for the shared neo-epitope is greater than a predetermined cutoff.

23. The method of any one of numbered paragraphs 7-20, wherein assessing the identified shared neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether the identified shared neo-epitopes engage regulatory T cells in vitro.

24. The method of numbered paragraph 23, wherein a shared neo-epitope is determined to engage regulatory T cells when said shared neo-epitope results in regulatory T cell activation, proliferation, and/or IL-10 or TGF-β production.

25. The method of any one of numbered paragraphs 20-22, further comprising determining whether the identified shared neo-epitopes engage regulatory T cells and/or other detrimental T cells (including T cells with potential host cross-reactivity and/or anergic T cells) in vitro.

26. The method of numbered paragraph 25, wherein a shared neo-epitope is determined to engage regulatory T cells when said shared neo-epitope results in regulatory T cell activation, proliferation, and/or IL-10 or TGF-β production.

27. The method of any one of numbered paragraphs 7-26, further comprising:

iii) designing at least one peptide or polypeptide, said peptide or polypeptide comprising at least one identified shared neo-epitope encoded by said shared neoplasia-specific mutations, provided said shared neo-epitope is not identified in step (ii) as being known or determined to engage regulatory T cells and/or other detrimental T cells (including T cells with potential host cross-reactivity and/or anergic T cells).

28. The method of numbered paragraph 27, further comprising:

iv) providing the at least one peptide or polypeptide designed in step (iii) or a nucleic acid encoding said peptides or polypeptides.

29. The method of numbered paragraph 28, further comprising:
v) providing a vaccine comprising the at least one peptide or polypeptide or nucleic acid provided in step (iv).

30. The method of any one of numbered paragraphs 1-29, wherein the one or more peptides or polypeptides comprising one or more identified shared neo-epitopes are administered with a pharmaceutically acceptable adjuvant and/or carrier.

31. The method of any one of numbered paragraphs 1-30, wherein one or more peptides or polypeptides comprising one or more identified shared neo-epitopes are administered in step (a) provided the one or more peptides or polypeptides comprising one or more identified shared neo-epitopes are:

encoded by a shared neoplasia-specific mutation that is detected in a neoplasia sample from the subject;

known or determined (e.g. predicted) to bind to a MHC protein of the subject; and/or

known or determined (e.g. predicted) to not bind to a MHC protein of the subject that could lead to a detrimental or suppressive immune response.

32. The method of any one of numbered paragraphs 1-31, wherein one or more peptides or polypeptides comprising one or more identified shared neo-epitopes are not administered in step (a) provided:

the one or more peptides or polypeptides are encoded by a shared neoplasia-specific mutation that is not detected in a neoplasia sample from the subject;

the one or more peptides or polypeptides are known or determined to not bind to a MEW protein of the subject; and/or

the one or more peptides or polypeptides are known or determined to bind to a MHC protein of the subject that could lead to a detrimental or suppressive immune response.

33. The method according to any one of numbered paragraphs 1-32, the method further comprising

detecting one or more tumor-specific mutations in a neoplasia sample from a subject and/or determining HLA allotypes present in the subject, and administering one or more peptides or polypeptides comprising one or more identified shared neo-epitopes, provided:

the one or more peptides or polypeptides are encoded by a shared neoplasia-specific mutation that is detected in a neoplasia sample from the subject;

the one or more peptides or polypeptides are known or determined (e.g. predicted) to bind to a MHC protein of the subject; and/or

the one or more peptides or polypeptides are known or determined (e.g. predicted) to not bind to a MEW protein of the subject that could lead to a detrimental or suppressive immune response.

34. The method of numbered paragraph 33, wherein one or more peptides or polypeptides comprising one or more identified shared neo-epitopes are not administered to a subject suspected of having or having a neoplasia provided:

the one or more peptides or polypeptides are encoded by a shared neoplasia-specific mutation that is not detected in a neoplasia sample from the subject;

the one or more peptides or polypeptides are known or determined to not bind to a MEW protein of the subject; and/or

the one or more peptides or polypeptides are known or determined to bind to a MHC protein of the subject that could lead to a detrimental or suppressive immune response.

35. The method of any one of numbered paragraphs 7-34, wherein the one or more peptides or polypeptides comprising one or more identified shared neo-epitopes are administered within 1 week of detecting one or more tumor-specific mutations in the neoplasia sample from a subject and/or determining HLA allotypes present in the subject.

36. The method of numbered paragraph 1, wherein the one or more of subject-specific peptides or polypeptides comprising one or more identified subject-specific neo-epitopes are identified by a method comprising:

i) identifying neoplasia-specific mutations in a neoplasia specimen of a subject;

ii) assessing the neoplasia-specific mutations identified in step (i) to identify known or determined neo-epitopes encoded by said mutations, wherein said neo-epitopes are known or determined (e.g. predicted) to bind to a MEW protein of the subject; and

iii) assessing the identified neo-epitopes encoded by said mutations from step (ii) to identify neo-epitopes that are known or determined to engage regulatory T cells and/or other detrimental T cells (including T cells with potential host cross-reactivity and/or anergic T cells), and excluding such identified neo-epitopes that are known or determined to engage regulatory T cells and/or other detrimental T cells (including T cells with potential host cross-reactivity and/or anergic T cells) from the subject-specific neo-epitopes.

37. The method of numbered paragraph 36, wherein identifying neoplasia-specific mutations in step (i) comprises identifying sequence differences between the full or partial genome, exome, and/or transcriptome of a neoplasia specimen from the subject diagnosed as having a neoplasia and a non-neoplasia specimen.

38. The method of any one of numbered paragraphs 36 or 37, wherein identifying neoplasia-specific mutations or identifying sequence differences comprises Next Generation Sequencing (NGS).

39. The method of any one of numbered paragraphs 36 or 37, wherein identifying neoplasia-specific mutations in step (i) comprises selecting from the neoplasia a plurality of nucleic acid sequences, each comprising mutations not present in a non-neoplasia sample.

40. The method of any one of numbered paragraphs 36 or 37, wherein identifying neoplasia-specific mutations or identifying sequence differences comprises sequencing genomic DNA and/or RNA of the neoplasia specimen.

41. The method of numbered paragraph 37, wherein said non-neoplasia specimen is derived from the subject diagnosed as having a neoplasia.

42. The method of any one of numbered paragraphs 36-41, wherein said neoplasia-specific mutations are neoplasia-specific somatic mutations.

43. The method of any one of numbered paragraphs 36-41, wherein said neoplasia-specific somatic mutations are single nucleotide variations (SNVs), in-frame insertions, in-frame deletions, out-of-frame insertions, and out-of-frame deletions.

44. The method of numbered paragraph 42 or 43, wherein said neoplasia-specific somatic mutations are mutations of proteins encoded in the neoplasia specimen of the subject diagnosed as having a neoplasia.

45. The method of any one of numbered paragraphs 36-44, wherein assessing the neoplasia-specific mutations in step (ii) to identify known or determined neo-epitopes encoded by said mutations comprises:

a) determining a binding score for a mutated peptide to one or more WIC molecules, wherein said mutated peptide is encoded by at least one of said neoplasia-specific mutations;

b) determining a binding score for a non-mutated peptide to the one or more WIC molecules, wherein the non-mutated peptide is identical to the mutated peptide except for the encoded at least one of said neoplasia-specific mutations;

c) determining the percentile rank of the binding scores of both the mutated peptide of step (a) and the non-mutated peptide of step (b) as compared to an expected distribution of binding scores for at least 10,000 randomly generated peptides using naturally observed amino acid frequencies;

d) determining the TCR facing amino acid residues of said mutated peptide and said non-mutated peptide; and

e) identifying the mutated peptide as a neo-epitope when:
1) the mutated peptide has a determined binding score in the top 5 percentile of the expected distribution and the non-mutated peptide has a determined binding score below the top 10 percentile of the expected distribution; or
2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide.

46. The method of numbered paragraph 45, wherein the mutated peptide and non-mutated peptide are both 9 amino acids in length or the mutated peptide and non-mutated peptide are both 10 amino acids in length.

47. The method of numbered paragraph 45, wherein the one or more MHC molecules are MHC class I molecules and/or MHC class II molecules.

48. The method of numbered paragraph 46, wherein the one or more MHC molecules are MHC class I molecules and/or MHC class II molecules.

49. The method of numbered paragraph 48, wherein the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal.

50. The method of any one of numbered paragraphs 36-49, wherein assessing the neoplasia-specific mutations in step (ii) to identify known or determined neo-epitopes encoded by said mutations comprises in silico testing.

51. The method of numbered paragraph 50, wherein said in silico testing to identify known or determined neo-epitopes encoded by said mutations in step (ii) comprises using an algorithm to screen protein sequences for putative T cell epitopes.

52. The method of any one of numbered paragraphs 36-51, wherein assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether said identified neo-epitopes encoded by said mutations share TCR contacts with proteins derived from either the human proteome or the human microbiome, wherein said identified neo-epitopes encoded by said mutations that are determined to share TCR contacts with proteins derived from either the human proteome or the human microbiome are identified as neo-epitopes that are known or determined to engage regulatory T cells and/or other detrimental T cells (including T cells with potential host cross-reactivity and/or anergic T cells).

53. The method of numbered paragraph 52, wherein TCR contacts for a 9-mer identified neo-epitope that bind to a WIC class II molecule are at position 2, 3, 5, 7, and 8 of the identified neo-epitope as counted from the amino terminal, wherein the TCR contacts for a 9-mer identified neo-epitope that binds to a WIC class I molecule are at position 4, 5, 6, 7, and 8 of the identified neo-epitope as counted from the amino terminal, and wherein the TCR contacts for a 10-mer identified neo-epitope that bind to a WIC class I molecule are at position 4, 5, 6, 7, 8, and 9 of the identified neo-epitope as counted from the amino terminal.

54. The method of any one of numbered paragraphs 36-53, wherein assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises in silico testing.

55. The method of numbered paragraph 54, wherein said in silico testing comprises analyzing whether the identified neo-epitopes are predicted to engage regulatory T cells and/or other detrimental T cells (including T cells with potential host cross-reactivity and/or anergic T cells) using an algorithm that predicts cross-reactivity with regulatory T cells.

56. The method of numbered paragraph 55, wherein an identified neo-epitope is predicted to engage regulatory T cells and/or other detrimental T cells (including T cells with potential host cross-reactivity and/or anergic T cells) if the score for the neo-epitope is greater than a predetermined cutoff.

57. The method of any one of numbered paragraphs 36-53, wherein assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether the identified neo-epitopes engage regulatory T cells and/or other detrimental T cells (including T cells with potential host cross-reactivity and/or anergic T cells) in vitro.

58. The method of numbered paragraph 57, wherein a neo-epitope is determined to engage regulatory T cells when said neo-epitope results in regulatory T cell activation, proliferation, and/or IL-10 or TGF-β production.

59. The method of any one of numbered paragraphs 54-56, further comprising determining whether the identified neo-epitopes engage regulatory T cells and/or other detrimental T cells (including T cells with potential host cross-reactivity and/or anergic T cells) in vitro.

60. The method of numbered paragraph 59, wherein a neo-epitope is determined to engage regulatory T cells when said neo-epitope results in regulatory T cell activation, proliferation, and/or IL-10 or TGF-β production.

61. The method of any one of numbered paragraphs 36-60, further comprising:

iv) designing at least one subject-specific peptide or polypeptide, said peptide or polypeptide comprising at least one identified neo-epitope encoded by said mutations, provided said neo-epitope is not identified in step (iii) as being known or determined to engage regulatory T cells and/or other detrimental T cells (including T cells with potential host cross-reactivity and/or anergic T cells).

62. The method of numbered paragraph 61, further comprising:

v) providing the at least one peptide or polypeptide designed in step (iv) or a nucleic acid encoding said peptides or polypeptides.

63. The method of numbered paragraph 62, further comprising:

vi) providing a vaccine comprising the at least one peptide or polypeptide or nucleic acid provided in step(v).

64. The method of any one of numbered paragraphs 1-63, wherein the one or more subject-specific peptides or polypeptides comprising one or more identified subject-specific neo-epitopes are administered with a pharmaceutically acceptable adjuvant and/or carrier.

65. The method of any one of numbered paragraphs 1-64, wherein the one or more subject-specific peptides or polypeptides comprising one or more identified subject-specific neo-epitopes are administered roughly three weeks are administering an effective amount of one or more peptides or polypeptides comp

66. The method of numbered paragraph 14, wherein TCR facing amino acid residues for a 9-mer identified neo-epitope that binds to a WIC class II molecule are at any combination of residues at positions 2, 3, 5, 7, and 8 as counted from the amino terminal, the TCR facing amino acid residues for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR facing amino acid residues for a 9-mer identified neo-epitope that binds to a MHC class I molecule are at any combination of residues at positions 1, 3, 4, 5, 6, 7, and 8 as counted from the amino terminal, the TCR facing amino acid residues for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR facing amino acid residues for a 10-mer identified neo-epitope that binds to a MHC class I molecule are at any combination of residues at positions 1, 3, 4, 5, 6, 7, 8, and 9 as counted from the amino terminal.

67. The method of numbered paragraph 18, wherein TCR contacts for a 9-mer identified neo-epitope that binds to a MHC class II molecule are at any combination of residues at positions 2, 3, 5, 7, and 8 as counted from the amino terminal, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR contacts for a 9-mer identified neo-epitope that binds to a MHC class I molecule are at any combination of residues at positions 1, 3, 4, 5, 6, 7, and 8 as counted from the amino terminal, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR contacts for a 10-mer identified neo-epitope that binds to a MHC class I molecule are at any combination of residues at positions 1, 3, 4, 5, 6, 7, 8, and 9 as counted from the amino terminal.

68. The method of numbered paragraph 48, wherein the TCR facing amino acid residues for a 9-mer identified neo-epitope that binds to a MHC class II molecule are at any combination of residues at positions 2, 3, 5, 7, and 8 as counted from the amino terminal, the TCR facing amino acid residues for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR facing amino acid residues for a 9-mer identified neo-epitope that binds to a MHC class I molecule are at any combination of residues at positions 1, 3, 4, 5, 6, 7, and 8 as counted from the amino terminal, the TCR facing amino acid residues for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR facing amino acid residues for a 10-mer identified neo-epitope that binds to a MHC class I molecule are at any combination of residues at positions 1, 3, 4, 5, 6, 7, 8, and 9 as counted from the amino terminal.

69. The method of numbered paragraph 52, wherein TCR contacts for a 9-mer identified neo-epitope that binds to a MHC class II molecule are at any combination of residues at positions 2, 3, 5, 7, and 8 as counted from the amino terminal, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR contacts for a 9-mer identified neo-epitope that binds to a MHC class I molecule are at any combination of residues at positions 1, 3, 4, 5, 6, 7, and 8 as counted from the amino terminal, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR contacts for a 10-mer identified neo-epitope that binds to a MHC class I molecule are at any combination of residues at positions 1, 3, 4, 5, 6, 7, 8, and 9 as counted from the amino terminal.

1. A method of treating a neoplasia in a subject, the method comprising (a) administering an effective amount of one or more peptides or polypeptides comprising one or more identified shared neo-epitopes; and subsequently (b) administering an effective amount of one or more subject-specific peptides or polypeptides comprising one or more identified subject-specific neo-epitopes, wherein the peptide or polypeptides comprising one or more identified shared neo-epitopes administered in step (a) and the subject-specific peptides or polypeptides comprising one or more identified subject-specific neo-epitopes administered in step (b) exclude neo-epitopes that are known or determined (e.g. predicted) to engage regulatory T cells and/or other detrimental T cells (including T cells with potential host cross-reactivity and/or anergic T cells).

2. The method of claim 1, wherein
I) the one or more peptides or polypeptides comprising one or more identified shared neo-epitopes comprise, consist of, or consist essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, provided:
the one or more peptides or polypeptides are encoded by a shared neoplasia-specific mutation that is detected in a neoplasia sample from the subject;
the one or more peptides or polypeptides are known or determined (e.g. predicted) to bind to a MHC protein of the subject; and/or
the one or more peptides or polypeptides are known or determined (e.g. predicted) to not bind to a MEW protein of the subject that could lead to a detrimental or suppressive immune response: or
II) the method further comprising
detecting one or more tumor-specific mutations in a neoplasia sample from a subject and/or determining HLA allotypes present in the subject, and administering one or more of peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, provided:
the one or more peptides or polypeptides are encoded by a shared neoplasia-specific mutation that is detected in a neoplasia sample from the subject;
the one or more peptides or polypeptides are known or determined (e.g. predicted) to bind to a MHC protein of the subject; and/or
the one or more peptides or polypeptides are known or determined (e.g. predicted) to not bind to a MEW protein of the subject that could lead to a detrimental or suppressive immune response; or
III) the one or more peptides or polypeptides comprising one or more identified shared neo-epitopes (including peptides or polypeptide comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C,) are administered within 1 week of detecting one or more tumor-specific mutations in the neoplasia sample from a subject (optionally, tumor tissue, optionally bladder cancer tumor tissue) and/or determining HLA allotypes present in the subject; or
IV) the one or more identified shared neo-epitopes are identified by a method comprising:
i) assessing identified shared neoplasia-specific mutations from a neoplasia specimen of a subject to identify known or determined shared neo-epitopes encoded by said shared neoplasia specific mutations; and
ii) assessing the identified shared neo-epitopes encoded by said mutations from step (i) to identify neo-epitopes that are known or determined to engage regulatory T cells, and excluding such identified neo-epitopes that are known or determined to engage regulatory T cells from the shared neo-epitopes; or
V) one or more peptides or polypeptides comprising one or more identified shared neo-epitopes are administered in step (a) provided the one or more peptides or polypeptides comprising one or more identified shared neo-epitopes are:
encoded by a shared neoplasia-specific mutation that is detected in a neoplasia sample from the subject;
known or determined (optionally predicted) to bind to a MEW protein of the subject and/or known or determined (optionally predicted) to not bind to a MHC protein of the subject that could lead to a detrimental or suppressive immune response; or
VI) one or more peptides or polypeptides comprising one or more identified shared neo-epitopes are not administered in step (a) provided:
the one or more peptides or polypeptides are encoded by a shared neoplasia-specific mutation that is not detected in a neoplasia sample from the subject;
the one or more peptides or polypeptides are known or determined to not bind to a MEW protein of the subject; and/or
the one or more peptides or polypeptides are known or determined to bind to a MHC protein of the subject that could lead to a detrimental or suppressive immune response; or
VII) method further comprising
detecting one or more tumor-specific mutations in a neoplasia sample from a subject and/or determining HLA allotypes present in the subject, and administering one or more peptides or polypeptides comprising one or more identified shared neo-epitopes, provided:
the one or more peptides or polypeptides are encoded by a shared neoplasia-specific mutation that is detected in a neoplasia sample from the subject;
the one or more peptides or polypeptides are known or determined (optionally predicted) to bind to a MEW protein of the subject; and/or
the one or more peptides or polypeptides are known or determined (optionally predicted) to not bind to a MEW protein of the subject that could lead to a detrimental or suppressive immune response; or
VIII) the one or more of subject-specific peptides or polypeptides comprising one or more identified subject-specific neo-epitopes are identified by a method comprising:
i) identifying neoplasia-specific mutations in a neoplasia specimen of a subject;
ii) assessing the neoplasia-specific mutations identified in step (i) to identify known or determined neo-epitopes encoded by said mutations, wherein said neo-epitopes are known or determined (optionally predicted) to bind to a MEW protein of the subject; and
iii) assessing the identified neo-epitopes encoded by said mutations from step (ii) to identify neo-epitopes that are known or determined to engage regulatory T cells and/or other detrimental T cells (including T cells with potential host cross-reactivity and/or anergic T cells), and excluding such identified neo-epitopes that are known or determined to engage regulatory T cells and/or other detrimental T cells (including T cells with potential host cross-reactivity and/or anergic T cells) from the subject-specific neo-epitopes; or
IX) the one or more of subject-specific peptides or polypeptides comprising one or more identified subject-specific neo-epitopes are identified by a method comprising:
i) identifying neoplasia-specific mutations in a neoplasia specimen of a subject;
ii) assessing the neoplasia-specific mutations identified in step (i) to identify known or determined neo-epitopes encoded by said mutations, wherein said neo-epitopes are known or determined (e.g. predicted) to bind to a MHC protein of the subject; and
iii) assessing the identified neo-epitopes encoded by said mutations from step (ii) to identify neo-epitopes that are known or determined to engage regulatory T cells and/or other detrimental T cells (including T cells with potential host cross-reactivity and/or anergic T cells), and excluding such identified neo-epitopes that are known or determined to engage regulatory T cells and/or other detrimental T cells (including T cells with potential host cross-reactivity and/or anergic T cells) from the subject-specific neo-epitopes; or
X) the one or more peptides or polypeptides comprising one or more identified shared neo-epitopes are administered with a pharmaceutically acceptable adjuvant and/or carrier.

3. The method of a claim 2 I), a wherein one or more peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, are not administered to a subject suspected of having or having a neoplasia provided:
the one or more peptides or polypeptides are encoded by a shared neoplasia-specific mutation that is not detected in a neoplasia sample from the subject;
the one or more peptides or polypeptides are known or determined to not bind to a MHC protein of the subject; and/or
the one or more peptides or polypeptides are known or determined to bind to a MHC protein of the subject that could lead to a detrimental or suppressive immune response.

4. (canceled)

5. The method of claim 4 II), wherein one or more peptides or polypeptides comprising, consisting of, or consisting essentially of one or more peptides or polypeptides from Table A (SEQ ID NOS: 105-163), Table B (SEQ ID NOS: 164-350), and/or C, and/or fragments and variants thereof, and optionally 1 to 12 additional amino acids distributed in any ratio on the N terminus and/or C-terminus of the polypeptide of Table A, Table B, and/or Table C, are not administered to a subject suspected of having or having a neoplasia provided:
the one or more peptides or polypeptides are encoded by a shared neoplasia-specific mutation that is not detected in a neoplasia sample from the subject;
the one or more peptides or polypeptides are known or determined to not bind to a MHC protein of the subject; and/or
the one or more peptides or polypeptides are known or determined to bind to a MHC protein of the subject that could lead to a detrimental or suppressive immune response.

6. (canceled)

7. (canceled)

8. The method of claim 1 IV), wherein
A) said shared neoplasia-specific mutations are shared neoplasia-specific somatic mutations; or
B) said shared neoplasia-specific somatic mutations are single nucleotide variations (SNVs), in-frame insertions, in-frame deletions, out-of-frame insertions, and out-of-frame deletions; or
C) said shared neoplasia-specific somatic mutations are mutations of proteins encoded in the neoplasia specimen of the subject diagnosed as having a neoplasia; or
D) assessing the shared neoplasia-specific mutations in step (i) to identify known or determined shared neo-epitopes encoded by said shared neoplasia-specific mutations comprises:
a) determining a binding score for a mutated peptide to one or more MHC molecules, wherein said mutated peptide is encoded by at least one of said shared neoplasia-specific mutations;
b) determining a binding score for a non-mutated peptide to the one or more MHC molecules, wherein the non-mutated peptide is identical to the mutated peptide except for the encoded at least one of said shared neoplasia-specific mutations;
c) determining the percentile rank of the binding scores of both the mutated peptide of step (a) and the non-mutated peptide of step (b) as compared to an expected distribution of binding scores for at least 10,000 randomly generated peptides using naturally observed amino acid frequencies;
d) determining the TCR facing amino acid residues of said mutated peptide and said non-mutated peptide; and
e) identifying the mutated peptide as a shared neo-epitope when:
1) the mutated peptide has a determined binding score in the top 5 percentile of the expected distribution and the non-mutated peptide has a determined binding score below the top 10 percentile of the expected distribution; or
2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide; or
E) assessing the shared neoplasia-specific mutations in step (i) to identify known or determined shared neo-epitopes encoded by said mutations comprises in silico testing; or
F) assessing the identified shared neo-epitopes encoded by said shared neoplasia-specific mutations to identify neo-epitopes that are known or determined to engage regulatory T cells and/or other detrimental T cells (including T cells with potential host cross-reactivity and/or anergic T cells) in step (ii) comprises determining whether said identified shared neo-epitopes encoded by said mutations share TCR contacts with proteins derived from either the human proteome or the human microbiome, wherein said identified shared neo-epitopes encoded by said mutations that are determined to share TCR contacts with proteins derived from either the human proteome or the human microbiome are identified as neo-epitopes that are known or determined to engage regulatory T cells and/or other detrimental T cells (including T cells with potential host cross-reactivity and/or anergic T cells); or
F) assessing the identified shared neo-epitopes encoded by said mutations to identify neo-epitopes that are known or determined to engage regulatory T cells and/or other detrimental T cells (including T cells with potential host cross-reactivity and/or anergic T cells) in step (ii) comprises in silico testing; or
G) assessing the identified shared neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether the identified shared neo-epitopes engage regulatory T cells in vitro; or
H) further comprising:
iii) designing at least one peptide or polypeptide, said peptide or polypeptide comprising at least one identified shared neo-epitope encoded by said shared neoplasia-specific mutations, provided said shared neo-epitope is not identified in step (ii) as being known or determined to engage regulatory T cells and/or other detrimental T cells (including T cells with potential host cross-reactivity and/or anergic T cells); or
I) the one or more peptides or polypeptides comprising one or more identified shared neo-epitopes are administered within 1 week of detecting one or more tumor-specific mutations in the neoplasia sample from a subject and/or determining HLA allotypes present in the subject.

9. (canceled)

10. (canceled)

11. (canceled)

12. The method of claim 8 D), wherein the mutated peptide and non-mutated peptide are both 9 amino acids in length or the mutated peptide and non-mutated peptide are both 10 amino acids in length; or the one or more MEW molecules are MEW class I molecules and/or MEW class II molecules.

13. (canceled)

14. The method of claim 12, wherein the one or more MEW molecules are MEW class I molecules and/or MHC class II molecules.

15. The method of claim 14, wherein the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MEW class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MEW class I molecule are at position 4, 5, 6, 7, 8, and 9 of the mutated and non-mutated peptide as counted from the amino terminal.

16. (canceled)

17. The method of claim 8 E), wherein said in silico testing to identify known or determined shared neo-epitopes encoded by said shared neoplasia-specific mutations in step (ii) comprises using an algorithm to screen protein sequences for putative T cell epitopes.

18. (canceled)

19. The method of claim 8 E), wherein TCR contacts for a 9-mer identified shared neo-epitope that bind to a MHC class II molecule are at position 2, 3, 5, 7, and 8 of the identified shared neo-epitope as counted from the amino terminal, wherein the TCR contacts for a 9-mer identified shared neo-epitope that binds to a MHC class I molecule are at position 4, 5, 6, 7, and 8 of the identified shared neo-epitope as counted from the amino terminal, and wherein the TCR contacts for a 10-mer identified shared neo-epitope that bind to a MHC class I molecule are at position 4, 5, 6, 7, 8, and 9 of the identified shared neo-epitope as counted from the amino terminal.

20. (canceled)

21. The method of claim 8 F), wherein said in silico testing comprises analyzing whether the identified shared neo-epitopes are predicted to engage regulatory T cells and/or other detrimental T cells (including T cells with potential host cross-reactivity and/or anergic T cells) using an algorithm that predicts cross-reactivity with regulatory T cells; or further comprising determining whether the identified shared neo-epitopes engage regulatory T cells and/or other detrimental T cells (including T cells with potential host cross-reactivity and/or anergic T cells) in vitro.

22. The method of claim 21, wherein an identified shared neo-epitope is predicted to engage regulatory T cells and/or other detrimental T cells (including T cells with potential host cross-reactivity and/or anergic T cells) if the score for the shared neo-epitope is greater than a predetermined cutoff; or wherein a shared neo-epitope is determined to engage regulatory T cells when said shared neo-epitope results in regulatory T cell activation, proliferation, and/or IL-10 or TGF-β production.

23. (canceled)

24. The method of claim 8 G), wherein a shared neo-epitope is determined to engage regulatory T cells when said shared neo-epitope results in regulatory T cell activation, proliferation, and/or IL-10 or TGF-β production.

25. (canceled)

26. (canceled)

27. (canceled)

28. The method of claim 8 H), further comprising:
iv) providing the at least one peptide or polypeptide designed in step (iii) or a nucleic acid encoding said peptides or polypeptides.

29. The method of claim 28, further comprising:
v) providing a vaccine comprising the at least one peptide or polypeptide or nucleic acid provided in step (iv).

30. (canceled)

31. (canceled)

32. (canceled)

33. (canceled)

34. The method of claim 2 VII), wherein one or more peptides or polypeptides comprising one or more identified shared neo-epitopes are not administered to a subject suspected of having or having a neoplasia provided:
the one or more peptides or polypeptides are encoded by a shared neoplasia-specific mutation that is not detected in a neoplasia sample from the subject;
the one or more peptides or polypeptides are known or determined to not bind to a MHC protein of the subject; and/or
the one or more peptides or polypeptides are known or determined to bind to a MHC protein of the subject that could lead to a detrimental or suppressive immune response.

35. (canceled)

36. (canceled)

37. The method of claim 2 VIII), wherein
identifying neoplasia-specific mutations in step (i) comprises identifying sequence differences between the full or partial genome, exome, and/or transcriptome of a neoplasia specimen from the subject diagnosed as having a neoplasia and a non-neoplasia specimen; or
identifying neoplasia-specific mutations or identifying sequence differences comprises Next Generation Sequencing (NGS); or
identifying neoplasia-specific mutations in step (i) comprises selecting from the neoplasia a plurality of nucleic acid sequences, each comprising mutations not present in a non-neoplasia sample; or
identifying neoplasia-specific mutations or identifying sequence differences comprises sequencing genomic DNA and/or RNA of the neoplasia specimen; or
said neoplasia-specific mutations are neoplasia-specific somatic mutations; or
said neoplasia-specific somatic mutations are single nucleotide variations (SNVs), in-frame insertions, in-frame deletions, out-of-frame insertions, and out-of-frame deletions;
or assessing the neoplasia-specific mutations in step (ii) to identify known or determined neo-epitopes encoded by said mutations comprises:
a) determining a binding score for a mutated peptide to one or more WIC molecules, wherein said mutated peptide is encoded by at least one of said neoplasia-specific mutations;
b) determining a binding score for a non-mutated peptide to the one or more WIC molecules, wherein the non-mutated peptide is identical to the mutated peptide except for the encoded at least one of said neoplasia-specific mutations;
c) determining the percentile rank of the binding scores of both the mutated peptide of step (a) and the non-mutated peptide of step (b) as compared to an expected distribution of binding scores for at least 10,000 randomly generated peptides using naturally observed amino acid frequencies;
d) determining the TCR facing amino acid residues of said mutated peptide and said non-mutated peptide; and
e) identifying the mutated peptide as a neo-epitope when:
1) the mutated peptide has a determined binding score in the top 5 percentile of the expected distribution and the non-mutated peptide has a determined binding score below the top 10 percentile of the expected distribution; or
2) the mutated peptide has a determined binding score in the top 5 percentile of the expected distribution, the non-mutated peptide has a determined binding score in the top 10 percentile of the expected distribution, and there is at least one mismatched TCR facing amino acid between the mutated peptide the non-mutated peptide; or
assessing the neoplasia-specific mutations in step (ii) to identify known or determined neo-epitopes encoded by said mutations comprises in silico testing; or
assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether said identified neo-epitopes encoded by said mutations share TCR contacts with proteins derived from either the human proteome or the human microbiome, wherein said identified neo-epitopes encoded by said mutations that are determined to share TCR contacts with proteins derived from either the human proteome or the human microbiome are identified as neo-epitopes that are known or determined to engage regulatory T cells and/or other detrimental T cells (including T cells with potential host cross-reactivity and/or anergic T cells); or
assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises in silco testing; or
assessing the identified neo-epitopes encoded by said mutations to identify neo-epitopes that are known or determined to engage regulatory T cells and/or other detrimental T cells (including T cells with potential host cross-reactivity and/or anergic T cells) in step (iii) comprises determining whether the identified neo-epitopes engage regulatory T cells and/or other detrimental T cells (including T cells with potential host cross-reactivity and/or anergic T cells) in vitro;
or;
further comprising:
iv) designing at least one subject-specific peptide or polypeptide, said peptide or polypeptide comprising at least one identified neo-epitope encoded by said mutations, provided said neo-epitope is not identified in step (iii) as being known or determined to engage regulatory T cells and/or other detrimental T cells (including T cells with potential host cross-reactivity and/or anergic T cells).

38. (canceled)

39. (canceled)

40. (canceled)

41. The method of claim 37, wherein said non-neoplasia specimen is derived from the subject diagnosed as having a neoplasia.

42. (canceled)

43. (canceled)

44. The method of claim 37, wherein said neoplasia-specific somatic mutations are mutations of proteins encoded in the neoplasia specimen of the subject diagnosed as having a neoplasia.

45. (canceled)

46. The method of claim 37, wherein the mutated peptide and non-mutated peptide are both 9 amino acids in length or the mutated peptide and non-mutated peptide are both 10 amino acids in length; or the one or more WIC molecules are WIC class I molecules and/or WIC class II molecules.

47. (canceled)

48. The method of claim 46, wherein the one or more MHC molecules are MHC class I molecules and/or MHC class II molecules.

49. The method of claim 48, wherein the TCR facing amino acid residues for a 9-mer mutated peptide and a 9-mer non-mutated peptide that bind to a MHC class II molecule are at position 2, 3, 5, 7, and 8 of the mutated and non-mutated peptide as counted from the amino terminal, wherein the TCR facing amino acid residues for a 9-mer mutated peptide and a 9-mer non-mutated peptide that bind to a MHC class I molecule are at position 4, 5, 6, 7, and 8 of the mutated and non-mutated peptide as counted from the amino terminal, and wherein the TCR facing amino acid residues for a 10-mer mutated peptide and 10-mer non-mutated peptide that bind to a MHC class I molecule are at position 4, 5, 6, 7, 8, and 9 of the mutated and non-mutated peptide as counted from the amino terminal.

50. (canceled)

51. The method of claim 37, wherein said in silico testing to identify known or determined neo-epitopes encoded by said mutations in step (ii) comprises using an algorithm to screen protein sequences for putative T cell epitopes.

52. (canceled)

53. The method of claim 37, wherein TCR contacts for a 9-mer identified neo-epitope that bind to a MHC class II molecule are at position 2, 3, 5, 7, and 8 of the identified neo-epitope as counted from the amino terminal, wherein the TCR contacts for a 9-mer identified neo-epitope that binds to a MHC class I molecule are at position 4, 5, 6, 7, and 8 of the identified neo-epitope as counted from the amino terminal, and wherein the TCR contacts for a 10-mer identified neo-epitope that bind to a MHC class I molecule are at position 4, 5, 6, 7, 8, and 9 of the identified neo-epitope as counted from the amino terminal.

54. (canceled)

55. The method of claim 37, wherein said in silico testing comprises analyzing whether the identified neo-epitopes are predicted to engage regulatory T cells and/or other detrimental T cells (including T cells with potential host cross-reactivity and/or anergic T cells) using an algorithm that predicts cross-reactivity with regulatory T cells.

56. The method of claim 55, wherein an identified neo-epitope is predicted to engage regulatory T cells and/or other detrimental T cells (including T cells with potential host cross-reactivity and/or anergic T cells) if the score for the neo-epitope is greater than a predetermined cutoff.

57. (canceled)

58. The method of claim 37, wherein a neo-epitope is determined to engage regulatory T cells when said neo-epitope results in regulatory T cell activation, proliferation, and/or IL-10 or TGF-β production.

59. The method of claim 54, further comprising determining whether the identified neo-epitopes engage regulatory T cells and/or other detrimental T cells (including T cells with potential host cross-reactivity and/or anergic T cells) in vitro.

60. The method of claim 59, wherein a neo-epitope is determined to engage regulatory T cells when said neo-epitope results in regulatory T cell activation, proliferation, and/or IL-10 or TGF-β production.

61. (canceled)

62. The method of claim 37, further comprising:
v) providing the at least one peptide or polypeptide designed in step (iv) or a nucleic acid encoding said peptides or polypeptides.

63. The method of claim 62, further comprising:
vi) providing a vaccine comprising the at least one peptide or polypeptide or nucleic acid provided in step(v).

64. (canceled)

65. (canceled)

66. The method of claim 14, wherein TCR facing amino acid residues for a 9-mer identified neo-epitope that binds to a MHC class II molecule are at any combination of residues at positions 2, 3, 5, 7, and 8 as counted from the amino terminal, the TCR facing amino acid residues for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR facing amino acid residues for a 9-mer identified neo-epitope that binds to a MHC class I molecule are at any combination of residues at positions 1, 3, 4, 5, 6, 7, and 8 as counted from the amino terminal, the TCR facing amino acid residues for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR facing amino acid residues for a 10-mer identified neo-epitope that binds to a MHC class I molecule are at any combination of residues at positions 1, 3, 4, 5, 6, 7, 8, and 9 as counted from the amino terminal.

67. The method of claim 8 F), wherein TCR contacts for a 9-mer identified neo-epitope that binds to a MHC class II molecule are at any combination of residues at positions 2, 3, 5, 7, and 8 as counted from the amino terminal, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR contacts for a 9-mer identified neo-epitope that binds to a MHC class I molecule are at any combination of residues at positions 1, 3, 4, 5, 6, 7, and 8 as counted from the amino terminal, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR contacts for a 10-mer identified neo-epitope that binds to a MHC class I molecule are at any combination of residues at positions 1, 3, 4, 5, 6, 7, 8, and 9 as counted from the amino terminal.

68. The method of claim 48, wherein the TCR facing amino acid residues for a 9-mer identified neo-epitope that binds to a MHC class II molecule are at any combination of residues at positions 2, 3, 5, 7, and 8 as counted from the amino terminal, the TCR facing amino acid residues for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR facing amino acid residues for a 9-mer identified neo-epitope that binds to a MHC class I molecule are at any combination of residues at positions 1, 3, 4, 5, 6, 7, and 8 as counted from the amino terminal, the TCR facing amino acid residues for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR facing amino acid residues for a 10-mer identified neo-epitope that binds to a MHC class I molecule are at any combination of residues at positions 1, 3, 4, 5, 6, 7, 8, and 9 as counted from the amino terminal.

69. The method of claim 37, wherein TCR contacts for a 9-mer identified neo-epitope that binds to a MHC class II molecule are at any combination of residues at positions 2, 3, 5, 7, and 8 as counted from the amino terminal, the TCR contacts for a 9-mer identified neo-epitope that binds to a MHC class I molecule are at positions 4, 5, 6, 7, and 8; 1, 4, 5, 6, 7 and 8; or 1, 3, 4, 5, 6, 7, and 8 of the identified neo-epitope as counted from the amino terminal, the TCR contacts for a 9-mer identified neo-epitope that binds to a MHC class I molecule are at any combination of residues at positions 1, 3, 4, 5, 6, 7, and 8 as counted from the amino terminal, the TCR contacts for a 10-mer identified neo-epitope that bind to a MHC class I molecule are at position 4, 5, 6, 7, 8, and 9; 1, 4, 5, 6, 7, 8, and 9; or 1, 3, 4, 5, 6, 7, 8, and 9 of the identified neo-epitope as counted from the amino terminal, the TCR contacts for a 10-mer identified neo-epitope that binds to a MHC class I molecule are at any combination of residues at positions 1, 3, 4, 5, 6, 7, 8, and 9 as counted from the amino terminal.


SEQUENCE LISTING (372 sequences)

SEQ ID NO: 1 - protein, 12 aa, Mus musculus
LQARLTSYETLK

SEQ ID NO: 2 - protein, 9 aa, Mus musculus
ARLTSYETL

SEQ ID NO: 3 - protein, 9 aa, Mus musculus
ARLTSYETL

SEQ ID NO: 4 - protein, 9 aa, Mus musculus
ARLTSYETL

SEQ ID NO: 5 - protein, 17 aa, Mus musculus
ETPEACRQARNYLEFSE

SEQ ID NO: 6 - protein, 9 aa, Mus musculus
EACRQARNY

SEQ ID NO: 7 - protein, 9 aa, Mus musculus
ACRQARNYL

SEQ ID NO: 8 - protein, 9 aa, Mus musculus
RQARNYLEF

SEQ ID NO: 9 - protein, 16 aa, Mus musculus
SSRVQYVVNPAVKIVF

SEQ ID NO: 10 - protein, 9 aa, Mus musculus
RVQYVVNPA

SEQ ID NO: 11 - protein, 10 aa, Mus musculus
QYVVNPAVKI

SEQ ID NO: 12 - protein, 15 aa, Mus musculus
TSKYYMRDVIAIESA

SEQ ID NO: 13 - protein, 9 aa, Mus musculus
SKYYMRDVI

SEQ ID NO: 14 - protein, 10 aa, Mus musculus
KYYMRDVIAI

SEQ ID NO: 15 - protein, 9 aa, Mus musculus
YYMRDVIAI

SEQ ID NO: 16 - protein, 9 aa, Mus musculus
YYMRDVIAI

SEQ ID NO: 17 - protein, 9 aa, Mus musculus
YYMRDVIAI

SEQ ID NO: 18 - protein, 14 aa, Mus musculus
PALLIKHMYNKLIS

SEQ ID NO: 19 - protein, 9 aa, Mus musculus
PALLIKHMY

SEQ ID NO: 20 - protein, 9 aa, Mus musculus
LLIKHMYNK

SEQ ID NO: 21 - protein, 9 aa, Mus musculus
LIKHMYNKL

SEQ ID NO: 22 - protein, 9 aa, Mus musculus
KHMYNKLIS

SEQ ID NO: 23 - protein, 17 aa, Mus musculus
LSWDTSKKNLTEYLSRF

SEQ ID NO: 24 - protein, 9 aa, Mus musculus
SWDTSKKNL

SEQ ID NO: 25 - protein, 10 aa, Mus musculus
TSKKNLTEYL

SEQ ID NO: 26 - protein, 9 aa, Mus musculus
SKKNLTEYL

SEQ ID NO: 27 - protein, 18 aa, Mus musculus
NNVHYLNDGDAIIYHTAS

SEQ ID NO: 28 - protein, 9 aa, Mus musculus
HYLNDGDAI

SEQ ID NO: 29 - protein, 10 aa, Mus musculus
HYLNDGDAII

SEQ ID NO: 30 - protein, 9 aa, Mus musculus
GDAIIYHTA

SEQ ID NO: 31 - protein, 14 aa, Mus musculus
PQPDLYRFVRRISI

SEQ ID NO: 32 - protein, 9 aa, Mus musculus
DLYRFVRRI

SEQ ID NO: 33 - protein, 10 aa, Mus musculus
LYRFVRRISI

SEQ ID NO: 34 - protein, 16 aa, Mus musculus
DTKCTKADCLFTHMSR

SEQ ID NO: 35 - protein, 9 aa, Mus musculus
TKCTKADCL

SEQ ID NO: 36 - protein, 9 aa, Mus musculus
KADCLFTHM

SEQ ID NO: 37 - protein, 14 aa, Mus musculus
EEDGIAVWTLLNGN

SEQ ID NO: 38 - protein, 9 aa, Mus musculus
DGIAVWTLL

SEQ ID NO: 39 - protein, 9 aa, Mus musculus
DGIAVWTLL

SEQ ID NO: 40 - protein, 13 aa, Mus musculus
ATVHSSMNKMLEE

SEQ ID NO: 41 - protein, 9 aa, Mus musculus
TVHSSMNKM

SEQ ID NO: 42 - protein, 9 aa, Mus musculus
VHSSMNKML

SEQ ID NO: 43 - protein, 16 aa, Mus musculus
ILGYRYWTGIGVLQSC

SEQ ID NO: 44 - protein, 10 aa, Mus musculus
GYRYWTGIGV

SEQ ID NO: 45 - protein, 9 aa, Mus musculus
RYWTGIGVL

SEQ ID NO: 46 - protein, 9 aa, Mus musculus
RYWTGIGVL

SEQ ID NO: 47 - protein, 10 aa, Mus musculus
RYWTGIGVLQ

SEQ ID NO: 48 - protein, 14 aa, Mus musculus
FCYVTYKGEIRGAS

SEQ ID NO: 49 - protein, 9 aa, Mus musculus
CYVTYKGEI

SEQ ID NO: 50 - protein, 12 aa, Mus musculus
VKICNMQKAAIL

SEQ ID NO: 51 - protein, 9 aa, Mus musculus
KICNMQKAA

SEQ ID NO: 52 - protein, 10 aa, Mus musculus
KICNMQKAAI

SEQ ID NO: 53 - protein, 16 aa, Mus musculus
RQFPVVEANWTMLHDE

SEQ ID NO: 54 - protein, 9 aa, Mus musculus
VVEANWTML

SEQ ID NO: 55 - protein, 13 aa, Mus musculus
MSYAEKSDEITKD

SEQ ID NO: 56 - protein, 9 aa, Mus musculus
SYAEKSDEI

SEQ ID NO: 57 - protein, 11 aa, Mus musculus
RIQEFVRSHFY

SEQ ID NO: 58 - protein, 9 aa, Mus musculus
RIQEFVRSH

SEQ ID NO: 59 - protein, 17 aa, Mus musculus
KVGLTVKTYEFLERNIP

SEQ ID NO: 60 - protein, 9 aa, Mus musculus
LTVKTYEFL

SEQ ID NO: 61 - protein, 9 aa, Mus musculus
KTYEFLERN

SEQ ID NO: 62 - protein, 10 aa, Mus musculus
KTYEFLERNI

SEQ ID NO: 63 - protein, 9 aa, Mus musculus
TYEFLERNI

SEQ ID NO: 64 - protein, 9 aa, Mus musculus
TYEFLERNI

SEQ ID NO: 65 - protein, 16 aa, Mus musculus
NSSTYWKGNPEMETLQ

SEQ ID NO: 66 - protein, 9 aa, Mus musculus
TYWKGNPEM

SEQ ID NO: 67 - protein, 10 aa, Mus musculus
TYWKGNPEME

SEQ ID NO: 68 - protein, 9 aa, Mus musculus
KGNPEMETL

SEQ ID NO: 69 - protein, 14 aa, Mus musculus
RKSYYMQKYFLDTV

SEQ ID NO: 70 - protein, 10 aa, Mus musculus
KSYYMQKYFL

SEQ ID NO: 71 - protein, 9 aa, Mus musculus
SYYMQKYFL

SEQ ID NO: 72 - protein, 10 aa, Mus musculus
SYYMQKYFLD

SEQ ID NO: 73 - protein, 10 aa, Mus musculus
YYMQKYFLDT

SEQ ID NO: 74 - protein, 14 aa, Mus musculus
PQKLQALQRALQSE

SEQ ID NO: 75 - protein, 9 aa, Mus musculus
KLQALQRAL

SEQ ID NO: 76 - protein, 25 aa, Mus musculus
PHSIKLSRRRSRSKNPFRKDKSPVR

SEQ ID NO: 77 - protein, 9 aa, Mus musculus
IKLSRRRSR

SEQ ID NO: 78 - protein, 9 aa, Mus musculus
KLSRRRSRS

SEQ ID NO: 79 - protein, 9 aa, Mus musculus
LSRRRSRSK

SEQ ID NO: 80 - protein, 9 aa, Mus musculus
SRRRSRSKN

SEQ ID NO: 81 - protein, 9 aa, Mus musculus
SKNPFRKDK

SEQ ID NO: 82 - protein, 14 aa, Mus musculus
HPWLKQRIDKVSTK

SEQ ID NO: 83 - protein, 9 aa, Mus musculus
WLKQRIDKV

SEQ ID NO: 84 - protein, 16 aa, Mus musculus
HSVEMLLKPRRSLDEN

SEQ ID NO: 85 - protein, 9 aa, Mus musculus
MLLKPRRSL

SEQ ID NO: 86 - protein, 14 aa, Mus musculus
KTPSSLENDSSNLD

SEQ ID NO: 87 - protein, 9 aa, Mus musculus
SSLENDSSN

SEQ ID NO: 88 - protein, 21 aa, Mus musculus
SRVQAAQAQHSKDSLYKRDND

SEQ ID NO: 89 - protein, 9 aa, Mus musculus
QAAQAQHSK

SEQ ID NO: 90 - protein, 9 aa, Mus musculus
HSKDSLYKR

SEQ ID NO: 91 - protein, 21 aa, Mus musculus
LEGVRLENEKSNVIAKKTGNK

SEQ ID NO: 92 - protein, 9 aa, Mus musculus
VRLENEKSN

SEQ ID NO: 93 - protein, 9 aa, Mus musculus
EKSNVIAKK

SEQ ID NO: 94 - protein, 9 aa, Mus musculus
KSNVIAKKT

SEQ ID NO: 95 - protein, 18 aa, Mus musculus
DTTLLASTKKAKKSVSKK

SEQ ID NO: 96 - protein, 9 aa, Mus musculus
LLASTKKAK

SEQ ID NO: 97 - protein, 9 aa, Mus musculus
STKKAKKSV

SEQ ID NO: 98 - protein, 20 aa, Mus musculus
LLVSSLKIWRKKRDRRCAIH

SEQ ID NO: 99 - protein, 9 aa, Mus musculus
SSLKIWRKK

SEQ ID NO: 100 - protein, 9 aa, Mus musculus
SLKIWRKKR

SEQ ID NO: 101 - protein, 9 aa, Mus musculus
LKIWRKKRD

SEQ ID NO: 102 - protein, 9 aa, Mus musculus
WRKKRDRRC

SEQ ID NO: 103 - protein, 15 aa, Mus musculus
PMNELDKVVKKHKES

SEQ ID NO: 104 - protein, 9 aa, Mus musculus
ELDKVVKKH

SEQ ID NO: 105 - protein, 12 aa, Homo sapiens
LDVLERCPHRPI

SEQ ID NO: 106 - protein, 15 aa, Homo sapiens
RDPLSEITKQEKDFL

SEQ ID NO: 107 - protein, 17 aa, Homo sapiens
RDPLSEITKQEKDFLWS

SEQ ID NO: 108 - protein, 18 aa, Homo sapiens
AISTRDPLSKITEQEKDF

SEQ ID NO: 109 - protein, 16 aa, Homo sapiens
YLSTDVGFCTLVCPLH

SEQ ID NO: 110 - protein, 20 aa, Homo sapiens
NYLSTDVGFCTLVCPLHNQE

SEQ ID NO: 111 - protein, 13 aa, Homo sapiens
SCMGGMNQRPILT

SEQ ID NO: 112 - protein, 14 aa, Homo sapiens
SCMGGMNQRPILTI

SEQ ID NO: 113 - protein, 18 aa, Homo sapiens
VRVCACPGTDRRTEEENL

SEQ ID NO: 114 - protein, 13 aa, Homo sapiens
LRLPALRFIGLKC

SEQ ID NO: 115 - protein, 18 aa, Homo sapiens
LRLPALRFIGLKCLEHLF

SEQ ID NO: 116 - protein, 12 aa, Homo sapiens
GRDRRTKEENLR

SEQ ID NO: 117 - protein, 11 aa, Homo sapiens
SAAALPASERG

SEQ ID NO: 118 - protein, 12 aa, Homo sapiens
MSAAALPASERG

SEQ ID NO: 119 - protein, 11 aa, Homo sapiens
FDEAHSIDNVC

SEQ ID NO: 120 - protein, 16 aa, Homo sapiens
KAVVVFDEAHSIDNVC

SEQ ID NO: 121 - protein, 14 aa, Homo sapiens
KNGRPDYIIVTHRP

SEQ ID NO: 122 - protein, 19 aa, Homo sapiens
KNGRPDYIIVTHRPLTDEE

SEQ ID NO: 123 - protein, 13 aa, Homo sapiens
ADEAGSVCAGILS

SEQ ID NO: 124 - protein, 14 aa, Homo sapiens
MGHVAAVGCVQYDG

SEQ ID NO: 125 - protein, 15 aa, Homo sapiens
GVREVFKTATRAALQ

SEQ ID NO: 126 - protein, 15 aa, Homo sapiens
LVVVGADGVGKSALT

SEQ ID NO: 127 - protein, 17 aa, Homo sapiens
YKLVVVGADGVGKSALT

SEQ ID NO: 128 - protein, 13 aa, Homo sapiens
KQMNDARHGGWTT

SEQ ID NO: 129 - protein, 17 aa, Homo sapiens
EYFMKQMNDARHGGWTT

SEQ ID NO: 130 - protein, 17 aa, Homo sapiens
LLGRNSFKVRVCACPGR

SEQ ID NO: 131 - protein, 20 aa, Homo sapiens
SGNLLGRNSFKVRVCACPGR

SEQ ID NO: 132 - protein, 16 aa, Homo sapiens
DGILYAFQKQTTEDSV

SEQ ID NO: 133 - protein, 18 aa, Homo sapiens
DGILYAFQKQTTEDSVML

SEQ ID NO: 134 - protein, 12 aa, Homo sapiens
MACGFCRAIACQ

SEQ ID NO: 135 - protein, 15 aa, Homo sapiens
MACGFCRAIACQLSR

SEQ ID NO: 136 - protein, 11 aa, Homo sapiens
KTLWKALAKKP

SEQ ID NO: 137 - protein, 13 aa, Homo sapiens
LVEADEACSVYAG

SEQ ID NO: 138 - protein, 15 aa, Homo sapiens
LVEADEACSVYAGIL

SEQ ID NO: 139 - protein, 15 aa, Homo sapiens
LGRNSFEVCVCACPG

SEQ ID NO: 140 - protein, 17 aa, Homo sapiens
RVCACPGKDRRTEEENL

SEQ ID NO: 141 - protein, 12 aa, Homo sapiens
NYMCNSFCMGGM

SEQ ID NO: 142 - protein, 15 aa, Homo sapiens
IHYNYMCNSFCMGGM

SEQ ID NO: 143 - protein, 17 aa, Homo sapiens
TGIVMDSGDRVTHTVPI

SEQ ID NO: 144 - protein, 16 aa, Homo sapiens
RQDIDLGVSGEVFDFS

SEQ ID NO: 145 - protein, 16 aa, Homo sapiens
RVVAQDPDAGKAGRLV

SEQ ID NO: 146 - protein, 14 aa, Homo sapiens
KKKKMPKLCFASRI

SEQ ID NO: 147 - protein, 15 aa, Homo sapiens
RDPLSEITQQEKDFL

SEQ ID NO: 148 - protein, 11 aa, Homo sapiens
GYVLVAINEFS

SEQ ID NO: 149 - protein, 14 aa, Homo sapiens
CTYSPALNNMFCQL

SEQ ID NO: 150 - protein, 12 aa, Homo sapiens
HMTEVVRHCPHH

SEQ ID NO: 151 - protein, 14 aa, Homo sapiens
HMTEVVRHCPHHER

SEQ ID NO: 152 - protein, 13 aa, Homo sapiens
SCMGGMNWRPILT

SEQ ID NO: 153 - protein, 17 aa, Homo sapiens
NSSCMGGMNWRPILTII

SEQ ID NO: 154 - protein, 13 aa, Homo sapiens
HQNSASDNAEASM

SEQ ID NO: 155 - protein, 11 aa, Homo sapiens
NSFVNDILERI

SEQ ID NO: 156 - protein, 10 aa, Homo sapiens
GPALLGLLSL

SEQ ID NO: 157 - protein, 14 aa, Homo sapiens
GPALLGLLSLEAEE

SEQ ID NO: 158 - protein, 15 aa, Homo sapiens
ITDQSLDKAQAKKHA

SEQ ID NO: 159 - protein, 17 aa, Homo sapiens
TITDQSLDKAQAKKHAL

SEQ ID NO: 160 - protein, 15 aa, Homo sapiens
VADIKVDGKQVELAL

SEQ ID NO: 161 - protein, 17 aa, Homo sapiens
NYVADIKVDGKQVELAL

SEQ ID NO: 162 - protein, 13 aa, Homo sapiens
HDCVPVLLEHPLY

SEQ ID NO: 163 - protein, 17 aa, Homo sapiens
HDCVPVLLEHPLYILYT

SEQ ID NO: 164 - protein, 10 aa, Homo sapiens
CVPVLLEHPL

SEQ ID NO: 165 - protein, 9 aa, Homo sapiens
VPVLLEHPL

SEQ ID NO: 166 - protein, 10 aa, Homo sapiens
VPVLLEHPLY

SEQ ID NO: 167 - protein, 9 aa, Homo sapiens
PVLLEHPLY

SEQ ID NO: 168 - protein, 9 aa, Homo sapiens
IVMDSGDRV

SEQ ID NO: 169 - protein, 10 aa, Homo sapiens
VMDSGDRVTH

SEQ ID NO: 170 - protein, 10 aa, Homo sapiens
MDSGDRVTHT

SEQ ID NO: 171 - protein, 9 aa, Homo sapiens
SGDRVTHTV

SEQ ID NO: 172 - protein, 9 aa, Homo sapiens
RPDYIIVTH

SEQ ID NO: 173 - protein, 10 aa, Homo sapiens
RPDYIIVTHR

SEQ ID NO: 174 - protein, 9 aa, Homo sapiens
DYIIVTHRP

SEQ ID NO: 175 - protein, 10 aa, Homo sapiens
SAAALPASER

SEQ ID NO: 176 - protein, 9 aa, Homo sapiens
AAALPASER

SEQ ID NO: 177 - protein, 10 aa, Homo sapiens
VVAQDPDAGK

SEQ ID NO: 178 - protein, 10 aa, Homo sapiens
DPDAGKAGRL

SEQ ID NO: 179 - protein, 9 aa, Homo sapiens
NSASDNAEA

SEQ ID NO: 180 - protein, 10 aa, Homo sapiens
SASDNAEASM

SEQ ID NO: 181 - protein, 9 aa, Homo sapiens
ASDNAEASM

SEQ ID NO: 182 - protein, 9 aa, Homo sapiens
NYLSTDVGF

SEQ ID NO: 183 - protein, 10 aa, Homo sapiens
NYLSTDVGFC

SEQ ID NO: 184 - protein, 9 aa, Homo sapiens
YLSTDVGFC

SEQ ID NO: 185 - protein, 10 aa, Homo sapiens
YLSTDVGFCT

SEQ ID NO: 186 - protein, 9 aa, Homo sapiens
LSTDVGFCT

SEQ ID NO: 187 - protein, 10 aa, Homo sapiens
LSTDVGFCTL

SEQ ID NO: 188 - protein, 9 aa, Homo sapiens
STDVGFCTL

SEQ ID NO: 189 - protein, 10 aa, Homo sapiens
STDVGFCTLV

SEQ ID NO: 190 - protein, 9 aa, Homo sapiens
TDVGFCTLV

SEQ ID NO: 191 - protein, 10 aa, Homo sapiens
TDVGFCTLVC

SEQ ID NO: 192 - protein, 10 aa, Homo sapiens
VGFCTLVCPL

SEQ ID NO: 193 - protein, 9 aa, Homo sapiens
FCTLVCPLH

SEQ ID NO: 194 - protein, 10 aa, Homo sapiens
GYVLVAINEF

SEQ ID NO: 195 - protein, 9 aa, Homo sapiens
YVLVAINEF

SEQ ID NO: 196 - protein, 9 aa, Homo sapiens
VVFDEAHSI

SEQ ID NO: 197 - protein, 9 aa, Homo sapiens
DEAHSIDNV

SEQ ID NO: 198 - protein, 10 aa, Homo sapiens
DEAHSIDNVC

SEQ ID NO: 199 - protein, 10 aa, Homo sapiens
HVAAVGCVQY

SEQ ID NO: 200 - protein, 9 aa, Homo sapiens
VAAVGCVQY

SEQ ID NO: 201 - protein, 10 aa, Homo sapiens
VAAVGCVQYD

SEQ ID NO: 202 - protein, 9 aa, Homo sapiens
VEADEACSV

SEQ ID NO: 203 - protein, 10 aa, Homo sapiens
VEADEACSVY

SEQ ID NO: 204 - protein, 9 aa, Homo sapiens
EADEACSVY

SEQ ID NO: 205 - protein, 10 aa, Homo sapiens
EADEACSVYA

SEQ ID NO: 206 - protein, 9 aa, Homo sapiens
ADEACSVYA

SEQ ID NO: 207 - protein, 10 aa, Homo sapiens
ADEACSVYAG

SEQ ID NO: 208 - protein, 9 aa, Homo sapiens
DEACSVYAG

SEQ ID NO: 209 - protein, 10 aa, Homo sapiens
VLERCPHRPI

SEQ ID NO: 210 - protein, 9 aa, Homo sapiens
LERCPHRPI

SEQ ID NO: 211 - protein, 9 aa, Homo sapiens
ADEAGSVCA

SEQ ID NO: 212 - protein, 9 aa, Homo sapiens
DEAGSVCAG

SEQ ID NO: 213 - protein, 10 aa, Homo sapiens
DEAGSVCAGI

SEQ ID NO: 214 - protein, 9 aa, Homo sapiens
EAGSVCAGI

SEQ ID NO: 215 - protein, 9 aa, Homo sapiens
NSFVNDILE

SEQ ID NO: 216 - protein, 10 aa, Homo sapiens
NSFVNDILER

SEQ ID NO: 217 - protein, 9 aa, Homo sapiens
SFVNDILER

SEQ ID NO: 218 - protein, 10 aa, Homo sapiens
SFVNDILERI

SEQ ID NO: 219 - protein, 9 aa, Homo sapiens
FVNDILERI

SEQ ID NO: 220 - protein, 10 aa, Homo sapiens
VVVGADGVGK

SEQ ID NO: 221 - protein, 9 aa, Homo sapiens
VVGADGVGK

SEQ ID NO: 222 - protein, 9 aa, Homo sapiens
VGADGVGKS

SEQ ID NO: 223 - protein, 9 aa, Homo sapiens
ADGVGKSAL

SEQ ID NO: 224 - protein, 9 aa, Homo sapiens
KTLWKALAK

SEQ ID NO: 225 - protein, 10 aa, Homo sapiens
KTLWKALAKK

SEQ ID NO: 226 - protein, 9 aa, Homo sapiens
TLWKALAKK

SEQ ID NO: 227 - protein, 10 aa, Homo sapiens
DIDLGVSGEV

SEQ ID NO: 228 - protein, 9 aa, Homo sapiens
IDLGVSGEV

SEQ ID NO: 229 - protein, 10 aa, Homo sapiens
IDLGVSGEVF

SEQ ID NO: 230 - protein, 10 aa, Homo sapiens
LGVSGEVFDF

SEQ ID NO: 231 - protein, 9 aa, Homo sapiens
GVSGEVFDF

SEQ ID NO: 232 - protein, 10 aa, Homo sapiens
GVSGEVFDFS

SEQ ID NO: 233 - protein, 9 aa, Homo sapiens
ITDQSLDKA

SEQ ID NO: 234 - protein, 10 aa, Homo sapiens
ITDQSLDKAQ

SEQ ID NO: 235 - protein, 9 aa, Homo sapiens
QSLDKAQAK

SEQ ID NO: 236 - protein, 10 aa, Homo sapiens
QSLDKAQAKK

SEQ ID NO: 237 - protein, 9 aa, Homo sapiens
SLDKAQAKK

SEQ ID NO: 238 - protein, 10 aa, Homo sapiens
SLDKAQAKKH

SEQ ID NO: 239 - protein, 9 aa, Homo sapiens
LDKAQAKKH

SEQ ID NO: 240 - protein, 10 aa, Homo sapiens
AISTRDPLSK

SEQ ID NO: 241 - protein, 9 aa, Homo sapiens
ISTRDPLSK

SEQ ID NO: 242 - protein, 10 aa, Homo sapiens
ISTRDPLSKI

SEQ ID NO: 243 - protein, 9 aa, Homo sapiens
STRDPLSKI

SEQ ID NO: 244 - protein, 10 aa, Homo sapiens
STRDPLSKIT

SEQ ID NO: 245 - protein, 10 aa, Homo sapiens
PLSKITEQEK

SEQ ID NO: 246 - protein, 9 aa, Homo sapiens
LSKITEQEK

SEQ ID NO: 247 - protein, 9 aa, Homo sapiens
KITEQEKDF

SEQ ID NO: 248 - protein, 10 aa, Homo sapiens
DPLSEITKQE

SEQ ID NO: 249 - protein, 10 aa, Homo sapiens
PLSEITKQEK

SEQ ID NO: 250 - protein, 9 aa, Homo sapiens
LSEITKQEK

SEQ ID NO: 251 - protein, 10 aa, Homo sapiens
LSEITKQEKD

SEQ ID NO: 252 - protein, 9 aa, Homo sapiens
SEITKQEKD

SEQ ID NO: 253 - protein, 10 aa, Homo sapiens
SEITKQEKDF

SEQ ID NO: 254 - protein, 9 aa, Homo sapiens
ITKQEKDFL

SEQ ID NO: 255 - protein, 10 aa, Homo sapiens
DPLSEITQQE

SEQ ID NO: 256 - protein, 10 aa, Homo sapiens
PLSEITQQEK

SEQ ID NO: 257 - protein, 9 aa, Homo sapiens
LSEITQQEK

SEQ ID NO: 258 - protein, 10 aa, Homo sapiens
LSEITQQEKD

SEQ ID NO: 259 - protein, 9 aa, Homo sapiens
SEITQQEKD

SEQ ID NO: 260 - protein, 10 aa, Homo sapiens
SEITQQEKDF

SEQ ID NO: 261 - protein, 9 aa, Homo sapiens
ITQQEKDFL

SEQ ID NO: 262 - protein, 10 aa, Homo sapiens
QMNDARHGGW

SEQ ID NO: 263 - protein, 9 aa, Homo sapiens
MNDARHGGW

SEQ ID NO: 264 - protein, 10 aa, Homo sapiens
GPALLGLLSL

SEQ ID NO: 265 - protein, 9 aa, Homo sapiens
MACGFCRAI

SEQ ID NO: 266 - protein, 10 aa, Homo sapiens
MACGFCRAIA

SEQ ID NO: 267 - protein, 9 aa, Homo sapiens
FCRAIACQL

SEQ ID NO: 268 - protein, 10 aa, Homo sapiens
VADIKVDGKQ

SEQ ID NO: 269 - protein, 9 aa, Homo sapiens
IKVDGKQVE

SEQ ID NO: 270 - protein, 9 aa, Homo sapiens
KVDGKQVEL

SEQ ID NO: 271 - protein, 9 aa, Homo sapiens
GVREVFKTA

SEQ ID NO: 272 - protein, 9 aa, Homo sapiens
VREVFKTAT

SEQ ID NO: 273 - protein, 10 aa, Homo sapiens
VREVFKTATR

SEQ ID NO: 274 - protein, 9 aa, Homo sapiens
REVFKTATR

SEQ ID NO: 275 - protein, 10 aa, Homo sapiens
REVFKTATRA

SEQ ID NO: 276 - protein, 9 aa, Homo sapiens
VFKTATRAA

SEQ ID NO: 277 - protein, 10 aa, Homo sapiens
VFKTATRAAL

SEQ ID NO: 278 - protein, 9 aa, Homo sapiens
FKTATRAAL

SEQ ID NO: 279 - protein, 9 aa, Homo sapiens
LRLPALRFI

SEQ ID NO: 280 - protein, 10 aa, Homo sapiens
LRLPALRFIG

SEQ ID NO: 281 - protein, 10 aa, Homo sapiens
RLPALRFIGL

SEQ ID NO: 282 - protein, 9 aa, Homo sapiens
LPALRFIGL

SEQ ID NO: 283 - protein, 9 aa, Homo sapiens
DGILYAFQK

SEQ ID NO: 284 - protein, 9 aa, Homo sapiens
ILYAFQKQT

SEQ ID NO: 285 - protein, 10 aa, Homo sapiens
ILYAFQKQTT

SEQ ID NO: 286 - protein, 9 aa, Homo sapiens
YAFQKQTTE

SEQ ID NO: 287 - protein, 10 aa, Homo sapiens
YAFQKQTTED

SEQ ID NO: 288 - protein, 9 aa, Homo sapiens
FQKQTTEDS

SEQ ID NO: 289 - protein, 10 aa, Homo sapiens
KKKKMPKLCF

SEQ ID NO: 290 - protein, 9 aa, Homo sapiens
KKKMPKLCF

SEQ ID NO: 291 - protein, 9 aa, Homo sapiens
KKMPKLCFA

SEQ ID NO: 292 - protein, 9 aa, Homo sapiens
KMPKLCFAS

SEQ ID NO: 293 - protein, 10 aa, Homo sapiens
KMPKLCFASR

SEQ ID NO: 294 - protein, 9 aa, Homo sapiens
MPKLCFASR

SEQ ID NO: 295 - protein, 10 aa, Homo sapiens
MPKLCFASRI

SEQ ID NO: 296 - protein, 9 aa, Homo sapiens
LLGRNSFKV

SEQ ID NO: 297 - protein, 9 aa, Homo sapiens
GRNSFKVRV

SEQ ID NO: 298 - protein, 10 aa, Homo sapiens
GRNSFKVRVC

SEQ ID NO: 299 - protein, 10 aa, Homo sapiens
RNSFKVRVCA

SEQ ID NO: 300 - protein, 9 aa, Homo sapiens
NSFKVRVCA

SEQ ID NO: 301 - protein, 9 aa, Homo sapiens
SFKVRVCAC

SEQ ID NO: 302 - protein, 10 aa, Homo sapiens
KVRVCACPGR

SEQ ID NO: 303 - protein, 9 aa, Homo sapiens
GRDRRTKEE

SEQ ID NO: 304 - protein, 10 aa, Homo sapiens
GRDRRTKEEN

SEQ ID NO: 305 - protein, 10 aa, Homo sapiens
RDRRTKEENL

SEQ ID NO: 306 - protein, 10 aa, Homo sapiens
DRRTKEENLR

SEQ ID NO: 307 - protein, 9 aa, Homo sapiens
RRTKEENLR

SEQ ID NO: 308 - protein, 10 aa, Homo sapiens
CTYSPALNNM

SEQ ID NO: 309 - protein, 9 aa, Homo sapiens
TYSPALNNM

SEQ ID NO: 310 - protein, 10 aa, Homo sapiens
TYSPALNNMF

SEQ ID NO: 311 - protein, 9 aa, Homo sapiens
YSPALNNMF

SEQ ID NO: 312 - protein, 10 aa, Homo sapiens
YSPALNNMFC

SEQ ID NO: 313 - protein, 9 aa, Homo sapiens
SPALNNMFC

SEQ ID NO: 314 - protein, 10 aa, Homo sapiens
PALNNMFCQL

SEQ ID NO: 315 - protein, 9 aa, Homo sapiens
ALNNMFCQL

SEQ ID NO: 316 - protein, 9 aa, Homo sapiens
MTEVVRHCP

SEQ ID NO: 317 - protein, 9 aa, Homo sapiens
TEVVRHCPH

SEQ ID NO: 318 - protein, 10 aa, Homo sapiens
TEVVRHCPHH

SEQ ID NO: 319 - protein, 9 aa, Homo sapiens
SCMGGMNQR

SEQ ID NO: 320 - protein, 10 aa, Homo sapiens
CMGGMNQRPI

SEQ ID NO: 321 - protein, 9 aa, Homo sapiens
MGGMNQRPI

SEQ ID NO: 322 - protein, 10 aa, Homo sapiens
MGGMNQRPIL

SEQ ID NO: 323 - protein, 9 aa, Homo sapiens
SCMGGMNWR

SEQ ID NO: 324 - protein, 9 aa, Homo sapiens
CMGGMNWRP

SEQ ID NO: 325 - protein, 10 aa, Homo sapiens
CMGGMNWRPI

SEQ ID NO: 326 - protein, 9 aa, Homo sapiens
MGGMNWRPI

SEQ ID NO: 327 - protein, 10 aa, Homo sapiens
MGGMNWRPIL

SEQ ID NO: 328 - protein, 9 aa, Homo sapiens
GMNWRPILT

SEQ ID NO: 329 - protein, 9 aa, Homo sapiens
GRNSFEVCV

SEQ ID NO: 330 - protein, 10 aa, Homo sapiens
GRNSFEVCVC

SEQ ID NO: 331 - protein, 10 aa, Homo sapiens
RNSFEVCVCA

SEQ ID NO: 332 - protein, 9 aa, Homo sapiens
NSFEVCVCA

SEQ ID NO: 333 - protein, 9 aa, Homo sapiens
FEVCVCACP

SEQ ID NO: 334 - protein, 10 aa, Homo sapiens
FEVCVCACPG

SEQ ID NO: 335 - protein, 10 aa, Homo sapiens
RVCACPGKDR

SEQ ID NO: 336 - protein, 9 aa, Homo sapiens
CACPGKDRR

SEQ ID NO: 337 - protein, 9 aa, Homo sapiens
CPGKDRRTE

SEQ ID NO: 338 - protein, 10 aa, Homo sapiens
CPGKDRRTEE

SEQ ID NO: 339 - protein, 9 aa, Homo sapiens
VRVCACPGT

SEQ ID NO: 340 - protein, 10 aa, Homo sapiens
RVCACPGTDR

SEQ ID NO: 341 - protein, 9 aa, Homo sapiens
CACPGTDRR

SEQ ID NO: 342 - protein, 9 aa, Homo sapiens
CPGTDRRTE

SEQ ID NO: 343 - protein, 10 aa, Homo sapiens
CPGTDRRTEE

SEQ ID NO: 344 - protein, 9 aa, Homo sapiens
GTDRRTEEE

SEQ ID NO: 345 - protein, 10 aa, Homo sapiens
GTDRRTEEEN

SEQ ID NO: 346 - protein, 9 aa, Homo sapiens
NYMCNSFCM

SEQ ID NO: 347 - protein, 10 aa, Homo sapiens
NYMCNSFCMG

SEQ ID NO: 348 - protein, 9 aa, Homo sapiens
YMCNSFCMG

SEQ ID NO: 349 - protein, 10 aa, Homo sapiens
YMCNSFCMGG

SEQ ID NO: 350 - protein, 10 aa, Homo sapiens
MCNSFCMGGM

SEQ ID NO: 351 - protein, 15 aa, Mus musculus
VLDFGCGSGLLGITA

SEQ ID NO: 352 - protein, 9 aa, Mus musculus
FGCGSGLLG

SEQ ID NO: 353 - protein, 9 aa, Mus musculus
CGSGLLGIT

SEQ ID NO: 354 - protein, 9 aa, Mus musculus
GSGLLGITA

SEQ ID NO: 355 - protein, 15 aa, Mus musculus
NMDTRPSSDSSLQHA

SEQ ID NO: 356 - protein, 9 aa, Mus musculus
TRPSSDSSL

SEQ ID NO: 357 - protein, 15 aa, Mus musculus
RRDVARSSLRLIIDC

SEQ ID NO: 358 - protein, 9 aa, Mus musculus
VARSSLRLI

SEQ ID NO: 359 - protein, 15 aa, Mus musculus
MSNNFVEIKESVFKK

SEQ ID NO: 360 - protein, 9 aa, Mus musculus
NFVEIKESV

SEQ ID NO: 361 - protein, 15 aa, Mus musculus
PSRAIPLGIIIAVAY

SEQ ID NO: 362 - protein, 9 aa, Mus musculus
RAIPLGIII

SEQ ID NO: 363 - protein, 9 aa, Mus musculus
AIPLGIIIA

SEQ ID NO: 364 - protein, 15 aa, Mus musculus
VHSSMNKMLEEGQEY

SEQ ID NO: 365 - protein, 9 aa, Mus musculus
SMNKMLEEG

SEQ ID NO: 366 - protein, 14 aa, Mus musculus
QHFGVEKTVSSLLN

SEQ ID NO: 367 - protein, 9 aa, Mus musculus
GVEKTVSSL

SEQ ID NO: 368 - protein, 15 aa, Mus musculus
IKCLMENLKDEISQA

SEQ ID NO: 369 - protein, 9 aa, Mus musculus
LMENLKDEI

SEQ ID NO: 370 - protein, 16 aa, Mus musculus
QQRKFKASRASILSEM

SEQ ID NO: 371 - protein, 9 aa, Mus musculus
KFKASRASI

SEQ ID NO: 372 - protein, 9 aa, Mus musculus
FKASRASIL